SEQUENCE LISTING (2 sequences)

SEQ ID NO: 1 - protein, 213 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 452 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0364190 A1
(43) Pub. Date: Nov. 19, 2026

(54) ANTIBODY DRUG CONJUGATE PLATFORMS

(71) Applicant: BEONE MEDICINES I GMBH, Basel (CH)

(72) Inventors: Charng-Sheng Tsai, Beijing (CN); Mei-Hsuan Tsai, Shanghai (CN); Zewei Wang, Shanghai (CN); Wei Luo, Shanghai (CN); Maomao He, Shanghai (CN); Bing Li, Shanghai (CN); Xiaodong Wei, Shanghai (CN)

(73) Assignee: BEONE MEDICINES I GMBH, Basel (CH)

(21) Appl. No.: 19/701,841

(22) Filed: Jun. 9, 2026

Related U.S. Application Data
(63) Continuation of application No. PCT/CN2024/138655, filed on Dec. 12, 2024.

(30) Foreign Application Priority Data
Dec. 13, 2023 (WO) PCT/CN2023/138423

(51) Int. Cl. A61K 47/68; C07K 16/28

(52) U.S. Cl. CPC A61K 47/68037; A61K 47/6849; A61K 47/6889; C07K 16/2827

(57) ABSTRACT

The present disclosure provides antibody drug conjugate platforms and antibody drug conjugates comprising antibodies or antigen-binding fragments thereof and linker-payloads derived from the platforms, as well as pharmaceutical compositions comprising the antibody drug conjugates.

### CROSS REFERENCE TO RELATED APPLICATION

This application is a continuation of International Application No. PCT/CN2024/138655, filed Dec. 12, 2024, which claims priority to International Application No. PCT/CN2023/138423, filed Dec. 13, 2023, the disclosure of each of which is hereby incorporated by reference in its entirety.

### SEQUENCE LISTING

This application contains a Sequence Listing, which has been submitted electronically in XML format. The XML file is entitled “01368-0059-00PCT.xml,” was created on Nov. 4, 2024, and is 3,422 bytes in size. The Sequence Listing is incorporated herein by reference in its entirety.

### FIELD

Disclosed herein are antibody drug conjugate platforms and antibody drug conjugates (ADCs) comprising the platforms and an antibody, or antigen-binding fragment thereof, as well as uses of the ADC platforms and ADCs.

### BACKGROUND

Chemotherapy remains one of the most important anti-cancer therapies along with surgery, radiotherapy, and targeted therapy. Although there are many types of highly efficient cytotoxins, the difference between tumor cells and normal cells is very small, which limits the broad clinical application of these anti-cancer compounds due to toxic side effects. Antibody drugs have become the frontline drugs for anti-tumor therapy because of the specificity of an anti-tumor monoclonal antibody for a tumor cell surface antigen. However, when the antibody is used alone as the anti-tumor drug, the efficacy is often unsatisfactory.

Antibody drug conjugates (ADCs) enable the combination of an antibody or an antibody fragment with a biologically active cytotoxin through a chemically stable linker, taking full advantage of the specificity of antibody binding to the surface antigens of normal cells or tumor cells and the high efficiency of the cytotoxin, while avoiding low efficacy of the antibody and toxic side effects of the cytotoxin. That means, compared with conventional chemotherapy drugs, ADCs can specifically bind to tumor cells and reduce the effect on normal cells.

Accordingly, there is a need for ADCs with efficacy in cancer.

### SUMMARY

Provided herein are antibody drug conjugate platforms and antibody drug conjugates (ADCs). Also provided are uses of the ADC platforms to prepare ADCs, e.g., for use in therapy.

In some embodiments, provided herein is a compound of Formula (I):

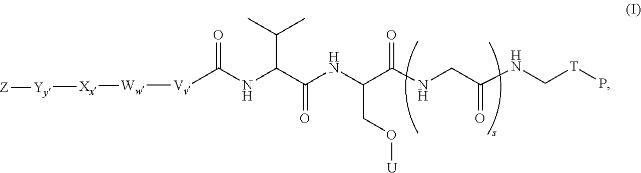

or a pharmaceutically acceptable salt, tautomer, solvate, or stereoisomer thereof, wherein values for the variables (e.g., Z, Y, X, W, V, U, T, P, s, v′, w′, x′, y′) are as described herein.

In other embodiments, provided herein is an antibody drug conjugate of Formula (I):

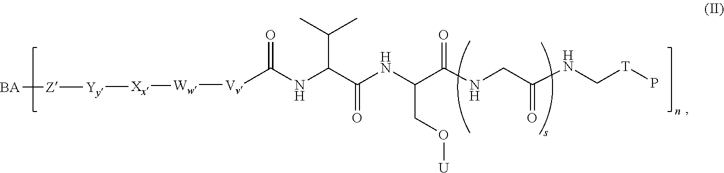

or a pharmaceutically acceptable salt, tautomer, solvate, or stereoisomer thereof, wherein values for the variables (e.g., BA, Z′, Y, X, W, V, U, T, P, n, s, v′, w′, x′, y′) are as described herein.

In other embodiments, provided herein is a pharmaceutical composition comprising an ADC described herein, e.g., a compound of Formula (II), or a pharmaceutically acceptable salt, tautomer, solvate, or stereoisomer thereof, and a pharmaceutically acceptable excipient.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate embodiments and together with the description serve to explain the principles described herein.

### BRIEF DESCRIPTION OF THE DRAWINGS

FIG. 1 shows payload cellular killing on A375 cell line.

FIG. 2 shows payload cellular killing on Calu-6 cell line.

FIG. 3 shows payload cellular killing on A375 cell line.

FIG. 4 shows payload cellular killing on Calu-6 cell line.

FIG. 5 shows ADC cellular killing on NCI-H358 cell line.

FIG. 6 shows ADC cellular killing on CAPAN-1 cell line.

FIG. 7 shows ADC cellular killing on MDA-MB-453 cell line.

FIG. 8 shows ADC cellular killing on NCI-H358 cell line.

FIG. 9 shows ADC cellular killing on CAPAN-1 cell line.

FIG. 10 shows ADC cellular killing on MDA-MB-453 cell line.

FIG. 11 shows ADC cellular killing on NCI-358 cell line.

FIG. 12 shows ADC cellular killing on CAPAN-1 cell line.

FIG. 13 shows ADC cellular killing on MDA-MB-453 cell line.

FIG. 14 shows ADC cellular killing on NCI-H358 cell line.

FIG. 15 shows ADC cellular killing on CAPAN-1 cell line.

FIG. 16 shows ADC cellular killing on MDA-MB-453 cell line.

FIG. 17 shows ADC cellular killing on NCI-H358 cell line.

FIG. 18 shows ADC cellular killing on CAPAN-1 cell line.

FIG. 19 shows ADC cellular killing on MDA-MB-453 cell line.

FIG. 20 shows ADC cellular killing on NCI-H358 cell line.

FIG. 21 shows ADC cellular killing on CAPAN-1 cell line.

FIG. 22 shows ADC cellular killing on MDA-MB-453 cell line.

FIG. 23 shows ADC cellular killing on NCI-H358 cell line.

FIG. 24 shows ADC cellular killing on CAPAN-1 cell line.

FIG. 25 shows ADC cellular killing on MDA-MB-453 cell line.

FIG. 26 shows ADC bystander killing assay.

FIG. 27 shows ADC bystander killing assay.

FIG. 28 shows ADC bystander killing assay.

FIG. 29 shows ADC bystander killing assay.

FIG. 30 shows ADC bystander killing assay.

FIG. 31 shows ADC bystander killing assay.

FIG. 32 shows ADC bystander killing assay.

FIG. 33 shows ADC bystander killing assay.

FIG. 34 shows ADC bystander killing assay.

FIG. 35 shows ADC bystander killing assay.

FIG. 36 shows ADC bystander killing assay.

FIG. 37 shows ADC bystander killing assay.

FIG. 38 shows ADC cellular killing on NCI-H358 cell line.

FIG. 39 shows ADC cellular killing on CAPAN-1 cell line.

FIG. 40 shows ADC cellular killing on MDA-MB-453 cell line.

FIG. 41 shows ADC bystander killing assay.

FIG. 42 shows ADC bystander killing assay.

### DESCRIPTION OF THE EMBODIMENTS

### Definitions

Unless specifically defined below or elsewhere in this document, all technical and scientific terms used herein have the meaning commonly understood by one of ordinary skill in the art.

As used herein, including in the appended claims, the singular forms of words such as “a,” “an,” and “the” include their corresponding plural forms unless the context clearly indicates otherwise.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within one or more than one standard deviation per the practice in the art. “About” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” should be assumed to be within an acceptable error range for that particular value or composition.

The term “or” is used to mean, and is used interchangeably with, the term “and/or” unless the context clearly indicates otherwise.

The terms “administration” and “administering,” as used herein, when applied to an animal, human, subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell.

The term “subject” herein includes any organism, preferably an animal, more preferably a mammal (e.g., rat, mouse, dog, cat, rabbit, primate) and most preferably a human (e.g., a patient comprising, or at risk of having, a disorder described herein).

“Treating” any disease or disorder refers in one aspect to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discemible by the patient. In yet another aspect, “treat,” “treating,” or “treatment” refers to modulating the disease or disorder, either physically (e.g., stabilization of a discernible symptom), physiologically (e.g., stabilization of a physical parameter), or both.

The terms “improve,” “increase,” “inhibit,” and “reduce” indicate values that are relative to a baseline or other reference measurement. In some embodiments, an appropriate reference measurement may comprise a measurement in a certain system (e.g., in a single individual) under otherwise comparable conditions absent presence of (e.g., prior to and/or after) an agent or treatment, or in presence of an appropriate comparable reference agent. In some embodiments, an appropriate reference measurement may comprise a measurement in a comparable system known or expected to respond in a comparable way, in presence of the relevant agent or treatment.

In some aspects, the present disclosure provides compositions, e.g., pharmaceutically acceptable compositions, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal, or epidermal administration (e.g., by injection or infusion).

The term “therapeutically effective amount” or “effective amount” as herein used refers to the amount of an agent that, when administered to a subject for treating a disease, or at least one of the clinical symptoms of a disease or disorder, is sufficient to effect such treatment for the disease, disorder, or symptom. The “therapeutically effective amount” can vary with the agent, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination components.

The term “toxin” or “payload” or “cytotoxic agent” is used herein to reference a molecule that inhibits or reduces the expression of molecules in cells, inhibits or reduces the function of cells, induces apoptosis of cells, and/or causes death of cells. The term includes radioactive isotopes, chemotherapeutic agents, and toxins such as small molecule toxins or enzymatically active toxins of bacterial, fungal, plant, or animal origin, including fragments and/or variants thereof. Examples of cytotoxic agents include, but are not limited to, auristatins (e.g., auristatin E, auristatin F, MMAE, and MMAF), auromycins, maytansinoids, pyrrolobenzodiazepine (PBD), ricin, ricin A-chain, combrestatin, duocarmycins, dolastatins, doxorubicin, daunorubicin, taxols, cisplatin, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, and calicheamicin, as well as radioisotopes such as At211, I131, I125, Y90, Re186, Re188, Sm153, Bi212 or 213, P32, and Lu177.

An “alkyl” group is a saturated straight chain or branched non-cyclic hydrocarbon having from 1 to 10 carbon atoms, typically from 1 to 8 carbons or, in some embodiments, from 1 to 6, 1 to 5, 1 to 4, or 2 to 6 or carbon atoms. Representative alkyl groups include -methyl, -ethyl, -n-propyl, -n-butyl, -n-pentyl and n-hexyl; while saturated branched alkyls include -isopropyl, -sec-butyl, -isobutyl, -tert-butyl, -isopentyl, 2-methylpentyl, 3methylpentyl, 4-methylpentyl, 2,3-dimethylbutyl and the like. An alkyl group can be substituted or unsubstituted. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “cycloalkyl” group is a saturated, or a partially saturated, cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or even 6 to 10 atoms in the ring portions of the groups. Suitable heteroatoms include oxygen, sulfur, and nitrogen. In certain embodiments, the heteroaryl ring system is monocyclic or bicyclic. Non-limiting examples include, but are not limited to, groups such as pyrrolyl, pyrazolyl, imidazolyl, triazolyl, tetrazolyl, oxazolyl, isoxazolyl, thiazolyl, pyrrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3H-imidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heterocyclyl” or “heterocyclic” is a non-aromatic cycloalkyl or aryl in which one to four of the ring carbon atoms are independently replaced with a heteroatom independently selected from the group consisting of O, S, and N. In some embodiments, heterocyclyl groups include 3 to 10 ring members, whereas other such groups have 3 to 5, 3 to 6, or 3 to 8 ring members. In some embodiments, heterocyclyl is aromatic. In some embodiments, heterocyclyl is non-aromatic. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated, and saturated ring systems, such as, for example, imidazolyl, imidazolinyl and imidazolidinyl groups. The phrase heterocyclyl includes fused ring species, including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, inidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridin-2(3H)-onyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzil and phenethyl groups and fused (cycloalkylaryl)alkyl groups such as 4-ethyl-indanyl.

A “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocyclylalkyl groups include but are not limited to 4-ethyl-morpholinyl, 4-propylmorpholinyl, furan-2-yl methyl, furan-3-yl methyl, pyridine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

An “alkoxy” or “alkoxyl” group is —O(alkyl), wherein alkyl is defined above.

An “alkoxyalkyl” group is -(alkyl)O(alkyl), wherein each alkyl is independently as defined above.

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g., C₆H₅—O—, for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C₆₋₁₅ aryloxy, and 6 to 10 ring carbon atoms, i.e., C₆₋₁₀ aryloxy. Examples of aryloxy moieties include, but are not limited to phenoxy, naphthoxy, and anthroxy.

An “amine” group is a radical of the formula: —NH₂.

A “hydroxyl amine” group is a radical of the formula: N(R^{#})OH or NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula: —N(R^{#})O-alkyl or —NHO-alkyl, wherein R^{#} is as defined above.

An “aralkoxyamine” group is a radical of the formula: N(R^{#})O-aryl or NH-Oaryl, wherein R^{#} is as defined above.

An “alkylamine” group is a radical of the formula: NHalkyl or N(alkyl)₂, wherein each alkyl is independently as defined above.

An “aminocarbonyl” group is a radical of the formula: —C(═O)N(R^{#})₂, —C(═O)NH(R^{#}), or C(═O)NH₂, wherein each R^{#} is as defined above.

An “acylamino” group is a radical of the formula: NHC(═O)(R^{#}) or N(alkyl)C(═O)(R^{#}), wherein each alkyl and R^{#} are independently as defined above.

An “O(alkyl)aminocarbonyl” group is a radical of the formula: —O(alkyl)C(═O)N(R^{#})₂, —O(alkyl)C(═O)NH(R^{#}) or —O(alkyl)C(═O)NH₂, wherein each R^{#} is independently as defined above.

An “N-oxide” group is a radical of the formula: —N⁺—O⁻.

A “carboxy” group is a radical of the formula: C(═O)OH.

A “ketone” group is a radical of the formula: C(═O)(R^{#}), wherein R^{#} is as defined above.

An “aldehyde” group is a radical of the formula: —CH(═O).

An “ester” group is a radical of the formula: C(═O)O(R^{#}) or OC(═O)(R^{#}), wherein R^{#} is as defined above.

A “urea” group is a radical of the formula: —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or NHC(═O)NH₂, wherein each alkyl and R^{#} are independently as defined above.

An “imine” group is a radical of the formula: —N═C(R^{#})₂ or —C(R^{#})═N(R^{#}), wherein each R^{#} is independently as defined above.

An “imide” group is a radical of the formula: —C(═O)N(R^{#})C(═O)(R^{#}) or N((C═O)(R^{#}))₂, wherein each R^{#} is independently as defined above.

A “urethane” group is a radical of the formula: —OC(═O)N(R^{#})₂, —OC(═O)NH(R^{#}), —N(R^{#})C(═O)O(R^{#}), or —NHC(═O)O(R^{#}), wherein each R^{#} is independently as defined above.

An “amidine” group is a radical of the formula: —C(═N(R^{#}))N(R^{#})₂, C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})═NH, or —NHC(R^{#})═NH, wherein each R^{#} is independently as defined above.

A “guanidine” group is a radical of the formula: —N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), —NHC(═NH)NH₂, —N═C(N(R^{#})₂)₂, —N═C(NIH(R^{#}))₂, or —N═C(NH₂)₂, wherein each R^{#} is independently as defined above.

An “enamine” group is a radical of the formula: —N(R^{#})C(R^{#})═C(R^{#})₂, NHC(R^{#})═C(R^{#})₂, —C(N(R^{#})₂)═C(R^{#})₂, —C(NH₄(R^{#}))═C(R^{#})₂, —C(NH₂)═C(R^{#})₂, C(R^{#})C(R^{#})(N(R^{#})₂), C(R^{#})═C(R^{#})(NH(R^{#})) or —C(R^{#})═C(R^{#})(NH₂), wherein each RV is independently as defined above.

An “oxime” group is a radical of the formula: —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula: —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or —C(═O)NHNH₂, wherein each R^{#} is independently as defined above.

A “hydrazine” group is a radical of the formula: —N(R^{#})N(R^{#})₂, —NHN(R^{#})₂, —N(R^{#})NH(R^{#}), —N(R^{#})NH₂, —NHNH(R^{#})₂, or —NHNH₂, wherein each R^{#} is independently as defined above.

A “hydrazone” group is a radical of the formula: —C(═N—N(R^{#})₂)(R^{#})₂, —C(═NNH(R^{#}))(R^{#})₂, —C(═N—NH₂)(R^{#})₂, —N(R^{#})(N═C(R^{#})₂), or —NH(N═C(R^{#})₂), wherein each R^{#} is independently as defined above.

An “azide” group is a radical of the formula: —N₃.

An “isocyanate” group is a radical of the formula: N═C═O.

An “isothiocyanate” group is a radical of the formula: N═C═S.

A “cyanate” group is a radical of the formula: OCN.

A “thiocyanate” group is a radical of the formula: SCN.

A “thioether” group is a radical of the formula; —S(R^{#}), wherein R^{#} is as defined above.

A “thiocarbonyl” group is a radical of the formula: —C(═S)(R^{#}), wherein R^{#} is as defined above.

A “sulfinyl” group is a radical of the formula: —S(═O)(R^{#}), wherein R^{#} is as defined above.

A “sulfone” group is a radical of the formula: —S(═O)₂(R^{#}), wherein R^{#} is as defined above.

A “sulfonylamino” group is a radical of the formula: —NHSO₂(R^{#}) or —N(alkyl)SO₂(R^{#}), wherein each alkyl and R^{#} are defined above.

A “sulfonamide” group is a radical of the formula: —S(═O)₂N(R^{#})₂, or —S(═O)₂NH(R^{#}), or —S(═O)₂NH₂, wherein each R^{#} is independently as defined above.

A “phosphonate” group is a radical of the formula: —P(═O)(O(R^{#}))₂, —P(═O)(OH)₂, —OP(═O)(O(R^{#}))(R^{#}), or —OP(═O)(OH)(R^{#}), wherein each R^{#} is independently as defined above.

A “phosphine” group is a radical of the formula: —P(R^{#})₂, wherein each R^{#} is independently as defined above.

When the groups described herein, with the exception of alkyl groups, are said to be “substituted” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone: aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base.

As used herein and unless otherwise indicated, the term “solvate” means a compound, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a compound, or a salt thereof, that further includes a stoichiometric or non-stoichiometric amount of water bound by non-covalent intermolecular forces.

All pharmaceutically acceptable salts solvates, and/or hydrates of compounds depicted herein are within the scope of the present disclosure.

As used herein and unless otherwise indicated, the term “prodrug” means a compound derivative that can hydrolyze, oxidize, or otherwise react under biological conditions (in vitro or in vivo) to provide an active compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (WileyInterscience, New York, 1981); Wilen, S. H., et al., Tetrahedron 33:2725 (1977); Eliel, E. L., Stereochemistry of Carbon Compounds (McGrawHill, NY, 1962); and Wilen, S. H., Tables of Resolving Agents and Optical Resolutions p. 268 (E. L. Eliel, Ed., Univ. of Notre Dame Press, Notre Dame., IN, 1972).

It should also be noted the compounds can include E and Z isomers, or a mixture thereof, and cis and trans isomers or a mixture thereof. In certain embodiments, the compounds are isolated as either the cis or trans isomer. In other embodiments, the compounds are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in an aqueous solution, pyrazoles may exhibit the following isomeric forms, which are referred to as tautomers of each other:

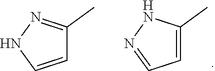

As readily understood by one skilled in the art, a wide variety of functional groups and other structures may exhibit tautomerism and all tautomers of the compounds are within the scope of the present disclosure.

It should also be noted the compounds can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

It should be noted that if there is a discrepancy between a depicted structure and a name for that structure, the depicted structure is to be accorded more weight.

As used herein, the term “residue” refers to the chemical moiety within a compound that remains after a chemical reaction. For example, the term “amino acid residue” or “N-alkyl amino acid residue” refers to the product of an amide coupling or peptide coupling of an amino acid or a N-alkyl amino acid to a suitable coupling partner; wherein, for example, a water molecule is expelled after the amide or peptide coupling of the amino acid or the N-alkylamino acid, resulting in the product having the amino acid residue or N-alkyl amino acid residue incorporated therein.

As used herein, “sugar” or “sugar group” or “sugar residue” refers to a carbohydrate moiety which may comprise 3-carbon (triose) units, 4-carbon (tetrose) units, 5-carbon (pentose) units, 6-carbon (hexose) units, 7-carbon (heptose) units, or combinations thereof, and may be a monosaccharide, a disaccharide, a trisaccharide, a tetrasaccharide, a pentasaccharide, an oligosaccharide, or any other polysaccharide. In some instances, a “sugar” or “sugar group” or “sugar residue” comprises furanoses (e.g., ribofuranose, fructofuranose) or pyranoses (e.g., glucopyranose, galactopyranose), or a combination thereof. In some instances, a “sugar” or “sugar group” or “sugar residue” comprises aldoses or ketoses, or a combination thereof. Non-limiting examples of monosaccharides include ribose, deoxyribose, xylose, arabinose, glucose, mannose, galactose, and fructose. Non-limiting examples of disaccharides include sucrose, maltose, lactose, lactulose, and trehalose. Other “sugars” or “sugar groups” or “sugar residues” include polysaccharides and/or oligosaccharides, including, and not limited to, amylose, amylopectin, glycogen, inulin, and cellulose. In some instances, a “sugar” or “sugar group” or “sugar residue” is an amino-sugar. In some instances, a “sugar” or “sugar group” or “sugar residue” is a glucamine residue (1-amino-1-deoxy-D-glucitol) linked to the rest of molecule via its amino group to form an amide linkage with the rest of the molecule (i.e., a glucamide).

Certain groups, moieties, substituents, and atoms are depicted with a wiggly line, e.g., that intersects a bond or bonds or extends from an atom, to indicate the atom through which the groups, moieties, substituents, atoms are bonded. For example, a phenyl group that is substituted with a propyl group depicted as:

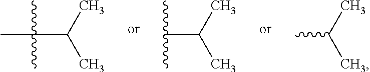

has the following structure:

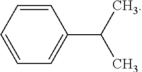

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains.

Illustrations showing substituents bonded to a non-cyclic group through a bond between two atoms are meant to indicate, unless specified otherwise, that the substituent may be bonded to either atom of the bond through which the substituent bond passes, according to techniques set forth herein or which are known in the field to which the instant disclosure pertains. Thus, for example,

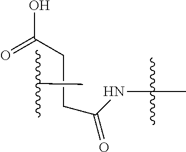

encompasses

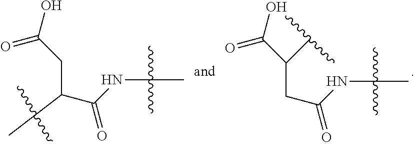 [SMILES: *NC(=O)C(*)CC(=O)O ; *NC(=O)CC(*)C(=O)O]

### Payloads

Provided herein are exatecan analogues, e.g., for use in a platform and/or ADC described herein.

One embodiment provides a compound of any one of the following formulas, or a pharmaceutically acceptable salt and/or solvate thereof:

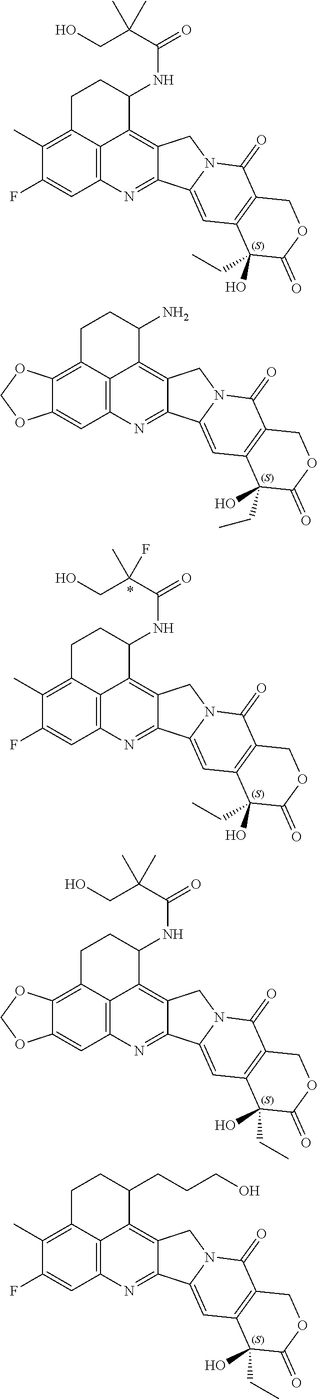

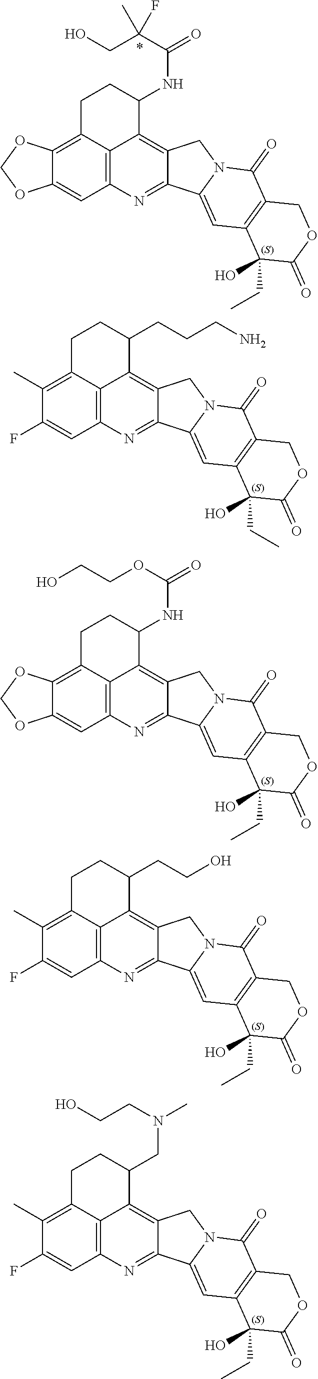

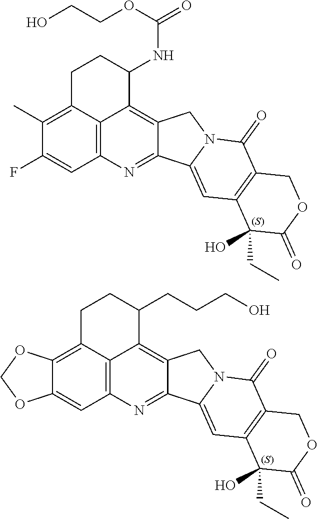

In one embodiment, the compound is

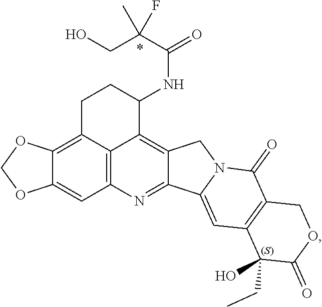

or a pharmaceutically acceptable salt, stereoisomer, and/or solvate thereof. In one embodiment, the compound is

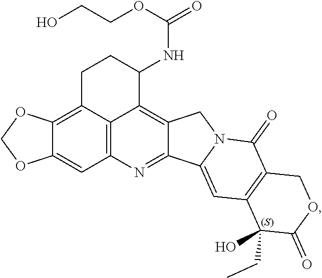

or a pharmaceutically acceptable salt, stereoisomer, and/or solvate thereof.

A residue of a payload described herein can be combined with any linker described herein to form a platform, or a residue of a payload described herein can be combined with any linker described herein and any antibody or antigen binding fragment thereof described herein to form an ADC.

### Covalent Linkers

Also provided herein are covalent linkers, e.g., for use in a platform and/or ADC described herein.

In some embodiments (e.g., of a linker for use in a platform), the linker is:

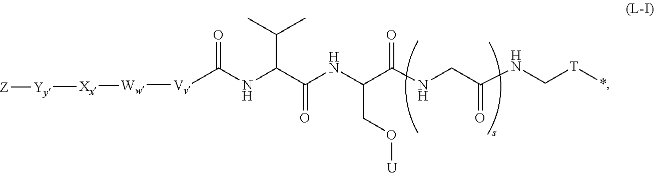

wherein:
   * indicates the bond where T attaches to a payload residue or drug unit (P);
   Z is a heterocyclic, optionally substituted with at least one carbonyl or methylsulfonyl;
   Y is an arylene or —C═C—(CH₂)ₐ—;
   subscript a is 1, 2, 3, or 4;
   subscript y′ is 0 or 1;
   X is a substituted or unsubstituted C₁₋₅ alkyl;
   subscript x′ is 0 or 1;
   W is oxygen;
   subscript w′ is 0 or 1;
   V is a C₁₋₄ alkyl or —C(═O)—NH—(CH₂)_{b} wherein the N is optionally substituted with a C₁₋₅ alkyl and subscript b is 1 or 2;
   subscript v′ is 0 or 1;
   U is a hydrophilic group;
   T is oxygen or a bond; and
   subscript s is 0 or 1.

In some embodiments (e.g., of a linker for use in an ADC), the linker is:

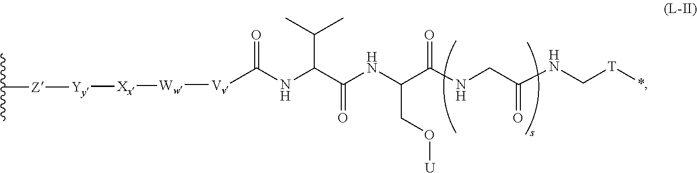

wherein:
   * indicates the bond where T attaches to a payload residue or drug unit (P);
   Z is a heterocyclic, optionally substituted with at least one carbonyl or methylsulfonyl;
   Y is an arylene or —C═C—(CH₂)ₐ—;
   subscript a is 1, 2, 3, or 4;
   subscript y′ is 0 or 1;
   X is a substituted or unsubstituted C₁₋₅ alkyl;
   subscript x′ is 0 or 1;
   W is oxygen;
   subscript w′ is 0 or 1;
   V is a C₁₋₄ alkyl or —C(═O)—NH—(CH₂)_{b} wherein the N is optionally substituted with a C₁₋₅ alkyl and subscript b is 1 or 2;
   subscript v′ is 0 or 1;
   U is a hydrophilic group;
   T is oxygen or a bond; and
   subscript s is 0 or 1.

In some embodiments, Z is

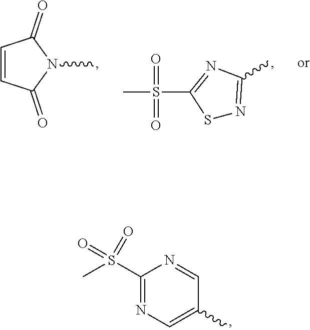

In some embodiments, Z′ is

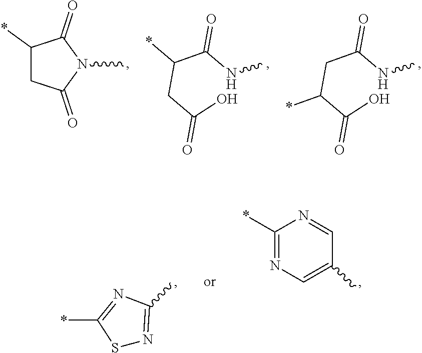

and * indicates the bond where Z attaches to BA (e.g., Ab).

In some embodiments, Y is phenylene or —C═C—(CH₂)₃—. In further embodiments, Y is phenylene or —C═C—(CH₂)₃—, and subscript y′ is 1.

In some embodiments, subscript y′ is 1. In other embodiments, subscript y′ is 0.

In some embodiments, X is —(CH₂)₅—, —CH(CH₂NH₂)(CH₂)₄—, —CH₂CH(NH₂)CH₂—, —CH(CH₂NH₂)CH₂—, or —CH(CH₂NH₂)(CH₂)₂—. In further embodiments, X is —(CH₂)₅—, —CH(CH₂NH₂)(CH₂)₄—, —CH₂CH(NH₂)CH₂—, —CH(CH₂NH₂)CH₂—, or —CH(CH₂NH₂)(CH₂)₂—, and subscript x′ is 1.

In some embodiments, subscript x′ is 1. In other embodiments, subscript x′ is 0.

In some embodiments, subscript w′ is 1. In other embodiments, subscript w′ is 0.

In some embodiments, V is ethyl or —C(═O)—NH—(CH₂)_{b}, wherein N is optionally substituted with a C₁₋₃ alkyl, and subscript b is 1 or 2. In further embodiments, V is ethyl or —C(═O)—NH—(CH₂)_{b}, wherein N is optionally substituted with a C₁₋₃ alkyl, and subscript b is 1 or 2; and subscript v′ is 1. In some embodiments, V is

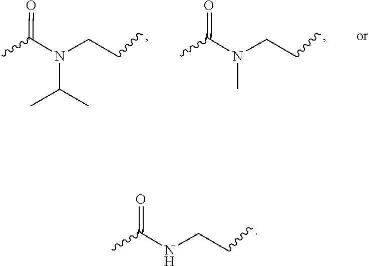

In further embodiments, V is

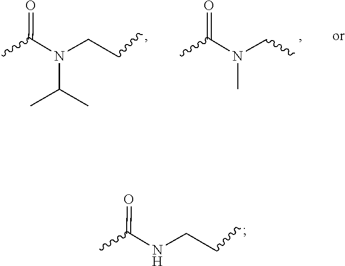

and subscript v′ is 1.

In some embodiments, subscript v′ is 1. In other embodiments, subscript v is 0.

In some embodiments, U is a saccharide, phosphate ester, sulfate ester, a phosphodiester, or a phosphonate. In further embodiments, U is a saccharide, and the saccharide is β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, a neuraminic acid, sulfate, phosphate, carboxyl, amino, or an O-acetyl modification thereof.

In some embodiments U is

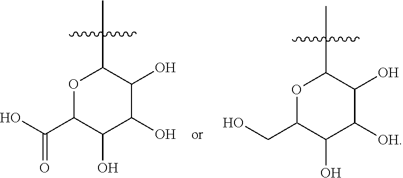 [SMILES: *C1OC(C(=O)O)C(O)C(O)C1O ; *C1OC(CO)C(O)C(O)C1O]

In some embodiments, U is

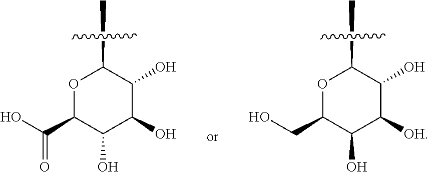

In some embodiments, T is oxygen. In some embodiments, T is a bond.

In some embodiments, subscript s is 1. In other embodiments, subscript s is 0.

Linkers with Payloads (Platforms)

Also provided herein are platforms, e.g., for use in preparing an ADC, such as an ADC described herein.

One embodiment is a compound of Formula (I):

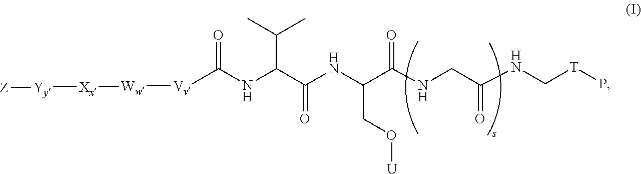

or a pharmaceutically acceptable salt, tautomer, solvate, or stereoisomer thereof, wherein:
Z is a heterocyclic, optionally substituted with at least one carbonyl or methylsulfonyl;
Y is an arylene or —C═C—(CH₂)ₐ—
subscript a is 1, 2, 3, or 4;
subscript y′ is 0 or 1;
X is a substituted or unsubstituted C₁₋₅ alkyl;
subscript x′ is 0 or 1;
W is oxygen;
subscript w′ is 0 or 1;
V is a C₁₋₄ alkyl or —C(═O)—NH—(CH₂)_{b} wherein the N is optionally substituted with a C₁₋₅ alkyl and subscript b is 1 or 2;
subscript v′ is 0 or 1;
U is a hydrophilic group;
T is oxygen or a bond;
P is a payload residue or a drug unit; and
subscript s is 0 or 1.

In some embodiments, the compound of Formula (I) is represented by Formula (Ia):

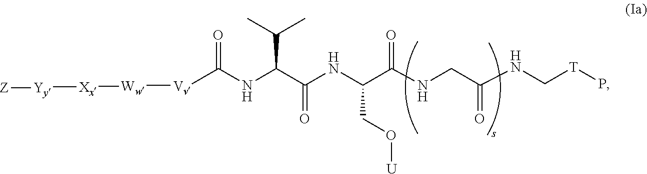

or a pharmaceutically acceptable salt, tautomer, solvate, or stereoisomer thereof, wherein values for the variables (e.g., Z, Y, y′, X, x′, W, w′, V, v, U, s, T, P) are as described herein, e.g., with respect to a compound of Formula (I).

In some embodiments, P is:

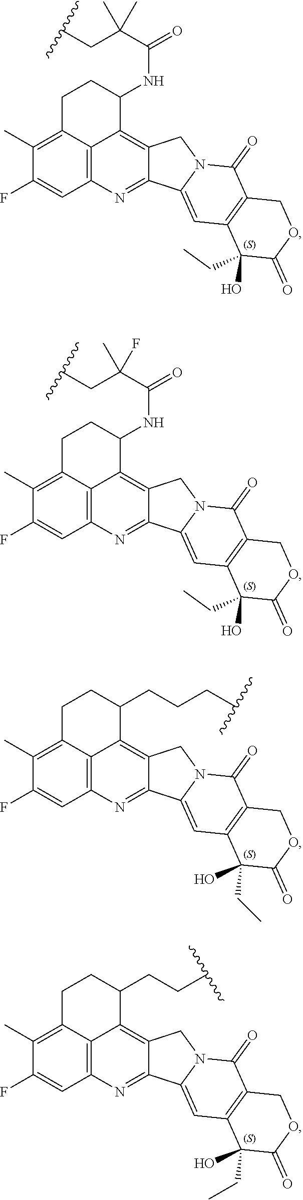

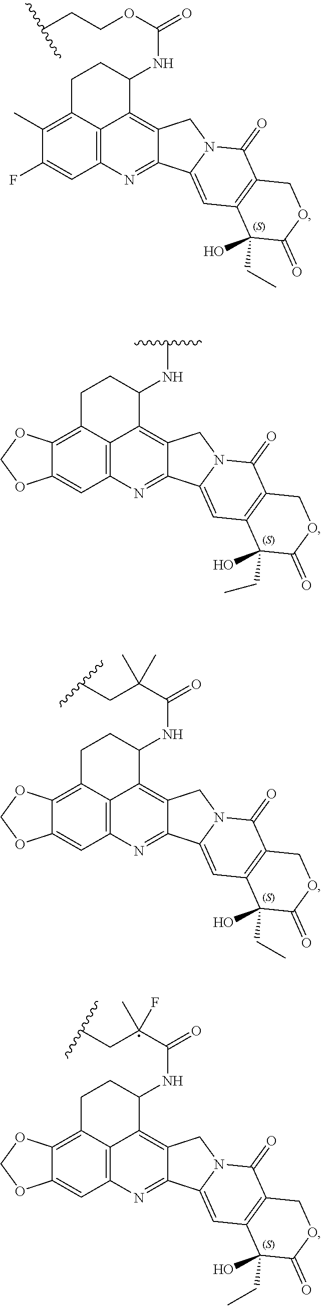

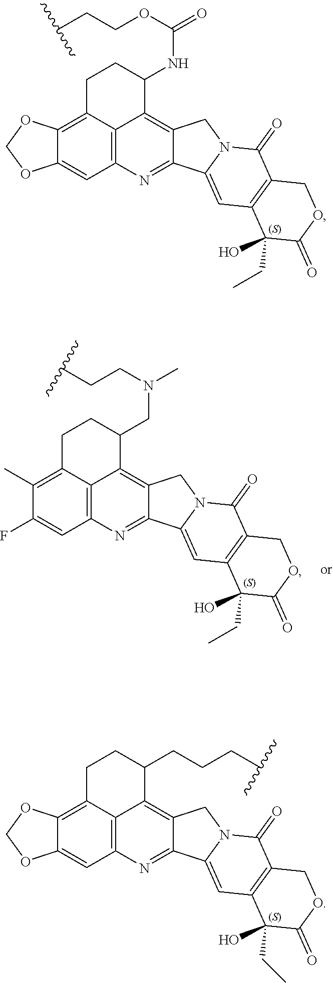

Alternative values for the variables (e.g., Z, Y, y′, X, x′, W, w′, V, v′, U s, T) are as described herein, e.g., with respect to Covalent Linkers.

Specific examples of platform compounds are described in the Examples. In some embodiments, the compound is selected from any one of the following, or a pharmaceutically acceptable salt, solvate, and/or stereoisomer thereof.

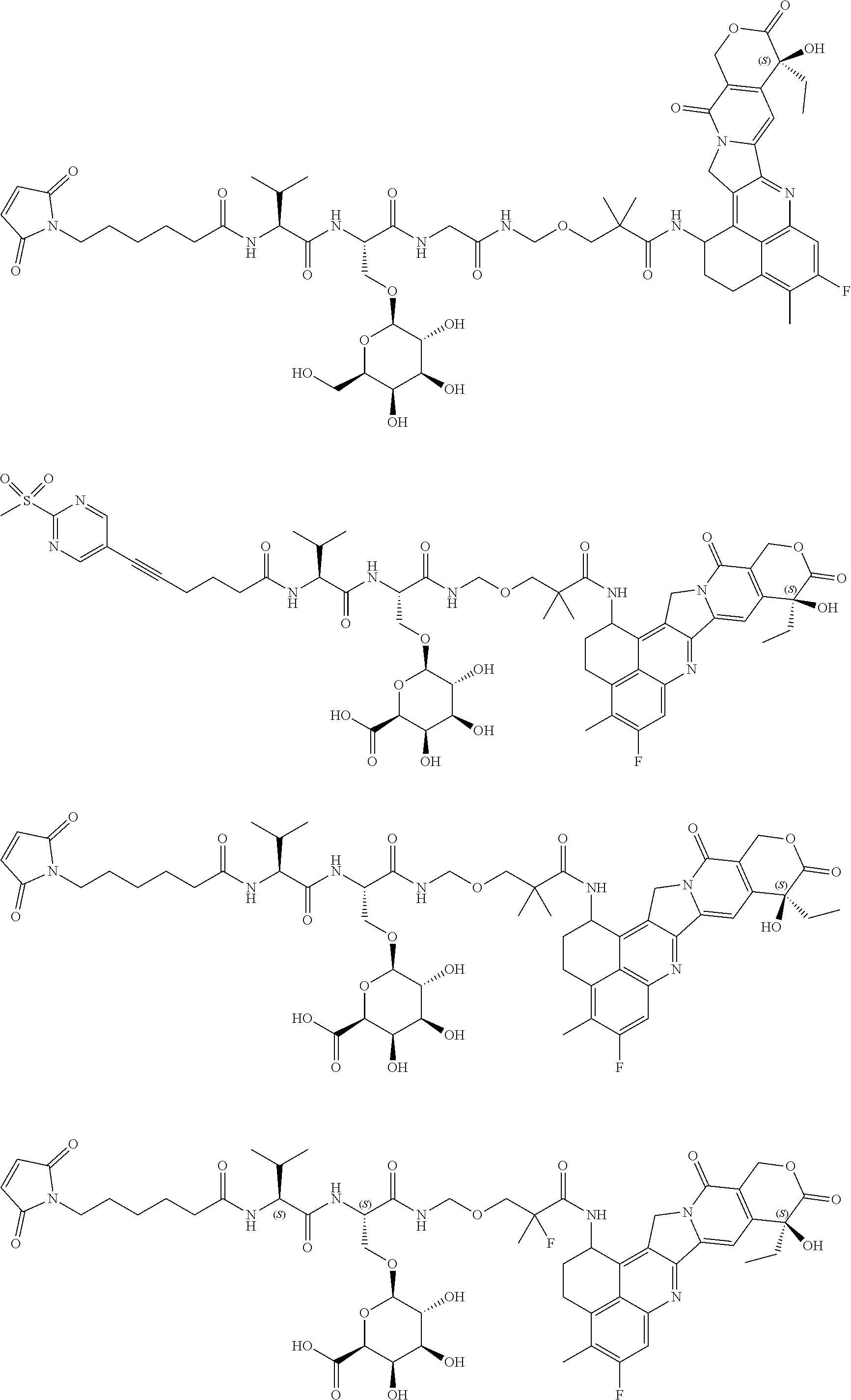

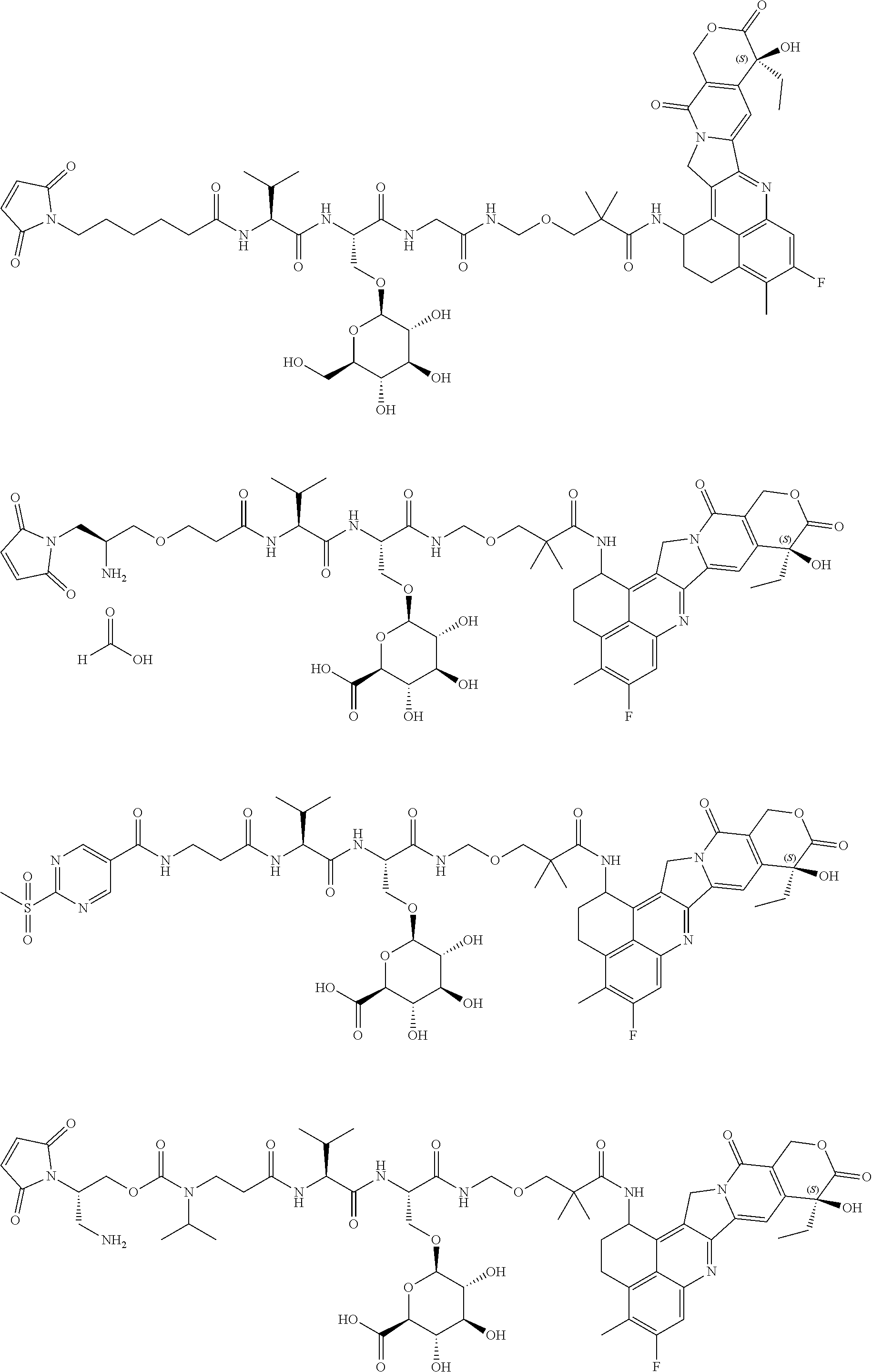

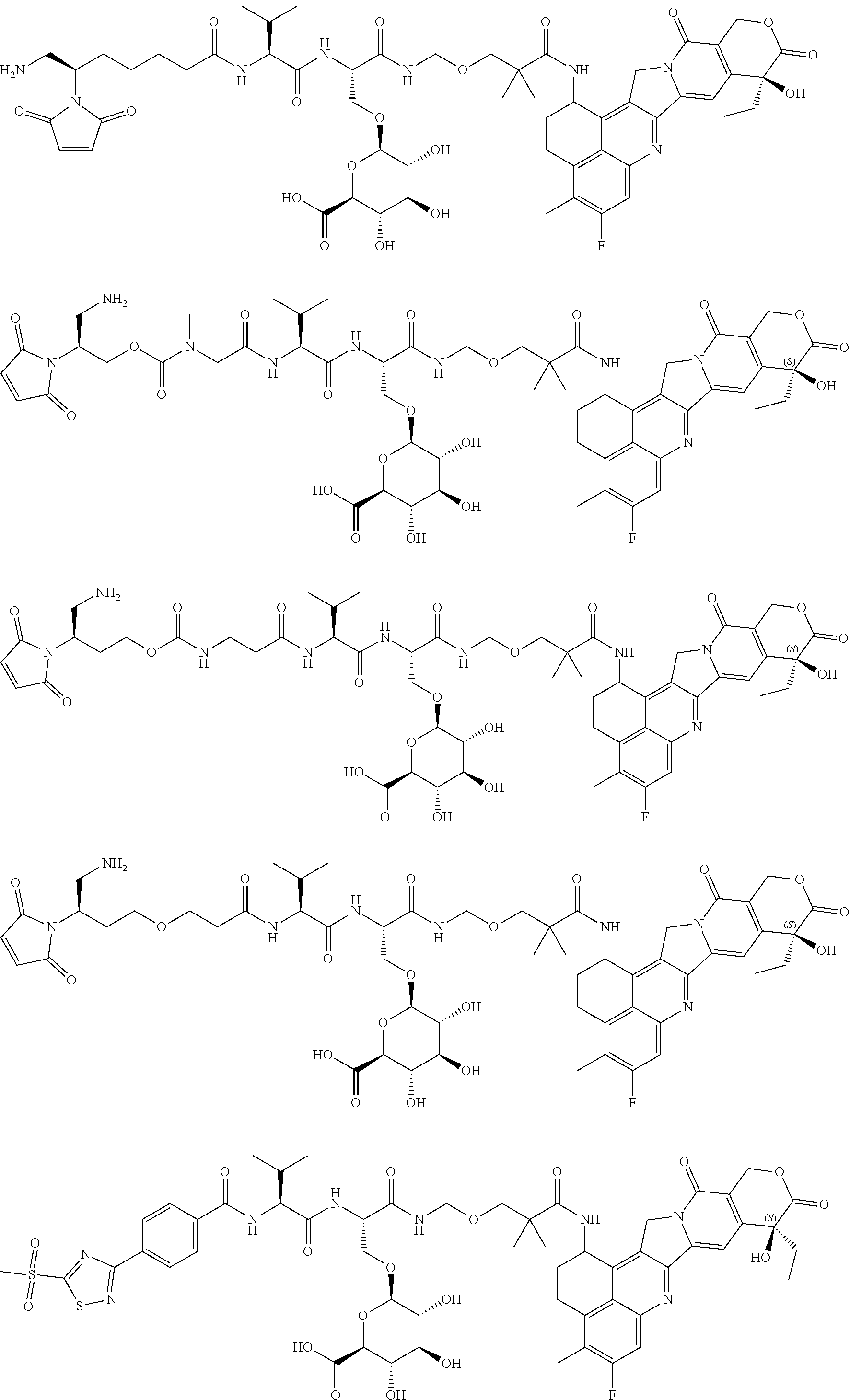

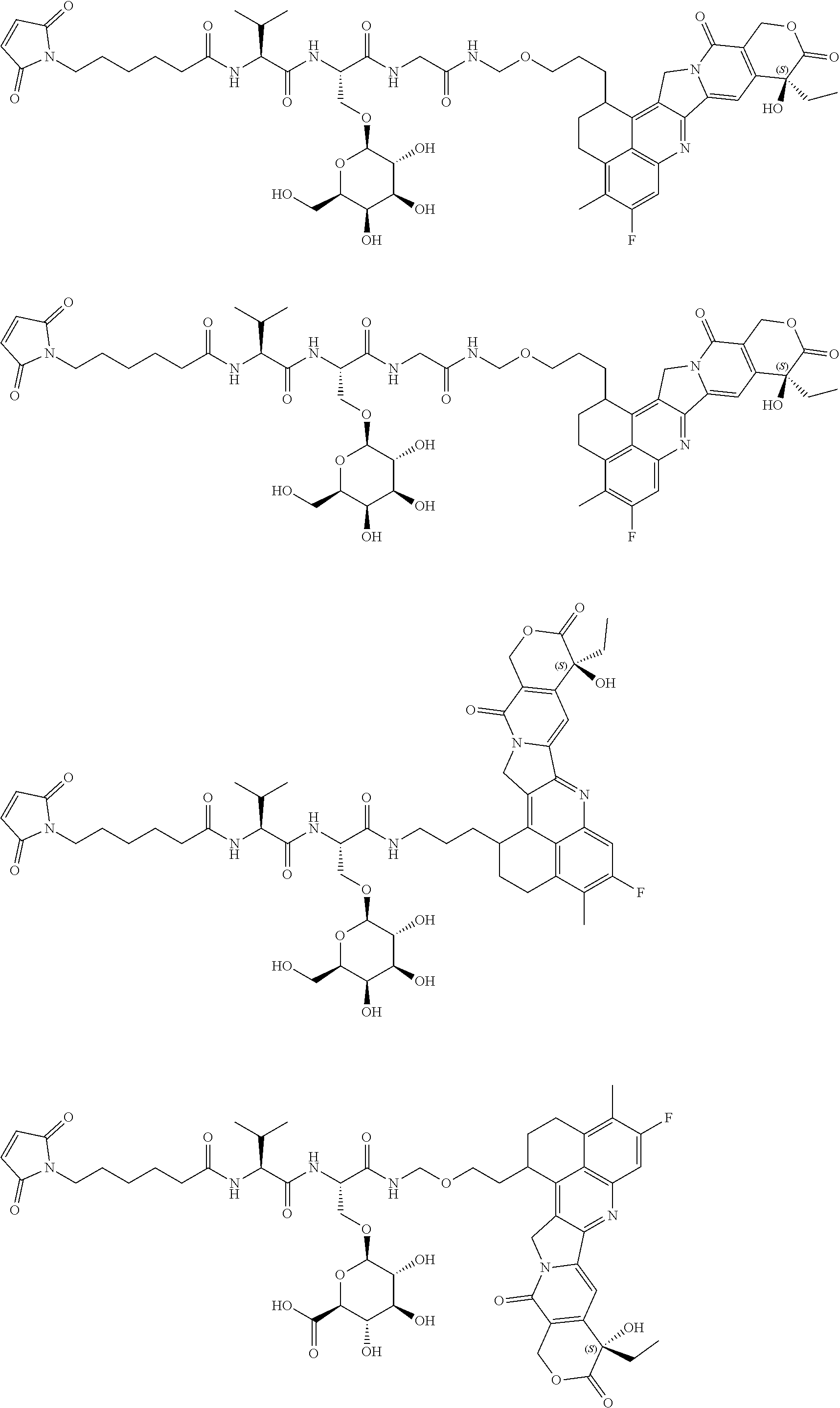

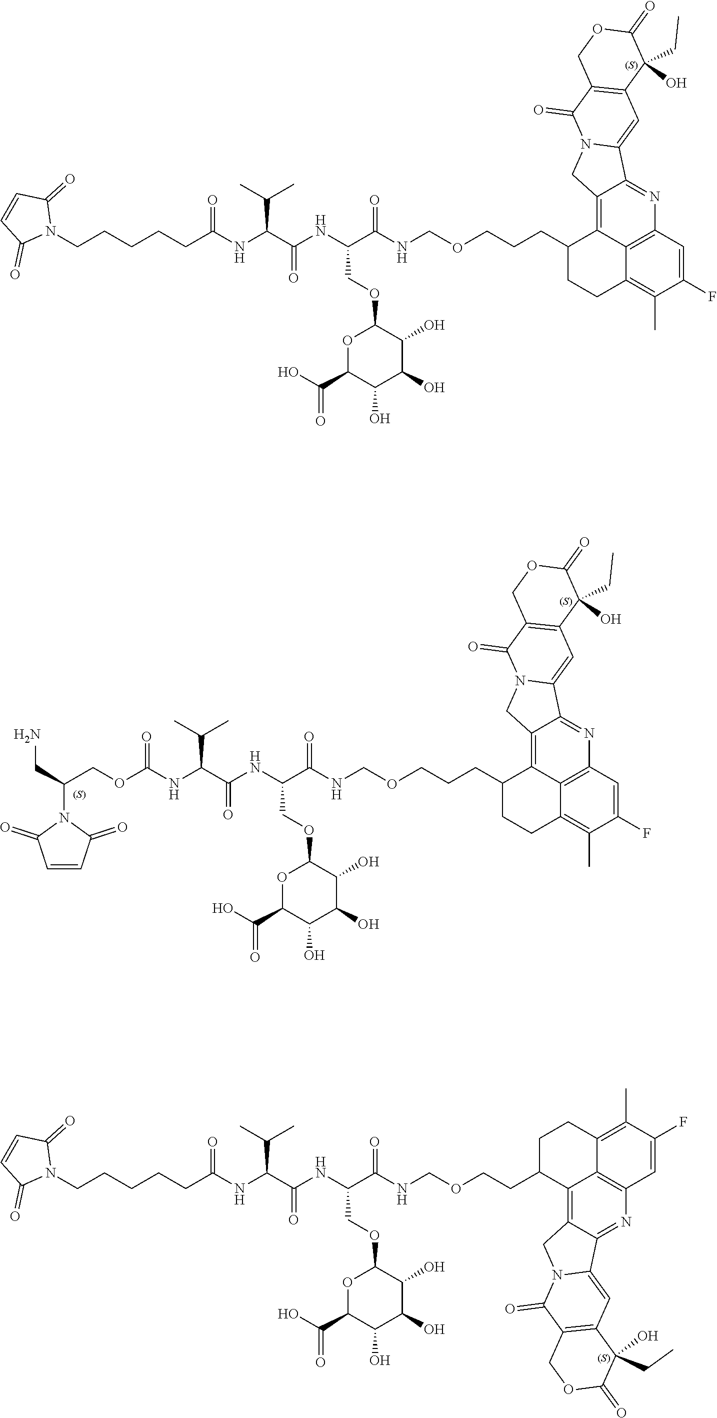

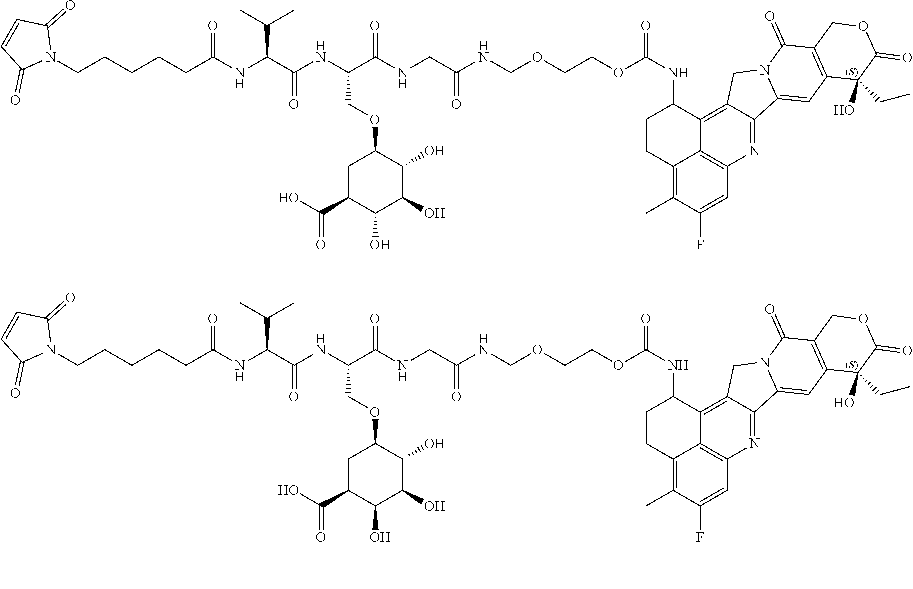

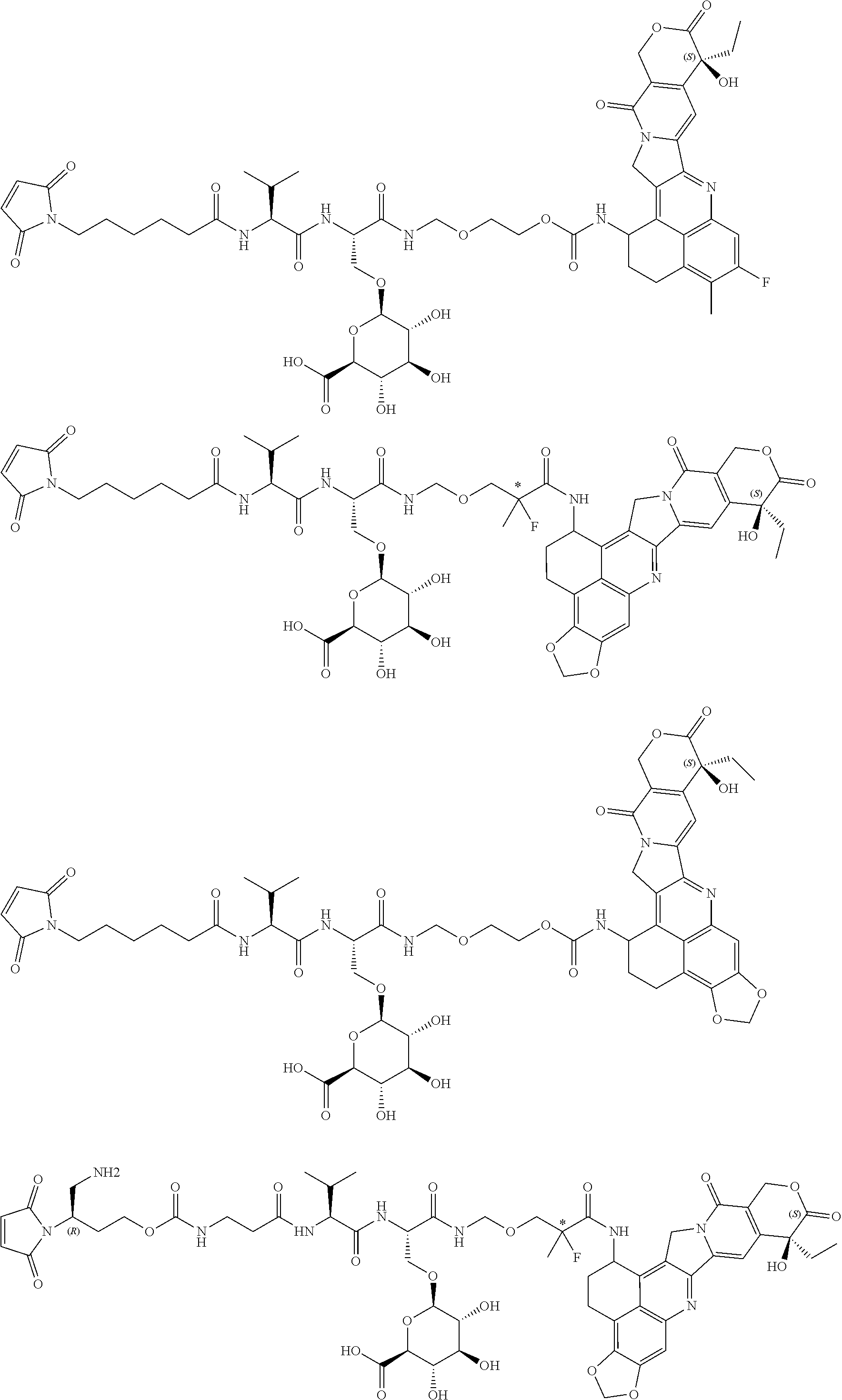

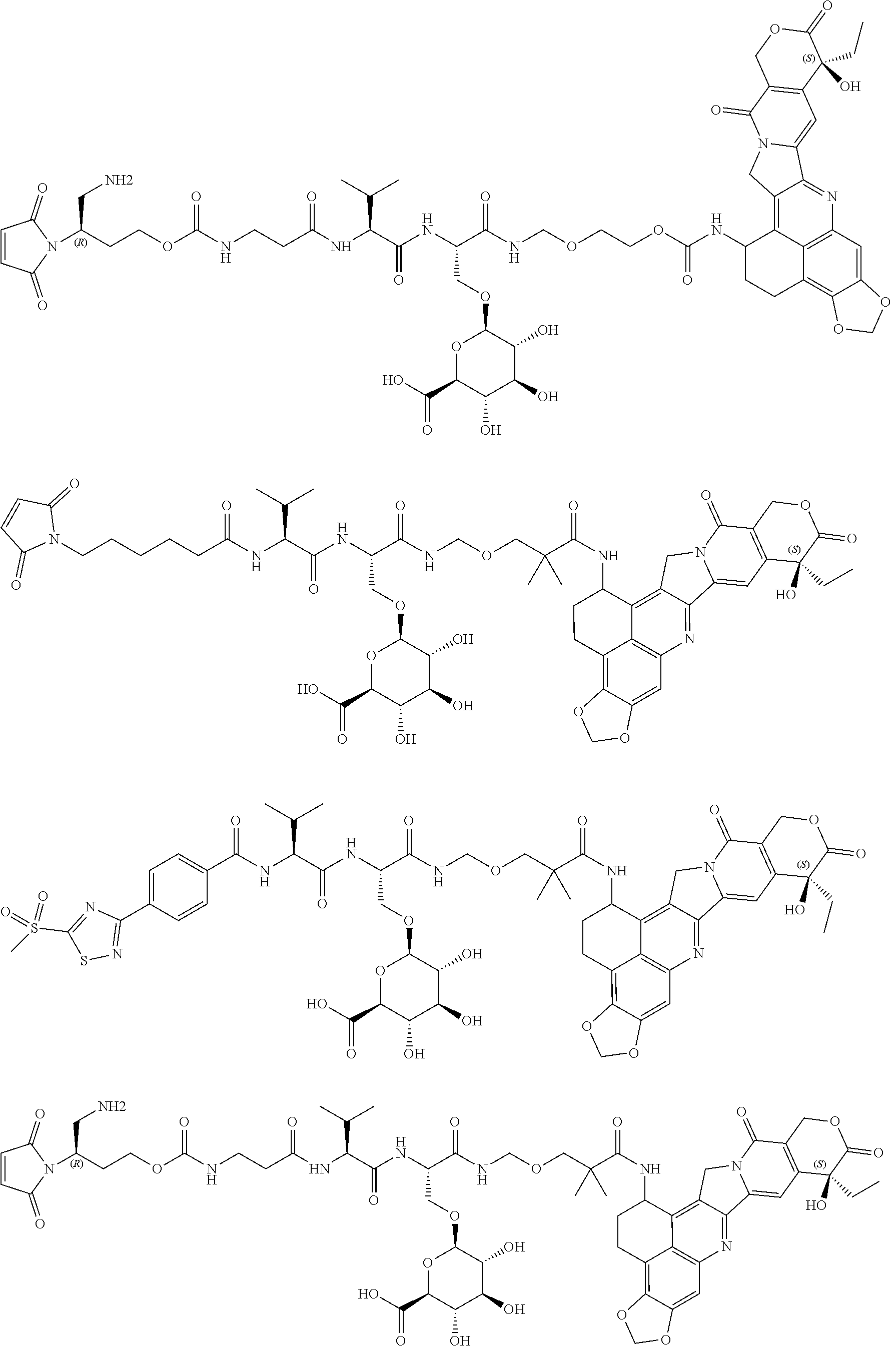

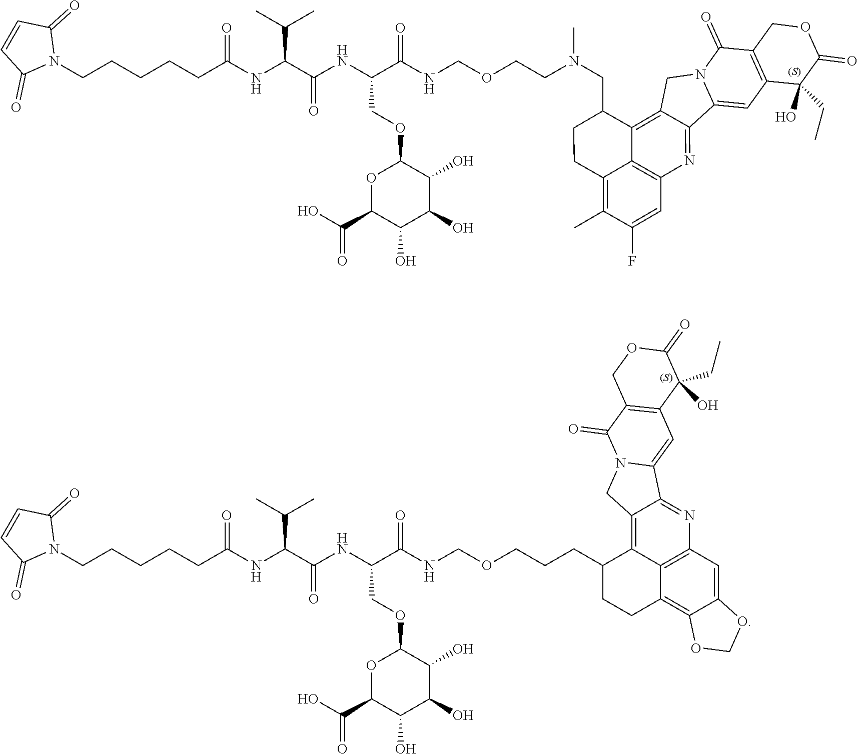

### Binding Agents, Including Antibodies

In some embodiments, BA is an antibody or antigen binding fragment thereof, e.g., a humanized, chimeric, or human antibody or an antigen binding fragment thereof.

In some embodiments, the antibody or antigen binding fragment thereof specifically binds human B7H3. In some embodiments, the antibody or antigen binding fragment thereof is ifinatimab.

In some embodiments, the antibody or antigen binding fragment thereof specifically binds human B7H3 and comprises i) a heavy chain variable region that comprises an amino acid sequence having at least 90%, 91%, 92%, 93%, 94%, 95%, 96%, 97%, 98%, 99%, 100% identity to the amino acid sequence of SEQ ID NO: 2; and a light chain variable region that comprises an amino acid sequence having at least 90%, 91%, 92%, 93%, 94% 95%, 96%, 97%, 98%, 99%, 100% identity to the amino acid sequence of SEQ ID NO: 1; optionally wherein the CDRs are the same as those of ifinatimab. In some embodiments, the amino acid sequence of the heavy chain variable region is SEQ ID NO: 2; and the amino acid sequence of the light chain variable region is SEQ ID NO: 1.

In some embodiments, one, two, three, four, five, six, seven, eight, nine, or ten amino acids are further incorporated (inserted, deleted or substituted) into the framework region of the above-mentioned antibodies, such as SEQ ID NO: 1 or SEQ ID NO: 2.

In some embodiments, the antibody or antigen binding fragment thereof comprises a heavy chain constant region of the subclass of IgG1, IgG2, IgG3, or IgG4, and/or a light chain constant region of the type of kappa or lambda.

In some embodiments, the antibody or antigen-binding fragment thereof is a monoclonal antibody, a chimeric antibody, a humanized antibody, a human engineered antibody, a single chain antibody (scFv), a Fab fragment, a Fab′ fragment, or a F(ab′)2 fragment.

In some embodiments, the antibody or antigen-binding fragment thereof has antibody dependent cellular cytotoxicity (ADCC) or complement dependent cytotoxicity (CDC).

In some embodiments, the Fc domain is an IgG1 with reduced effector function.

### Antibody Drug Conjugates (ADCs)

Also provided herein are ADCs, e.g., for use in therapy, such as cancer therapy.

One embodiment is an ADC of Formula (II):

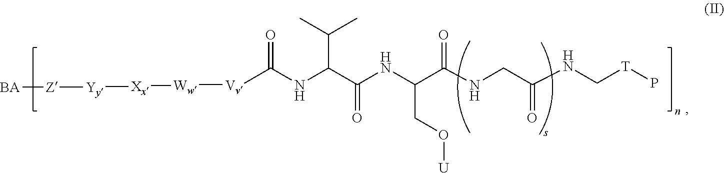

or a pharmaceutically acceptable salt, tautomer, solvate, or stereoisomer thereof, wherein:
BA is a binding agent selected from a humanized, chimeric, or human antibody or an antigen binding fragment thereof;
Z′ is a heterocyclic, optionally substituted with at least one carbonyl or methylsulfonyl;
Y is an arylene or —C═C—(CH₂)ₙ—;
subscript a is 1, 2, 3, or 4;
subscript y′ is 0 or 1;
X is a substituted or unsubstituted C₁₋₅ alkyl;
subscript x′ is 0 or 1;
W is oxygen;
subscript w′ is 0 or 1;
V is a C₁₋₄ alkyl or —C(═O)—NH—(CH₂)_{b} wherein the N is optionally substituted with a C₁₋₅ alkyl and subscript b is 1 or 2;
subscript v′ is 0 or 1;
U is a hydrophilic group;
T is oxygen or a bond;
P is a payload residue or a drug unit;
subscript s is 0 or 1; and
subscript n is from 1 to 15.

In some embodiments, the ADC of Formula (II) is represented by Formula (IIa):

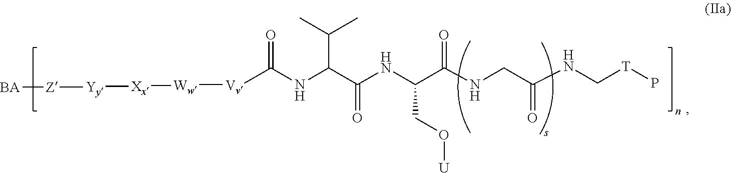

or a pharmaceutically acceptable salt, tautomer, solvate, or stereoisomer thereof, wherein values for the variables (e.g., BA, Z, Y, y′ X, x′, W, w′, V, v′, U, s, T, P, n) are as described herein, e.g., with respect to a compound of Formula (II).

In some embodiments, subscript n is from 3 to 12, e.g., from 4 to 11, from 5 to 10.

In some embodiments, subscript n is from 7 to 9. In some embodiments, subscript n is about 8.

Alternative values for the variables (e.g., BA, Z Y, y′, X, x′, W, w′, V, v′, U, s, T, P, n) are as described herein. For example, alternative values for variables Z′, Y, y′, X, x′, W, W V, v′, U, s, and T are as described herein with respect to Covalent Linkers. For example, alternative values for variable P are as described herein with respect to Payloads and/or Platforms. For example, alternative values for variables BA are as described herein, e.g., with respect to Binding Agents, Including Antibodies.

In some embodiments, an ADC is represented by one of the following formulas, or a pharmaceutically acceptable salt, solvate, and/or stereoisomer thereof:

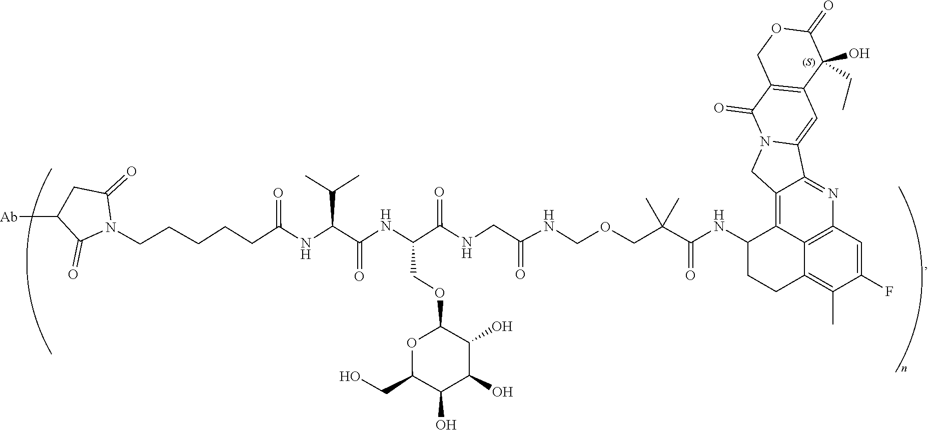

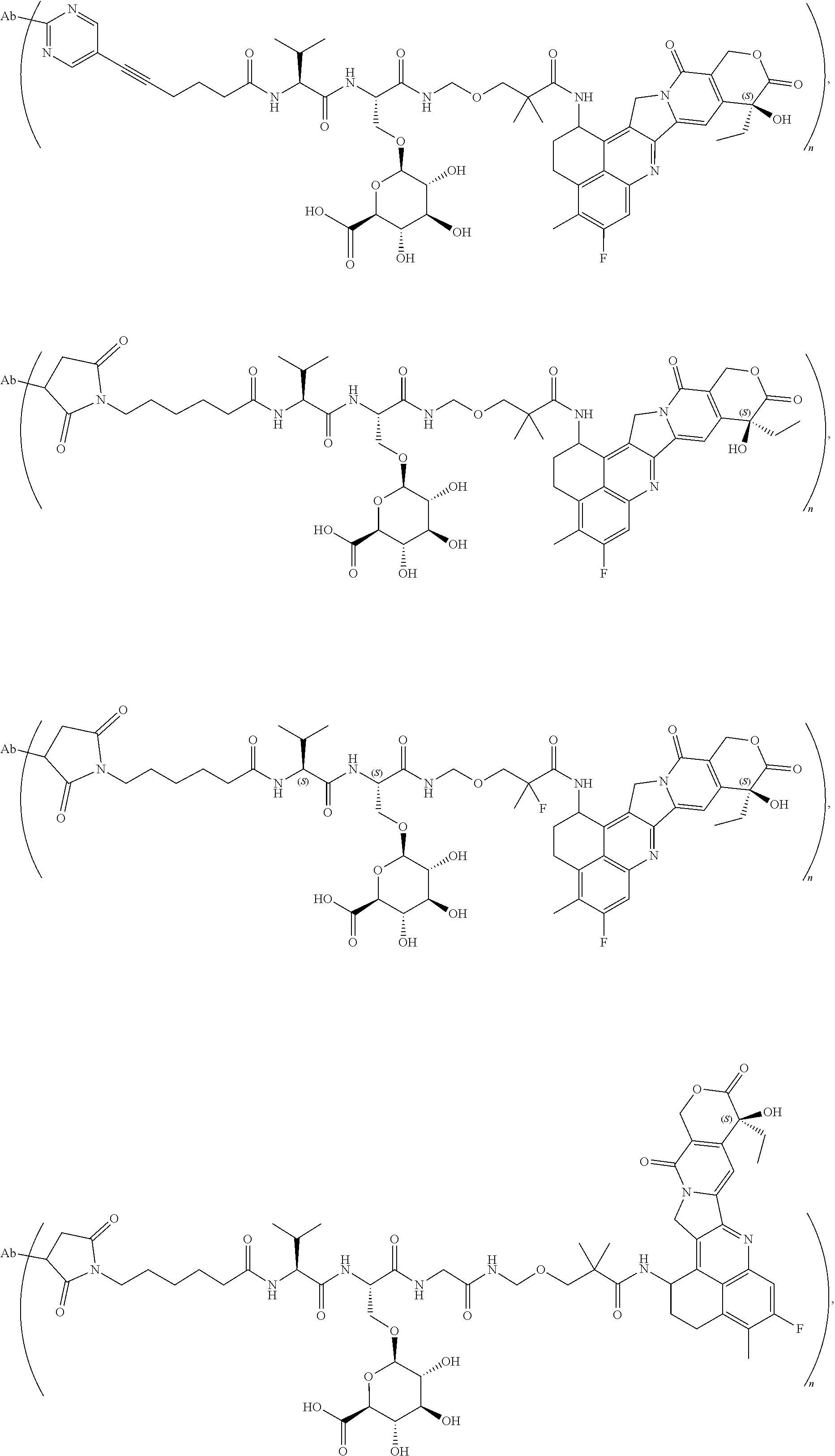

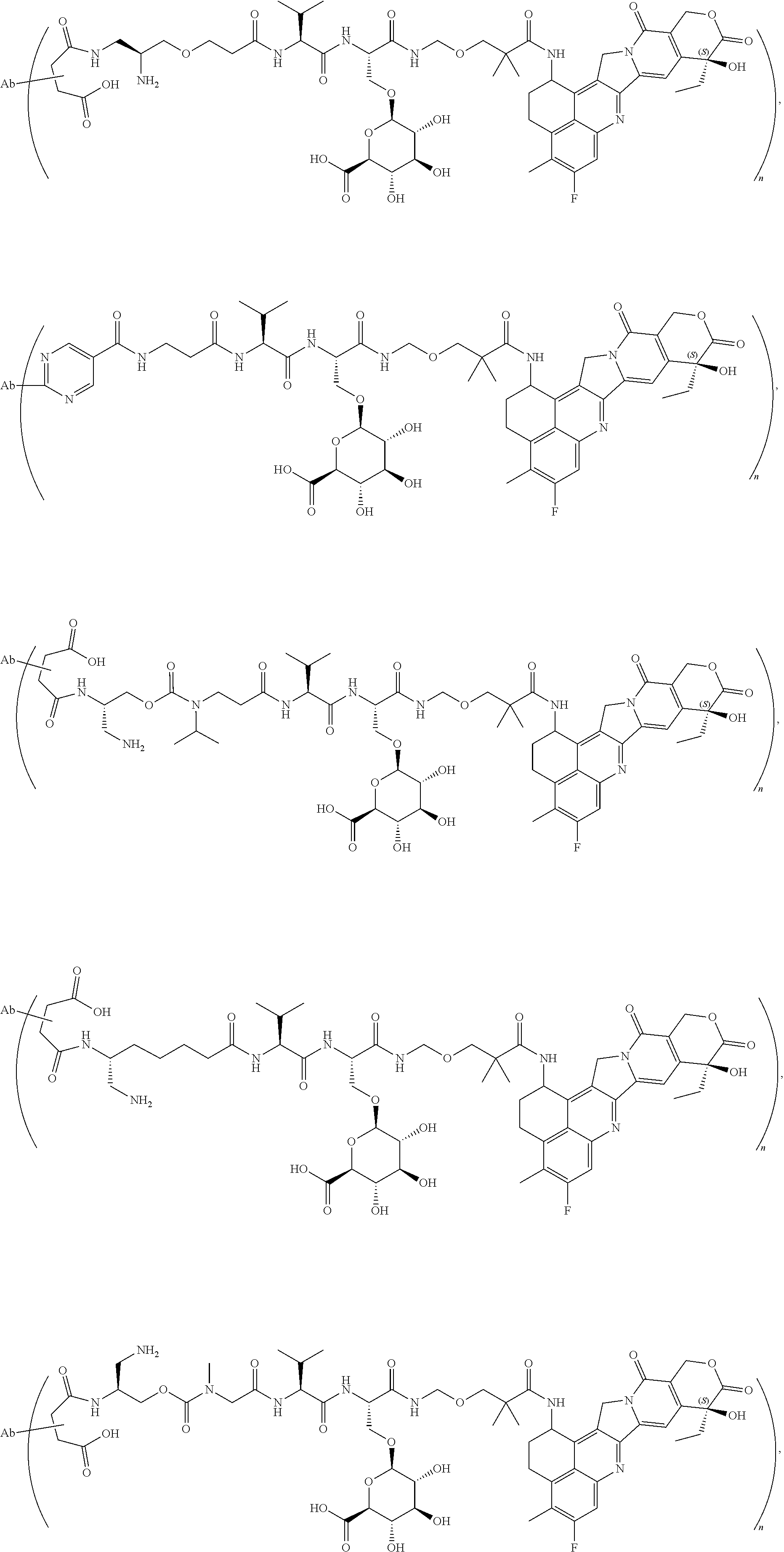

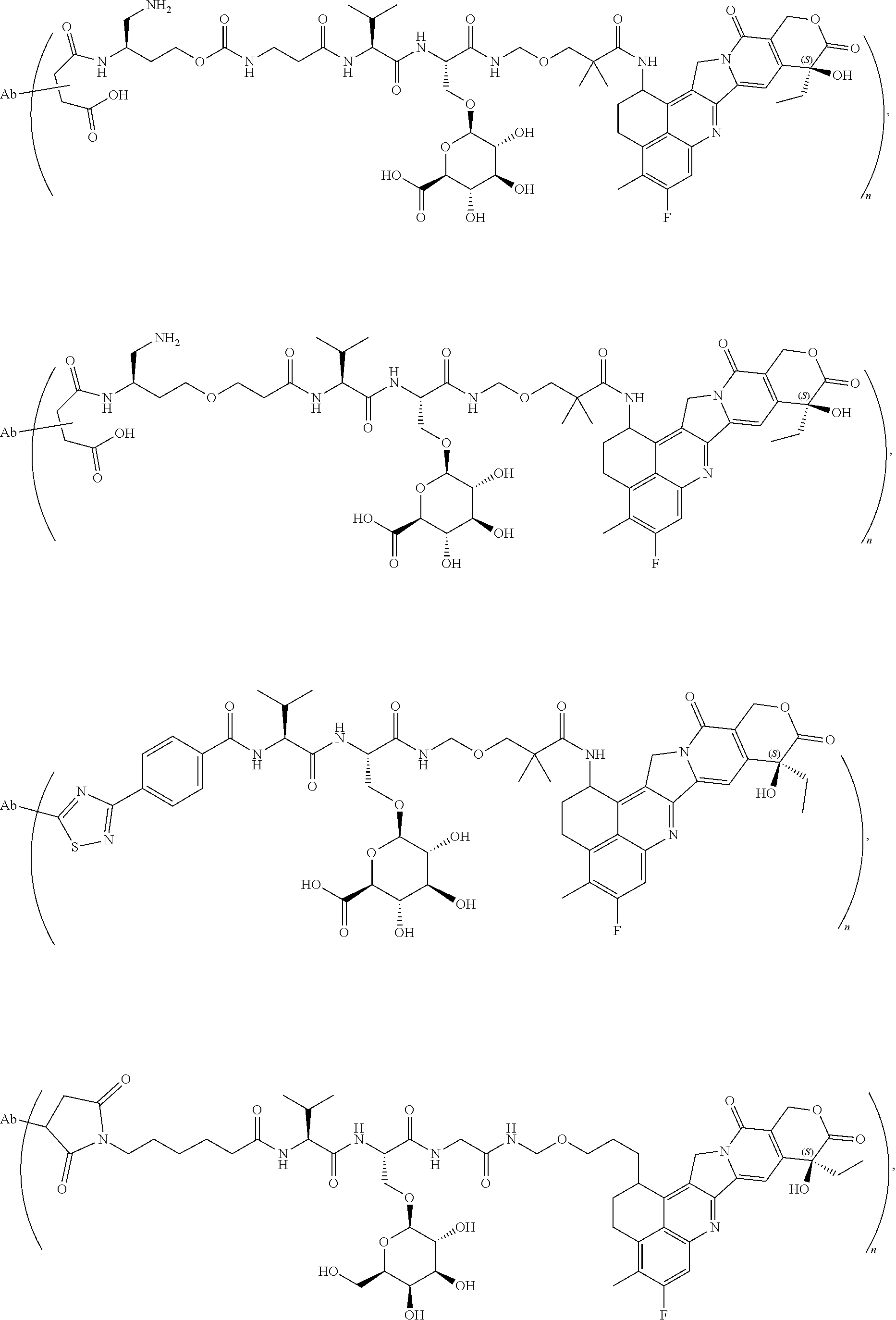

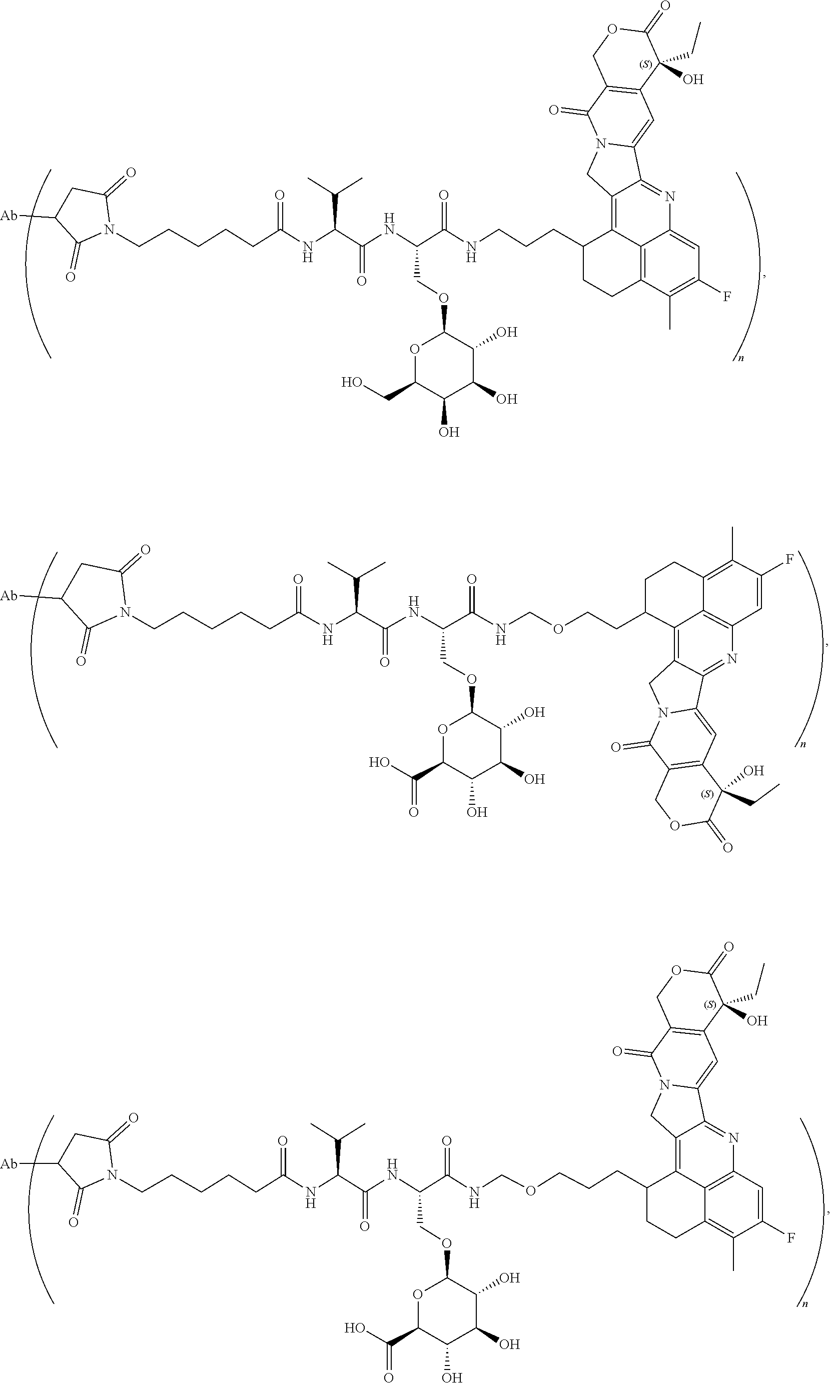

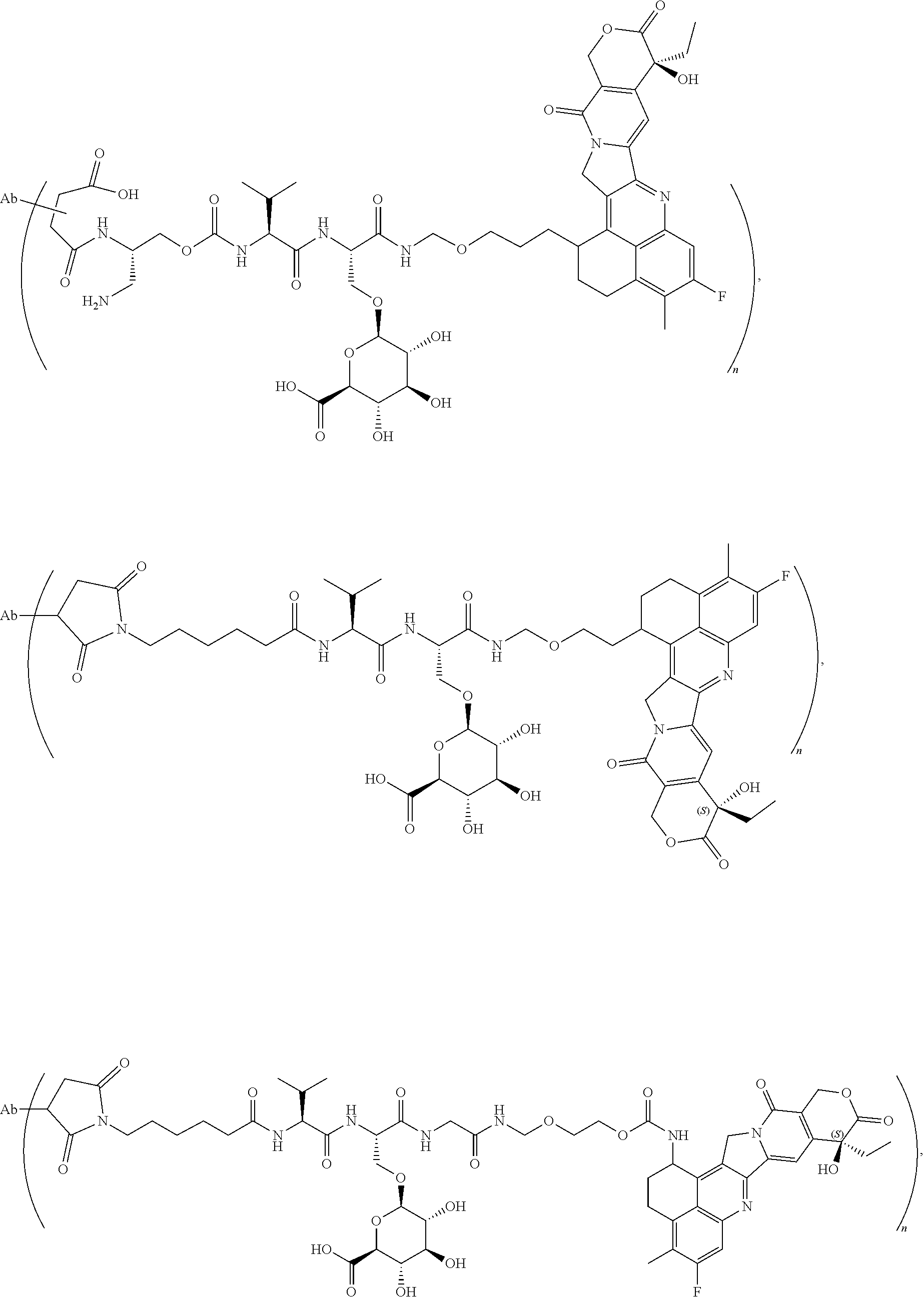

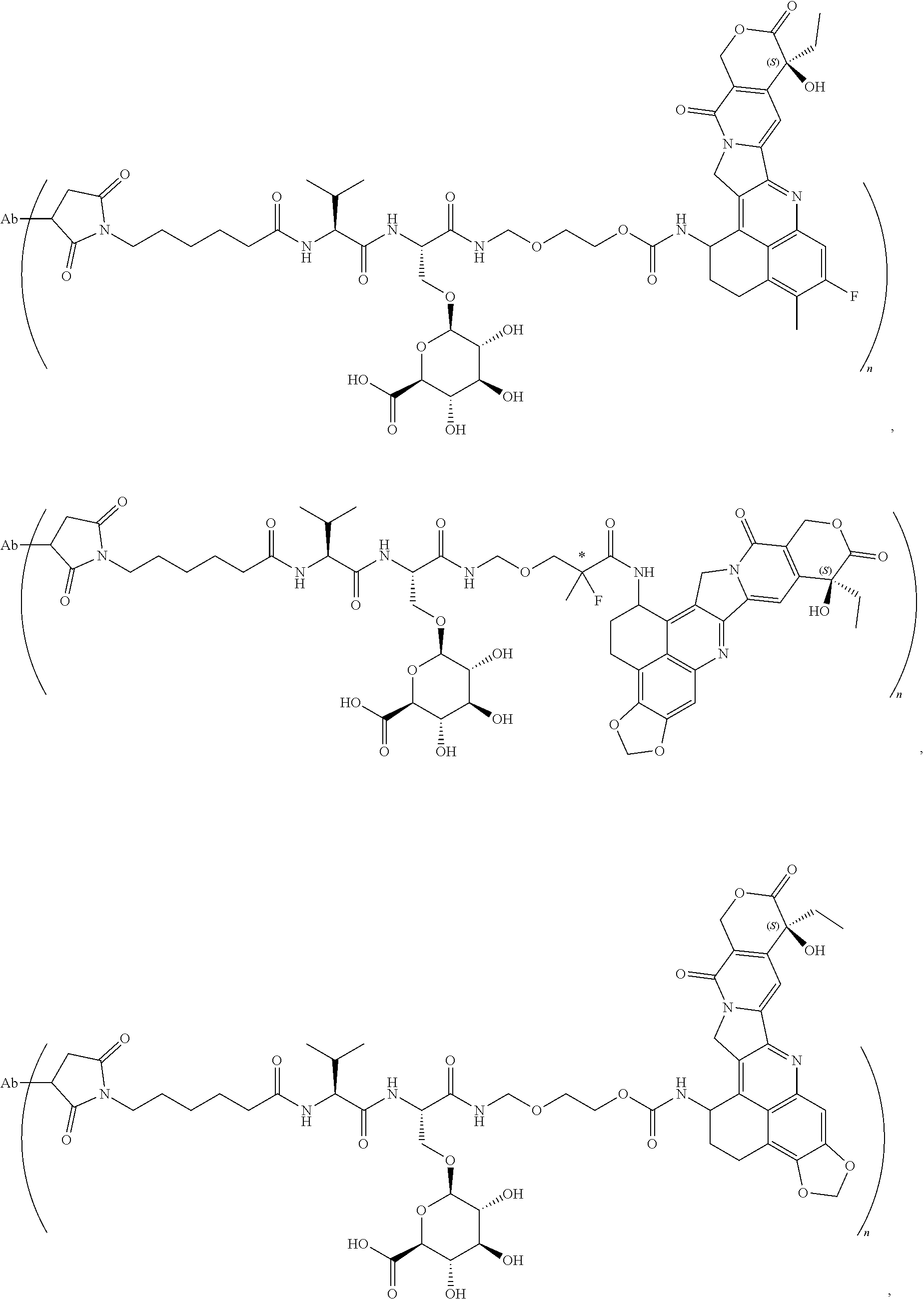

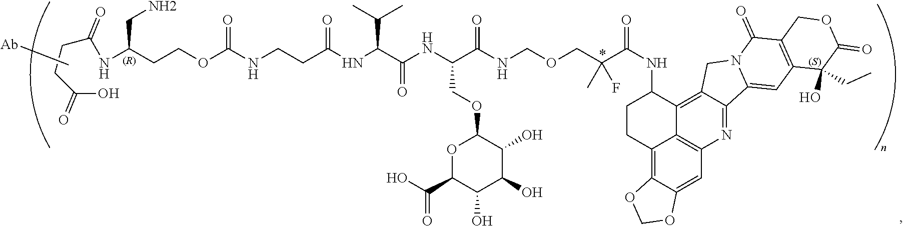

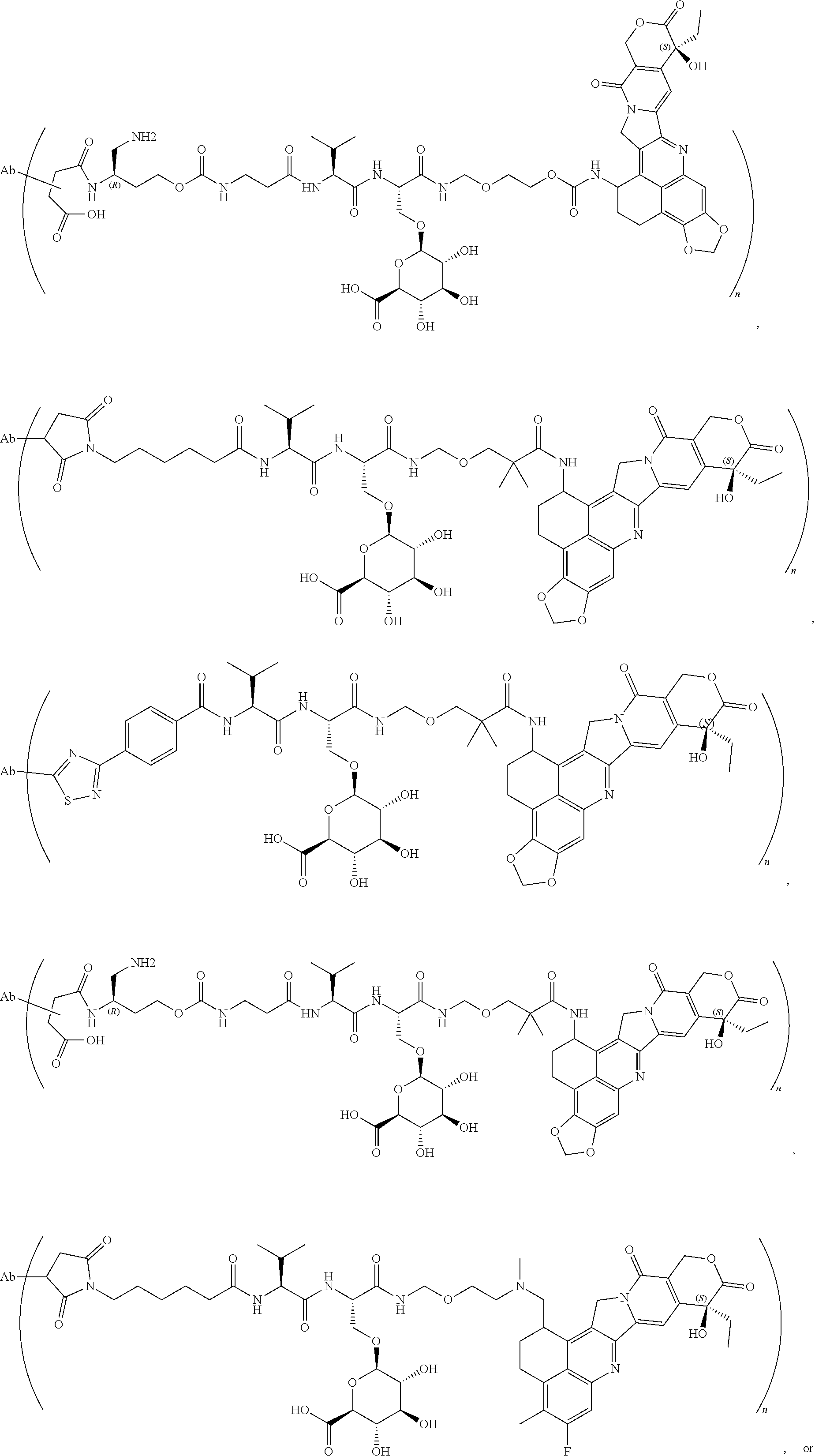

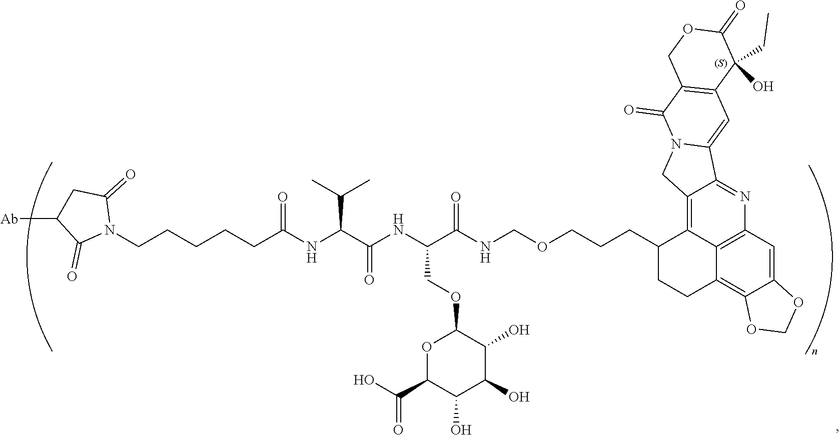

wherein Ab is a humanized, chimeric, or human antibody or an antigen binding fragment thereof, and subscript n is from 1 to 15. Alternative values for variable subscript n are as set forth herein, e.g., with respect to compounds of Formulas (II) and (Ila).

In some embodiments, Ab is ifinatamab. Alternative values for Ab are as set forth herein, e.g., with respect to Binding Agents, Including Antibodies.

Antibody drug conjugates disclosed herein may be produced by any method known in the art. In one embodiment, a host cell that has been transformed by an isolated nucleic acid comprising a sequence encoding an antibody or antigen-binding fragment thereof is cultured under suitable culturing conditions. The antibody or antigen-binding fragment thereof is thereby expressed and may be recovered from the cell culture. A payload, such as a payload described herein, is conjugated to the antibody or antigen-binding fragment thereof using a linker, such as a linker described herein, to produce the antibody drug conjugate. In some embodiments, an ADC platform, such as a platform described herein, is conjugated to the antibody or antigen-binding fragment thereof to produce the antibody drug conjugate.

Specific methods of producing antibody drug conjugates of the present disclosure are described in the Examples.

### Compositions and Formulations

Also provided herein are compositions, including pharmaceutical compositions, comprising an ADC set forth herein. In some embodiments, the compositions (e.g., pharmaceutical compositions) further comprise a pharmaceutically acceptable excipient.

Pharmaceutical compositions in accordance with the present disclosure can be prepared by mixing an antibody drug conjugate having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to, buffers such as phosphate, citrate, and other organic acids; antioxidants including ascorbic acid and methionine; preservatives (such as octadecyl dimethyl benzyl ammonium chloride; hexamethonium chloride: benzalkonium chloride: benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized formulations are described in U.S. Pat. No. 6,267,958. Aqueous formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody drug conjugate, which matrices are in the form of shaped articles, e.g. films, or microcapsules.

The formulations to be used for in vivo administration are generally sterile. Sterility can be readily accomplished, e.g., by filtration through sterile filtration membranes.

### Methods of Using ADCs

In some embodiments, set forth herein is a method of treating a cancer in a subject (e.g., patient) in need thereof, comprising administering to the subject an effective amount of an ADC set forth herein, or a pharmaceutical composition thereof.

The antibody drug conjugates disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injection, such as intravenous or subcutaneous injection, depending in part on whether the administration is brief or chronic. Various dosing schedules, including but not limited to, single or multiple administrations over various time-points, bolus administration, and pulse infusion are contemplated herein.

Antibody drug conjugates of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

### EXAMPLES

### List of Abbreviations

| | |
|---|---|
| DCM | Dichloromethane |
| DIPEA/DIEA | N,N-Diisopropylethylamine |
| DIAD | Diisopropyl azodicarboxylate |
| DMAP | 4-Dimethylaminopyridine |
| DMF | N,N-Dimethylformamide |
| DSC | N,N′-Disuccinimidyl carbonate |
| EtOAc | Ethyl acetate |
| EtOH | Ethanol |
| FA | Formic acid |
| HATU | O-(7-Azabenzotriazol-1-yl)-N,N,N′,N′- |
| | tetraMethyluronium hexafluorophosphate |
| HOAc | Acetic acid |
| HOBt | 1-Hydroxybenzotriazole |
| IPA | Isopropyl alcohol |
| LCMS | Liquid chromatography mass spectrometry |
| LDA | Lithium diisopropylamide |
| m-CPBA | m-Chloroperbenzoic Acid |
| MeCN/ACN | Acetonitrile |
| MeOH | Methanol |
| MsOH | Methanesulfonic acid |
| MTBE | Methyl tert-butyl ether |
| NPC | 4-nitrophenyl chloroformate |
| PPTS | Pyridinium p-toluenesulfonate |
| Prep-HPLC | Preparative high performance liquid chromatography |
| r.t. | Room temperature |
| TBAB | Tetrabutylammonium bromide |
| TBAF | Tetrabutylammonium fluoride |
| TBSCl | tert-Butyldimethylsilyl chloride |
| TEA | Triethyl amine |
| TFA | Trifluoroacetic acid |
| THF | Tetrahydrofuran |
| TLC | thin-layer chromatography |
| Tol | toluene |
| TSTU | N,N,N′,N′-Tetramethyl-O-(N-succinimidyl)uronium |
| | tetrafluoroborate |
| UPLC | Ultra-performance liquid chromatography |

### HPLC Analysis Methods

Method A: Mobile phase A: 0.1% FA in water, B: MeCN; Gradient: 10% B maintain 0.2 min, 10%-95% B, 5.8 min, 95% kB maintain 0.5 min; Flow rate: 0.6 mL/min; Column: ACQUITY UPLC® BEH C18 1.7 μm.

Method B: Mobile phase A: 0.1% FA in water. B: MeCN; Gradient. 10% B maintain 0.5 min, 10%-90% B, 2.5 min, 90% B maintain 0.2 min; Flow rate: 0.6 mL/min; Column: ACQUITY UPLC® BEH C18 1.7 μm.

Method C: Mobile phase A: 0.1% FA in water, B: MeCN; Gradient: 10% B maintain 0.2 min, 10%-90% B, 1.3 min, 90% B maintain 0.3 min; Flow rate: 0.6 mL/min; Column: ACQUITY UPLC® BEH C18 1.7 μm.

### Example 1

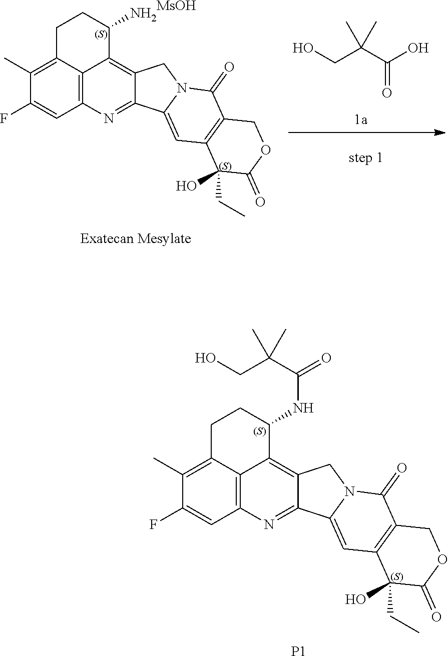

Step 1: N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxy-2,2-dimethylpropanamide (P1). To a mixture of 1a (5 mg, 0.042 mmol) and HATU (16 mg, 0.042 mmol) in DMF (1 mL) were added DIPEA (21 μL, 16 mg, 0.13 mmol) and exatecan mesylate (23 mg, 0.043 mmol). The resulting brown mixture was stirred at r.t. for 2 h. On completion of the reaction, the mixture was purified by prep-HPLC (TFA) (Method: column: XBridge Prep C18 OBD 5 μm 19*150 mm; Mobile phase: A-water (0.1% TFA): B-acetonitrile; Flow rate: 20 mL/min), and the fraction was lyophilized to give P1 (15 mg, 65.5% yield) as a white powder. ¹H NMR (400 MHz, DMSO-d6): (δ 8.00 (d, J=8.4 Hz, 1H), 7.79 (d, J=11.2 Hz, 1H), 7.31 (s, 1H), 6.52 (s, 1H), 5.59-5.54 (m, 1H), 5.42 (s, 2H), 5.18 (q, J=19.2 Hz, 2H), 4.87 (t, J=5.2 Hz, 1H), 3.45 (dd, J=10.2, 4.8 Hz, 1H), 3.41-3.28 (m, 1H), 3.15 (t, J=5.6 Hz, 2H), 2.40 (s, 3H), 2.24-2.07 (m, 2H), 1.92-1.80 (m, 2H), 1.11 (d, J=7.6 Hz, 6H), 0.87 (t, J=7.2 Hz, 3H). MS (ESI) m/z: 536.4 [M+H]⁺.

### Example 2

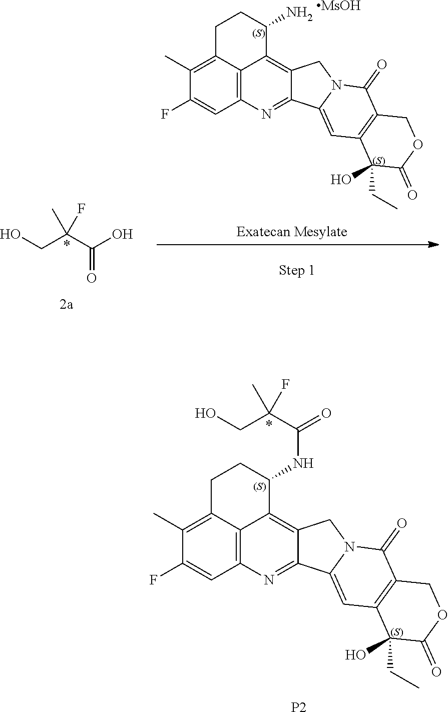

Step 1: N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-fluoro-3-hydroxy-2-methylpropanamide (P2). To a solution of compound 2a (23 mg, 0.19 mmol) in DMF (2 mL) were added exatecan mesylate (50 mg, 0.094 mmol), HATU (54 mg, 141 mmol), and DIEA (36 mg, 0.28 mmol). The mixture was stirred at r.t. for 1 h. The mixture was purified by prep-HPLC (FA) (Method: column: XBridge Prep C18 OBD 5 μm 19*150 mm; Mobile phase: A-water (0.1% formic acid): B-acetonitrile; Flow rate: 20 mL/min), and the fraction was lyophilized to give P2 as a white solid (8.4 mg, 16.6% yield). UPLC-MS, RT=3.86 min. ¹H NMR (400 MHz, DMSO-d₆) δ 8.72 (dd, J:=8.4, 2.4 Hz, 1H1), 7.78 (d, J=11.2 Hz, 111), 7.31 (s, 1H), 6.52 (s, 1H), 5.58 (d, J=8.0 Hz, 1H), 5.42 (s, 2H), 5.32-5.05 (m, 3H), 3.83 (dd, J=26.8, 12.0 Hz, 1H), 3.61 (dd, J=21.6, 12.0 Hz, 1H), 3.22-3.07 (m, 2H), 2.46-2.30 (m, 3H), 2.28-2.05 (n, 211), 2.02-1.74 (m, 2H), 1.45 (d, J=21.4 Hz, 3H), 0.87 (t, J=7.2 Hz, 3H). MS (ESI) m/z: 540.3 [M+H]⁺.

### Example 3

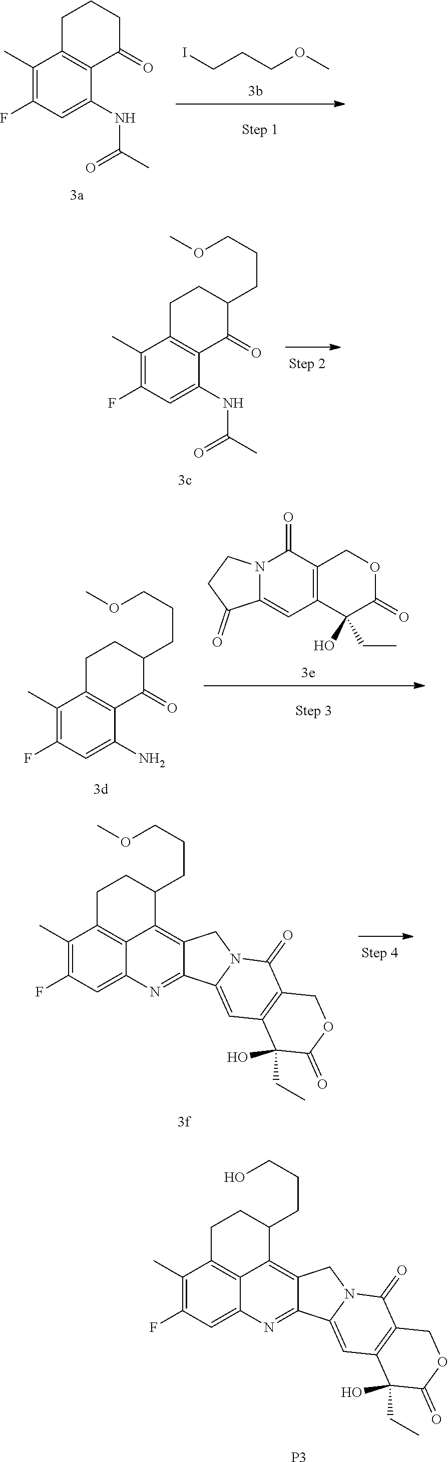

Step 1: N-(3-fluoro-7-(3-methoxypropyl)-4-methyl-8-oxo-5,6,7,8-tetrahydronaphthalen-1-yl)acetamide (3c). To a solution of LDA (1.40 mL, 2.81 mmol, 2M in THF) in THF (19 mL) was added 3a (300.00 mg, 1.28 mmol) at −78 °C, and the mixture was stirred at the same temperature for 2 h before a solution of 3b (0.38 g, 1.91 mmol) in THF (1 mL) was added dropwise. The reaction mixture was then slowly warmed up to 0 °C and stirred continuously for 4 h. The reaction was subsequently quenched with saturated aqueous NH₄Cl solution (50 mL), and the organic materials were extracted with EtOAc (30 mL*3) The combined organic layers were washed with brine (50 mL) and dried over MgSO₄ before the combined extracts were concentrated in vacuo. The resulting crude residue was purified by flash column chromatography (silica gel, Hexane:EtOAc=97:3) to give 3c (80.00 mg, 20.41% yield) as a colorless oil. MS (ESI) m/z: 308.2 [M+H]⁺.

Step 2: 8-amino-6-fluoro-2-(3-methoxypropyl)-5-methyl-3,4-dihydronaphthalen-1(2H)-one (3d). Compound 3d (69.06 mg, crude) was synthesized according to synthetic procedure of Example 5-1 in EP3677568A1. MS (ESI) m/z: 266.2 [M+H]⁺.

Step 3: (9S)-9-ethyl-5-fluoro-9-hydroxy-1-(3-methoxypropyl)-4-methyl-1,2,3,9,12,15-hexahydro-10H,13H-benzo[de]pyrano[3′,4′:6,7]indolizino[1,2-b]quinoline-10,13-dione (31). Compound 3f (80.00 mg, 62.40% yield) was synthesized according to synthetic procedure of Example 6-1 in EP3677568A1. MS (ESI) m/z: 4931 [M+H]⁺.

Step 4: (9S)-9-ethyl-5-fluoro-9-hydroxy-1-(3-hydroxypropyl)-4-methyl-1,2,3,9,12,15-hexahydro-10H,13H-benzo[de]pyrano[3′,4′:6,7]indolizino[1,2-b]quinoline-10,13-dione (P3). To a solution of 3f (70.00 mg, 0.14 mmol) in CH₂Cl₂ (20 mL) was added BBr₃ (71.26 mg, 0.28 mmol) at −0 °C, and the mixture was stirred at the same temperature for 1 h. The reaction mixture was then slowly warmed up to 25 °C and stirred continuously for 3 h. The reaction was subsequently quenched with saturated aqueous NaH—CO₃ solution and the organic materials were extracted thrice with EtOAc (30 mL*3). The combined organic layers were washed with brine (50 mL) and dried over MgSO₄ before the combined extracts were concentrated in vacuo. The resulting crude residue was purified by flash column chromatography (silica gel, CH₂Cl₂: MeOH=90:10) to give mixture P3 (4.20 mg, 6.18% yield) as a gray solid. MS (ESI) m/z: 479.4 [M+H]⁺.

UPLC analysis: P3-1, peak 1, retention time=4.49 min; P3-2, peak 2, retention time=4.65 min (Mobile phase A: 0.1% FA in water, B: MeCN; Gradient: 15% B maintain 1 min, 15%-95% B, 9 min, 95% B maintain 2 min; Flow rate: 0.6 mL/min; Column: ACQUITY UPLC BEH C18 1.7 μm).

P3-1: ¹H NMR (400 MHz, DMSO-d6) δ 7.74 (d, J=11.1 Hz, 1H), 7.30 (s, 1H), 6.51 (s, 1H), 5.43 (s, 2H), 5.36 (d, J=18.7 Hz, 1H), 5.22 (d, J=18.7 Hz, 1H), 3.52-3.39 (m, 2H), 3.18-2.97 (m, 2H), 2.38 (s, 3H), 2.34-2.25 (m, 2H), 2.01-1.78 (m, 3H), 1.78-1.51 (m, 4H), 0.87 (t, J=7.3 Hz, 3H).

P3-2: ¹H NMR (400 MHz, DMSO-d6) δ 7.74 (d, J=11.1 Hz, 1H), 7.30 (s, 1H), 6.52 (s, 1H), 5.43 (s, 2H), 5.37 (d, J=18.8 Hz, 1H) 5.23 (d, J=18.7 Hz, 1H), 351-3.40 (m, 2H), 3.16-2.98 (m, 2H), 2.38 (s, 3H), 2.35-2.24 (m, 2H), 1.99-1.81 (m, 3H), 1.78-1.53 (in 4H), 0.87 (t, J=7.3 Hz, 311).

### Example 4

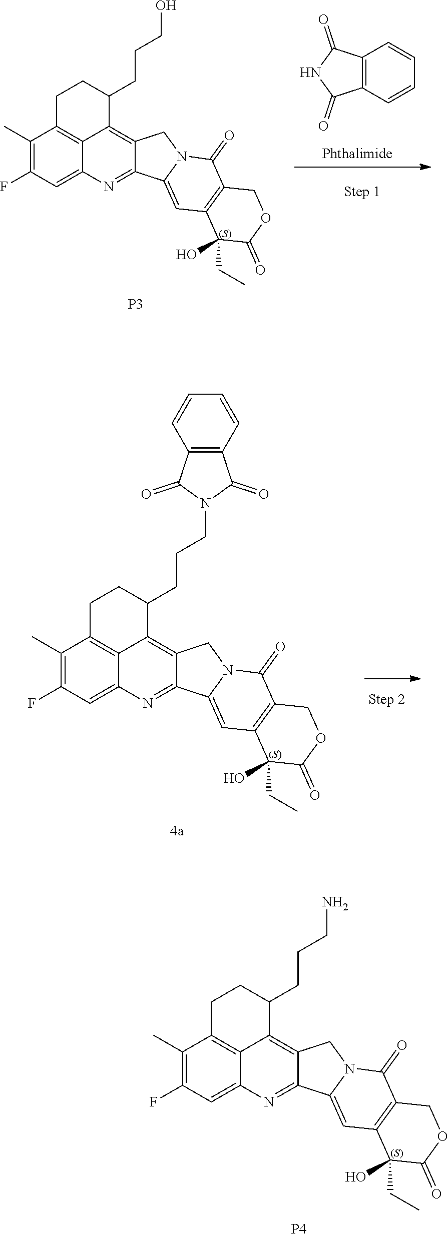

Step 1: (9S)-1-(3-(1,3-dioxoisoindolin-2-yl)propyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (4a). A mixture of PPh₃ (49.3 mg, 0.188 mmol) and DIAD (38.0 mg, 0.188 mmol) in THF (1 mL) was stirred at r.t. for 5 min and then added to a mixture of compound P3 (60 mg, 0.125 mmol) and phthalimide (20.3 mg, 0.138 mmol) in THF (1 mL). The resulting yellow mixture was stirred at r.t. for 1 h. The reaction was quenched by addition of water (10 mL), and extracted with EtOAc (10 mL*2). The combined organic layer was washed with brine (10 mL), dried over Na₂SO₄, filtered, and the filtrate was concentrated under vacuum to give a residue. It was purified by silica gel chromatography (SiO₂ 5 g, EtOAc/Petroleum ether=20%70%), and the fraction was concentrated under vacuum to give a crude product (130 mg, crude) as a yellow solid. MS (ESI) m/z: 608.5 [M+H]⁺.

Step 2: (9S)-1-(3-aminopropyl)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (P4) formate. To a solution of 4a (130 mg, crude) in EtOH (2 mL) was added N₂H₄·H₂O (80%, 20 μL). The mixture was stirred at 60 °C for 60 min. The mixture was acidified with 3N HCl to pH=2 then purified by prep-HPLC (FA). Fraction was lyophilized to give target product P4 (12.2 mg, 99.68% purity) as a white solid. MS (ESI) rm/z: 478.4 [M+H]⁺. ¹H NMR (400 MHz, DMSO-d6) δ 7.75 (d, J=10.8 Hz, 1H), 7.31 (s, 1H), 5.44 (s, 2H), 5.34 (q, J=18.8 Hz, 3H), 3.10 (m, 1H), 2.76 (s, 2H), 2.38 (s, 3H), 2.27 (d, J=12.8 Hz, 2H), 1.91 (m, 6H), 1.62 (m, 3H), 0.87 (t, J=7.4 Hz, 4H).

### Example 5

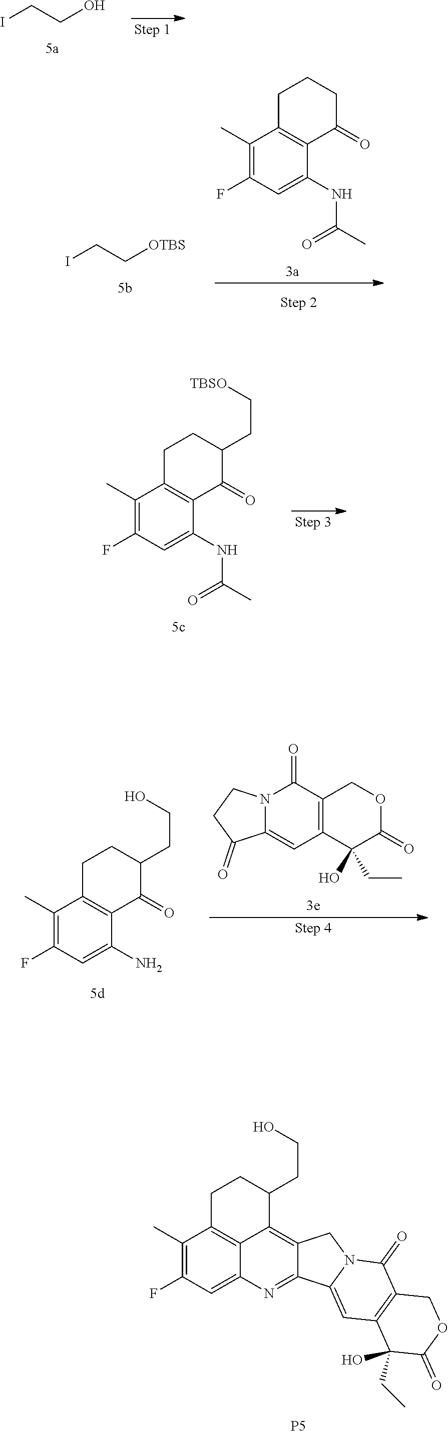

Step 1: tert-butyl(2-iodoethoxy)dimethylsilane (5b). A solution of compound 5a (3000 mg, 17.44 mmol) in CH₂Cl₂ (40 mL) was cooled to 0 °C, then imidazole (1779 mg, 26.16 mmol) and TBSCl (3139 mg, 20.93 mmol) was added portionwise at 0 °C. The mixture was stirred at 20 °C for 16 h. TLC (SiO₂, Petroleum ether) showed the reaction was completed. The reaction was poured into water (40 mL) and extracted with CH₂Cl₂ (50 mL*3). The combined organic layer was dried over Na₂SO₄ and concentrated to give the residue which was purified by silica column gel chromatography (eluent: Petroleum ether=100) to afford 5b (3.5 g, 70.2% yield) as a colorless oil.

Step 2: N-(7-(2-((tert-butyldimethylsilyl)oxy)ethyl)-3-fluoro-4-methyl-8-oxo-5,6,7,8-tetrahydronaphthalen-1-yl)acetamide (5c). To a solution of compound 3a (2350 mg, 10 mmol) in THF (40 mL) was added KO^{t}Bu (IM in THF) (22 mL, 22 mmol) at 0 °C dropwise and the internal temperature was maintained at 0 °C. The mixture was stirred at 0 °C for 30 min, then 5b (3432 mg, 12 mmol) was added slowly. LCMS showed the reaction was completed. 1N HCl was added to the mixture dropwise to adjust pH to 2. The mixture was poured into saturated NaHCO₃ aq. (50 mL) and was extracted with EtOAc (60 mL*3). The combined organic layer was dried over anhydrous Na₂SO₄, filtered and concentrated to give the crude product which was purified by silica column gel chromatography (eluent: Petroleum ether/EtOAc=100/0 to 83/17) to afford 5c (700 mg, 17.8% yield) as a yellow solid. MS (ESI) m/z: 394.2 [M+H]⁺. ¹H NMR (400 MHz, DMSO-d6) δ=7.40 (s, 2H), 6.34 (d, J=12.6, 1H), 6.34 (d, J=12.6, 1H), 4.45 (t, J=5.2, 1H4), 4.45 (t, J=5.2, 1H), 3.65-3.35 (m, 2H), 3.55-3.45 (m, 2H1), 2.83 (s, 1H), 2.83 (s, 1H), 2.72-2.61 (m, 1H), 2.46 (d, J=5.0, 1H), 2.11-1.99 (m, 3H), 1.97 (d, J=1.0, 3H), 1.72-1.60 (m, 1H), 1.43 (d, J=6.2, 1H).

Step 3: 8-amino-6-fluoro-2-(2-hydroxyethyl)-5-methyl-3,4-dihydronaphthalen-1(2H)-one (5d). To a solution of compound 5c (700 mg, 1.78 mmol) in MeOH (3 mL) was added 2 N HCl (3 mL). The mixture was stirred at 60 °C for 16 h. LCMS showed the reaction was completed. The mixture was poured into aqueous saturated NaHCO₃ (20 mL) and was extracted with EtOAc (30 mL*3). The combined organic layer was dried over anhydrous Na₂SO₄, filtered and concentrated to give the crude product which was purified by silica column gel chromatography (eluent: Petroleum ether/EtOAc=100/0 to 76/24) to afford 5d (200 mg, 47% yield) as an off-white solid. MS (ESI) m/z: 238.2 [M+H]⁺.

Step 4: (9S)-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (P5). To a solution of compound 5d (100 mg, 0.42 mmol) in Toluene (6 mL) and o-cresol (0.35 mL) was added 3e (110 mg, 0.42 mmol) and PPTS (16 mg, 0.06 mmol). The mixture was refluxed at 140 °C for 5 h. L CMS showed the reaction was completed. Solvent was evaporated and the residue was purified by silica column gel chromatography (eluent: CH₂Cl₂/MeOH=100/0 to 92/8) to afford P5 (80 mg, 41% yield) as an orange solid. MS (ESI) m/z: 465.2 [M+H]⁺. ¹H NMR (400 MHz, DMSO-d6) δ=7.74 (d, J=11.0, 1H), 7.31 (s, 1H), 6.53 (d, J=1.9, 1H), 5.44 (s, 21H), 5.30 (s, 21H), 4.77 (t, J=4.8, 1H), 3.69-3.56 (m, 4H), 3.10 (t, J=18.6, 2H), 2.38 (s, 31H), 2.29 (d, J=9.7, 1H), 1.99-1.81 (m, 3H), 1.73 (d, J=5.4, 2H), 0.93-0.81 (m, 3H).

P5 (22 mg) was separated by SFC (Mobile Phase A: CO₂, Mobile Phase B: MeOH, Gradient: 5%-40% B for 5 min, 40% maintain for 2 min; Flow rate: 2 mL/min; Column: Daicel_ChiralPAK-IBN_100×3.0 mm_3 μm; Column temperature: 35 °C) to give P5-1 (6 mg, peak 1, retention time=4.806 min) and P5-2 (8.8 mg, peak 2, retention time=5.363 min), which are isomers at the ethanol substituent.

### Example 6

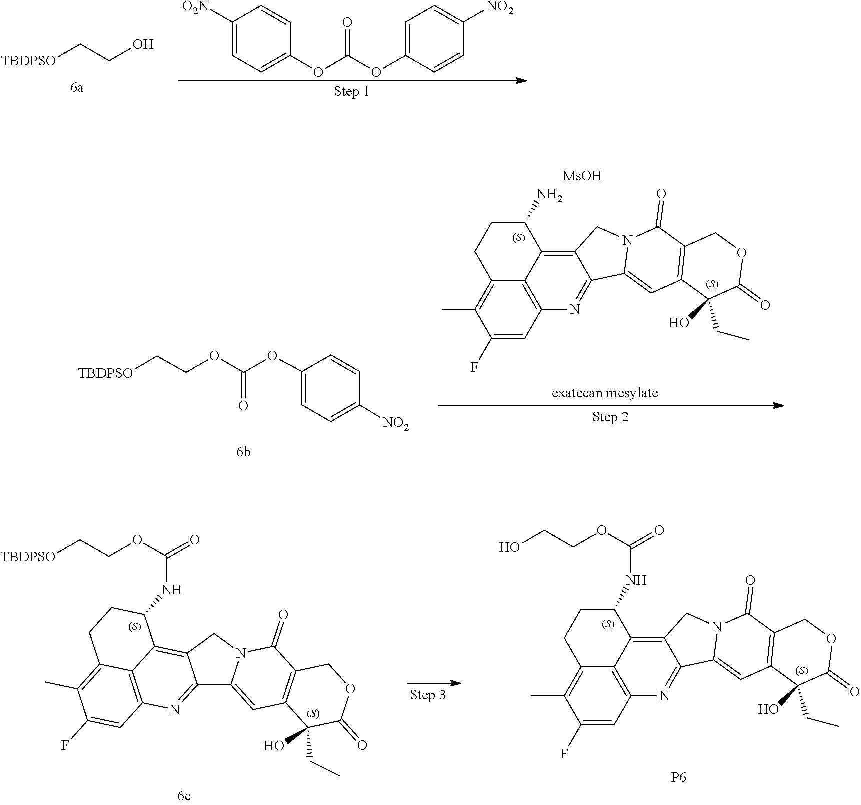

Step 1: 2-((tert-butyldiphenylsilyl)oxy)ethyl (4-nitrophenyl) carbonate (6b). To a solution of 6a (100 mg, 0.33 mmol) and bis(4-nitrophenyl) carbonate (123 mg, 0.40 nmol) in dry CH₂Cl₂(2 mL) was added DIEA (176 μL, 1.0 mmol) and DMAP (4.1 mg, 0.033 mmol), stirred at r.t. overnight. The solution was poured into TN HCl (2 mL), extracted with CH₂Cl₂(2 mL*3). The organic phase was concentrated and purified by flash column chromatography (silica gel, petroleum ether/ethyl acetate=5:1) to give compound 6b (148 mg, 95.5% yield) as a colorless oil. ¹H NMR (400 MHz, CDCl₃) δ 8.27 (d, J=9.1 Hz, 2H), 7.69 (d, J=6.5 Hz, 4H), 7.50-7.31 (m, 8H), 4.47-4.36 (m, 2H), 3.99-3.89 (m, 2H), 1.07 (s, 9H).

Step 2: 2-((tert-butyldiphenylsilyl)oxy)ethyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (6c). To a solution of exatecan mesylate (50 mg, 0.094 mmol), 6b (53 mg, 0.11 mmol) and HOBt (1.3 mg, 0.009 mmol) in dry DMF (1 mL) was added DIEA (50 μL, 0.28 mmol), and stirred at r.t. overnight. The solution was poured into sat. NH₄Cl (5 mL), extracted with EtOAc (5 mL*3). The organic phase was concentrated and purified by flash column chromatography (silica gel, petroleum ether/ethyl acetate=1:3) to give compound 6c (68 mg, 94.9% yield) as a yellow solid. MS (ESI) m/z: 762.4 [M+H]⁺. ¹H NMR (400 MHz, CDCl₃) δ 7.70-7.60 (m, 51), 7.59-7.52 (m, 1H), 7.36-7.27 (m, 7H), 5.68 (d, J=16.2 Hz, 1H), 5.33 (d, J=16.3 Hz, 1H), 5.25-5.15 (m, 1H), 5.09-4.96 (m, 2H), 4.56-4.44 (m, 1H), 4.33-4.21 (m, 1H), 4.04-3.89 (m, 2H), 3.70 (s, 1H), 3.17-2.99 (m, 2H), 2.47-2.34 (m, 4H), 2.19-2.07 (m, 1H), 2.05-1.88 (m, 2H), 1.08 (t, J=73 Hz, 3H), 1.04 (s, 9H).

Step 3: 2-hydroxyethyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (P6). To the solution of 6c (65 mg, 0.085 mmol) in dry THF (2 mL) was added 1M TBAF in THF (102 μL, 0.102 mmol) at 0 °C, and stirred at r.t. for 40 min. The solution was poured into sat. NH C₁ (5 mL), extracted with CH₂Cl₂/MeOH (5/1, 6 mL*3). The organic phase was concentrated and purified by flash column chromatography (silica gel, CH₂Cl₂/MeOH=10:1) to give compound P6 (39 mg, 87.3% yield) as a pale yellow solid. MS (ESI) m/z: 524.4 [M+H]⁺. ¹H NMR (400 MHz, DMSO-d₆) δ 7.98 (d, J=8.8 Hz, 1H), 7.77 (d, J=10.9 Hz, 1H), 7.31 (s, 1H), 6.52 (s, TH), 5.43 (s, 2H), 5.33-5.17 (m, 3H), 4.78 (t, J=5.4 Hz, 1H), 4.19-4.01 (m, 2H), 3.68-3.57 (m, 2H), 3.30-3.20 (m, 1H), 3.17-3.06 (m, 1H), 2.38 (s, 3H), 2.26-2.07 (m, 2H), 1.95-1.78 (m, 2H), 0.87 (t, J=7.4 Hz, 3H).

### Example 7

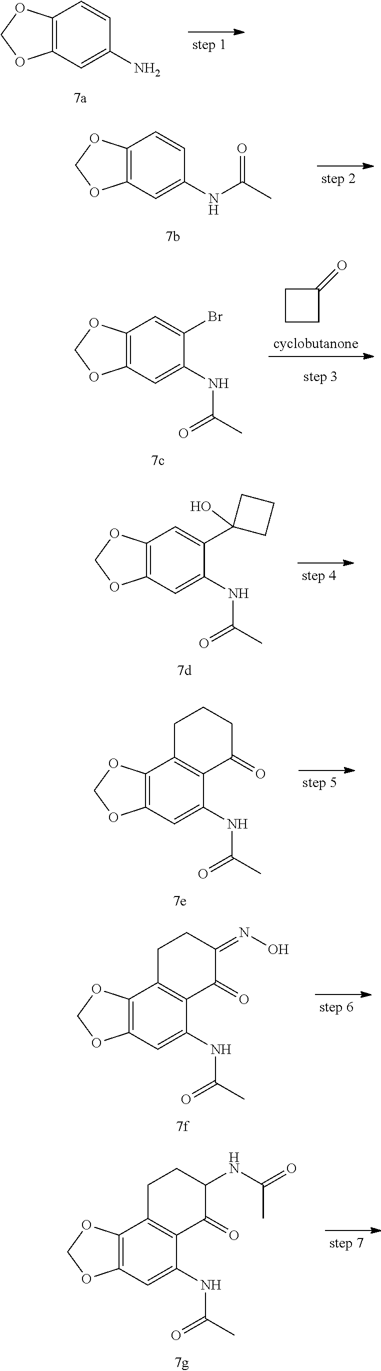

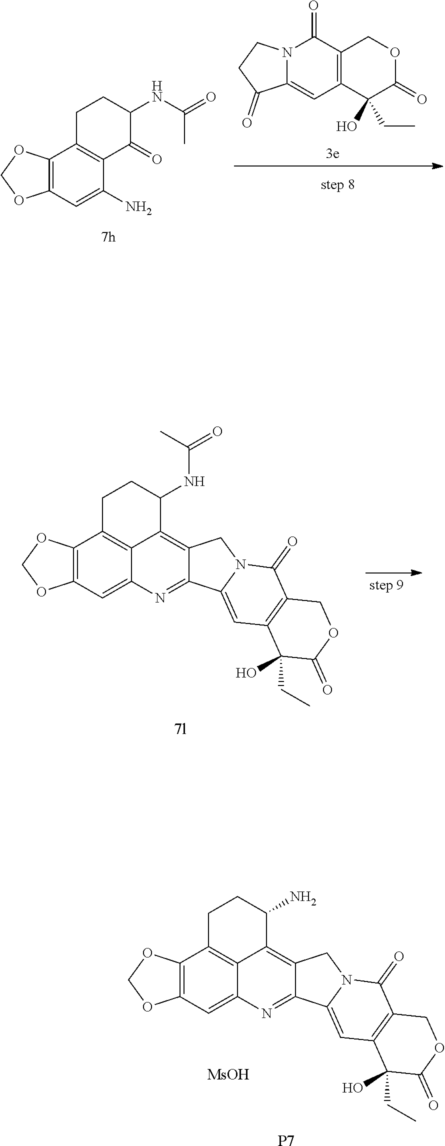

Step 1: N-(benzo[d][1,3]dioxol-5-yl)acetamide (7b). To a solution of 7a (25.00 g, 182 mmol) in CH₂Cl₂ (200 mL) was added Ac₂O (27.85 g. 273 mmol) and Et₃N (36.76 g, 364 mmol) at 0 °C. The reaction mixture was reacted at 20 °C for 3 h. LCMS showed the reaction was completed. The reaction mixture was concentrated to 50 mL. To the residual was added EtOAc (200 mL) and washed with saturated NaHCO₃ (200 mL*3). The organic layer was dried over Na₂SO₄ and filtered. The filtrate was concentrated under vacuum and the residue was used in the next step without further workup and purification (28.25 g, crude). MS (ESI) m/z: 180.1 [M+H]⁺.

Step 2: N-(6-bromobenzo[d][1,3]dioxol-5-yl)acetamide (7c). A solution of 7b (28.25 g, crude) and sodium acetate (15.4 g, 188.3 mmol) in acetic acid (100 mL) was heated to 60 °C, then a mixture of bromine (30.1 g, 188.3 mmol) and acetic acid (60 mL) was added to the reaction solution dropwise. The reaction temperature was raised to 80 °C and stirred at 80 °C for 2 h. LCMS showed the reaction was completed, the reaction solution was poured into ice water, and a yellow solid was generated. The solid was filtered and washed with water three times to obtain a crude product which was recrystallized with EtOH to afford 7c (17.1 g, 42% yield). MS (EST) m/z: 258.1/260.1 [M+H]⁺. 1H NMR (400 MHz, DMSO) δ 9.36 (s, 1H), 7.22 (s, 1H), 7.08 (d, J=3.7 Hz, 1H), 6.07 (s, 2H), 2.02 (s, 3H).

Step 3: N-(6-(1-hydroxycyclobutyl)benzo[d][1,3]dioxol-5-yl)acetamide (7d). A solution of compound 7c (8.5 g, 33.01 mmol) in THF (200 ml) was cooled to −90 °C, n-BuLi (15.84 mL, 39.60 mmol) was added into the mixture over 2 h under N₂ protection. The internal temperature was kept under −85 °C. The mixture was stirred at −85 °C for 20 min. A solution of cyclobutanone (2.7 g, 39.60 mmol) in TIF (50 mL) was added into the mixture dropwise. The mixture was stirred at −85 °C for 30 min and warmed to room temperature for 1 h. The mixture was quenched by sat. NH₄Cl solution (200 mL), extracted with EtOAc (150 mL*3). The combined organic layer was dried over Na₂SO₄. After filtration and evaporation, the residue was washed with MTBE (5 mL*3) and recrystallized with EtOH to afford 7d (3.1 g, 37.5% yield). MS (ESI) m/z: 250.1 [M+H]⁺.

Step 4: N-(6-oxo-6,7,8,9-tetrahydronaphtho[1,2-d][1,3]dioxol-5-yl)acetamide (7e). To a solution of compound 7d (3.1 g, 12.44 mmol) in CH₂Cl₂ (20 mL) and H₂O (20 mL) were added AgNO₃ (12.4 mL, 6.22 mmol), K₂S₂O₈ (8.4 g, 31.1 mmol). The mixture was stirred at 20 °C for 16 h. The mixture was filtered through celite and the filtration residue was washed with CH₂Cl₂:MeOH:=1:1 (50 mL*3). The filtrate was poured into water and extracted with CH₂Cl₂ (300 mL*3). The combined organic layer was dried over Na₂SO₄. After filtration and evaporation, the residue was purified by silica column gel chromatography (eluent: Petroleum ether/CH₂Cl₂:=100/0 to 0/100) to afford 7e (1.9 g, 61.8% yield) as light-yellow solid. MS (EST) m/z: 2481 [M+H]⁺.

Step 5: (Z)—N-(7-(hydroxyimino)-6-oxo-6,7,8,9-tetrahydronaphtho[1,2-d][1,3]dioxol-5-yl)acetamide (7f). To a solution of compound 7e (1.9 g, 7.69 mmol) in THF (20 mL) and t-BuOH (5 ml) was added t-BuOK (1 M in THF, 9.28 ml, 9.28 mmol) dropwise at 0 °C, The atmosphere was purged with N₂ and the mixture was cooled to 0 °C using and ice water bath. After 5 mins, to the stirred mixture was added isopentyl nitrite (1.09 g, 9.28 mmol). LCMS showed the reaction was completed and the reaction mixture was allowed to warm to r.t. 1 N HCl was added to adjusted pH to 1. The aqueous layer was extracted with CH₂Cl₂: THF (2:1, v/v, 50 mL*3). The combined organic layer was washed with brine (100 mL) and dried over tit Na₂SO₄. After filtration and evaporation, the residue was trituration with MTBE (20 mL*2) to afford 7f (1.5 g, 70.6% yield). MS (ESI) m/z: 277.1 [M+H]⁺.

Step 6: N,N′-(6-oxo-6,7,8,9-tetrahydronaphtho[1,2-d][1,3]dioxole-5,7-diyl)diacetamide (7g). To a solution of compound 7f (1.5 g, 5.43 mmol) in Ac₂O (5 ml) was added PtO₂ (150 mg). The mixture was stirred at 20 °C under H₂ (15 psi) for 16 h. LCMS showed the reaction was completed. The mixture was filtered through a pad of celite. The filtrate was diluted with EtOAc (100 mL) and washed by sat. NaHCO₃ (100 mL*3). The organic layer was dried over Na₂SO₄. After filtration and evaporation, the residue 7g (1.3 g, crude) was used in the next step without further workup and purification. MS (EST) m/z: 305.2 [M+H]⁺.

Step 7: N-(5-amino-6-oxo-6,7,8,9-tetrahydronaphtho[1,2-d][1,3]dioxol-7-yl)acetamide (7h). To a solution of compound 7g (1.3 g, crude) in MeOH (10 mL) was added HCl (2 N, 10 mL). The mixture was stirred at 60 °C for 3 h. The mixture was cooled to r.t. and adjusted pH to 8 using sat. NaHCO₃. The mixture was extracted with EtOAc (50 mL*3). The organic layer was dried and concentrated. The residue was purified by a silica gel column chromatography (eluent: CH₂Cl₂MeOH=100/0 to 90/10) to give compound 7h (670 mg, 59.8% yield) as off-white solid. MS (ESI) m/z: 263.1 [M+H]⁺.

Step 8: N-((10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)acetamide (7i). To a solution of compound 7h (400 mg, 1.52 mmol) in toluene (40 mL) and o-cresol (0.5 mL) was added 3e (479 mg, 1.82 mmol) and PPTS (38 mg, 0.15 mmol). The mixture was refluxed at 140 °C for 24 h. LCMS showed the reaction was completed and black solid precipitated. After filtration, the filter cake was washed with acetone (10 mL*3) to give a dark brown solid 7i (580 mg, crude) which was used in the next step without further workup and purification. MS (ESI) m/z: 490.3 [M+H]⁺.

Step 9: (1S,10S)-1-amino-10-ethyl-10-hydroxy-1,2,3,10,13,16-hexahydro-11H,14H-benzo[de][1,3]dioxolo[4,5-g]pyrano[3′,4′:6,7]indolizino[1,2-b]quinoline-11,14-dione (P7). A solution of compound 7i (580 mg, crude) in MsOH (10 ml) and H₂O (10 ml) was refluxed at 110 °C for 5 h. LCMS showed the reaction was completed. The mixture was filtered and purified by prep-HPLC (Method: column: XBridge Prep C18 OBD 5 μm 19*150 mm; Mobile phase: A-water (0.1% TFA): B-acetonitrile; Flow rate: 20 mL/min, the fraction was lyophilized to give compound P7 (110 mg, 20.8% yield) as a yellow solid. MS (ESI) m/z: 448.2 [M+H]⁺. Retention time (0.82 min).

### Example 8

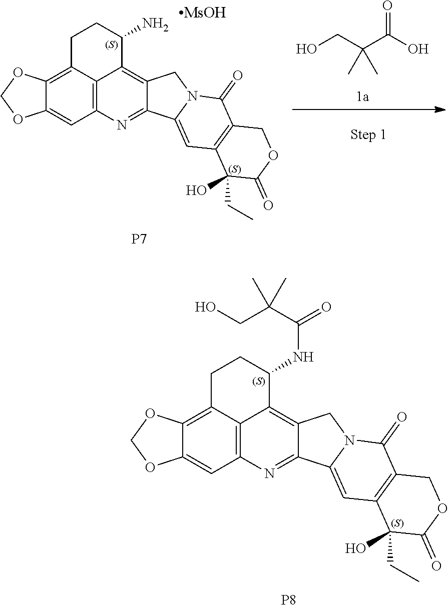

Step 1: N-((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)-3-hydroxy-2,2-dimethylpropanamide (P8). Compound P8 (1.8 mg, 17.9% yield) was synthesized according to the synthetic procedure of step 1 of Example 1. MS (ESI) m/z: 548.4 [M+1]⁺.

### Example 9

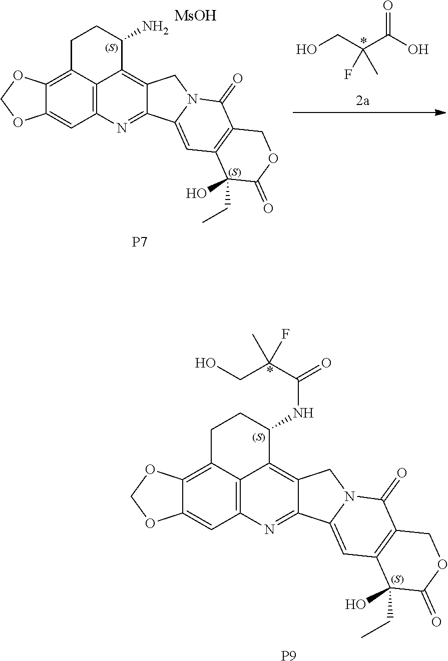

Step 1: N-((1S,10S)-10-ethyl-10-hydroxy-11,14-dioxo-2,3,10,11,14,16-hexahydro-1H,13H-benzo[de][1,3]dioxolo[4,5-g]pyrano[3′,4′:67]indolizino[1,2-b]quinolin-1-yl)-2-fluoro-3-hydroxy-2-methylpropanamide (P9). To the solution of 2a (17 mg, 0.14 mmol) and P7 (30 mg, 0.055 mmol) were added HATU (25 mg, 0.066 mmol) and DIPEA (0.02 ml, 0.11 mmol), stirred at r.t. for 10 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min), and the fraction was lyophilized to give P9 (14.5 mg, 47.6% yield) as a yellow solid. MS (ESI) m/z: 552.5 [M+H]⁺.

### Example 10

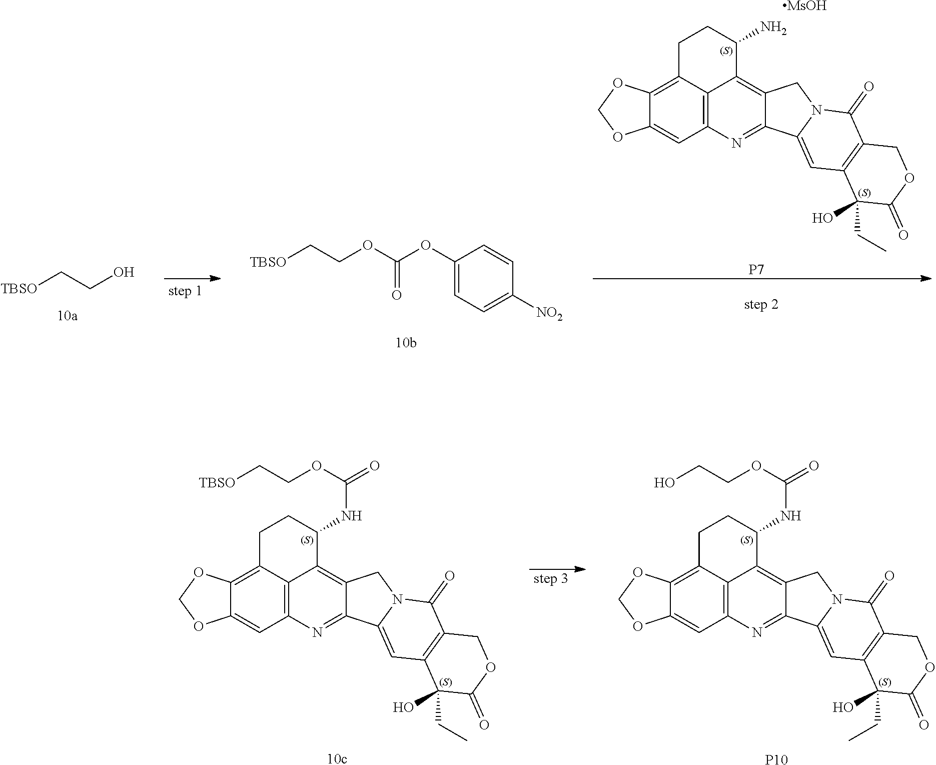

Step 1: 2-((tert-butyldimethylsilyl)oxy)ethyl (4-nitrophenyl) carbonate (10b). To a solution of 10a (1000 mg, 5.67 mmol) in dry DCM (10 mL) were added NPC (2092 mg, 6.81 mmol) and DIPEA (1.5 mL, 8.51 mmol) at 0 °C, stirred at r.t. for 1.5 h. The solution was quenched with IN HCl (5 mL), extracted with CH₂Cl₂ (50 mL*3), and purified by silica gel column chromatography (petroleum ether/ethyl acetate=85/15) to give 10b as a yellow oil (1890 mg, 97.6% yield). MS (EST) m/z: 342.4 [M+H]⁺.

Step 2: 2-((tert-butyldimethylsilyl)oxy)ethyl ((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)carbamate (10c). To the solution of 10b (103 mg, 0.3 mmol) and P7 (135 mg, 0.25 mmol) in dry DMF (2 mL) were added HOBt (10.2 mg, 0.075 mmol) and DIPEA (0.13 mL, 0.75 mmol), stirred at r.t. for 5 h. The resulting solution was concentrated and purified by silica column gel chromatography (CH₂Cl₂/ethyl acetate=50/50) to give 10c (148 mg, 91.7% yield) as a white solid. MS (ESI) m/z: 650.4 [M+H]⁺.

Step 3: 2-hydroxyethyl ((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)carbamate (P10). To the solution of 10c (140 mg, 0.22 mmol) in MeOH (2 mL) was added 2N HCl (2 ml) stirred at r.t. for 2 h. The solution was extracted with CH₂Cl₂(20 mL, 60*3). The organic phase was concentrated and purified by silica gel column chromatography (MeOH/CH₂Cl₂=90/10) to give P10 (60 mg, 52% yield) as a yellow solid. MS (ESI) m/z: 536.4 [M+H]⁺.

### Example 11

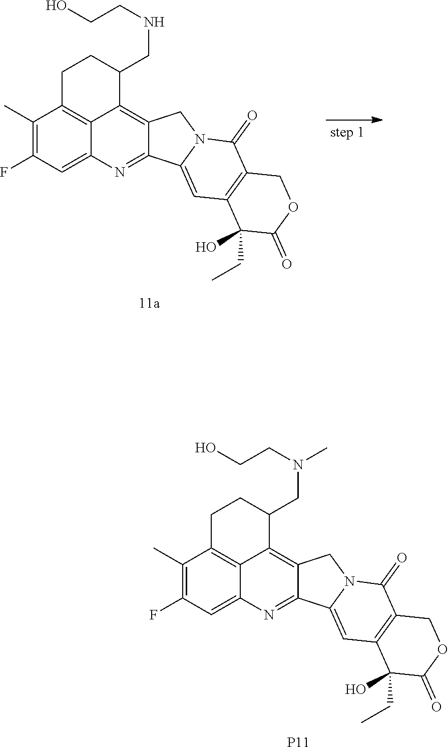 [SMILES: CC[C@@]1(O)C(=O)OCc2c1cc1n(c2=O)Cc2c-1nc1cc(F)c(C)c3c1c2C(CNCCO)CC3 ; CC[C@@]1(O)C(=O)OCc2c1cc1n(c2=O)Cc2c-1nc1cc(F)c(C)c3c1c2C(CN(C)CCO)CC3 ; "P11"]

Step 1: (9S)-9-ethyl-5-fluoro-9-hydroxy-1-(((2-hydroxyethyl)(methyl)amino)methyl)-4-methyl-1,2,3,9,12,15-hexahydro-10H,13H-benzo[de]pyrano[3′,4′:6,7]indolizino[1,2-b]quinoline-10,13-dione (P11). To a solution of 11a (10 mg, 0.02 mmol) in MeOH (1 mL) was added 37% formaldehyde solution (2.8 μL, 0.04 mmol). The solution was then cooled to 0 °C and NaBH₃CN (2 mg, 0.03 mmol) was then added in one portion. The solution was then warmed to r.t. and stirred for another 2 h. The mixture was then quenched with H₂O and purified by prep-HPLC (FA) (Method: column: XBridge Prep C18 OBD 5 μm 19*150 mm; Mobile phase: A-water (0.1% FA): B-acetonitrile; Flow rate: 20 mL/min), the fraction was lyophilized to give P11 as a white solid (5.5 mg, 54.2% yield). MS (ESI) m/z: 508.5 [M+H]⁺.

### Example 12

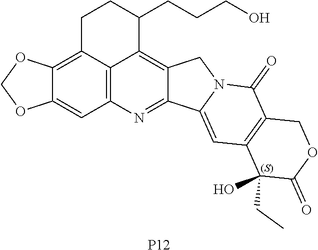

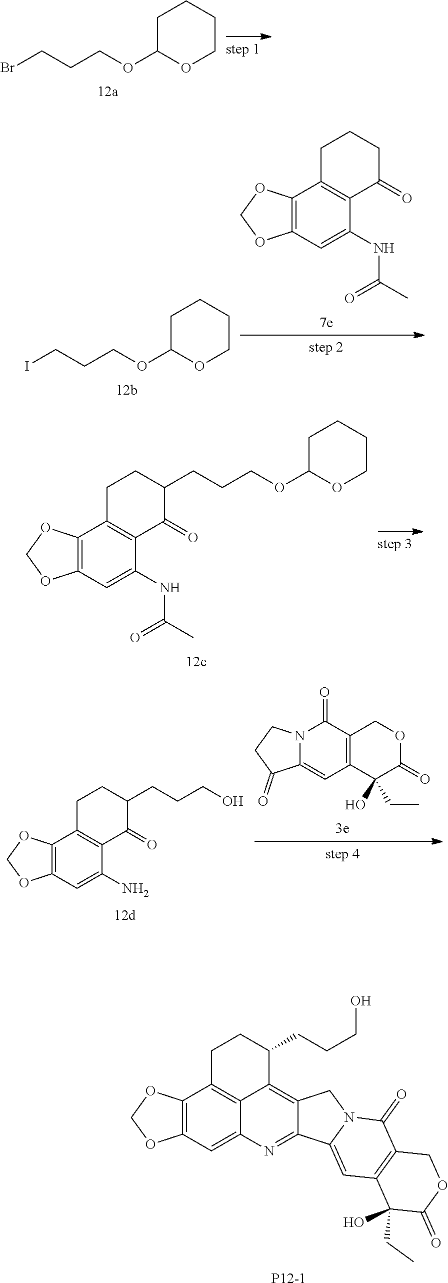

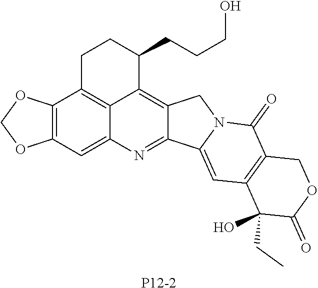 [SMILES: CC[C@@]1(O)C(=O)OCc2c1cc1n(c2=O)Cc2c-1nc1cc3c(c4c1c2[C@H](CCCO)CC4)OCO3 ; "P12-2"]

Step 1: 2-(3-iodopropoxy)tetrahydro-2H-pyran (12b). To a solution of compound 12a (2.0 g, 9.01 mmol) in acetone (20 mL) was added NaI (4.0 g, 27 mmol). The mixture was stirred at 60 °C for 3 h. The mixture was diluted with hexane (40 mL) and washed with water (40 mL) and brine (40 mL). The combined organic layer was dried over Na₂SO₄ and concentrated to afford 12b (1.3 g, 54% yield) as a colorless oil. ¹H NMR (400 MHz, CDCl₃) δ 4.61 (dd, J=4.4, 2.8 Hz, 1H), 3.91-3.83 (m, 1H), 3.83-3.77 (m, 1H), 3.56-3.49 (m, 1H), 3.45 (dt, J=10.0, 5.9 Hz, 1H), 3.30 (td, J=6.8, 1.0 Hz, 2H), 2.10 (ddd, J=12.7, 6.8, 5.9 Hz, 2H), 1.90-1.63 (m, 3H), 1.61-1.48 (m, 5H).

Step 2: N-(6-oxo-7-(3-((tetrahydro-2H-pyran-2-yl)oxy)propyl)-6,7,8,9-tetrahydronaphtho[1,2-d][1,3]dioxol-5-yl)acetamide (12c). To a solution of compound 7e (100 mg, 3.52 mmol) in THF (5 mL) was drop-wised added t-BuOK (1.2 mL, 1.212 mmol, 1 M in THF) at −40 °C under N₂. The mixture was stirred at −40 °C for 30 mi under N₂. Then compound 12b (164 mg, 0.606 mmol, dissolved in 0.2 mL THF) was dropwise added to the mixture. The mixture was stirred for 16 h with gradual heating to room temperature under N₂.

The mixture was quenched with sat. NH₄C₁ (3 mL) and extracted with EtOAc (3 mL*3). The combined organic layer was dried over anhydrous Na₂SO₄, filtered, and concentrated to give the residue, which was purified by silica gel column chromatography (Petroleum ether/EtOAc=80:20) to afford the compound 12c (20 mg, 12.7% yield) as yellow solid. MS (ESI) m/z: 248.1 [M+H]⁺. ¹H NMR (400 MHz, CDCl₃) δ 12.44 (s, 1H), 8.26 (s, 1H), 6.01-5.98 (m, 2H), 4.59-4.53 (m, 1H), 3.90-3.72 (m, 2H), 3.53-3.36 (m, 2H), 3.00-2.88 (m, 1H), 2.79-2.67 (m, 1H), 2.55-2.44 (m, 1H), 2.19 (s, 3H), 2.17-2.09 (m, 1H). 2.06-1.86 (m, 2H), 1.86-1.65 (m, 6H), 1.60-1.54 (m, 2H).

Step 3: 5-amino-7-(3-hydroxypropyl)-8,9-dihydronaphtho[1,2-d][1,3]dioxol-6(7H)-one (12d). To a solution of compound 12c (20 mg, 0.05 mmol) in MeOH (5 mL) was added 2 N HCl (5 mL). The mixture was stirred at 60 °C for 3 h. The mixture was concentrated in vacuo to remove MeOH. Then the mixture was adjusted to pH=8 using sat. Na₂CO₃ and extracted with DCM (5 mL*3). The combined organic phase was concentrated in vacuo to give the residue, which was purified by silica column gel chromatography (eluent: Petroleum ether/EtOAc=0/100) to afford 12d (10 mg, 74% yield) as yellow oil. MS (EST) m/z: 264.2 [M+H]⁺.

Step 4: (1R,10S)-10-ethyl-10-hydroxy-1-(3-hydroxypropyl)-1,2,3,10,13,16-hexahydro-11H,14H-benzo[de][1,3]dioxolo[4,5-g]pyrano[3′,4′:6,7]indolizino[1,2-b]quinoline-11,14-dione (P12-1) and (1S,10S)-10-ethyl-10-hydroxy-1-(3-hydroxypropyl)-1,2,3,10,13,16-hexahydro-11H,14H-benzo[de][1,3]dioxolo[4,5-g]pyrano[3′,4′:6,7]indolizino[1,2-b]quinoline-11,14-dione (P12-2). To a solution of compound 12d (80 mg, 0.304 mmol) in toluene (14 mL) and o-cresol (2 mL) was added 3e (88 mg, 0.334 mmol) and PPTS (23 mg, 0.091 mmol). The mixture was refluxed at 140 °C for 16 h. The mixture was concentrated in vacuo to remove toluene and purified by silica column gel chromatography (eluent: DCM/MeOH=90/10) to crude product (mixture of P12-1 and P12-2), which was purified by prep-HPLC (FA) (Method: column: XBridge Prep C18 OBD 5 μm 19*150 mm; Mobile phase: A-water (0.1% formic acid): B-acetonitrile; Flow rate: 20 mL/min). The fraction was lyophilized to give P12-1 (15 mg, 710% yield) as a white solid and P12-2 (20 mg, 13% yield) as a white solid.

Compound P12-1. MS (ESI) m/z: 491.5 [M+H]⁺. ¹H NMR (400 MHz, DMSO-d₆) δ 7.37 (s, 1H), 7.23 (s, 1H), 6.48 (s, 1H), 6.27 (d, J=2.9 Hz, 2H), 5.42 (s, 2H), 5.31 (d, J=18.9 Hz, 1H), 5.19 (d, J=18.8 Hz, 1H), 4.42 (t, J=5.1 Hz, 1H), 3.48-3.42 (m, 21H), 3.01-2.91 (m, 2H), 2.22 (d, J=13.4 Hz, 1H), 1.93-1.75 (m, 4H), 1.73-1.63 (m, 2H), 1.61-1.54 (m, 2H), 0.87 (t, J=7.3 Hz, 3H).

Compound P12-2. MS (ESI) m/z: 491.5 [M+H]⁺. ¹H NMR (400 MHz, DMSO-d4) δ 7.37 (s, 1H), 7.23 (s, 1H), 6.49 (s, 1H), 6.27 (s, 2H), 5.42 (s, 2H), 5.31 (d, J=18.9 Hz, 1H), 5.20 (d, J=18.9 Hz, 1H), 4.43 (t, J=5.2 Hz, 1H), 3.47-3.43 (m, 2H), 3.02-2.86 (m, 2H), 2.22 (d, J=13.3 Hz, 1H), 1.94-1.78 (m, 3H), 1.73-1.63 (m, 2H), 1.63-1.53 (m, 3H), 0.87 (t, J=7.3 Hz, 3H).

### Example 1-1

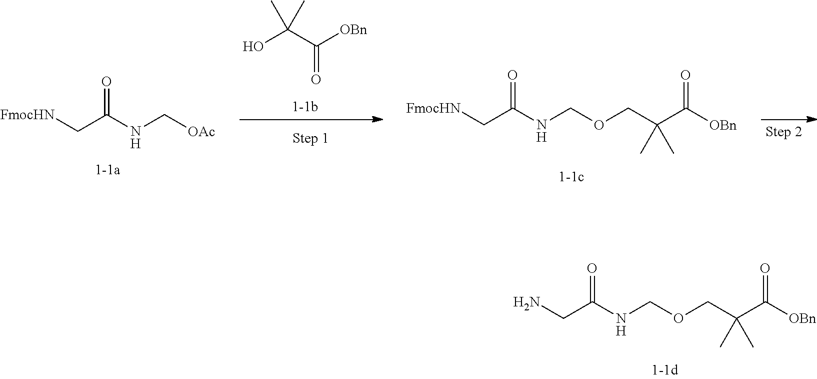

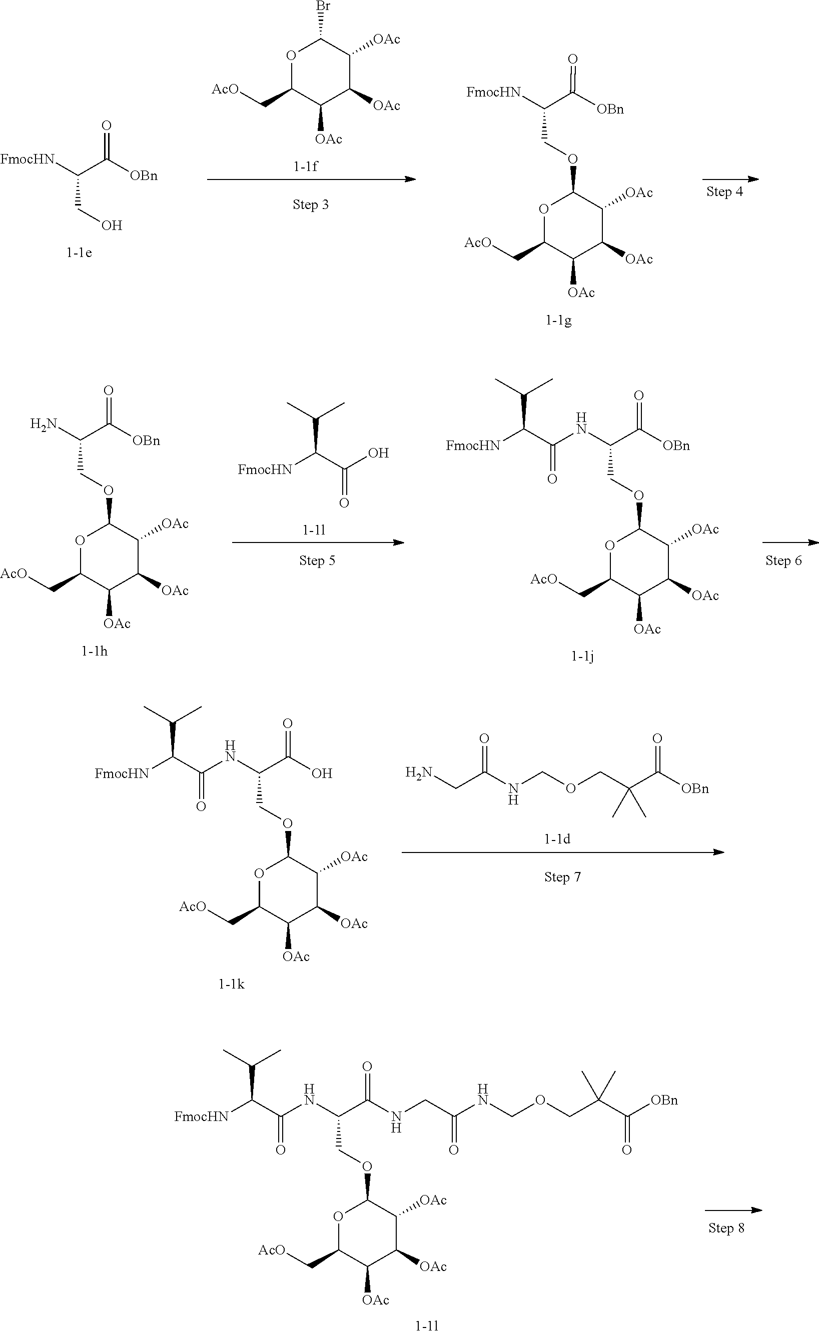

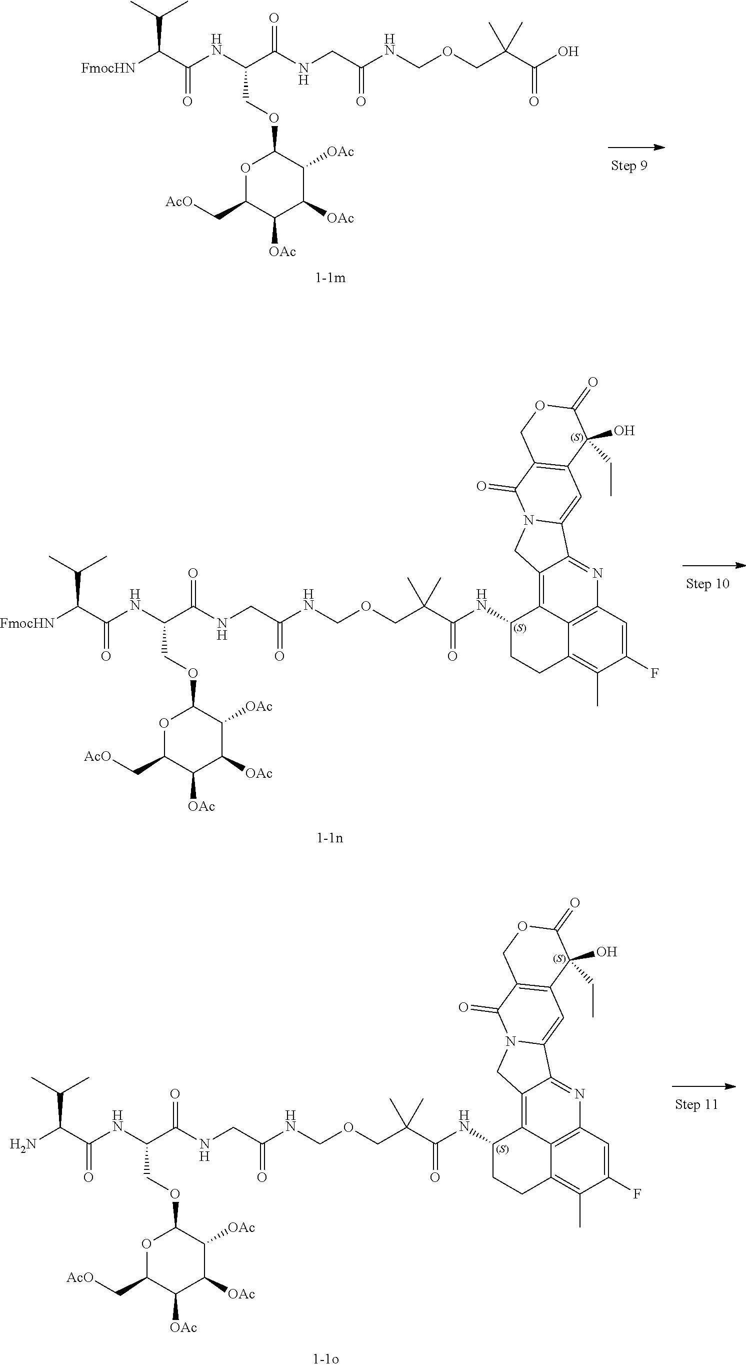

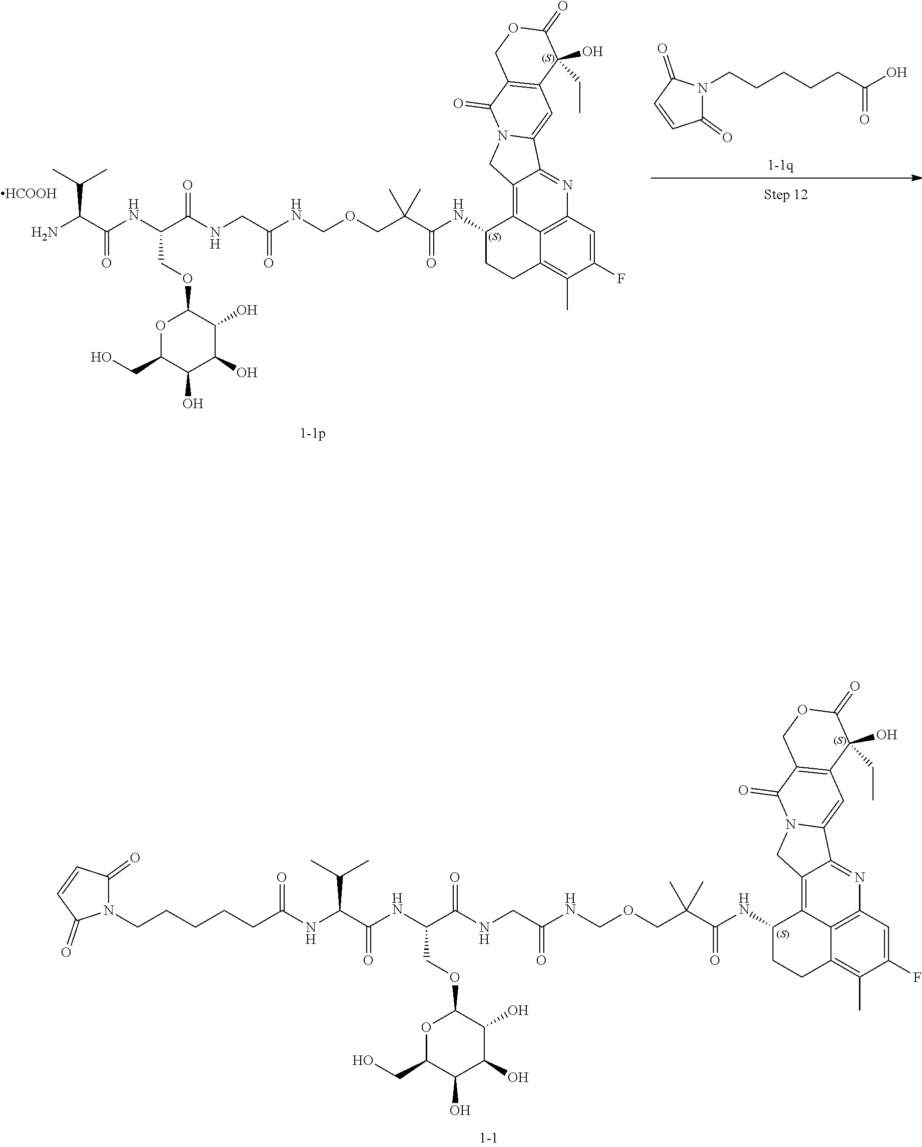

Step 1: benzyl 1-(9H-fluoren-9-yl)-11,11-dimethyl-3,6-dioxo-2,9-dioxa-4,7-diazadodecan-12-oate (1-1c). To a mixture of 1-1a (1000 mg, 2.71 mmol), 1-1b (1580 mg, 813 mmol) and dried 4 Å molecular sieves (3.0 g) in dry THE (40 ml) was added scandium trifluoromethanesulfonate (2.0 g, 2.87 mmol), stirred at r.t. under N₂ atmosphere overnight. The solution was filtered through Celite, diluted with EtOAc (200 mL), and washed by saturated NaHCO₃ (50 mL*3). The organic layer was dried over anhydrous Na₂SO₄, filtered, and concentrated. The residue was purified by silica column gel chromatography (eluent: Petroleum ether/EtOAc=100/0 to 50/50) to afford compound 1-1c (950 mg, 67.9% yield) as a white solid. MS (EST) m/z: 539.4 [M+Na]⁺.

Step 2: benzyl 3-((2-aminoacetamido)methoxy)-2,2-dimethylpropanoate (1-1d). To a solution of compound 1-1c (950 mg, 1.84 mmol) in DMF (6 mL) was added acetyl-L-cysteine (450 mg, 2.76 mmol). Then Et₂NH (2.69 g, 36.8 mmol) was added. The mixture was stirred at r.t. for 30 min. The mixture was concentrated under high vacuum and purified by prep-HPLC (Method: column: XBridge Prep C18 OBD 5 μm 19*150 mm; Mobile phase: A-water (Neutral condition): B-acetonitrile; Flow rate: 20 mL/min), the fraction was lyophilized to give compound 1-id (340 mg, 62.6%) as colorless oil. MS (ESI) m/z: 317.3 [M+Na]+.

Step 3: (2R,3R,4S,5S,6R)-2-((S)-2-((((9H-fluoren-9-yl)methoxy)carbonyl)amino)-3-(benzyloxy)-3-oxopropoxy)-6-(acetoxymethyl)tetrahydro-2H-pyran-3,4,5-triyl triacetate (1-1g). To a solution of compound 1-1e (2.0 g, 4.79 mmol) in CH₂Cl₂ (30 mL) were added 1-1f (3350 mg, 8.14 mmol) and 4 A molecular sieve (4.0 g), the mixture was stirred at room temperature for 30 min under N₂ atmosphere. Then AgOTf (1.8 g, 7.19 mmol) was added into the mixture. The mixture was stirred at room temperature for 16 h. The mixture was filtered, the filtrate was diluted with EtOAc (300 mL), washed by sat. NaHCO₃ (100 mL*3) and brine (100 mL*3), the organic layer was dried over anhydrous Na₂SO₄, filtered, and concentrated. The residue was purified by silica column gel chromatography (eluent: Petroleum ether/EtOAc=100/0 to 40/60) to afford 1-1g (1300 mg, 36.3% yield). MS (ESI) m/z: 770.4 [M+Na]⁺.

Step 4: (2R,3S,4S,5R,6R)-2-(acetoxymethyl)-6-((S)-2-amino-3-(benzyloxy)-3-oxopropoxy)tetrahydro-2H-pyran-3,4,5-triyl triacetate (1-1h). To a solution of compound 1-1g (1.3 g, 1.74 mmol) in DMF (10 mL) was added Et₂NH (2.5 g, 34.77 mmol). The mixture was stirred at r.t. for 30 min. The mixture was concentrated under high vacuum and co-evaporated with toluene (5 mL*3) to give compound 1-1h (914 mg, theoretical yield) as a brown solid. MS (ESI) m/z: 526.4 [M+H]⁺.

Step 5: (2R,3R,4S,5S,6R)-2-((S)-2-((S)-2-((((9H-fluoren-9-yl)methoxy)carbonyl)amino)-3-methylbutanamido)-3-(benzyloxy)-3-oxopropoxy)-6-(acetoxymethyl)tetrahydro-2H-pyran-3,4,5-triyl triacetate (1-1j). To a solution of compound 1-th (914 mg, 1.739 mmol) in DMF (8 mL) were added compound 1-1h (708 mg, 2.086 mmol), HATU (992 mg, 2.61 mmol), and DIEA (450 mg, 3.48 mmol). The mixture was stirred at r.t. for 30 min. The mixture was concentrated and purified by flash column chromatography (eluted with Petroleum ether/EtOAc=100/0 to 50/50). Compound 1-1j (1210 mg, 82.3%) was obtained as a white solid. MS (ESI) m/z: 869.5 [M+Na]⁺.

Step 6: N-((((9H-fluoren-9-yl)methoxy)carbonyl)-L-valyl)-O-((2R,3R,4S,5S,6R)-3,4,5-triacetoxy-6-(acetoxymethyl)tetrahydro-2H-pyran-2-yl)-L-serine (1-1k). To a solution of compound 1-1j (1210 mg, 1.43 mmol) in MeOH (10 mL) was added Pd/C (10%, 242 mg). The mixture was stirred at r.t. under H₂ for 2 h. The mixture was filtered and concentrated to give crude product 1-1k (784 mg, crude) as a white solid. MS (ESI) m/z: 779.5 [M-Na]⁺.

Step 7: (2R,3R,4S,5S,6R)-2-(((S)-13-((S)-2-((((9H-fluoren-9-yl)methoxy)carbonyl)amino)-3-methylbutanamido)-4,4-dimethyl-3,9,12-trioxo-1-phenyl-2,6-dioxa-8,11-diazatetradecan-14-yl)oxy)-6-(acetoxymethyl)tetrahydro-2H-pyran-3,4,5-triyl triacetate (1-11). To a solution of compound 1-1k (250 mg, 0.33 mmol) in DMF (4 mL) were added compound 1-1d (117 mg, 0.396 mmol), HATU (188 mg, 0.496 mmol), and DIEA (85 mg, 0.661 mmol). The mixture was stirred at r.t. for 30 min. The mixture was concentrated and purified by prep-HPLC (FA) (Method: column: XBridge Prep C18 OBD 5 μm 19*150 mm; Mobile phase: A-water (0.1% formic acid): B-acetonitrile; Flow rate: 20 mL/min), the fraction was lyophilized to give compound 1-11 (248 mg, 84.8% yield) as an off-white solid. MS (ESI) m/z: 1056.7 [M+Na]⁺.

Step 8: (5S,8S)-1-(9H-fluoren-9-yl)-5-isopropyl-17,17-dimethyl-3,6,9,12-tetraoxo-8-((((2R,3R,4S,5S,6R)-3,4,5-triacetoxy-6-(acetoxymethyl)tetrahydro-2H-pyran-2-yl)oxy)methyl)-2,15-dioxa-4,7,10,13-tetraazaoctadecan-18-oic acid (1-1m). Compound 1-1m (264 mg, crude) was synthesized according to the synthetic procedure of step 3 of Example 1-1.

Step 9: (2R,3R,4S,5S,6R)-2-((S)-2-((S)-2-((((9H-fluoren-9-yl)methoxy)carbonyl)amino)-3-methylbutanamido)-3-((2-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-2-oxoethyl)amino)-3-oxopropoxy)-6-(acetoxymethyl)tetrahydro-2H-pyran-3,4,5-triyl triacetate (1-1n). To a solution of compound 1-1m (264 mg, 0.28 mmol) in DMF (6 mL) were added exatecan mesylate (156 mg, 0.29 mmol), HATU (160 mg, 0.42 mmol), and DIEA (109 mg, 0.84 mmol). The mixture was stirred at r.t. for 30 min. The mixture was purified by prep-HPLC (FA) (Method: column: XBridge Prep C18 OBD 5 μm 19*150 mm; Mobile phase: A-water (0.1% formic acid): B-acetonitrile; Flow rate: 20 mL/min), the fraction was lyophilized to give compound 1-in (248 mg, 69.3% yield) as an off-white solid. MS (ESI) m/z: 1260.7 [M+H]⁺.

Step 10: (2R,3S,4S,5R,6R)-2-(acetoxymethyl)-6-((S)-2-((S)-2-amino-3-methylbutanamido)-3-((2-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-2-oxoethyl)amino)-3-oxopropoxy)tetrahydro-2H-pyran-3,4,5-triyl triacetate (1-1o). To a solution of compound 1-1n (248 mg, 0.18 mmol) in DMF (6 mL) was added Et₂NH (133 mg, 1.82 nmol). The mixture was stirred at r.t. for 30 min. The mixture was concentrated under high vacuum to give compound 1-1o (207 mg, crude) as an off-white solid, which was used directly without further purification. MS (ESI) m/z: 1138.7 [M+H]⁺.

Step 11: (S)-2-amino-N—((S)-1-((2-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)-3-methylbutanamide formate (1-1p). To a solution of compound 1-1o (207 mg, crude) in MeOH (6 ml) was added K₂CO₃ (50 mg, 0.36 mmol). The mixture was stirred at r.t. for 60 min. The mixture was purified by prep-HPLC (FA) (Method: column: XBridge Prep C18 OBD 5 μm 19*150 mm; Mobile phase: A-water (0.1% formic acid): B-acetonitrile; Flow rate: 20 m/min), the fraction was lyophilized to give compound 1-1p (105 mg, 56.8% yield) as white solid. MS (ESI) m/z: 970.7 [M+H]⁺.

Step 12: 6-(2,5-dioxo-2,5-dihydro-1H-pyrrol-1-yl)-N-((11S,14S)-1-(((1S,9S)-9-ethyl-5-fluoro-9-hydroxy-4-methyl-10,13-dioxo-2,3,9,10,13,15-hexahydro-11H,12H-benzo[de]pyrano[3′,4′:6,7]indolizino[1,2-b]quinolin-1-yl)amino)-2,2,15-trimethyl-1,7,10,13-tetraoxo-11-((((2R,3R,4S,5R,6R)-3,4,5-trihydroxy-6-(hydroxymethyl)tetrahydro-2H-pyran-2-yl)oxy)methyl)-4-oxa-6,9,12-triazahexadecan-14-yl)hexanamide (1-1). To a solution of compound 1-1p (40 mg, 0.041 mmol) in DMF (2 mL) were added compound 1-1q (13 mg, 0.062 mmol), HATU (24 mg, 0.062 mmol), and DIEA (11 mg, 0.082 mmol). The mixture was stirred at r.t. for 30 mi. The mixture was purified by prep-HPLC (FA) (Method: column: XBridge Prep C18 OBD 5 μm 19*150 mm; Mobile phase: A-water (0.1% formic acid): B-acetonitrile; Flow rate: 20 ml/min), the fraction was lyophilized to give compound 1-1 (18.5 mg, 33.7% yield) as a white solid. MS (ESI) m/z: 1185.8 [M+Na]⁺.

### Example 1-2

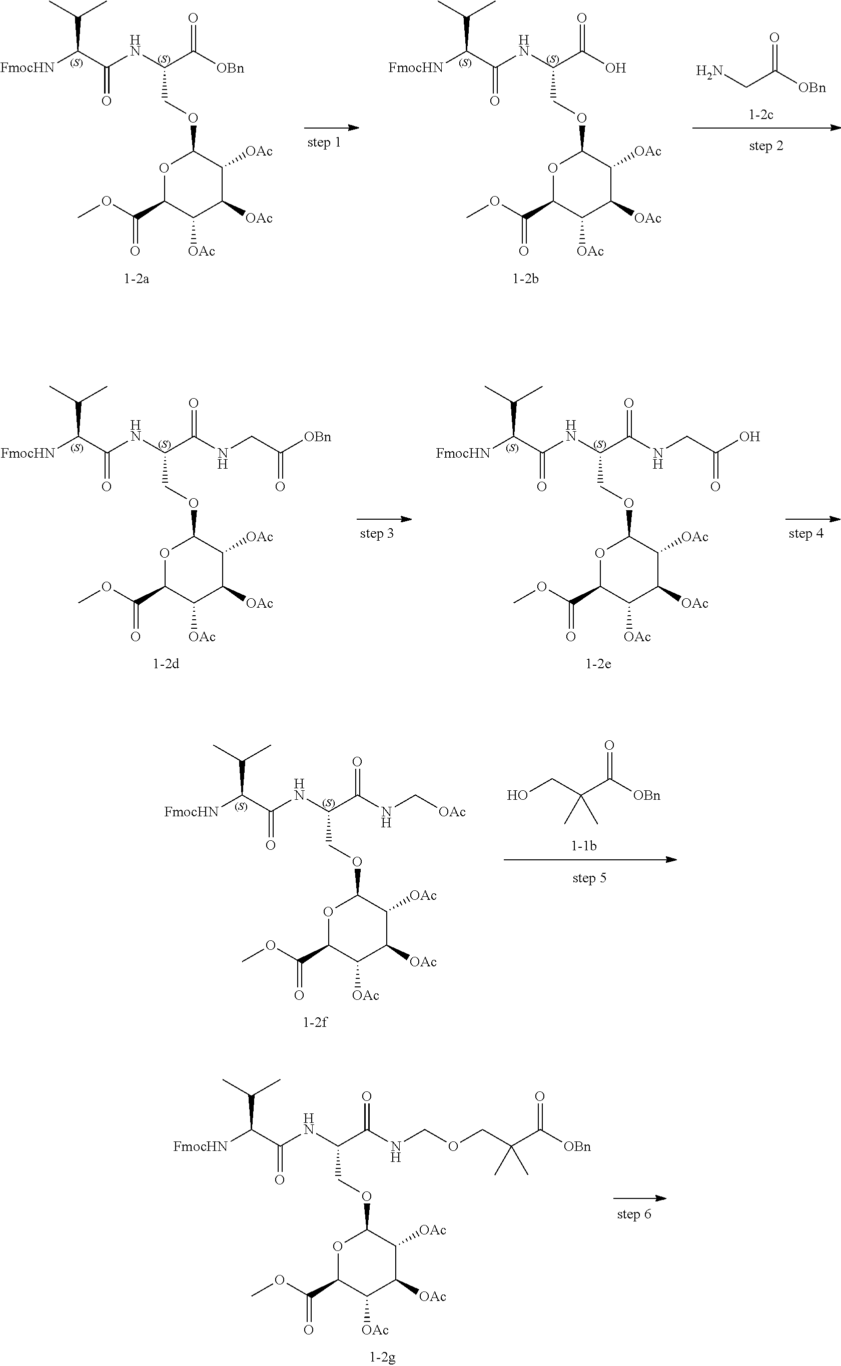

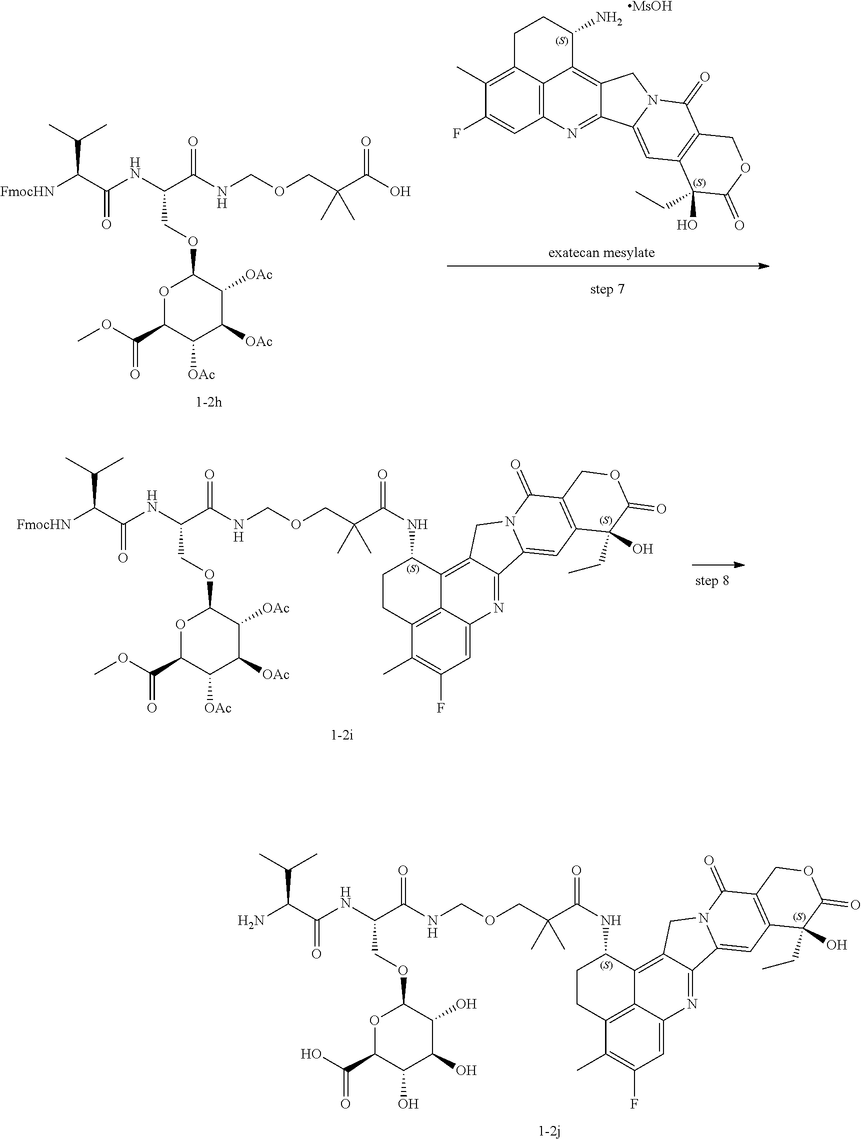

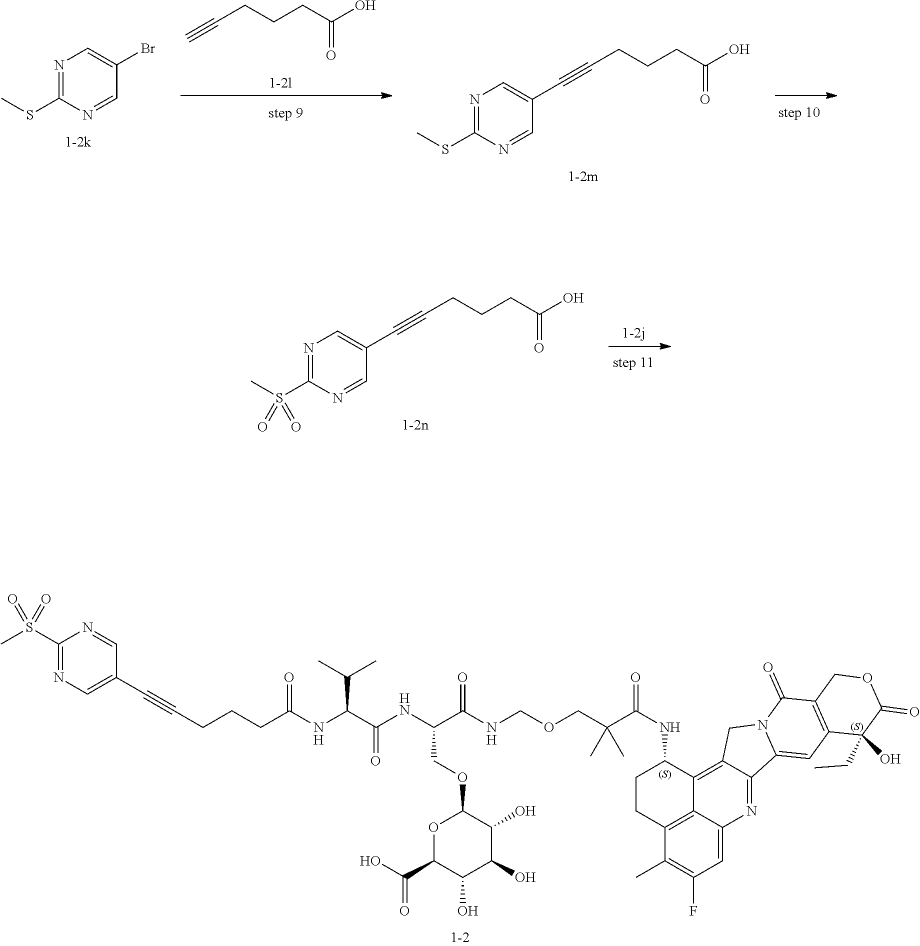

Step 1: N-((((9H-fluoren-9-yl)methoxy)carbonyl)-L-valyl)-O-((2R,3R,4S,5S,6S)-3,4,5-triacetoxy-6-(methoxycarbonyl)tetrahydro-2H-pyran-2-yl)-L-serine (1-2b). To a mixture of 1-2a (4.1 g, 4.92 mmol, purchase from MCE) in MeOH (50 mL), THF (10 mL) and DCM (20 mL) was added wet Pd/C (400 mg, 10% purity). The black suspension was purged with H₂ balloon for three times and then stirred at r.t. for 1 h. The black suspension was filtered through a pad of celite, washed with MeOH (200 mL). Combined organic layers and concentrated under vacuum to give 1-2b (3650 mg, 99.8% yield) as an off-white solid. MS (ESI) m/z: 743.6 [M+H]⁻.

Step 2: (2R,3R,4S,5S,6S)-2-((S)-2-((S)-2-((((9H-fluoren-9-yl)methoxy)carbonyl)amino)-3-methylbutanamido)-3-((2-(benzyloxy)-2-oxoethyl)amino)-3-oxopropoxy)-6-(methoxy carbonyl)tetrahydro-2H-pyran-3,4,5-triyl triacetate (1-2d). To a solution of 1-2b (3.65 g, 4.92 mmol) and 1-2c (1.66 g, 4.92 mmol) in DMF (50 mL) were added HATU (1.87 g, 4.92 mmol) and DIPEA (1.59 g, 12.29 mmol). The mixture was stirred at r.t. for 30 min. The mixture was purified by flash column chromatography (MeOH/DCM=0-10%), the fraction was concentrated under vacuum to give 1-2d (3.8 g, 86.9% yield) as an off-white foamed solid. MS (ESI) m/z: 890.7 [M+H]⁺.

Step 3: N-((((9H-fluoren-9-yl)methoxy)carbonyl)-IL-valyl)-O-((2R,3R,4S,5S,6S)-3,4,5-triacetoxy-6-(methoxycarbonyl)tetrahydro-2H-pyran-2-yl)-L-serylglycine (1-2e). To a mixture of 1-2d (3.8 g, 4.27 mmol) in MeOH (150 mL) and DCM (50 mL) was added wet Pd/C (400 mg, 10% purity). The black suspension was purged with H₂ balloon for three times and then stirred at r.t. for 40 min. The black suspension was filtered through a pad of celite, washed with MeOH (150 mL). Organic layers were combined and concentrated under vacuum to give 1-2e (3.3 g, 96.6% yield) as an off-white solid. MS (ESI) m/z: 800.7 [M+H]⁺.

Step 4: (2R,3R,4S,5S,6S)-2-((S)-2-((S)-2-((((9H-fluoren-9-yl)methoxy)carbonyl)amino)-3-methylbutanamido)-3-((acetoxymethyl)amino)-3-oxopropoxy)-6-(methoxycarbonyl)tetrahydro-2H-pyran-3,4,5-triyl triacetate (1-2f). To a solution of 1-2e (3.3 g, 4.13 mmol) in DMF (30 mL) were added Pb(OAc)₄ (2.74 g, 6.19 mmol), Cu(OAc)₂ (74.9 mg, 0.41 mmol), and HOAc (247.8 mg, 4.13 mmol). The resulting dark color mixture was purged with N₂ balloon for three times and then stirred at 65 °C for 40 min. and the mixture turned deep blue. The mixture was diluted with EtOAc (300 mL), washed with brine (100 mL*3), dried over Na₂SO₄, filtered, and concentrated under vacuum to give a residue. It was purified by flash column chromatography (MeOH/DCM=0/100~10/90), the fraction was concentrated under vacuum to give 1-2f (2.8 g, 83.4% yield) as a pale-yellow solid. MS (ESI) m/z: 836.6 [M+Na]⁺.

Step 5: (2R,3R,4S,5S,6S)-2-((S)-2-((S)-2-((((9H-fluoren-9-yl)methoxy)carbonyl)amino)-3-methylbutanamido)-3-(((3-(benzyloxy)-2,2-dimethyl-3-oxopropoxy)methyl)amino)-3-oxopropoxy)-6-(methoxycarbonyl)tetrahydro-2H-pyran-3,4,5-triyl triacetate (1-2g). A white suspension mixture of 1-1b (300 mg, 0.37 mmol), 1-2f (154 mg, 0.74 mmol) and 4 A molecular sieve (200 mg) in anhydro THF (10 mL) was stirred at r.t. for 10 min. Sc(OTf)₃ (217.9 mg, 0.44 mmol) was added and the resulting yellow suspension was stirred at r.t. for 4 h. The yellow suspension mixture was filtered through a pad of celite and washed with EtOAc (30 mL). Combined organic layers were washed with sat. NaHCO₃ (30 mL) and brine (30 mL), dried over Na₂SO₄, filtered and the filtrate was concentrated under vacuum to give a residue. It was purified by silica gel column (MeOH/DCM=0/100~-5/95), fraction was concentrated under vacuum to give 1-2g (275 mg, 77.5% yield) as a white foam solid. MS (ESI) m/z: 984.8 [=M+Na]⁺.

Step 6: (5S,8S)-1-(9H-fluoren-9-yl)-5-isopropyl-14,14-dimethyl-3,6,9-trioxo-8-((((2R,3R,4S,5S,6S)-3,4,5-triacetoxy-6-(methoxycarbonyl)tetrahydro-2H-pyran-2-yl)oxy)methyl)-2,12-dioxa-4,7,10-triazapentadecan-15-oic acid (1-2h). To a solution of 1-2g (275 mg, 0.29 mmol) in MeOH (10 mL) was added wet Pd/C (55 mg, 10% purity). The black suspension was purged with H₂ balloon for three times then stirred at r.t. for 2 h. The mixture was filtered through syringe head and washed with MeOH (15 mL), concentrated under vacuum to give 1-2h (230 mg, crude) as a white foam solid. MS (ESI) m/z: 894.6 [M+Na]⁺.

Step 7: (2R,3R,4S,5S,6S)-2-((S)-2-((S)-2-((((9H-fluoren-9-yl)methoxy)carbonyl)amino)-3-methylbutanamido)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-3-oxopropoxy)-6-(methoxycarbonyl)tetrahydro-2H-pyran-3,4,5-triyl triacetate (1-2i). To a mixture of 1-2h (230 mg, crude), exatecan mesylate (140 mg, 0.26 mmol) and HATU (100.3 mg, 0.26 mmol) in DMF (5 mL) was added DIPEA (102 mg, 0.79 mmol). The resulting brown mixture was stirred at r.t. for 1 h. The mixture was diluted with EtOAc (20 mL), washed with brine (20 mL*3), dried over Na₂SO₄, filtered, and concentrated under vacuum to give a residue. It was purified by flash column chromatography (MeOH/DCM=0%~3%), concentrated under vacuum to give 1-2i (325 mg, 95.6% yield) as an off-white foam solid. MS (ESI) m/z: 1289.9 [M+H]⁺.

Step 8: (2S,3S,4S,5R,6R)-6-((S)-2-((S)-2-amino-3-methylbutanamido)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-3-oxopropoxy)-3,4,5-trihydroxytetrahydro-2H-pyran-2-carboxylic acid (1-2j). To a solution of 1-2i (325 mg, 0.25 mmol) in DMF (5 mL) was added Et₂NH (523.2 mg, 5.06 mmol). The mixture was stirred at r.t. for 20 min. Then concentrated under vacuum to give a crude product. The product was dissolved in MeOH (6 mL) to which K₂CO₃ (174.7 mg, 1.26 mmol) was added and stirred at r.t. for 10 min, then H₂O (2 mL) was added to the mixture and stirred at r.t. for 30 min. The mixture was acidified with sat. KHSO₄ at 0 °C to pH=3, filtered and purified by prep-HPLC (0.1% FA), fraction was lyophilized to give 1-2j (140 mg, 59.7% yield) as a pale yellow solid. MS (ESI) m/z: 927.4 [M+H]⁺. ¹H NMR (400 MHz, DMSO) δ 9.56 (s, 1H), 8.39 (s, 1H), 8.07 (d, J=8.4 Hz, 11H), 7.77 (d, J=11.2 Hz, 11H), 7.31 (s, 11H), 6.52 (s, 1H), 5.54 (dd, J=13.2, 7.2 Hz, 1H), 5.43 (s, 2H), 5.18 (dd, J=41.6, 18.8 Hz, 2H), 5.09-5.02 (m, 1H), 4.96 (s, 1H), 4.62 (dd, J=10.0, 6.8 Hz, 1H), 4.56-4.44 (m, 2H), 4.19 (d, J=7.6 Hz, 11H), 3.82 (dd, J=10.8, 6.8 Hz, 1H), 3.61 (dd, J=11.6, 6.4 Hz, 2H), 3.17-3.05 (m, 4H), 2.94 (t, J=8.0 Hz, 1H), 2.39 (s, 3H), 2.11 (dt, J=21.3, 7.6 Hz, 2H), 2.03-1.93 (m, 2H), 1.92-1.78 (m, 3H), 1.12 (d, J=8.0 Hz, 6H), 0.87 (dd, J=13.0, 6.6 Hz, 9H).

Step 9: 6-(2-(methylthio)pyrimidin-5-yl)hex-5-ynoic acid (1-2m). To a mixture of 1-2k (2.0 g, 9.751 mmol) and 1-21 (1.31 g, 11.702 mmol) in DMF (30 mL) were added CuI (185.7 mg, 0.975 mmol), Pd(PPh₃)Cl₂ (684.4 mg, 0.975 mmol) and TEA (4.1 mL, 2.960 g, 29.254 mmol). The mixture was degassed with nitrogen atmosphere for three times then stirred at 95 °C for 2 h. The mixture was diluted with EtOAc (30 mL), washed with brine (30 mL*2), dried over Na₂SO₄, filtered and concentrated under vacuum to give a residue. It was purified by flash column chromatography (MeOH/DCM=0/100-3/97), fraction was concentrated under vacuum to give 1-2m (1.6 g, ~65% yield) as a yellow solid. MS (ESI) m/z: 237.1 [M+H]⁺.

Step 10: 6-(2-(methylsulfonyl)pyrimidin-5-yl)hex-5-ynoic acid (1-2n). To a solution of 1-2m (520 mg, 1.613 mmol) in DCM (10 mL) was added m-CPBA (1113.4 mg, 6.452 mmol). The resulting yellow suspension was stirred at R.T. for 1.5 h. The mixture was diluted with EtOAc (50 mL) and quenched by addition of sat. Na₂S₂O₃ (30 ml), stirred at r.t. for 10 min, extracted with DCM (30 mL*3). Combined organic layers were washed with brine (50 mL), dried over Na₂SO₄, filtered, and concentrated under vacuum to give crude product as a yellow solid. It was purified by prep-HPLC (FA), fraction was freeze-dried to give 1-2n (78.0 mg, 18% yield) as a white solid. MS (ESI) m/z: 269.1 [M+H]⁺. ¹H NMR (400 MHz, DMSO) § 12.17 (s, 1H), 9.12 (s, 2H), 3.41 (s, 3H), 2.60 (t, J=7.2 Hz, 2H), 2.41 (t, J=7.2 Hz, 2H), 1.82 (p, J=6.8 Hz, 2H).

Step 11: (2S,3S,4S,5R,6R)-6-((S)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-2-((S)-3-methyl-2-(6-(2-(methylsulfonyl)pyrimidin-5-yl)hex-5-ynamido)butanamido)-3-oxopropoxy)-3,4,5-trihydroxytetrahydro-2H-pyran-2-carboxylic acid (1-2). To a solution of 1-2n (5.2 mg, 0.019 mmol) and 1-2j (15 mg, 0.016 mmol) in DMF (2 mL) were added HATU (8.6 mg, 0.024 mmol) and DIPEA (7 μL, 5.2 mg, 0.04 mmol). The mixture was stirred at r.t. for 30 min. The mixture was purified by prep-HPLC (FA 0.1%), fraction was freeze-dried to give 1-2 (7.1 mg, 37.3% yield) as a white solid. MS (ESI) m/z: 1177.9 [M+H]⁺.

### Example 1-3

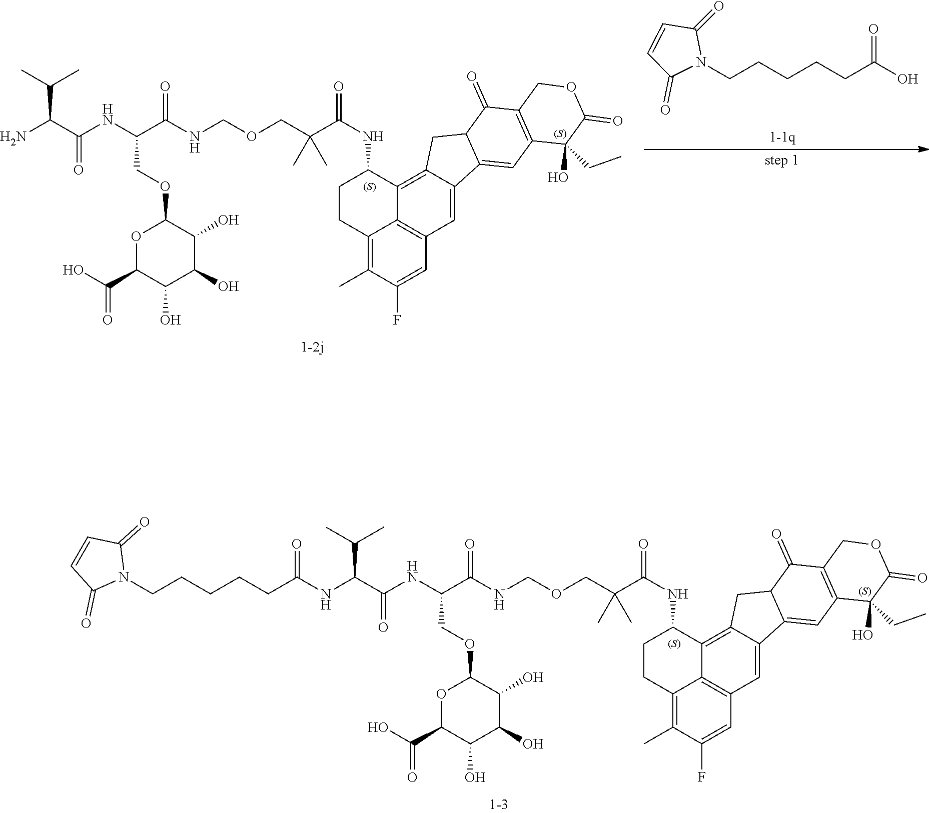

Step 1: (2S,3S,4S,5R,6R)-6-((S)-2-((S)-2-(6-(2,5-dioxo-2,5-dihydro-1H-pyrrol-1-yl)hexanamido)-3-methylbutanamido)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-3-oxopropoxy)-3,4,5-trihydroxytetrahydro-2H-pyran-2-carboxylic acid (1-3). 1-3 (55 mg, 52% yield) was synthesized according to the same procedures as that of step 12, Example 1-1. MS (ESI) m/z: 1120.9 [M+H]⁻

### Example 1-4

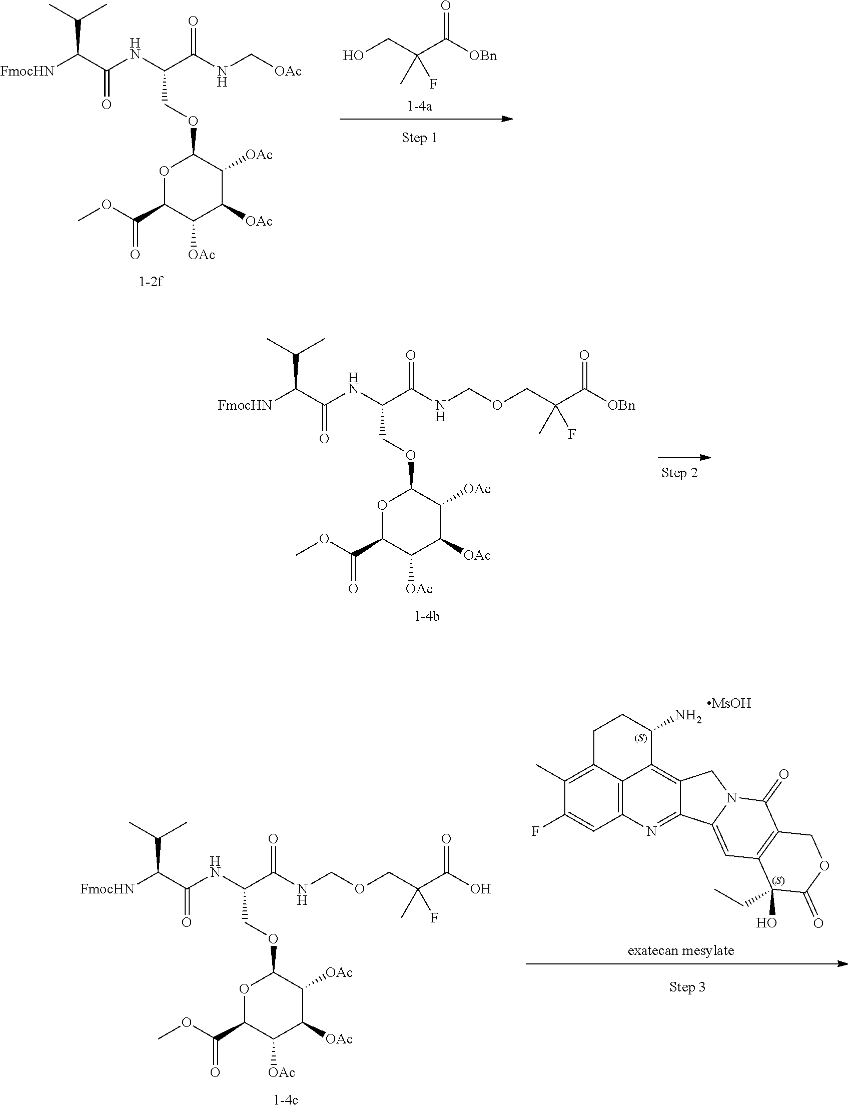

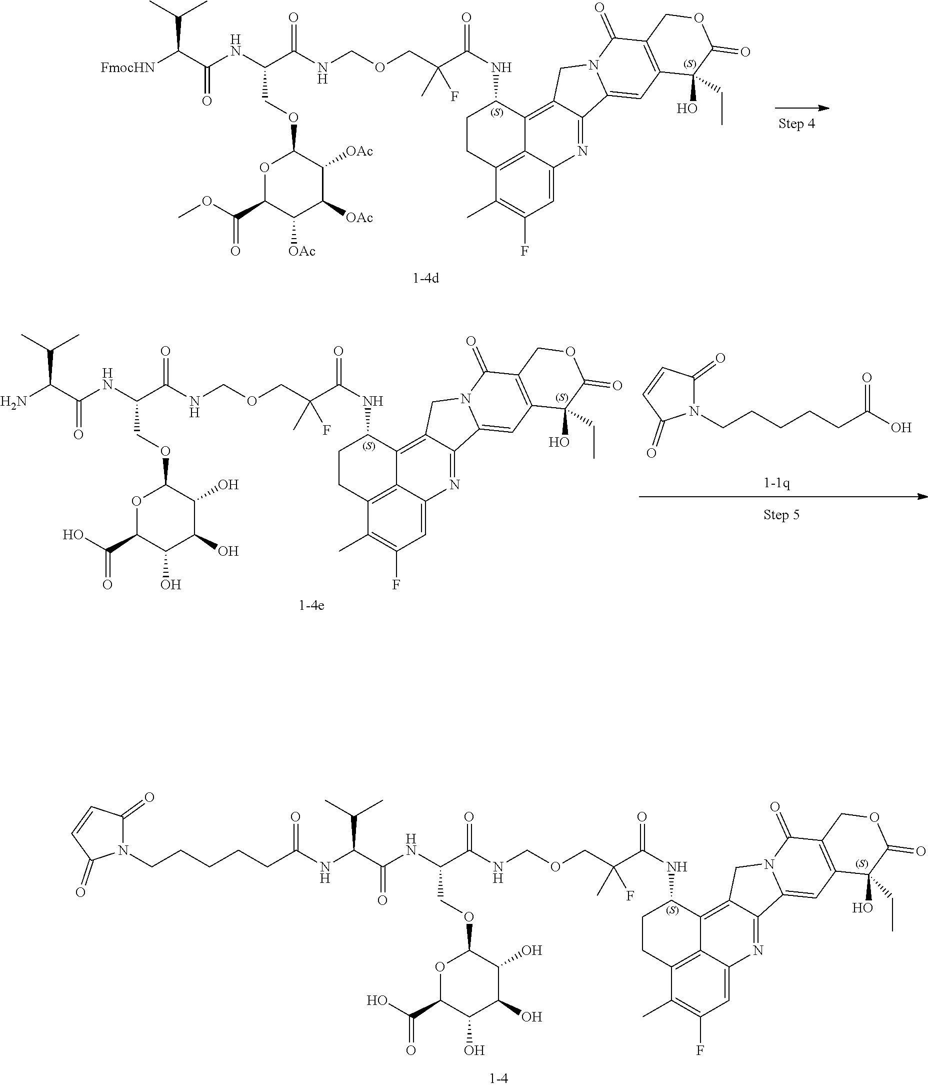

Steps 1-4: (2S,3S,4S,5R,6R)-6-((2S)-2-((S)-2-amino-3-methylbutananido)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]7]uinoline-1-yl)amino)-2-fluoro-2-methyl-3-oxopropoxy)methyl)amino)-3-oxopropoxy)-3,4,5-trihydroxytetrahydro-2H-pyran-2-carboxylic acid (1-4e). 1-4e (94 mg, 54.6% yield) was synthesized according to similar procedures as those of 1-2j. MS (ESI) rm/z: 931.7 [M+H]⁺.

Step 5: (2S,3S,4S,5R,6R)-6-((2S)-2-((S)-2-(6-(2,5-dioxo-2,5-dihydro-1H-pyrrol-1-yl)hexanamido)-3-methylbutanamido)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-]1-yl)amino)-2-fluoro-2-methyl-3-oxopropoxy)methyl)amino)-3-oxopropoxy)-3,4,5-trihydroxytetrahydro-2H-pyran-2-carboxylic acid (1-4). 1-4 (17.2 mg, 53.7% yield) was synthesized according to the same procedures as that of step 12, Example 1-1. MS (ESI) m/z: 1124.9 [M+H]⁺.

### Example 1-5

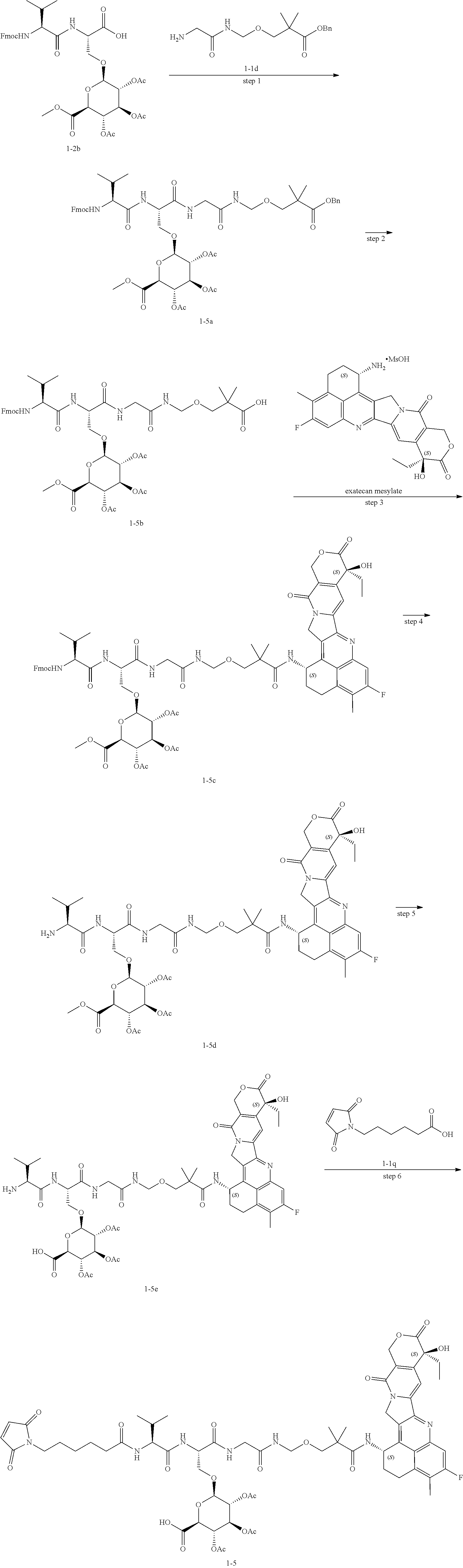

Step 1: (2R,3R,4S,5S,6S)-2-(((S)-13-((S)-2-((((9H-fluoren-9-yl)methoxy)carbonyl)amino)-3-methylbutanamido)-4,4-dimethyl-3,9,12-trioxo-1-phenyl-2,6-dioxa-8,11-diazatetradecan-14-yl)oxy)-6-(methoxycarbonyl)tetrahydro-2H-pyran-3,4,5-triyl triacetate (1-5a). To a solution of compound 1-2b (150 mg, 0.19 mmol) in DMF (4 mL) were added compound 1-Id (58.8 mg, 0.2 mmol), HATU (70.6 mg, 0.496 mmol), and DIEA (49.2 mg, 0.38 mmol). The mixture was stirred at r.t. for 30 min. The reaction mixture was diluted with EtOAc (20 mL) and washed with brine (30 mL*3). The organic layer was dried over Na₂SO₄. After filtration and concentration, the residue was purified by silica column gel chromatography (eluent: Petroleum ether/EtOAc=100/0 to 30/70) to afford 1-a (140 mg, 72.4% yield) as a white solid. MS (ESI) m/z: 1019.8 [M+H]⁺.

Step 2: (5S,8S)-1-(9H-fluoren-9-yl)-5-isopropyl-17,17-dimethyl-3,6,9,12-tetraoxo-8-((((2R,3R,4S,5S,6S)-3,4,5-triacetoxy-6-(methoxycarbonyl)tetrahydro-2H-pyran-2-yl)oxy)methyl)-2,15-dioxa-4,7,10,13-tetraazaoctadecan-18-oic acid (1-5b). To a solution of compound 1-5a (140 mg, 0.14 mmol) in MeOH (10 ml) and THF (5 ml) was added Pd/C (10%, 14 mg). The mixture was stirred at r.t. under H₂ atmosphere for 1 h. The mixture was filtered and concentrated to give crude product 1-5b (125 mg, 92.9% yield) as a white solid. MS (ESI) m/z: 929.4 [M+H]⁺.

Step 3: (2R,3R,4S,5S,6S)-2-((S)-2-((S)-2-((((9H-fluoren-9-yl)methoxy)carbonyl)amino)-3-methylbutanamido)-3-((2-(((3-(((1S,9S)-9-ethyl-5-fluoro-9-hydroxy-4-methyl-10,13-dioxo-2,3,9,10,13,15-hexahydro-1H,121H-benzo[de]pyrano[3′,4′:6,7]indolizino[1,2-b]quinolin-1-yl)amino)-2,2-dimethyl-3-oxopropoxy)methyl)amino)-2-oxoethyl)amino)-3-oxopropoxy)-6-(methoxycarbonyl)tetrahydro-2H-pyran-3,4,5-triyl triacetate (1-5c). To a solution of compound 1-5b (125 mg, 0.13 mmol) in DMF (3 mL) were added exatecan mesylate (79 mg, 0.15 mmol), HATU (54.4 mg, 0.16 mmol), and DIEA (33.54 mg, 0.26 mmol). The mixture was stirred at r.t. for 30 min. The reaction mixture was diluted with EtOAc (20 mL) and washed with brine (30 mL*3). The organic layer was dried over Na₂SO₄. After filtration and concentration, the residue was purified by silica column gel chromatography (eluent: Petroleum ether/EtOAc=100/0 to 100/0) to afford 1-5c (130 mg, 74.4% yield) as a white solid. MS (ESI) m/z: 1346.5 [M+H]⁺.

Step 4: (2R,3R,4S,5S,6S)-2-((S)-2-((S)-2-amino-3-methylbutanamido)-3-((2-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-2-oxoethyl)amino)-3-oxopropoxy)-6-(methoxycarbonyl)tetrahydro-2H-pyran-3,4,5-triyl triacetate (1-5d). To a solution of compound 1-5c (130 mg, 0.10 mmol) in DMF (3 mL) was added Et₂NH (142 mg, 2.0 mmol). The mixture was stirred at r.t. for 1 h. The mixture was concentrated under high vacuum to give compound 1-5d (110 mg, crude) as an off-white solid, which was used directly without further purification. MS (ESI) m/z: 1124.8 [M+H]⁺.

Step 5: (2S,3S,4S,5R,6R)-6-((S)-2-((S)-2-amino-3-methylbutanamido)-3-((2-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-2-oxoethyl)amino)-3-oxopropoxy)-3,4,5-trihydroxytetrahydro-2H-pyran-2-carboxylic acid (1-5e). To a solution of compound 1-5d (110 mg, 0.1 mmol) in MeOH (10 mL) and H₂O (3 mL) was added Na₂CO₃ (74 mg 0.7 mmol). The mixture was stirred at r.t. for 2 h. The mixture was acidified with HOAc and pH was adjusted to 6. After being filtered, the mixture was purified by prep-HPLC (FA) (Method: column: XBridge Prep C18 OBD 5 μm 19*150 mm; Mobile phase: A-water (0.1% formic acid): B-acetonitrile; Flow rate: 20 mL/min), the fraction was lyophilized to give compound 1-Se (80 mg, 80.2% yield) as off-white solid. MS (ESI) m/z: 985.3 [M+H]⁺.

Step 6: (2S,3S,4S,5R,6R)-6-((S)-2-((S)-2-(6-(2,5-dioxo-2,5-dihydro-1H-pyrrol-1-yl)hexanamido)-3-methylbutanamido)-3-((2-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-2-oxoethyl)amino)-3-oxopropoxy)-3,4,5-trihydroxytetrahydro-2H-pyran-2-carboxylic acid (1-5). To a solution of compound 1-5e (25 mg, 0.025 mmol) in DMF (2 mL) were added compound 1-1q (5.8 mg, 0.028 mmol), HATU (8.45 mg, 0.025 mmol), and DIEA (1.45 mg, 0.05 nmol). The mixture was stirred at r.t. for 30 min. The mixture was purified by prep-HPLC (FA) (Method: column: XBridge Prep C18 OBD 5 μm 19*150 mm; Mobile phase: A-water (0.1% formic acid): B-acetonitrile; Flow rate: 20 mL/min), the fraction was lyophilized to give compound 1-5 (15 mg, 50.9% yield) as an off-white solid. MS (ESI) m/z: 1178.2 [M+H]⁺.

### Example 1-6

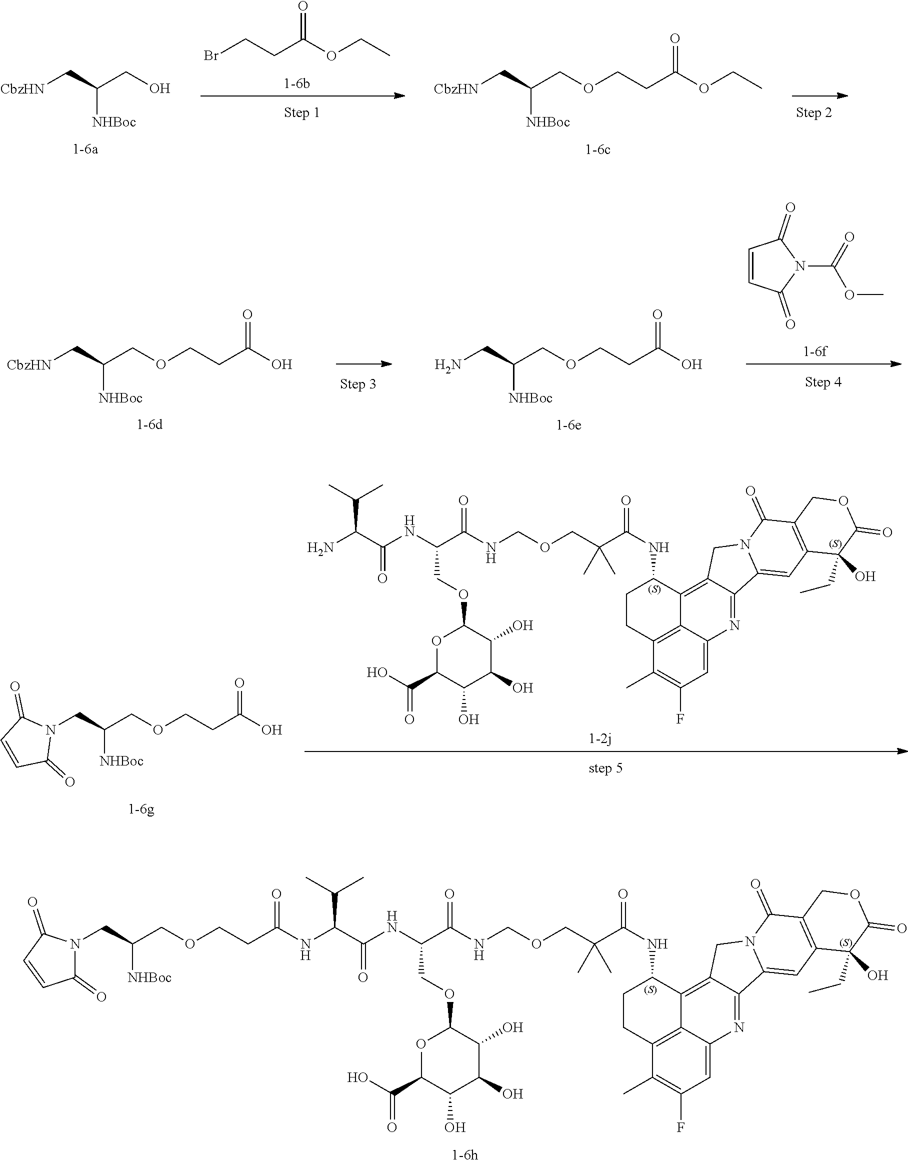

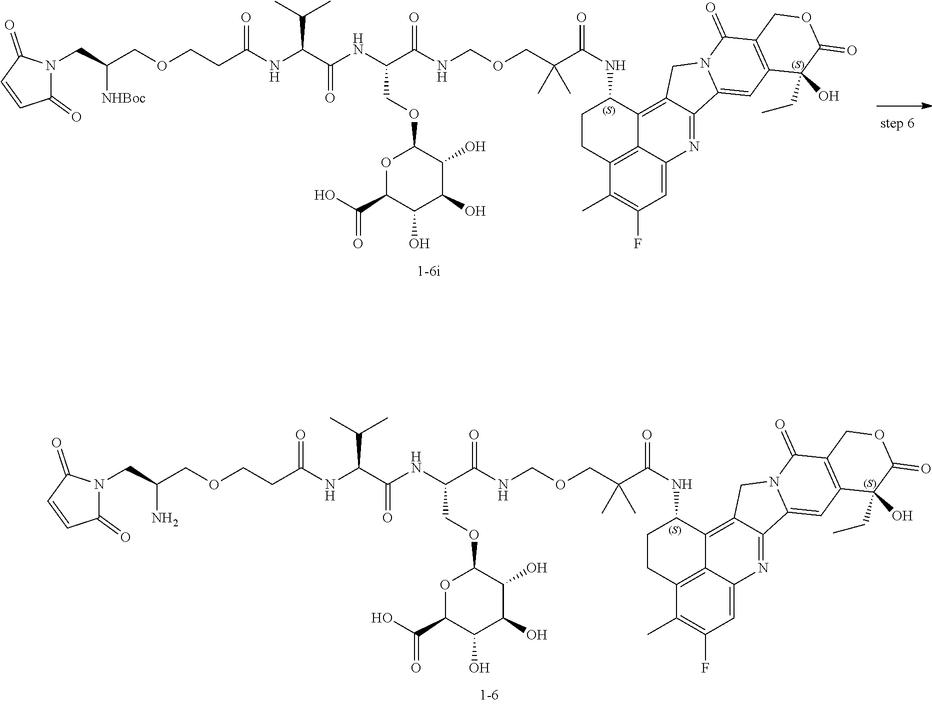

Step 1: Ethyl (S)-3-(3-(((benzyloxy)carbonyl)amino)-2-((tert-butoxycarbonyl)amino)propoxy)propanoate (1-6c). To a mixture of 1-6a (5.0 g, 15.41 mmol) (purchase from WUXI), TBAB (496.9 mg, 1.54 mmol) and 1-6b (14.0 g, 77.07 mmol) in DCM (50 mL) was added dropwise 50% wt NaOH aqueous (7.2 g, 77.07 mmol). The mixture was stirred at r.t. overnight. The mixture was diluted with DCM (100 mL), extracted with DCM (50 mL*2). Combined organic layers were washed with brine (50 mL*2), dried over Na₂SO₄, filtered, and concentrated under vacuum to give a residue. It was purified by silica gel column chromatography (Petroleum ether/EtOAc=0%-30%), fraction was concentrated under vacuum to give 1-6c (3.06 g, ~-46.8% purity) as a colorless transparent oil. MS (ESI) m/z: 447.4 [M+Na]⁺, ¹H NMR (400 MHz, cdcl₃) δ 7.32 (m, 5H), 5.35 (s, 1H), 5.18-5.04 (m, 2H), 4.19-4.09 (dq, J=6.8, 1.6 Hz 2H), 3.82 (m, 1H), 3.69 (i, 2H), 3.56 (dd, J=9.2, 3.6 Hz, 1H), 3.47 (dd, J=9.6, 5.2 Hz, 1H), 3.45-3.37 (m, 1H), 3.36-3.25 (m, 1H), 2.55 (t, J=6.0 Hz, 2H), 1.43 (s, 9H), 1.24 (t, J=7.2 Hz, 3H).

Step 2: (S)-3-(3-(((benzyloxy)carbonyl)amino)-2-((tert-butoxycarbonyl)amino)propoxy)propanoic acid (1-6d). To a solution of 1-6c (3.06 g, 7.21 mmol) in MeOH (30 mL) was added 4N NaOH (2.7 mL, 10.8 mmol) then stirred at r.t. for 12 h. The mixture was diluted with water (30 mL) and acidified with IN KHSO₄ to pH=3 in an ice-bath, extracted with EtOAc (50 mL*4). Combined organic layers were washed with brine (50 mL*3), dried over Na₂SO₄ filtered and the filtrate was concentrated under vacuum to give 1-6d (2.8 g, 98% yield) as a colorless transparent oil. MS (ESI) m/z: 419.3 [M+Na]⁺.

Step 3: (S)-3-(3-amino-2-((tert-butoxycarbonyl)amino)propoxy)propanoic acid (1-6e). To a solution of 1-6d (2.8 g, 7.1 mmol) in MeOH (30 mL) was added wet Pd/C (250 mg) then stirred at r.t. for 30 min. The black suspension was filtered through syringe head washed with 1-120 then concentrated under vacuum to give 1-6e (10.85 g) as a white solid. MS (EST) m/z: 263.2 [M+1]⁺. ¹H NMR (400 MHz, d₂O) δ 4.1-3.9 (m, 11H), 3.73 (t, J=6.0 Hz, 21H), 3.64 (dd, J=10.4, 4.4 Hz, 1H), 3.57 (dd, J=10.4, 5.2 Hz, 1H), 3.22 (dd, J=13.2, 4.4 Hz, 1H), 3.08 (dd, J=13.2, 8.0 Hz, 1H), 2.45 (t, J=6.0 Hz, 2H), 1.44 (s, 9H).

Step 4: (S)-3-(2-((tert-butoxycarbonyl)amino)-3-(2,5-dioxo-2,5-dihydro-1H-pyrrol-1-yl)propoxy)propanoic acid (1-6g). To a solution of 1-6e (500 mg, 1.91 mmol) in IN NaHCO₃ (10 mL) was added 1-6f (397.7 mg, 2.86 mmol). The mixture was stirred at 0 °C for 30 min then slowly warmed to r.t. for 2 h. The mixture was acidified with IN KHSO₄ to pH=3 in an ice-bath, extracted with EtOAc (20 mL*2). Combined organic layers were washed with brine (20 mL), dried over Na₂SO₄, filtered and the filtrate was concentrated under vacuum to give 1-6g (560 mg, 85.8% yield) as a white solid. MS (ESI) m/z: 365.3 [M+Na]⁺. ¹H NMR (400 MHz, cdcl₃) δ 6.69 (s, 2H), 5.06 (d, J=9.2 Hz, 1H), 4.08-3.97 (m, 1H1), 3.82~3.68 (m, 31H), 3.59 (dd, J=14.0, 3.6 Hz, 2H), 3.51 (dd, J=9.6, 4.4 Hz, 1H), 2.64 (t, J=6.2 Hz, 1H), 1.38 (s, 9H).

Step 5: (2S,3S,4S,5R,6R)-6-(((6S,13S,16S)-6-((2,5-dioxo-2,5-dihydro-1H-pyrrol-1-yl)methyl)-16-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)carbamoyl)-13-isopropyl-2,2-dimethyl-4,11,14-trioxo-3,8-dioxa-5,12,15-triazaheptadecan-17-yl)oxy)-3,4,5-trihydroxytetrahydro-2H-pyran-2-carboxylic acid (1-6h). To a solution of 1-6g (6.7 mg, 0.02 mmol) in DMF (2 ml) were added HATU (8.6 mg, 0.02 mmol) and DIPEA (5.2 mg, 0.04 mmol). The mixture was stirred at r.t. for 10 min. 1-2j (15 mg, 0.02 mmol) was added and stirred at r.t. for 30 min. The mixture was purified by prep-HPLC (FA) to give 1-6h (9.1 mg, 44.4% yield) as an off-white solid. MS (ESI) m/z: 1252.1 [M+H]⁺.

Step 6: (2S,3S,4S,5R,6R)-6-((S)-2-((S)-2-(3-((S)-2-amino-3-(2,5-dioxo-2,5-dihydro-1H-pyrrol-1-yl)propoxy)propanamido)-3-methylbutanamido)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4:6,7]indolizino[1,2-b]quinolin-1-yl)amino)-2,2-dimethyl-3-oxopropoxy)methyl)amino)-3-oxopropoxy)-3,4,5-trihydroxytetrahydro-2H-pyran-2-carboxylic acid (1-6). To a suspension of 1-6h (9.1 mg, 0.007 mmol) in DCM (1.5 ml) was added ZnBr₂ (32.4 mg, 0.14 mmol) then stirred at 45 °C for 8 h. The mixture was concentrated under vacuum and dissolved in DMSO then purified by prep-HPLC (0,1% FA) (Method: column: XBridge Prep C18 OBD 5 μm 19*150 mm; Mobile phase: A-water (0.1% FA): B-acetonitrile; Flow rate: 20 ml/min), the fraction was lyophilized to give 1-6 (7.1 mg, 85.7% yield) as a white solid. MS (EST) m/z: 1151.9 [M+H]⁺.

### Example 1-7

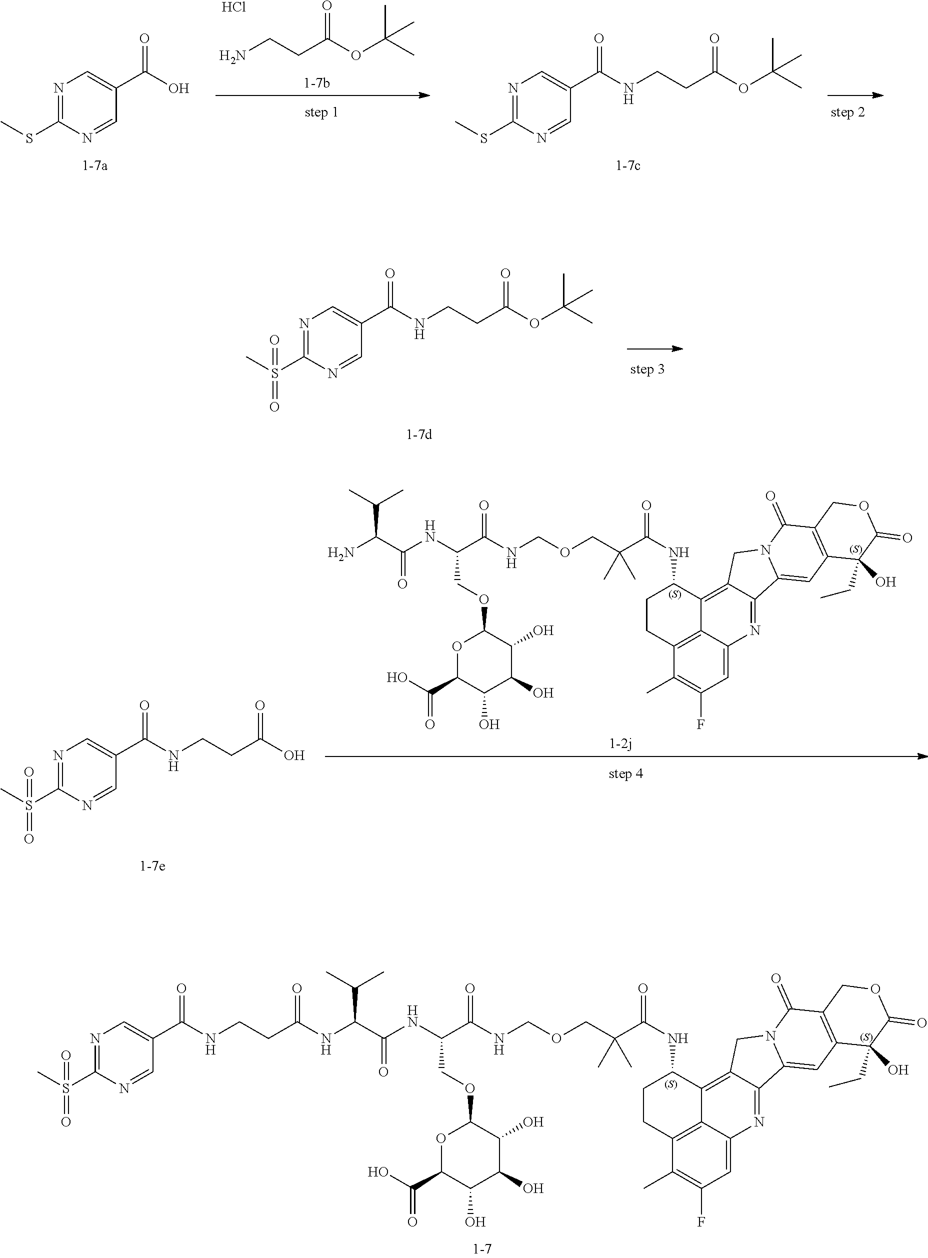

Step 1: tert-butyl 3-(2-(methylthio)pyrimidine-5-carboxanido)propanoate (1-7c). To a mixture of 1-7a (600 mg, 3.53 mmol) in DMF (15 mL) were added HATU (1622 mg, 4.27 mmol) and DIPEA (1.9 mL, 1504 mg, 11.63 mmol). The mixture was stirred at 45 °C for 10 min, 1-7b (704.6 mg, 3.878 mmol) was added and stirred at 45 °C for 40 min. The mixture was diluted with EtOAc (50 mL), washed with brine (30 mL*3), dried over Na₂SO₄, filtered, and concentrated under vacuum to give residue. It was purified by flash column chromatography (MeOH/DCM=0/100~10/90) to give 1-7c (1.3 g crude) as a brown oil. MS (ESI) m/z: 298.4 [M+H]⁺.

Step 2: tert-butyl 3-(2-(methylsulfonyl)pyrimidine-5-carboxanido)propanoate (1-7d). To a solution of 1-7c (500 mg, crude) in DCM (10 mL) was added m-CPBA (1160.5 mg, 6.725 mmol). The white suspension was stirred at r.t. for 1 h. The mixture was diluted with EtOAc (50 ml) and quenched by addition of sat. Na₂S₂O³ (30 mL), stirred at r.t. for 10 min, extracted with DCM (30 mL*3). Combined organic layers were washed with brine (50 mL), dried over Na₂SO₄, filtered, and concentrated under vacuum to give crude product as a yellow solid. It was purified by flash column chromatography (MeOH/DCM=0/100-10/90), fraction was concentrated under vacuum to give 1-7d (303 mg, ~50% yield) as a white solid. MS (ESI) m/z: 330.3 [M+H]⁺. ¹H NMR (400 MHz, CDCl₃) δ 9.27 (s, 21H), 7.38 (s, 1H), 3.74 (dd, J=11.6, 6.0 Hz, 1H), 3.40 (s, 21), 2.60 (t, 11H), 1.47 (s, 51H).

Step 3: 3-(2-(methylsulfonyl)pyrimidine-5-carboxamido)propanoic acid (1-7e). A mixture of 1-7d (303.0 mg, 0.92 mmol) in TFA (1 mL) and DCM (2 mL) stirred at r.t. for 1.5 h. The mixture was concentrated under vacuum and co-evaporated with Tol for three time to give 1-7e (255 mg) as an off-white solid. MS (ESI) m/z: 274.1 [M+H]⁺.

Step 4: (2S,3S,4S,5R,6R)-6-((S)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-2-((S)-3-methyl-2-(3-(2-(methylsulfonyl)pyrimidine-5-carboxamido)propanamido)butanamido)-3-oxopropoxy)-3,4,5-trihydroxytetrahydro-2H-pyran-2-carboxylic acid (1-7). To a solution of 1-7e (7.1 mg, 0.026 mmol) and 1-2j (20.0 mg, 0.022 mmol) in DMF (2 mL) were added HATU (11.5 mg, 0.030 mmol) and DIPEA (9 μL, 7.0 mg, 0.054 mmol). The mixture was stirred at r.t. for 30 min. The mixture was purified by prep-HPLC (FA 0.1%) (Method: column: XBridge Prep C18 OBD 5 μm 19*150 mm; Mobile phase: A-water (0.1% FA): B-acetonitrile; Flow rate: 20 ml/min), the fraction was lyophilized give 1-7 (13.4 mg, ~52.5% yield) as a white solid. MS (ESI) m/z: 1182.8 [M+H]⁺.

### Example 1-8

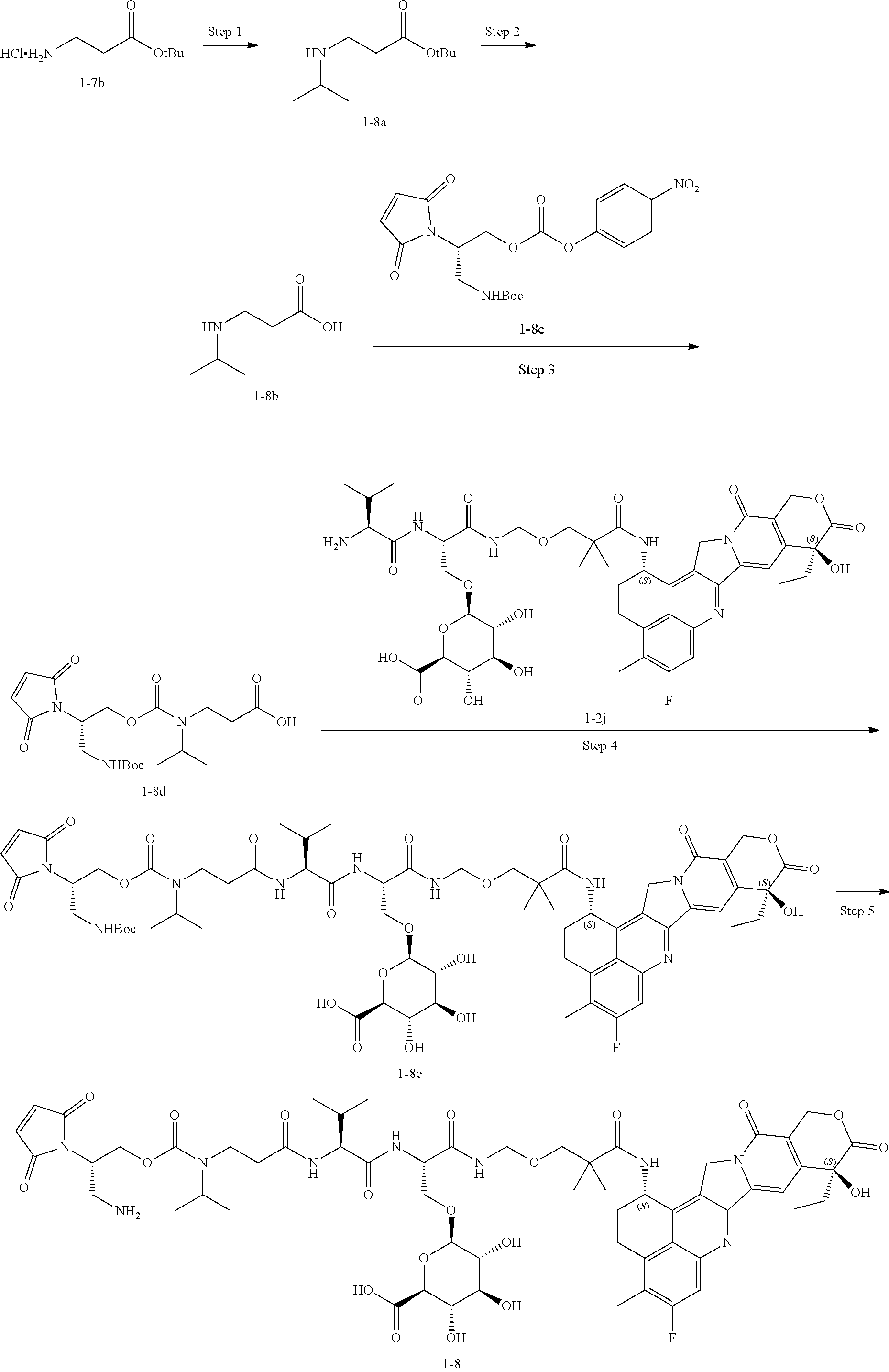

Step 1: tert-Butyl 3-(isopropylamino)propanoate (1-8a). To a solution of 1-7b (908 mg, 5.0 mmol) and acetone (580 mg, 10.0 mmol) in MeOH—H₂O (1:1, 20 mL) was added NaBH₃CN (1.28 g, 20.0 mmol) at r.t. The reaction was stirred at r.t. for 18 h. The mixture was concentrated in vacuo and the residue was re-dissolved in DCM (20 mL) and washed with sat. NaHCO₃ (10 mL), H₂O (mL), brine (10 mL), dried over Na₂SO₄, filtered and the filtrate was concentrated under vacuum to give a crude target product 1-8a (1.21 g) as a colorless oil, which was used for next step directly. MS (ESI) m/z: 188.2 [M+H]⁺.

Step 2: 3-(Isopropylamino)propanoic acid (1-8b). A solution of 1-8a (600 mg, 2.5 mmol) in TFA/DCM (5:2, 7 mL) was stirred at r.t. overnight. The mixture was concentrated under vacuum, and the residue was re-dissolved in water. The aqueous layer was washed by EtOAc and lyophilized to give 1-8b (444.4 mg, 77.9%) yield) as a white solid. MS (ESI) m/z: 132.1 [M+H]⁺.

Step 3: S)-7-(2,5-Dioxo-2,5-dihydro-1H-pyrrol-1-yl)-It-isopropyl-2,2-dimethyl-4,10-dioxo-3,9-dioxa-5,11-diazatetradecan-14-oic acid (1-8d). To a solution of 1-8b (12.3 mg, 0.094 mmol) and 1-Sc (20 mg, 0.046 mmol) in DMF (1 mL) was added DIPEA (30 mg, 0.23 mmol) and HOBt (2 mg, 0.014 mmol) at r.t. The reaction was stirred at r.t. for 16 min. Reaction mixture was diluted by DMF and acidified by 0.1 N HCl. The mixture was purified by prep-HPLC (FA), the fraction was lyophilized to give 1-8d (6.8 mg, 34.6% yield) as a pale-yellow solid. MS (ESI) m/z: 450.48 [M+Na]⁺.

Step 4: (2S,3S,4S,5R,6R)-6-(((7S,16S,19S)-7-(2,5-Dioxo-2,5-dihydro-H-pyrrol-1-yl)-19-(((3-(((1S,95)-9-ethyl-5-fluoro-9-hydroxy-4-methyl-10,13-dioxo-2,3,9,10,13,15-hexahydro-1H,12H-benzo[de]pyrano[3,4′:6,7]indolizino[1,2-b]quinolin-1-yl)amino)-2,2-dimethyl-3-oxopropoxy)methyl)carbamoyl)-11,16-diisopropyl-2,2-dimethyl-4,10,14,17-tetraoxo-3,9-dioxa-5,11,15,18-tetraazaicosan-20-yl)oxy)-3,4,5-trihydroxytetrahydro-2H-pyran-2-carboxylic acid (1-8e). To a solution of 1-8d (6.8 mg, 0.0159 mmol) in DMF (0.5 mL) were added HATU (6.0 mg, 0.0159 mmol), DIPEA (4.7 mg, 0.0363 mmol), and 1-2j (13.4 mg, 0.0145 mmol). The reaction was stirred at r.t. for 30 min. The mixture was diluted with DMF (1.5 mL) and acidified by HOAc. It was purified by prep-HPLC (FA), the fraction was lyophilized to give 1-8e (5.1 mg, 26.4% yield) as a white solid. MS (ESI) m/z: 1358.9 [M+Na]⁺.

Step 5: (2S,3S,4S,5R,6R)-6-(((2S,5S,14S)-15-Amino-14-(2,5-dioxo-2,5-dihydro-1H-pyrrol-1-yl)-2-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)carbamoyl)-5,10-diisopropyl-4,7,11-trioxo-12-oxa-3,6,10-triazapentadecyl)oxy)-3,4,5-trihydroxytetrahydro-2R-pyran-2-carboxylic acid (1-8). To a suspension of 1-Se (5.1 mg, 0.007 mmol) in DCM (1.5 mL) was added ZnBr₂ (32.4 mg, 0.14 mmol) then stirred at 45 °C for 21 h. The mixture was concentrated under vacuum and dissolved in 0.1% FA in water/ACN (80:20, 1 mL) then purified by prep-HPLC (0.1% TFA), fraction was lyophilized to give 1-8 TFA (2.2 mg, 46.6% yield) as a white solid. MS (ESI) m/z: 1236.8 [M+H]⁺.

### Example 1-9

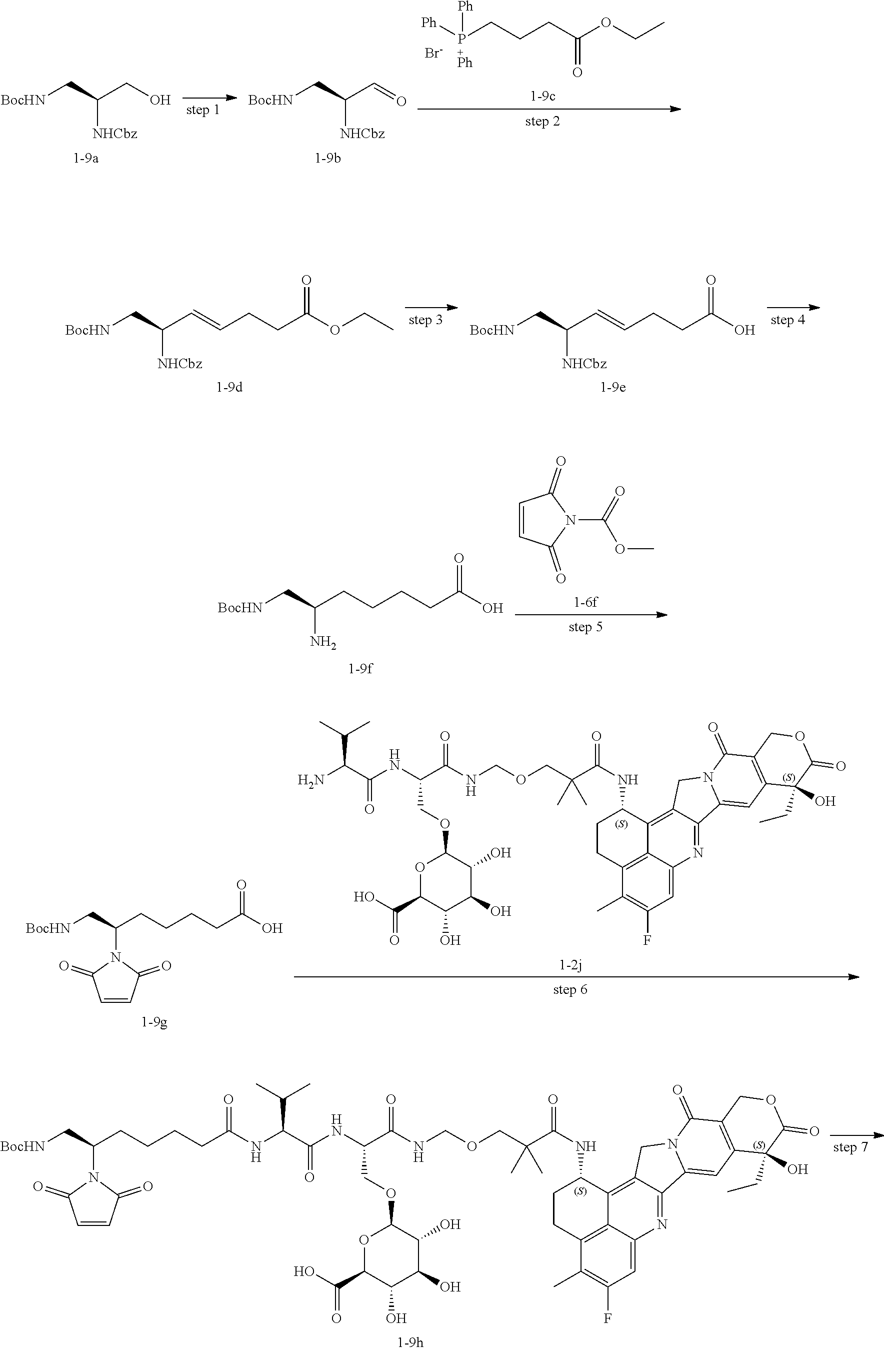

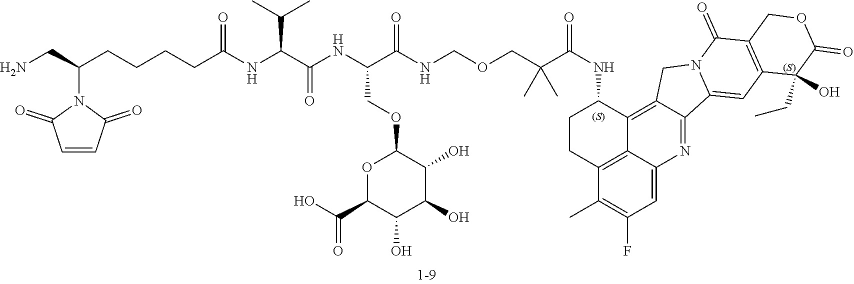

Step 1: benzyl tert-butyl (3-oxopropane-1,2-diyl)(S)-dicarbamate (1-9b). A solution of (COCl)₂ (3.9 g, 30.83 mmol) in DCM (50 mL) was cooled to −70 °C, DMSO (3.6 g, 46.24 mmol) was added dropwise. Then stirred at −70 °C for 30 min. 1-9a (5.0 g, 15.41 mmol) dissolved in DMSO (10 mL) was added through syringe then stirred at −70 °C for 30 min. TEA (7.8 g, 77.1 mmol) was added dropwise and then slowly warmed to r.t. The mixture was poured into water (100 mL), extracted with EtOAc (50 mL*2). Combined organic layers were washed with brine (100 mL*2), dried over Na₂SO₄, filtered and the filtrate was concentrated under vacuum to give a residue. It was purified by flash column chromatography (EtOAc/Petroleum ether=1/99-50/50) as mobile phase, fraction was concentrated under vacuum to give 1-9b (4.86 g, 97.8% yield) as a yellow oil. ¹H NMR (400 MHz, cdcl₃) δ 9.64 (s, 1H), 7.39-7.30 (m, 5H), 5.93 (s, 1H), 4.86 (s, 11H), 4.35-4.22 (m, 11H), 3.77-3.64 (m, 1H), 3.56 (dt, J=14.8, 4.8 Hz, 1H), 1.40 (s, 9H).

Step 2: ethyl (R^{#})-6-(((benzyloxy)carbonyl)amino)-7-((tert-butoxycarbonyl)amino)hept-4-enoate (1-9d). To a suspension of 1-9b (6.5 g, 14.3 mmol) in Tol (30 mL) was added dropwise —BuOK (1M solution in THF, 14.5 mL, 14.5 mmol) at 0 °C, the mixture turned a deep color over 30 min. 1-9c (2.0 g, 6.2 mmol) in Tol (20 mL) was added dropwise at −70 °C, then slowly warmed to r.t. The mixture was quenched by addition of sat. NH₄Cl (50 mL). After separation the Tol phase was washed with brine (30 mL*2), dried over Na₂SO₄. filtered and the filtrate was concentrated under vacuum to give a residue. It was purified by flash column chromatography (EtOAc/Petroleum ether=0/100~30/70~50/50) to give 1-9d (808 mg, 30.8% yield) as a white solid. MS (ESI) m/z: 443.4 [M+Na]⁺. ¹H NMR (400 MHz, cdcl₃) δ 7.40-7.27 (m, 5H1), 5.72~5.60 (m, 0.2H), 5.60-5.46 (i, 0.8H), 5.40 (dd, J=16.0 Hz, 6.4 Hz, 0.2H), 5.34-5.20 (m, 1.8H), 4.95-4.70 (m, 1H), 5.16-5.01 (m, 2H), 4.60-4.40 (m, 1H),4.12 (q, J=7.2 Hz, 2H), 3.39-3.08 (m, 2H), 2.6~2.2 (m, 4H), 1.42 (s, 9H), 1.24 (t, J=7.2 Hz, 1H).

Step 3: (R^{#})-6-(((benzyloxy)carbonyl)amino)-7-((tert-butoxycarbonyl)amino)hept-4-enoic acid (1-9e). To a solution of 1-9d (808.0 mg, 1.92 mmol) in MeOH (10 mL) was added 4N NaOH (1.92 mL, 7.69 mmol). The mixture was stirred at r.t. for 3.5 h. The mixture was diluted with water (20 mL), acidified with sat. KHSO₄ to pH=3, extracted with EtOAc (30 mL*3). Combined organic layers were washed with brine (50 mL), dried over Na₂SO₄, filtered and the filtrate was concentrated under vacuum to give 1-9e (781 mg) as a white solid. MS (ESI) m/z: 415.4 [M-Na]⁺.

Step 4: (R^{#})-6-amino-7-((tert-butoxycarbonyl)amino)heptanoic acid (1-9f). To a solution of 1-9e (781.0 mg, 1.99 mmol) in MeOH (8 mL) was added wet Pd/C (67 mg) then stirred at r.t. for 24 h. The black suspension was filtered through syringe head washed with MeOH/H₂O (5:1, 30 mL) then concentrated under vacuum to give 1-9f (510 mg, 98.5% yield) as a white solid. MS (ESI) m/z: 261.3 [M+H]⁺.

Step 5: (R^{#})-7-((tert-butoxycarbonyl)amino)-6-(2,5-dioxo-2,5-dihydro-1H-pyrrol-1-yl)heptanoic acid (1-9g). To a mixture of 1-9f (510 mg, 1.96 mmol) in 1N NaHCO₃ (10 mL) was added 1-6e (455.8 mg, 2.94 mmol) at 0 °C, then stirred at 0 °C for 30 min and allowed to warm to r.t. for 3 h. The reaction was acidified with sat. KHSO₄ to pH=3, extracted with EtOAc (30 mL*3). Combined organic layers were washed with brine (50 mL), dried over Na₂SO₄, filtered and the filtrate was concentrated to give a residue. It was purified by flash column chromatography (MeOH/DCM=0/100-5/95), fraction was concentrated under vacuum to give 1-9g (590 mg, 85% yield) as a pale-yellow oil. MS (ESI) m/z: 363.3 [M+Na]⁺.

Step 7: (2S,3S,4S,5R,6R)-6-((S)-2-((S)-2-((R^{#})-7-amino-6-(2,5-dioxo-2,5-dihydro-1H-pyrrol-1-yl)heptanamido)-3-methylbutanamido)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-3-oxopropoxy)-3,4,5-trihydroxytetrahydro-2H-pyran-2-carboxylic acid (1-9). 1-9 (5.8 mg, 45% yield) as a white solid was synthesized according to the synthetic procedure of Example 1-6.

### Example 1-10

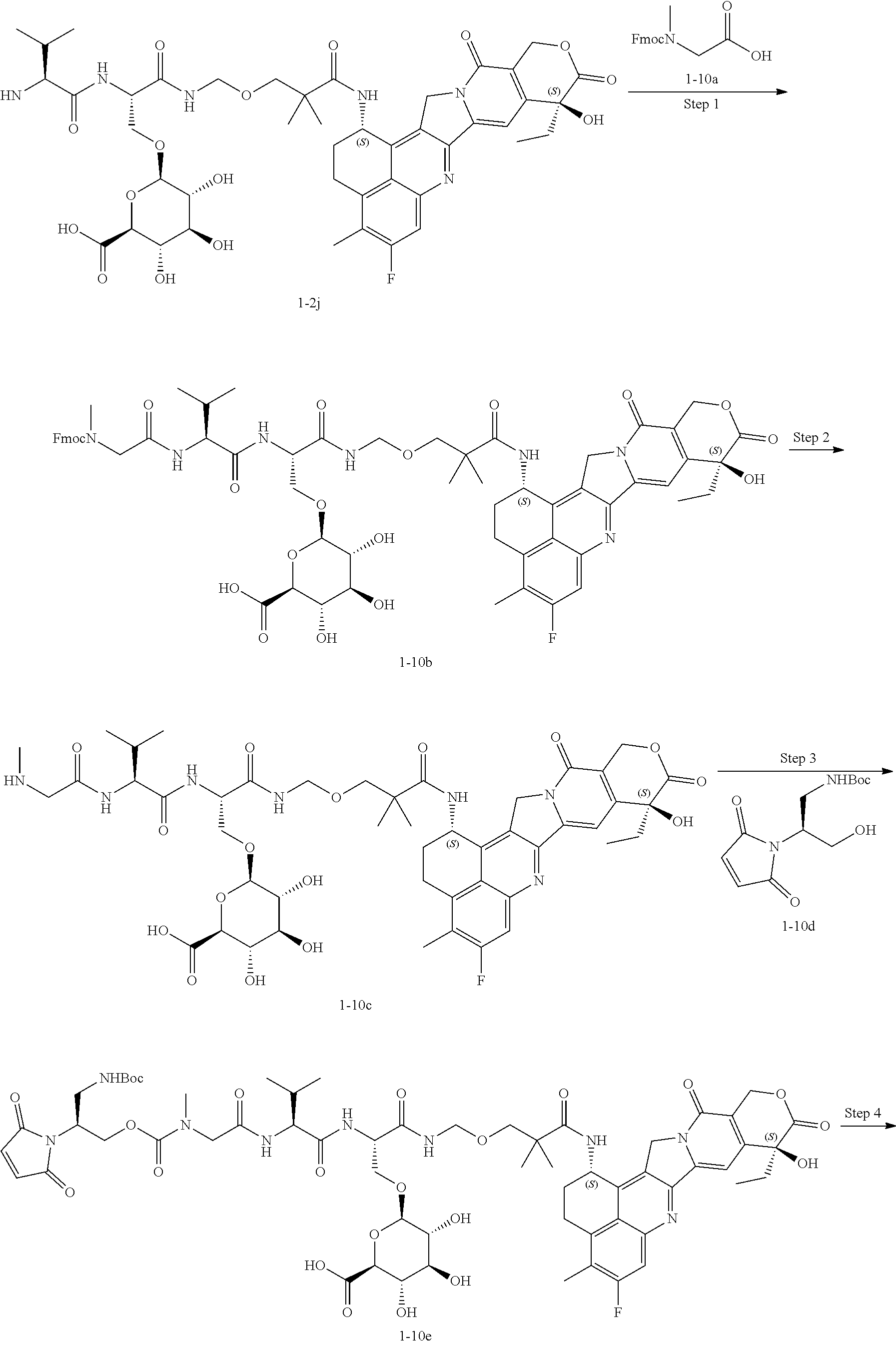

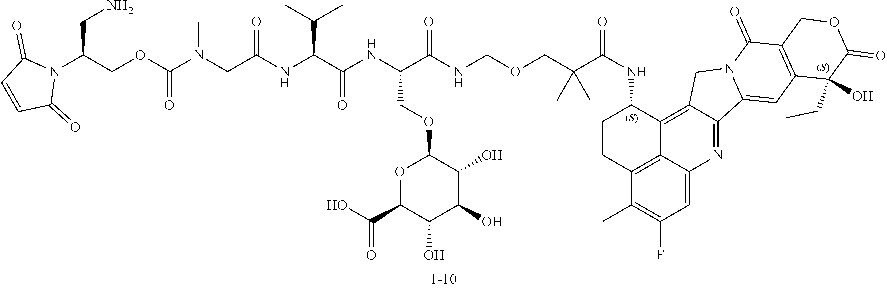

Step 1: (2S,3S,4S,5R,6R)-6-(((8S,11S)-11-(((3-(((1S,9S)-9-Ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)carbamoyl)-1-(9H-fluoren-9-yl)-8-isopropyl-4-methyl-3,6,9-trioxo-2-oxa-4,7,10-triazadodecan-12-yl)oxy)-3,4,5-trihydroxytetrahydro-2H-pyran-2-carboxylic acid (1-10b). To a solution of 1-10a (11.1 mg, 0.0356 mmol) in DMF (1 mL) was added HATU (13.5 mg, 0.0356 mmol), DIPEA (10.5 mg, 0.081 mmol), and 1-2j (30 mg, 0.0323 mmol). The reaction was stirred at r.t. for 30 min. The mixture was diluted with DMF (1.5 mL) and acidified by HOAc. It was purified by prep-HPLC (FA), the fraction was lyophilized to give 1-10b (14.6 mg, 36.9% yield) as a pale yellow solid. MS (ESI) nm/z: 1221.0 [M+H]⁺.

Step 2: (2S,3S,4S,5R,6R)-6-((S)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-2-((S)-3-methyl-2-(2-(methylamino)acetamido)butanamido)-3-oxopropoxy)-3,4,5-trihydroxytetrahydro-2H-pyran-2-carboxylic acid (1-10c). To a solution of 1-10b (14.6 mg, 0.012 mmol) in DMF (0.3 mL) was added Et₂NH (8.8 mg, 0.12 mmol). The mixture was stirred at r.t. for 20 min. Reaction mixture was concentrated under vacuum and co-evaporated with toluene for two times to give 1-10c as a brown solid, which was used for the next step directly.

Step 3: (2S,3S,4S,5R,6R)-6-(((7S,15S,18S)-7-(2,5-dioxo-2,5-dihydro-1H-pyrrol-1-yl)-18-(((3-(((1S,9S)-9-ethyl-5-fluoro-9-hydroxy-4-methyl-10,13-dioxo-2,3,9,10,13,15-hexahydro-11H,12H-benzo[de]pyrano[3′,4′:6,7]indolizino[1,2-b]quinolin-1-yl)amino)-2,2-dimethyl-3-oxopropoxy)methyl)carbamoyl)-15-isopropyl-2,2,11-trimethyl-4,10,13,16-tetraoxo-3,9-dioxa-5,11,14,17-tetraazanonadecan-19-yl)oxy)-3,4,5-trihydroxytetrahydro-2H-pyran-2-carboxylic acid (1-10e). To a mixture of 1-10d (3.6 mg, 0.0132 mmol) in DMF (0.2 mL) were added DSC (3.5 mg, 0.0136 mmol) and DIPEA (2.3 mg, 0.018 mmol). The mixture was stirred at r.t. for 3 h. Crude solid 1-10c was then added and stirred at r.t. for another 30 min. The mixture was diluted with DMF (1.5 mL) and acidified by HOAc. It was purified by prep-HPLC (FA) to give 1-10e (5.4 mg, 34.8% yield) as a pale-yellow solid. MS (ESI) m/z: 1294.9 [M+H]⁺.

Step 4: (2S,3S,4S,5R,6R)-6-(((2S,5S,13S)-14-amino-13-(2,5-dioxo-2,5-dihydro-1H-pyrrol-1-yl)-2-(((3-(((1S,9S)-9-ethyl-5-fluoro-9-hydroxy-4-methyl-10,13-dioxo-2,3,9,10,13,15-hexahydro-11H,12H-benzo[de]pyrano[3′,4′:6,7]indolizino[1,2-b]quinolin-1-yl)amino)-2,2-dimethyl-3-oxopropoxy)methyl)carbamoyl)-5-isopropyl-9-methyl-4,7,10-trioxo-11-oxa-3,6,9-triazatetradecyl)oxy)-3,4,5-trihydroxytetrahydro-2H-pyran-2-carboxylic acid (1-10). To a suspension of 1-10e (5.4 mg, 0.0042 mmol) in DCM (0.5 mL) was added ZnBr₂ (24 mg, 0.11 mmol) then stirred at 45 °C for 24 b. The mixture was concentrated under vacuum and dissolved in 0.1% FA in water/ACN (80:20, 1 mL) then purified by prep-HPLC (0.1% TFA), fraction was lyophilized to give 1-10 (1.3 mg, 26% yield) as a pale-yellow solid. MS (ESI) m/z: 1194.9 [M+H]⁺.

### Example 1-11

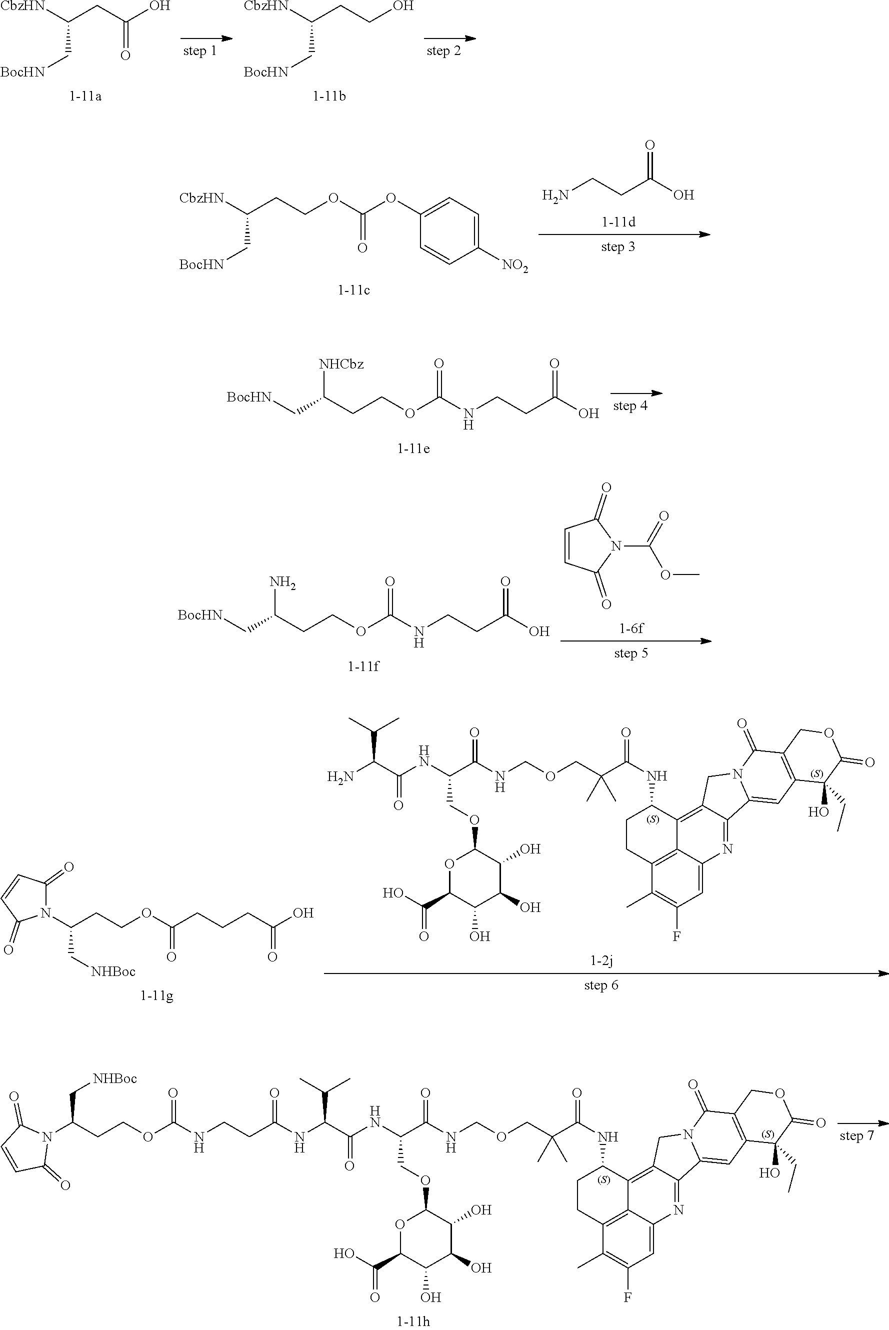

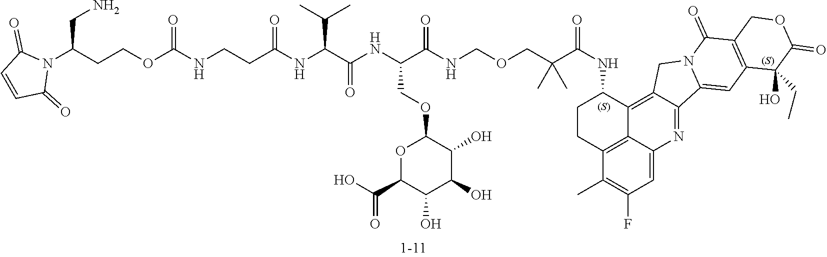

Step 1: benzyl tert-butyl (4-hydroxybutane-1,2-diyl)(R^{#})-dicarbamate (1-11b). 1-11a (2.00 g, 5.68 mmol) and K₂CO₃ (863 mg 6.24 nmol) ere added into DMF (10 mL) followed by the dropwise addition of CH₃I (161 g, 11.35 mmol) at 0 °C. The resulting mixture was stirred at 0 °C for 20 min and allowed to warm to 25 °C and further stirred at 25 °C for 60 min. The reaction process was monitored by TLC (Petroleum ether/EtOAc) and LCMS. After complete reaction, the reaction mixture was diluted with EtOAc (80 mL) and washed with brine (30 mL*3) and H₂O (30 mL*2). The organic layer was dried over anhydrous Na₂SO₄, filtered, and concentrated under reduced pressure to afford the methyl ester (2.08 g, quant.) as a light-yellow solid.

The methyl ester (1.00 g, 2.73 mmol) was dissolved in MeOH (15 mL) followed by the addition of LiBH₄ (2 M stock solution in THF, 6.80 mL) at 0 °C. The resulting mixture was stirred at 25 °C for 2 h. Reaction process was monitored by LCMS and TLC. After complete reaction, saturated aqueous NH₄Cl (10 mL) was added to quench the reaction. The reaction mixture was diluted with H₂O (80 mL) and extracted with EtOAc (50 mL*3). The combined organic layers were washed with brine (40 mL*2) and water (40 mL*2) dried over anhydrous Na₂SO₄, filtered, and concentrated under reduced pressure and further purified by flash column chromatography (Petroleum ether/EtOAc) to afford 1-11b (760 mg, 82.3% yield) as a white solid. MS (ESI) m/z: 239.2 [M-Boc+H]⁺.

Step 2: benzyl tert-butyl (4-(((4-nitrophenoxy)carbonyl)oxy)butane-1,2-diyl)(R)-dicarbamate (1-1c). 1-11b (300 mg, 0.89 mmol) and bis(4-nitrophenyl) carbonate (405 mg, 1.33 mmol) were dissolved in DMF (5 mL) followed by the addition of DIEA (229 mg, 1.77 mmol). The resulting mixture was stirred at 25 °C for 3 h. After complete reaction, the reaction mixture was diluted with EtOAc (100 mL) and washed with brine (35 mL*3) and water (35 mL*3). The organic layer was dried over anhydrous Na₂SO₄, filtered, concentrated under reduced pressure, and further purified by flash column chromatography (Petroleum ether/EtOAc) to afford 1-11c as a white solid (371 mg, 83.1% yield). MS (EST) m/z: 404.4 [M-Boc+H]⁺.

Step 3: (R^{#})-7-(((benzyloxy)carbonyl)amino)-2,2-dimethyl-4,11l-dioxo-3,10-dioxa-5,12-diazapentadecan-15-oic acid (1-11e). 1-11c (420 mg, 0.83 mmol) and 1-11d (149 mg, 1.67 mmol) were dissolved in DMF (5 mL) followed by the addition of aqueous NaHCO₃(1M, 5 mL). The resulting mixture was stirred at 25 °C for 2 h. Reaction was monitored by LCMS. After complete reaction, the reaction mixture was concentrated and purified by flash column chromatography (DCM:MeOH) to afford 1-11e as a pale-yellow solid (339 mg, 89.6% yield). MS (ESI) m/z: 354.4 [M-Boc+H]⁺.

Step 4: (R^{#})-7-amino-2,2-dimethyl-4,11-dioxo-3,10-dioxa-5,12-diazapentadecan-15-oic acid (1-11f). 1-11e (360 mg, 0.79 mmol) was dissolved in MeOH (18 mL) followed by the addition of Pd/C (wet base, 108 mg) and NH₃-MeOH (7 M, 2 drops). The resulting mixture was stirred at r.t. under H₂ (15 psi) for 2.5 h. After complete reaction, the reaction mixture was filtered and concentrated under reduced pressure to afford 1-11f as a clear syrup (252 mg, quant.). The crude product was used directly in the next step without purification. MS (ESI) m/z: 320.3 [M+H]⁺.

Step 5: (R^{#})-7-(2,5-dioxo-2,5-dihydro-1H-pyrrol-1-yl)-2,2-dimethyl-4,11-dioxo-3,10-dioxa-5,12-diazapentadecan-15-oic acid (1-11g). 1-11f (250 mg, 0.78 mmol) and 1-6f (243 mg, 1.57 mmol) were dissolved in a mixed solvent of ACN (8 mL) and aqueous NaHCO₃ (1 M, 16 mL) at 0 °C. The resulting mixture was stirred at 0 °C for 0.5 h and further stirred at 25 °C until the reaction was complete. Then the reaction mixture was acidified with aq. KHSO₄ (1M) to pH~3 and extracted with EtOAc (40 mL*3). The combined organic layers were dried over anhydrous Na₂SO₄, filtered, and concentrated under reduced pressure to afford a yellow oil as the crude product which was purified by flash column chromatography (DCM:MeOH) to afford 1-11g (280 mg, 89.6% yield) as a white solid. MS (ESI) ma/z: 422.3 [M+Na]⁺.

Step 6: (2S,3S,4S,5R,6R)-6-(((7R,17S,20S)-7-(2,5-dioxo-2,5-dihydro-1H-pyrrol-1-yl)-20-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]87uinoline-1-yl)amino)-2,2-dimethyl-3-oxopropoxy)methyl)carbamoyl)-17-isopropyl-2,2-dimethyl-4,11,15,18-tetraoxo-3,10-dioxa-5,12,16,19-tetraazahenicosan-21-yl)oxy)-3,4,5-trihydroxytetrahydro-2H-pyran-2-carboxylic acid (1-11h). To a solution of compound 1-11g (230 mg, 0.58 mmol) in DMF (6 mL) were added HATU (200 mg, 0.53 mmol) and DIEA (186 mg, 1.43 mmol). The mixture was stirred at r.t. for 10 min. Compound 1-2j (500 mg, 0.48 mmol) was added into the mixture and stirred at r.t. for 1 h. The reaction mixture was poured into MTBE (50 ml) and i-PrOH (5 mL), stirred for 10 min, filtered and dried. The crude product 1-11h (628 mg, theoretical yield) was used for the next step without further purification. MS (ESI) m/z: 1331.0 [M+Na]⁺.

Step 7: (2S,3S,4S,5R,6R)-6-(((2S,5S,15R)-16-amino-15-(2,5-dioxo-2,5-dihydro-1H-pyrrol-1-yl)-2-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)carbamoyl)-5-isopropyl-4,7,11-trioxo-12-oxa-3,6,10-triazahexadecyl)oxy)-3,4,5-trihydroxytetrahydro-2H-pyran-2-carboxylic acid (1-11). To a solution of compound 1-1th (628 mg, 0.48 mmol) in DCM (10 mL) were added ZnBr₂ (5.4 g, 24 mmol). The mixture was stirred at 39~40 °C for 16 h. The mixture was concentrated and dissolved by H₂O (0.1% FA, 9 mL). The solution was purified by prep-HPLC (Method: column: XBridge Prep C18 OBD 5 μm 19*150 mm; Mobile phase: A-water (0.1% TFA): B-acetonitrile; Flow rate: 20 mL/min) to give compound 1-11 (TFA salt, 240 mg, 38.2% yield for 2 step) as a light-yellow solid. MS (ESI) m/z: 1208.9 [M+H]⁺.

### Example 1-12

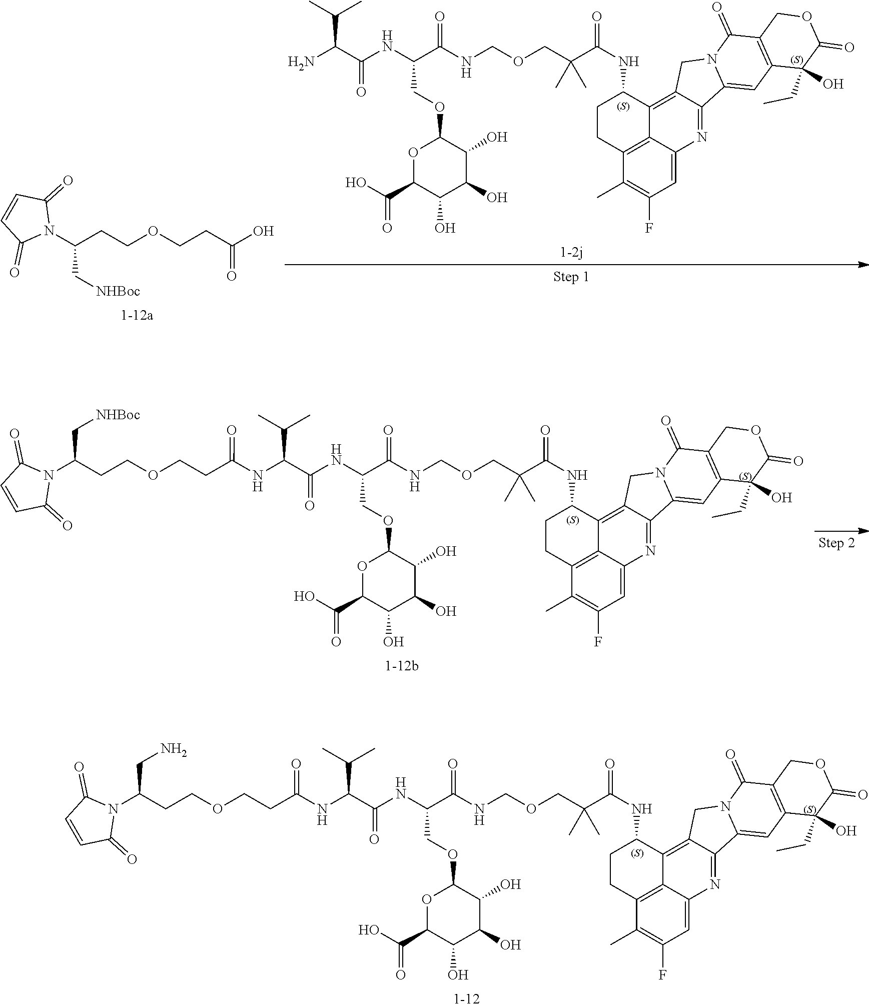

Steps 1 and 2: (2S 3S,4S,5R,6R)-6-((S)-2-((S)-2-(3-((R^{#})-4-amino-3-(2,5-dioxo-2,5-dihydro-1H-pyrrol-1-yl)butoxy)propanamido)-3-methylbutanamido)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-3-oxopropoxy)-3,4,5-trihydroxytetrahydro-2H-pyran-2-carboxylic acid (1-12). 1-12 (13.4 mg, 79.1% yield) as a white solid was synthesized according to the synthetic procedure of Example 1-6. MS (ESI) m/z: 1165.7 [M+H]⁺.

### Example 1-13

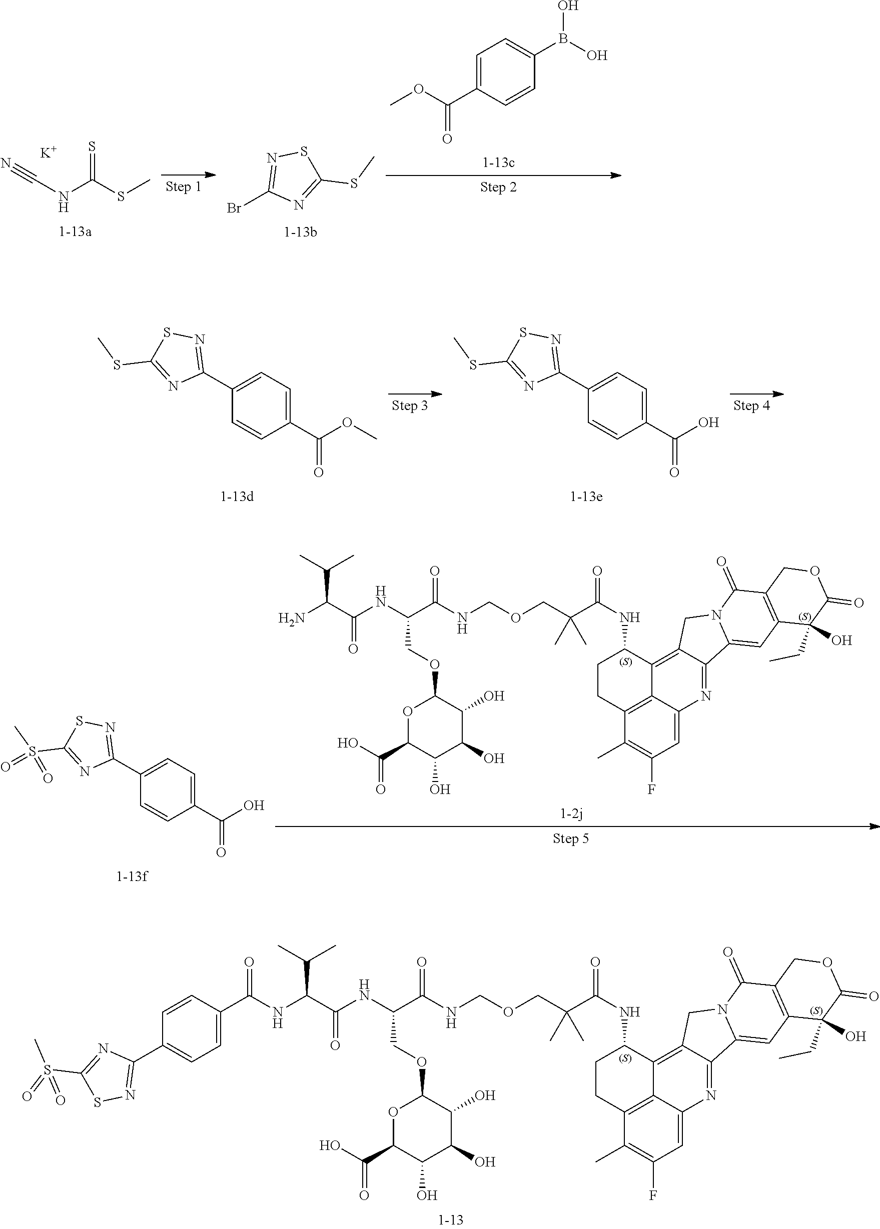

Step 1: 3-bromo-5-(methylthio)-1,2,4-thiadiazole (1-13b). To a solution of compound 1-13a (5 g, 29.4 mmol) in dichloromethane (100 mL) was added dropwise Br₂ (5.2 g, 32.9 mmol) at 0 °C. After addition, the resulting mixture was stirred at room temperature overnight. To the reaction mixture was added excess Na₂SO₃ and water (50 mL) to decompose the excess Br₂. The mixture was separated, and the separated organic layer was washed with brine (50 mL×3), dried over Na₂SO₄ and concentrated in vacuo. The residue was purified by flash column chromatography (eluted with Petroleum ether:EtOAc=0%-40%) to give compound 1-13b (4.2 g, 68.2% yield) as a light-yellow solid. MS (ESI) m/z: 212.9 [M+H]⁺.

Step 2: methyl 4-(5-(methylthio)-1,2,4-thiadiazol-3-yl)benzoate (1-13d). To a solution of compound 1-13b (100 mg, 0.47 mmol) in toluene (4 mL) and H₂O (1 mL) were added compound 1-13c (109.72 mg, 0.568 mmol), K₂CO₃ (168 mg, 0.947 mmol), and Pd(dppf)Cl₂·DCM (34.6 mg, 0.047 mmol). The mixture was stirred at 110 °C for 3 h under N₂ atmosphere. The mixture was filtered through a pad of celite, diluted with EtOAc (100 mL), washed by brine (50 mL*4). The organic layer was dried over anhydrous Na₂SO₄, filtered and concentrated. The crude product was purified by flash column chromatography (eluted with Petroleum ether:EtOAc:=0-40%). Compound 1-13d (56 mg, 44.4% yield) was obtained as an off-white solid. MS (ESI) m/z: 267.1 [M+H]⁺.

Step 3: 4-(5-(methylthio)-1,2,4-thiadiazol-3-yl)benzoic acid (1-13e). To a solution of compound 1-13d (54 mg, 0.20 mmol) in MeOH (3 mL) and 1120 (1 mL) was added LiOH (17 mg, 0.41 mmol). The mixture was stirred at r.t. for 2 h. The mixture was adjusted to pH 7 and purified by prep-HPLC (FA condition) to give compound 1-13e (36 mg, 70.3% yield) as a white solid, MS (ESI) m/z: 253.1 [M+H]+.

Step 4: 4-(5-(methylsulfonyl)-1,2,4-thiadiazol-3-yl)benzoic acid (1-13f). To a solution of compound 1-13e (35 mg, 0.14 mmol) in DCM (3 mL) and THF (3 mL) was added m-CPBA (96 mg, 0.55 mmol). The mixture was stirred at room temperature for 16 h. The mixture was concentrated and purified by prep-HPLC (Method: column: XBridge Prep C18 OBD 5 μm 19*150 mm; Mobile phase: A-water (0.1% TFA): B-acetonitrile; Flow rate: 20 mL/min). Compound 1-13f (12 mg, 99% purity) was obtained as a white solid.

Step 5: (2S,3S,4S,5R,6R)-6-((S)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-2-((S)-3-methyl-2-(4-(5-(methylsulfonyl)-1,2,4-thiadiazol-3-yl)benzamido)butamanido)-3-oxopropoxy)-3,4,5-trihydroxytetrahydro-2H-pyran-2-carboxylic acid (1-13). To a solution of compound 1-13f (7.36 mg, 0.026 mmol) in DMF (2 mL) were added HATU (9.02 mg, 0.024 mmol) and DIEA (5.58 mg, 0.043 mmol). The mixture was stirred at r.t. for 30 min. Compound 1-2j (20 mg, 0.022 mmol) was added to the mixture and stirred at r.t. for 15 min. The reaction was purified by prep-HPLC (Method: column: XBridge Prep C18 OBD 5 μm 19*150 mm; Mobile phase: A-water (0.1% TFA): B-acetonitrile; Flow rate: 20 mL/min) to give compound 1-13 (7.6 mg, 29.5% yield) as a white solid. MS (ESI) m/z: 1193.5 [M+H]⁺.

### Example 1-14

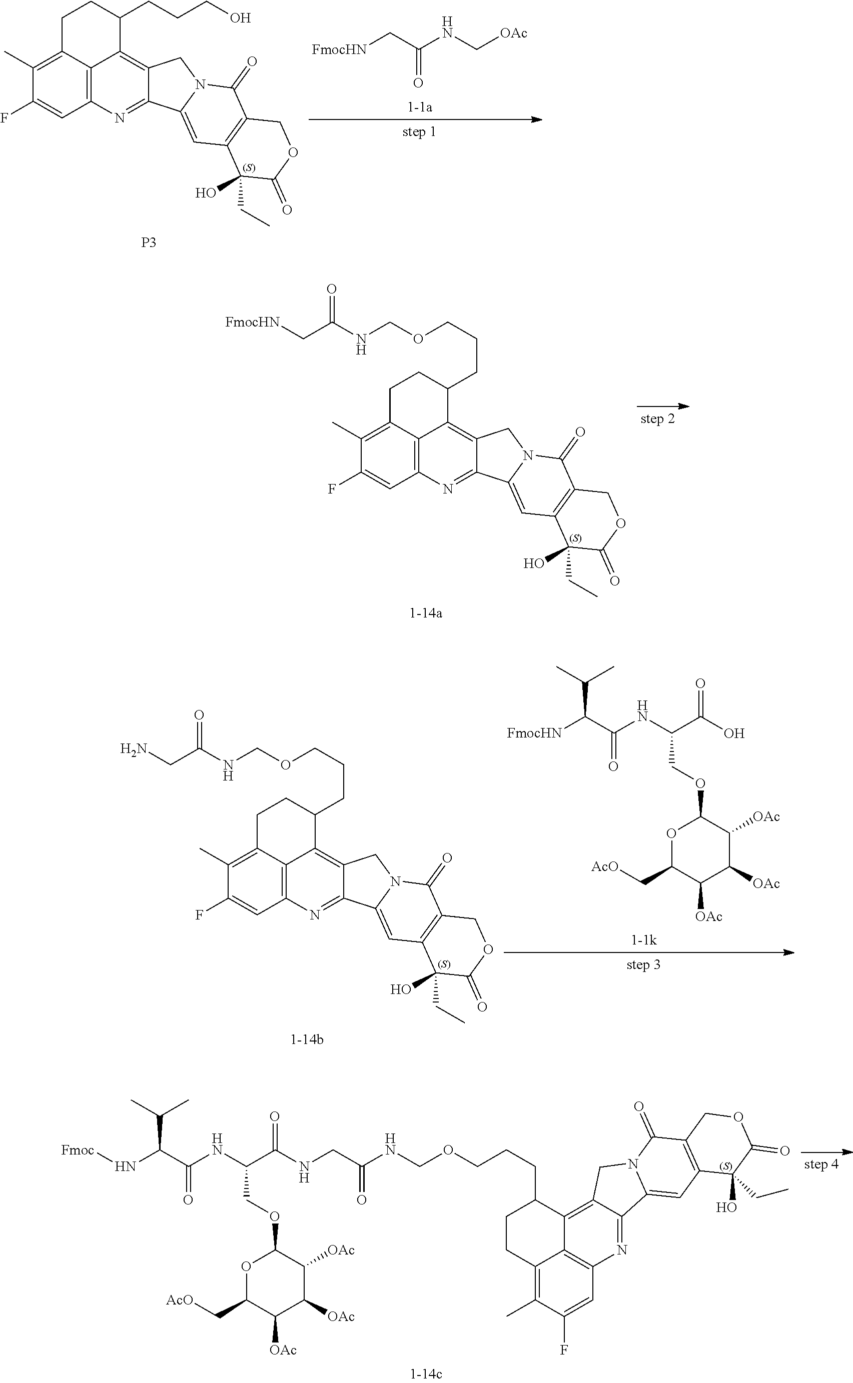

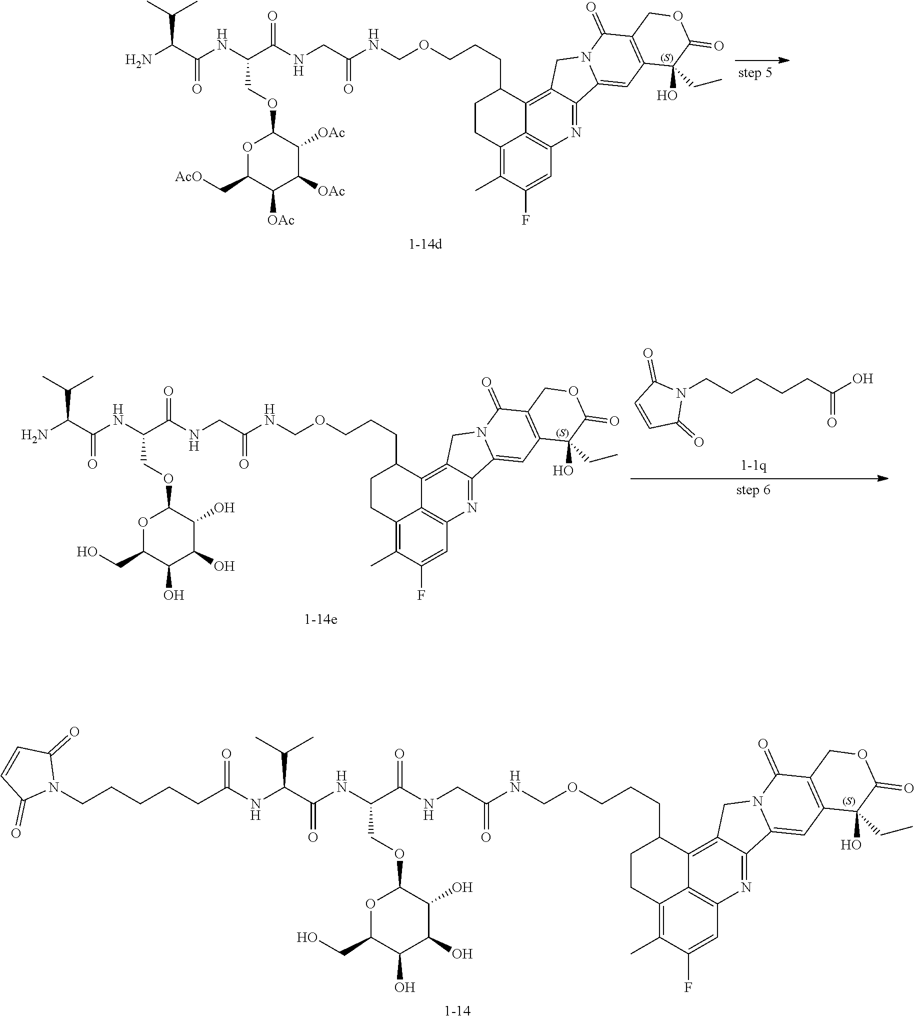

Step 1: (9H-fluoren-9-yl)methyl (2-(((3-((9S)-9-ethyl-5-fluoro-9-hydroxy-4-methyl-10,13-dioxo-2,3,9,10,13,15-hexahydro-1H,2H-benzo[de]pyrano[3′,4′:6,7]indolizino[1,2-b]9]uinoline-1-yl)propoxy)methyl)amino)-2-oxoethyl)carbamate (1-14b). 1-14 was synthesized according to the same procedures as that of step 10, Example 1-1. MS (ESI) m/z: 787.3 [M+H]⁺.

Step 3: (2R,3R,4S,5S,6R)-2-((2S)-2-(S)-2-(((9H-fluoren-9-yl)methoxy)carbonyl)amino)-3-methylbutanamido)-3-((2-(((3-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propoxy)methyl)amino)-2-oxoethyl)amino)-3-oxopropoxy)-6-(acetoxymethyl)tetrahydro-2H-pyran-3,4,5-triyl triacetate (1-14c). 1-14c (45 mg, 32% yield, two steps) was synthesized according to the same procedures as that of step 12, Example 1-1. MS (ESI) m/z: 1304.7 [M+H]⁺.

Step 4: (2R,3S,4S,5R,6R)-2-(acetoxymethyl)-6-((2S)-2-((S)-2-amino-3-methylbutanamido)-3-((2-(((3-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propoxy)methyl)amino)-2-oxoethyl)amino)-3-oxopropoxy)tetrahydro-2H-pyran-3,4,5-triyl triacetate (1-14d). 1-14d was synthesized according to the same procedures as that of step 10, Example 1-1. MS (ESI) m/z: 1081.6 [M+H]⁺.

Step 5: (2S)-2-amino-N-((2S)-1-((2-(((3-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propoxy)methyl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)-3-methylbutanamide (1-14e). 1-14e (19.2 mg, 60% yield, two steps) was synthesized according to the same procedures as that of step 11, Example 1-1. MS (ESI) m/z: 913.5 [M+H]⁺.

Step 6: 6-(2,5-dioxo-2,5-dihydro-1H-pyrrol-1-yl)-N-((11S,14S)-1-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-15-methyl-7,10,13-trioxo-11-((((2R,3R,4S,5R,6R)-3,4,5-trihydroxy-6-(hydroxymethyl)tetrahydro-2H-pyran-2-yl)oxy)methyl)-4-oxa-6,9,12-triazahexadecan-14-yl)hexanamide (1-14f. 1-14f (6.4 mg, 28% yield) was synthesized according to the same procedures as that of step 12, Example 1-1. MS (ESI) m/z: 1106.7 [M+H]⁺.

### Example 1-15

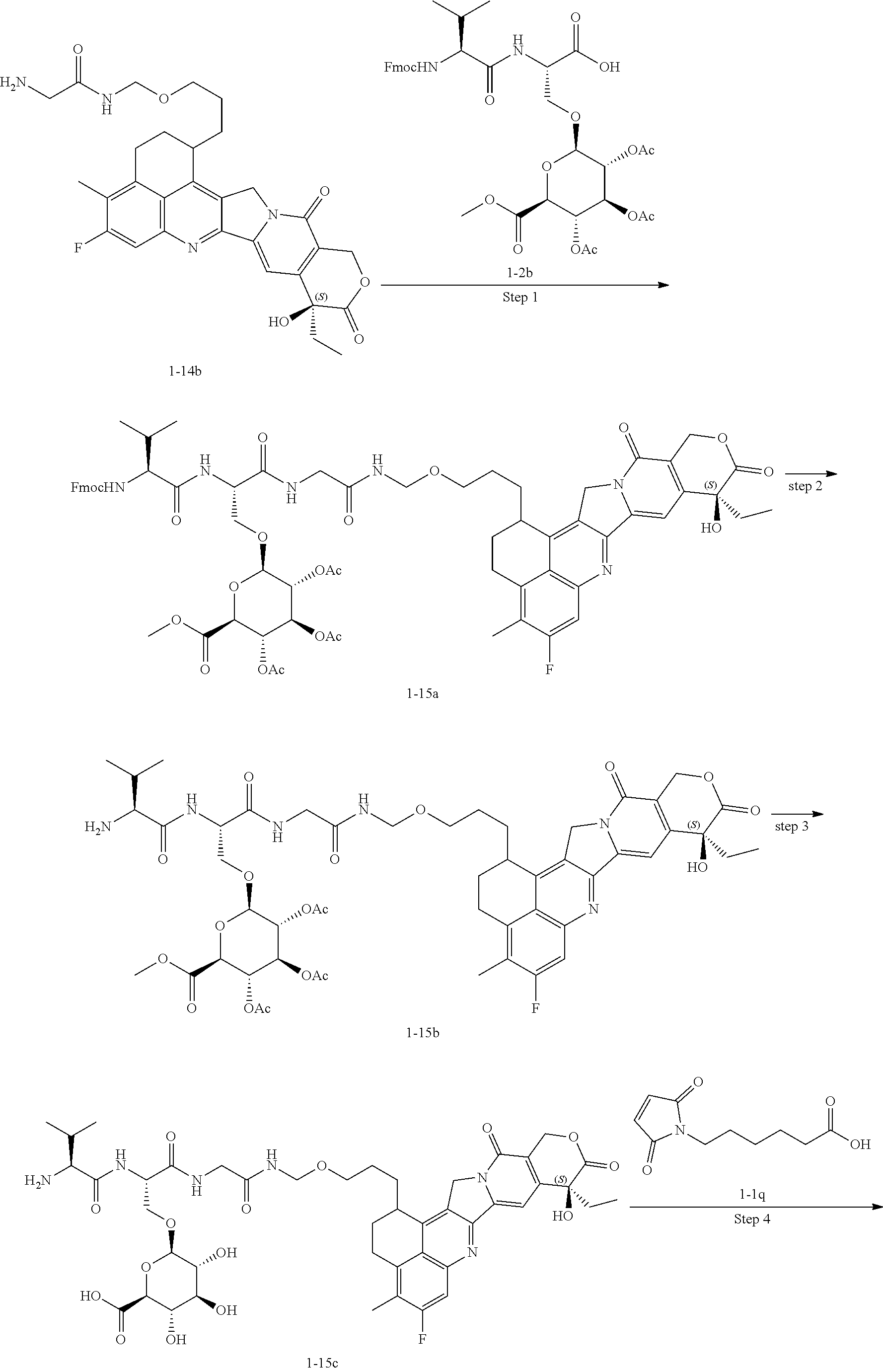

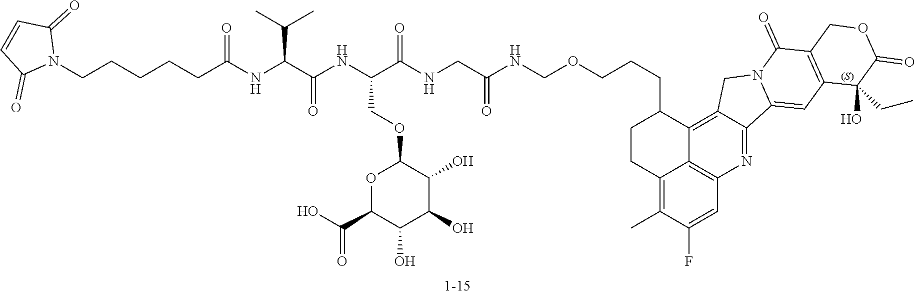

Step 1: (2R,3R,4S,5S,6S)-2-((2S)-2-((S)-2-((((9H-fluoren-9-yl)methoxy)carbonyl)amino)-3-methylbutanamido)-3-((2-(((3-((9S)-9-ethyl-5-fluoro-9-hydroxy-4-methyl-10,13-dioxo-2,3,9,10,13,15-hexahydro-1H,121H-benzo[de]pyrano[3′,4′:6,7]indolizino[1,2-b]quinolin-1-yl)propoxy)methyl)amino)-2-oxoethyl)amino)-3-oxopropoxy)-6-(methoxycarbonyl)tetrahydro-2H-pyran-3,4,5-triyl triacetate (1-15a). Compound 1-15a (45 mg, 36.5% yield) was synthesized according to the synthetic procedure of step 9 of Example 1-1 MS (ESI) m/z: 1290.9 [M+H]

Step 2: (2R,3R,4S,5S,6S)-2-((2S)-2-((S)-2-amino-3-methylbutanamido)-3-((2-(((3-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propoxy)methyl)amino)-2-oxoethyl)amino)-3-oxopropoxy)-6-(m-ethoxycarbonyl)tetrahydro-2H-pyran-3,4,5-triyl triacetate(1-15b). Compound 1-15b (37 mg, crude) was synthesized according to the synthetic procedure of step 10 of Example 1-1. MS (ESI) m/z: 1067.7 [M+H]⁺.

Step 3: (2S,3S,4S,5R,6R)-6-((2S)-2-((S)-2-amino-3-methylbutanamido)-3-((2-(((3-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propoxy)methyl)amino)-2-oxoethyl)amino)-3-oxopropoxy)-3,4,5-trihydroxytetrahydro-2H-pyran-2-carboxylic acid(1-15c). Compound 1-15c (18 mg, 56.0% yield) was synthesized according to the synthetic procedure of step 11 of Example 1-1. MS (ESI) m/z: 927.7 [M+H]⁺.

Step 4: (2S,3S,4S,5R,6R)-6-((25)-2-((S)-2-(6-(2,5-dioxo-2,5-dihydro-1H-pyrrol-1-yl)hexanamido)-3-methylbutanamido)-3-((2-(((3-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propoxy)methyl)amino)-2-oxoethyl)amino)-3-oxopropoxy)-3,4,5-trihydroxytetrahydro-2H-pyran-2-carboxylic acid(1-15). Compound 1-15 (3.2 mg, 14.7% yield) was synthesized according to the synthetic procedure of step 5 of Example 1-13. MS (ESI) m/z: 1120.8 [M+H]⁺.

### Example 1-16

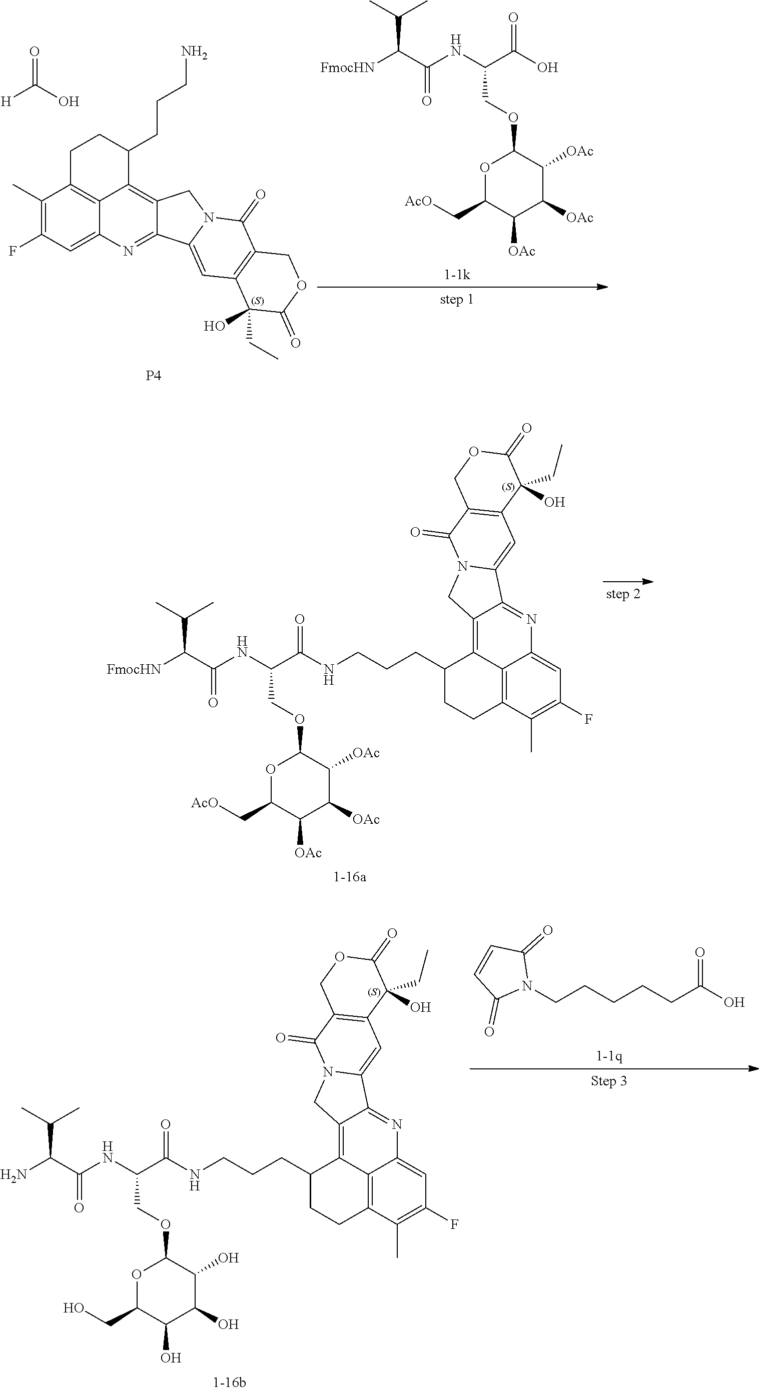

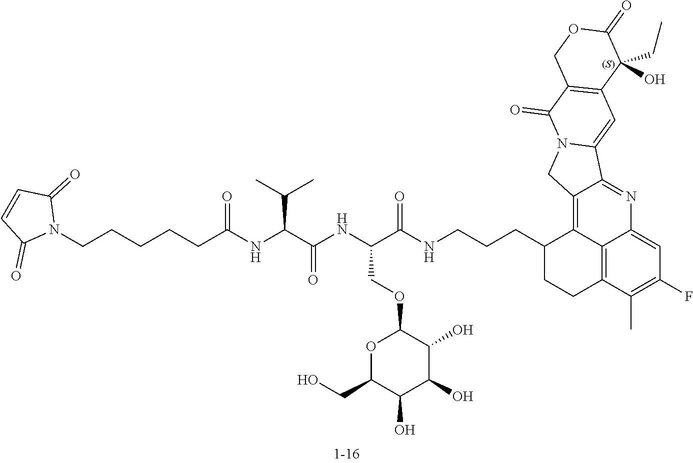

Step 1: (2R,3R,4S,5S,6R)-2-((2S)-2-((S)-2-((((9H-fluoren-9-yl)methoxy)carbonyl)amino)-3-methylbutanamido)-3-((3-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propyl)amino)-3-oxopropoxy)-6-(acetoxymethyl)tetrahydro-2H-pyran-3,4,5-triyl triacetate (1-16a). To a mixture of 1-1k (27 mug, 0.035 mmol) and TSTU (11 mg, 0.035 mmol) in DMF (1 mL) was added DIPEA (14 μL, 0.082 mmol). The mixture was stirred at r.t. for 10 min then P4 (13 mg, 0.027 mmol) was added. The mixture was stirred at r.t. for 30 min. It was purified by prep-HPLC (FA) (Method: column: XBridge Prep C18 OBD 5 μm 19*150 mm; Mobile phase: A-water (0.1% FA): B-acetonitrile; Flow rate: 20 mL/min), the fraction was lyophilized to give 1-16a (20 mg, 60% yield) as a white solid. MS (ESI) m/z: 1216.9 [M+H]⁺.

Step 2: (2S)-2-amino-N-((2S)-1-((3-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propyl)amino)-1-oxo-3-(((2R,3R,4S,5R,6R)-3,4,5-trihydroxy-6-(hydroxymethyl)tetrahydro-2H-pyran-2-yl)oxy)propan-2-yl)-3-methylbutanamide (1-16b). To a solution of 1-16a (20 mg, 0.016 mmol) in DMF (1 mL) was added Et₂NH₄ (34 μL, 0.33 mmol). The mixture was stirred at r.t. for 20 min. Reaction mixture was concentrated under vacuum and co-evaporated with Tol for two times then dissolved in MeOH (1 mL), K₂CO₃ (6 mg, 0.04 mmol) was added. The mixture was stirred at r.t. for 40 min. The mixture was cooled to 0 °C with ice-bath then acidified with 3N HCl to pH==3, filtered and purified by prep-HPLC (FA) (Method: column: XBridge Prep C18 OBD 5 μm 19*50 mm; Mobile phase: A-water (0.1% TFA): B-acetonitrile; Flow rate: 20 mL/min), the fraction was lyophilized to give 1-16b (10 mg, 75% yield) as a white solid. MS (ESI) m/z: 826.7 [M+H]⁺.

Step 3: 6-(2,5-dioxo-2,5-dihydro-1H-pyrrol-1-yl)-N-((2S)-1-(((2S)-1-((3-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propyl)amino)-1-oxo-3-(((2R,3R,4S,5R,6R)-3,4,5-trihydroxy-6-(hydroxymethyl)tetrahydro-2H-pyran-2-yl)oxy)propan-2-yl)amino)-3-methyl-1-oxobutan-2-yl)hexanamide (1-16). To a solution of 1-1q (3 mg, 0.012 mmol) in DMF (1 mL) were added HATU (5 mg, 0.012 mmol) and DIPEA (2 μL, 0.013 mmol). The mixture was stirred at r.t. for 10 min then 1-16b (32 mg, 0.035 mmol) was added. The resulting yellow mixture was stirred at r.t. for 15 min. The reaction mixture was purified by prep-HPLC (FA) (Method: column: XBridge Prep C18 OBD 5 μm 19*150 mm; Mobile phase: A-water (0.1% TFA): B-acetonitrile; Flow rate: 20 mL/min), the fraction was lyophilized to give 1-16 (8 mg, 50% yield) as a white solid. MS (ESI) m/z: 1019.8 [M+H]⁺.

### Example 1-17

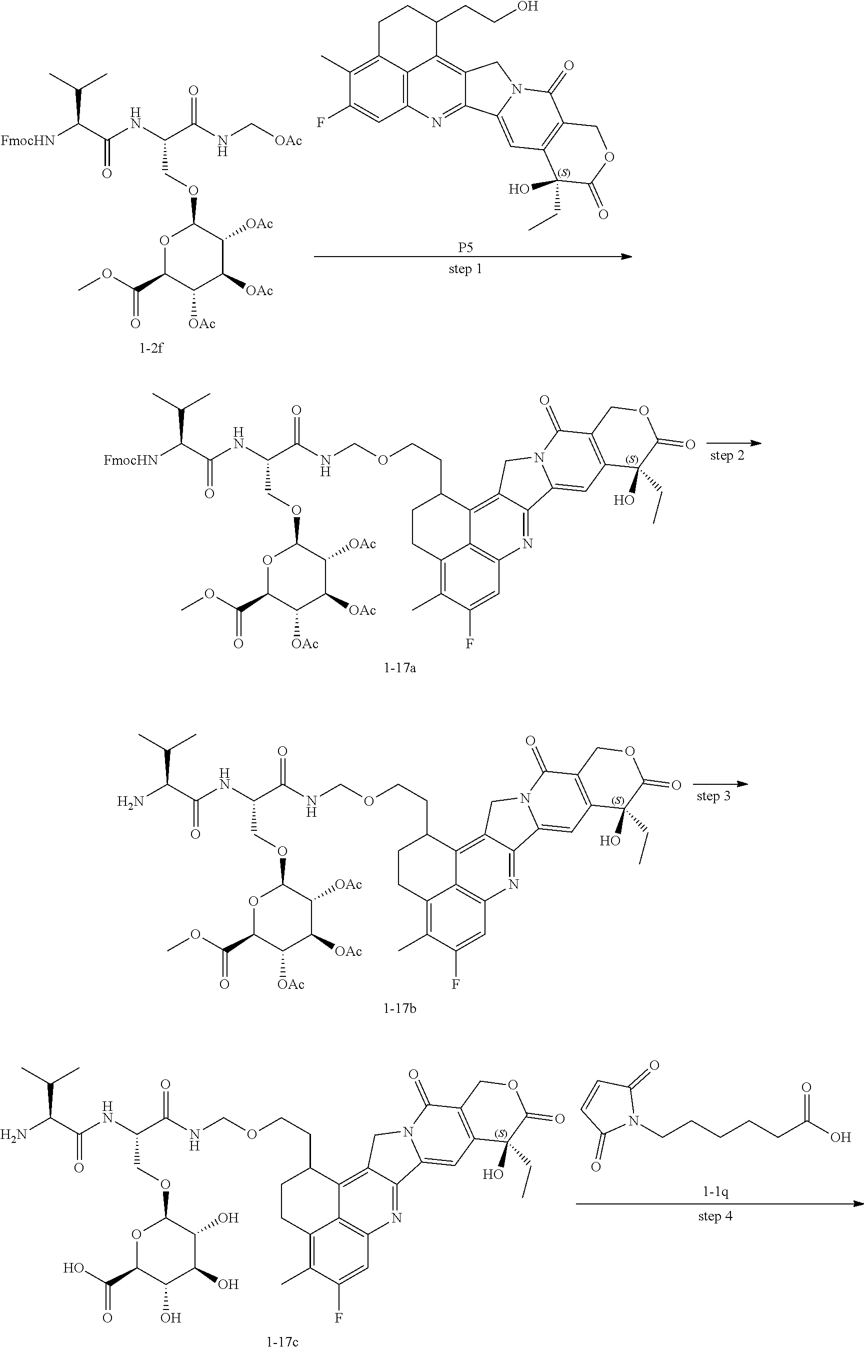

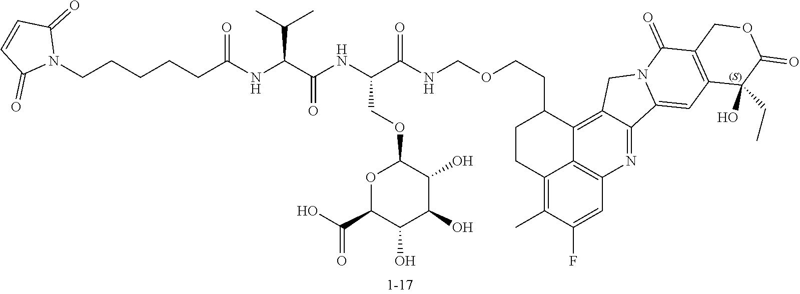

Step 1: (2R,3R,4S,5S,6S)-2-((2S)-2-((S)-2-((((9H-fluoren-9-yl)methoxy)carbonyl)amino)-3-methylbutanamido)-3-(((2-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)ethoxy)methyl)amino)-3-oxopropoxy)-6-(methoxycarbonyl)tetrahydro-2H-pyran-3,4,5-triyl triacetate (1-17a). 1-17a (110 mg, 69.2% yield) was synthesized according to the same procedures as that of step 1, Example 1-1. MS (EST) m/z: 1218.5 [M+H]⁺.

Step 2: (2R,3R,4S,5S,6S)-2-((2S)-2-((S)-2-amino-3-methylbutanamido)-3-(((2-((9S)-9-ethyl-5-fluoro-9-hydroxy-4-methyl-10,13-dioxo-2,3,9,10,13,15-hexahydro-11,121-benzo[de]pyrano[3′,4′:6,7]indolizino[1,2-b]quinolin-1-yl)ethoxy)methyl)amino)-3-oxopropoxy)-6-(methoxycarbonyl)tetrahydro-2H-pyran-3,4,5-triyl triacetate (1-17b). 1-17b (90 ng, crude product) was synthesized according to the same procedures as that of step 4, Example 1-5. MS (EST) m/z: 997.1 [M+H]⁺.

Step 3: (2S,3S,4S,5R,6R)-6-((2S)-2-((S)-2-amino-3-methylbutanamido)-3-(((2-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)ethoxy)methyl)amino)-3-oxopropoxy)-3,4,5-trihydroxytetrahydro-2H-pyran-2-carboxylic acid (1-17c). 1-17c (50 mg, 65.0%) was synthesized according to the same procedures as that of step 5, Example 1-5. MS (EST) m/z: 856.3[M+H]⁺.

Step 4: (2S,3S,4S,5R,6R)-6-((2S)-2-((S)-2-(6-(2,5-dioxo-2,5-dihydro-1H-pyrrol-1-yl)hexanamido)-3-methylbutanamido)-3-(((2-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)ethoxy)methyl)amino)-3-oxopropoxy)-3,4,5-trihydroxytetrahydro-2H-pyran-2-carboxylic acid (1-17). 1-17 (25 mg, 41.4%) was synthesized according to the same procedures as that of step 6, Example 1-5. MS (EST) m/z: 1050.1 [M+H]⁺.

### Example 1-18

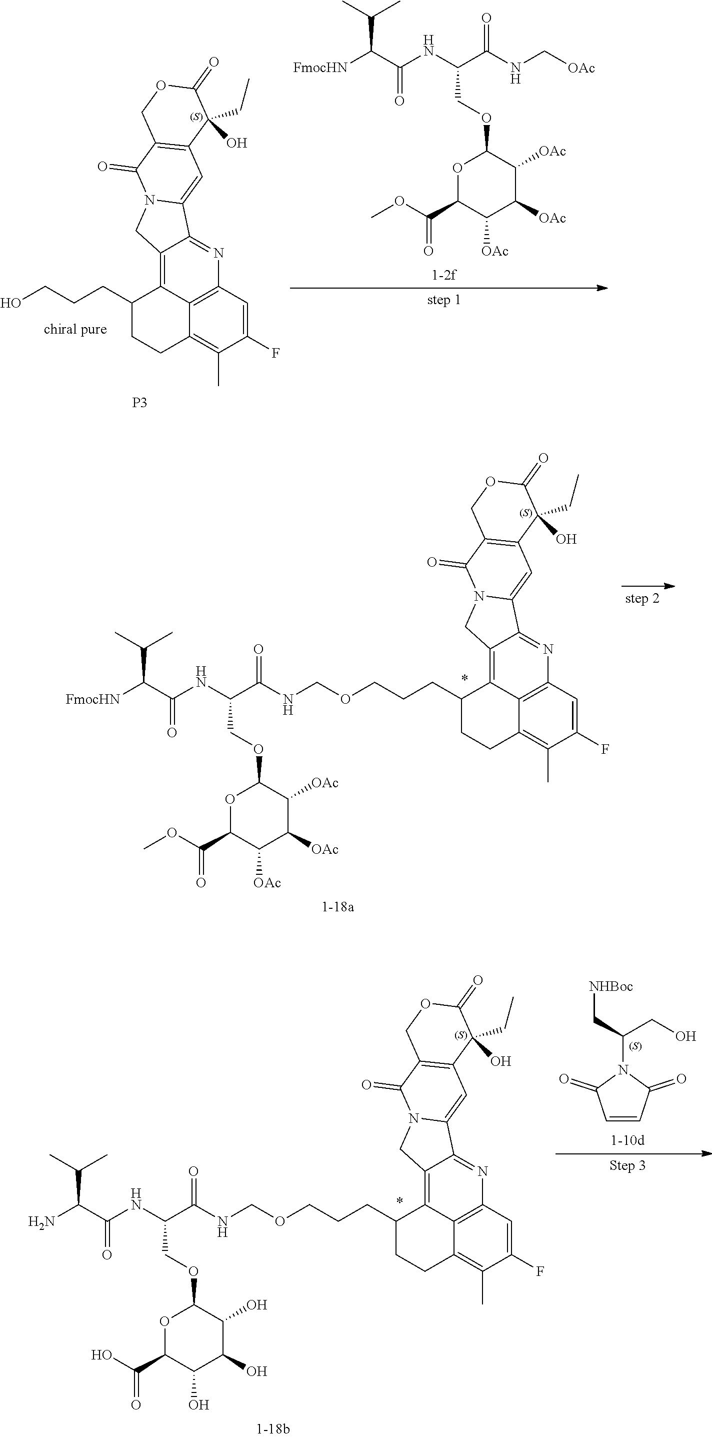

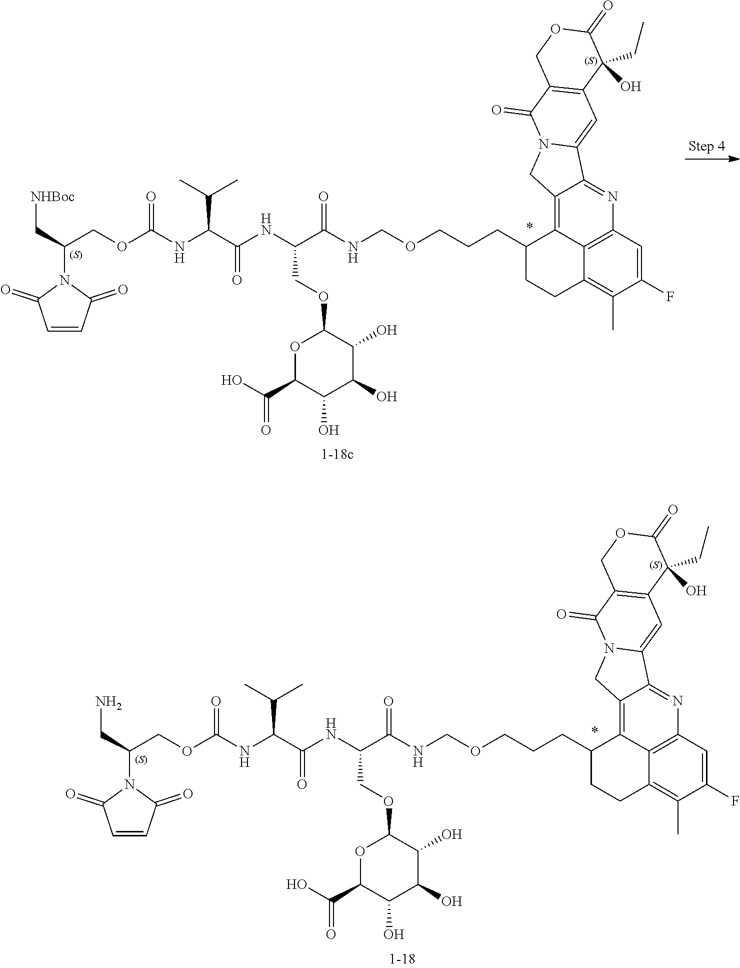

Step 1: (2R,3R,4S,5S,6S)-2-((2S)-2-((S)-2-((((9H-fluoren-9-yl)methoxy)carbonyl)amino)-3-methylbutanamido)-3-(((3-((9S)-9-ethyl-5-fluoro-9-hydroxy-4-methyl-10,13-dioxo-2,3,9,10,13,15-hexahydro-1,12H-benzo[de]pyrano[3′,4′:6,7]indolizino[1,2-b]quinolin-1-yl)propoxy)methyl)amino)-3-oxopropoxy)-6-(methoxycarbonyl)tetrahydro-2H-pyran-3,4,5-triyl triacetate (1-18a). A white suspension mixture of 1-2f (102 mg, 0.13 mmol), P3 (60 mg, 0.13 mmol) and 4 A molecular sieve (50 mg) in anhydro THF (3 mL) was stirred at r.t. for 10 min. Sc(OTf)₃ (80 mg, 0.16 mmol) was added and the resulting yellow suspension was stirred at r.t. for 4 h. The yellow suspension mixture was filtered through a pad of celite and washed with EtOAc (20 mL). Combined organic layers were washed with sat. NaHCO₃ (30 mL) and brine (10 mL), dried over Na₂SO₄, filtered and the filtrate was concentrated under vacuum to give a residue. It was purified by silica gel column (MeOH/DCM=0%~-5%), fraction was concentrated under vacuum to give 1-18a (147 mg, 95% yield) as a white foam solid. MS (ESI) m/z: 1232.9 [M+H]⁺.

Step 2: (2S,3S,4S,5R,6R)-6-((2S)-2-((S)-2-amino-3-methylbutanamido)-3-(((3-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propoxy)methyl)amino)-3-oxopropoxy)-3,4,5-trihydroxytetrahydro-2H-pyran-2-carboxylic acid (1-18b). To a solution of 1-18a (147 mg, 0.12 mmol) in DMF (3 mL) was added Et₂NH (197 μL, 1.9 mmol). The mixture was stirred at r.t. for 20 min. Reaction mixture was concentrated under vacuum and co-evaporated with Tol for two times then dissolved in MeOH (3 mL) and H₂O (0.5 mL), K₂CO₃ (50 mg, 0.36 mmol) was added. The mixture was stirred at r.t. for 30 min. The mixture was cooled to 0 °C with ice-bath then acidified with 3N HCl to pH=3, filtered and purified by prep-HPLC (FA) (Method: column: XBridge Prep C18 OBD 5 μm 19*150 mm; Mobile phase: A-water (0.1% FA): B-acetonitrile; Flow rate: 20 mL/min), the fraction was lyophilized to give 1-18b (65 mg, 61% yield) as a white solid. MS (ESI) m/z: 870.7 [M+H]⁺.

Step 3: (2S,3S,4S,5R,6R)-6-(((7S,12S,15S)-7-(2,5-dioxo-2,5-dihydro-1H-pyrrol-1-yl)-15-(((3-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propoxy)methyl)carbamoyl)-12-isopropyl-2,2-dimethyl-4,10,13-trioxo-3,9-dioxa-5,11,14-triazahexadecan-16-yl)oxy)-3,4,5-trihydroxytetrahydro-2H-pyran-2-carboxylic acid (1-18c). To a solution of 1-10d (14 mg, 0.052 mmol) in DMF (1 mL) were added DSC (14 mg, 0.052 mmol) and DIPEA (18 μL, 0.11 mmol). The mixture was stirred at r.t. overnight. 1-18b (32 mg, 0.035 mmol) was added then stirred at r.t. for 10 min. The mixture was purified by prep-HPLC (FA) (Method: column: XBridge Prep C18 OBD 5 μm 19*150 mm; Mobile phase: A-water (0.1% FA): B-acetonitrile; Flow rate: 20 mL/min), the fraction was lyophilized to give 1-18c (18 mg, 44% yield) as a pale-yellow solid. MS (ESI) m/z: 1166.9 [M+H]⁺.

Step 4: (2S,3S,4S,5R,6R)-6-((2S)-2-((S)-2-((((S)-3-anno-2-(2,5-dioxo-2,5-dihydro-1H-pyrrol-1-yl)propoxy)carbonyl)amino)-3-methylbutanamido)-3-(((3-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propoxy)methyl)amino)-3-oxopropoxy)-3,4,5-trihydroxytetrahydro-2H-pyran-2-carboxylic acid (1-18). To a mixture of 1-18c (10 mg, 0.009 mmol) in DCM (1 mL) was added ZnBr₂ (79 mg, 0.35 mmol), the mixture was stirred at 40 °C for 2 h. The mixture was concentrated under vacuum to remove DCM then dissolved in DMSO and purified by prep-HPLC (FA) (Method: column: XBridge Prep C18 OBD 5 μm 19*150 mm; Mobile phase: A-water (0.1% FA): B-acetonitrile; Flow rate: 20 mL/min), the fraction was lyophilized to give 1-18 (4.7 mg, 50% yield) as a white solid. MS (ESI) rm/z: 1066.8 [M+H]⁺.

### Example 1-19

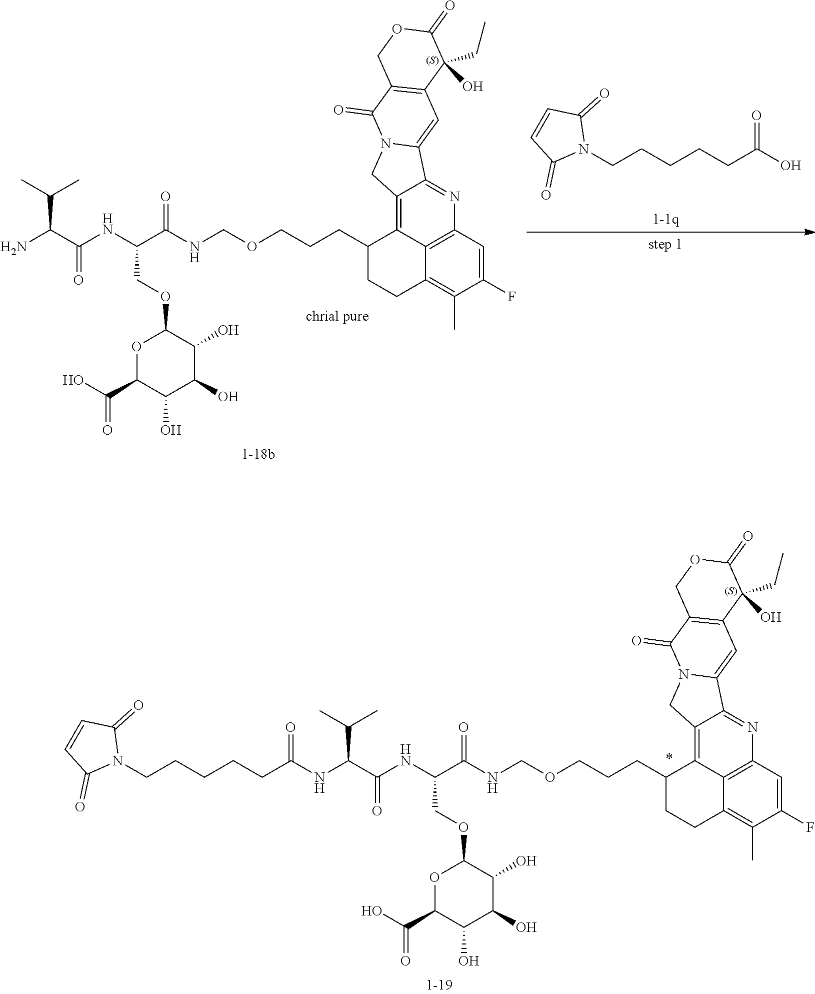

Step 1: (2S,3S,4S,5R,6R)-6-((2S)-2-((S)-2-(6-(2,5-dioxo-2,5-dihydro-1H-pyrrol-1-yl)hexanamido)-3-methylbutanamido)-3-(((3-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propoxy)methyl)amino)-3-oxopropoxy)-3,4,5-trihydroxytetrahydro-2H-pyran-2-carboxylic acid (1-19). To a solution of 1-1q (8 mg, 0.038 mmol) in DMF (1.5 mL) were added HATU (14 mg, 0.037 mmol) and DIPEA (17 μl, 0.11 mmol). The mixture was stirred at r.t. for 10 min then 1-18b (32 mg, 0.035 mmol) was added. The resulting yellow mixture was stirred at r.t. for 15 min. The reaction mixture was purified by prep-HPLC (FA) (Method: column: XBridge Prep C18 OBD 5 μm 19*150 mm; Mobile phase: A-water (0.1% TFA): B-acetonitrile; Flow rate: 20 mL/min), the fraction was lyophilized to give 1-19 (17.9 mg, 48% yield) as a white solid. MS (ESI) m/z: 1063.8 [M+H]⁺

### Example 1-20

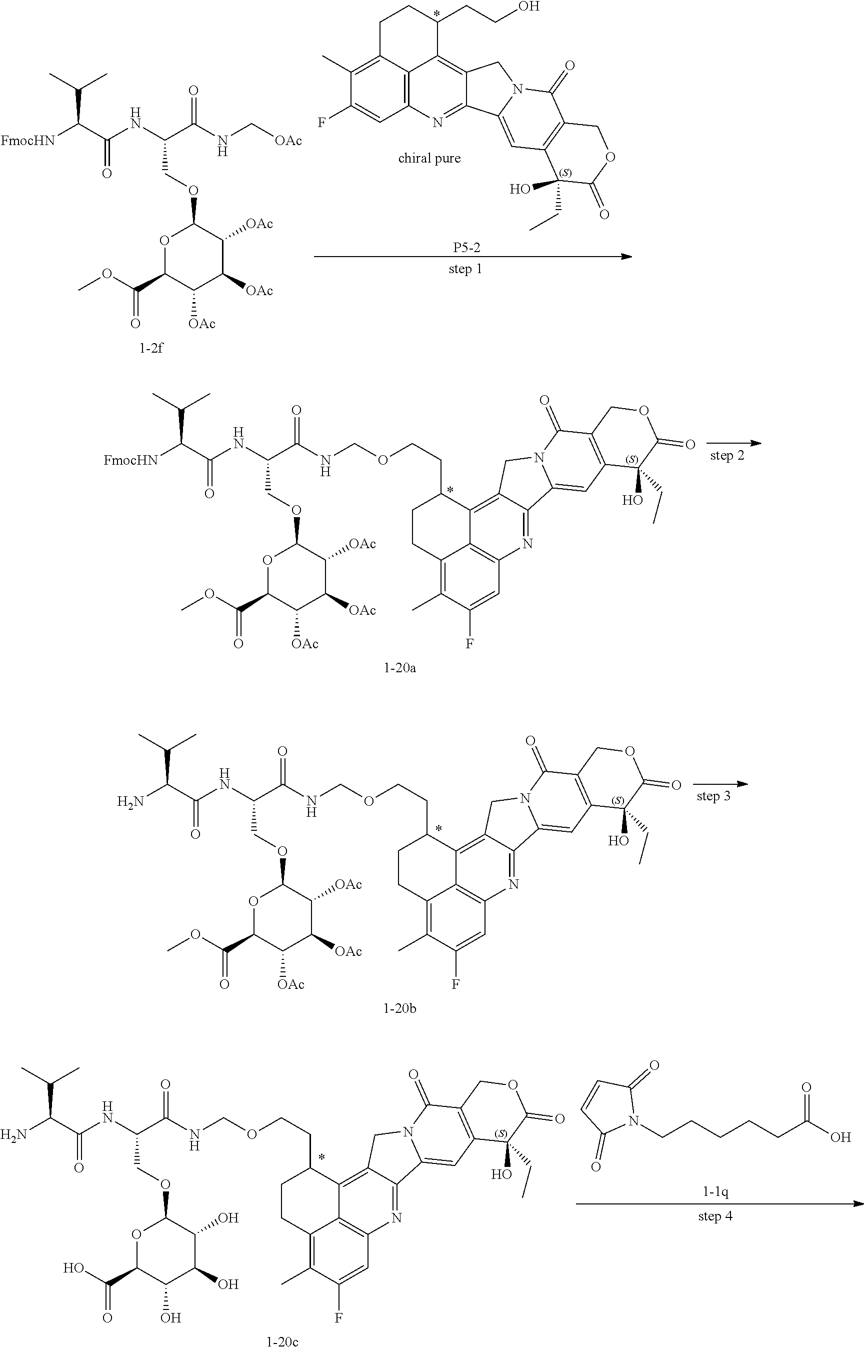

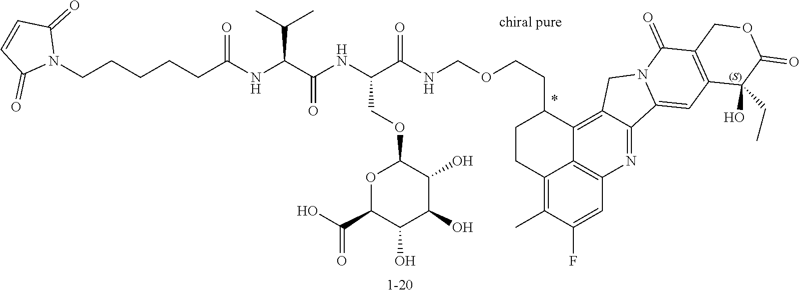

Example 1-20 (25 mg, 41.4%) was synthesized according to the same procedures as that of Example 1-17.

### Example 1-21

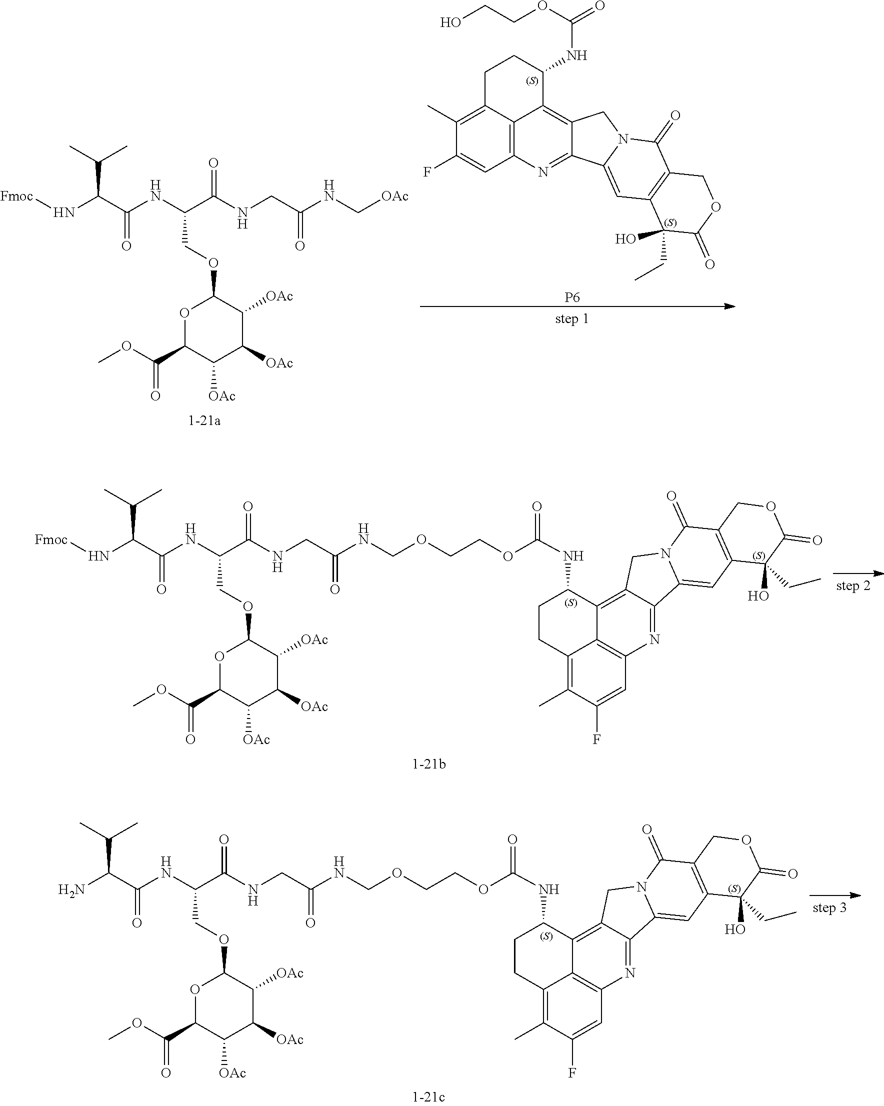

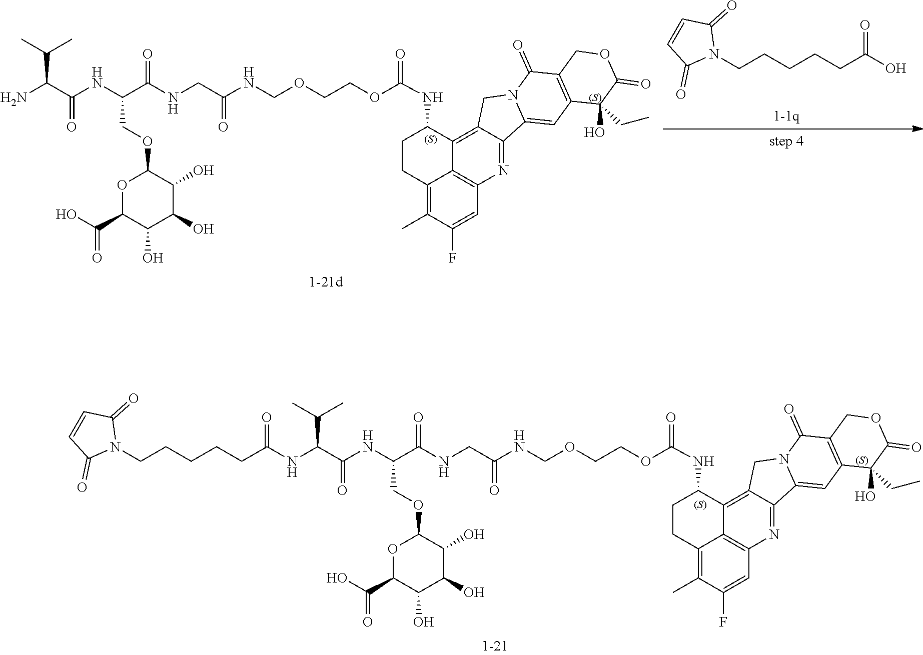

Step 1: (2R,3R,4S,5S,6S)-2-(((S)-12-((S)-2-((((9H-fluoren-9-yl)methoxy)carbonyl)amino)-3-methylbutanamido)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,8,11-trioxo-2,5-dioxa-7,10-diazatridecan-13-yl)oxy)-6-(methoxycarbonyl)tetrahydro-2H-pyran-3,4,5-triyl triacetate (1-21b). To the solution of 1-21a (90 mg, 0.1 mmol), P6 (56 mg, 0.1 mmol) and 4 A MS (200 ng) in dry THF (2 mL) was added Sc(OTf)₃ (62 mg, 0.12 mmol) under N₂ atmosphere, stirred at r.t. for 16 h. The solution was filtrated and washed with THF (50 mL). The pH of the filtrate was adjusted to 8-9 with aq. NaHCO₃ (10 mL) and extracted with CH₂Cl₂ (20 mL*3). The organic phase was concentrated and purified by silica gel column chromatography (CH₂Cl₂/MeOH=90/10) to give 1-21b (100 mg, 72.5% yield) as a yellow solid, MS (ESI) m/z: 1335.3 [M+H]⁺.

Step 2: (2R,3R,4S,5S,6S)-2-(((S)-12-((S)-2-amino-3-methylbutanamido)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,8,11-trioxo-2,5-dioxa-7,10-diazatridecan-13-yl)oxy)-6-(methoxycarbonyl)tetrahydro-2H-pyran-3,4,5-triyl triacetate (1-21c). To the solution of 1-21b (100 mg, 0.075 mmol) in dry DMF (3 mL) was added Et₂NH (111 mg, 1.5 mmol), stirred at r.t. for 20 min. 1-21c (80 mg, crude) was co-evaporated with toluene five times to remove H₂O. MS (ESI) m/z: 1113.1 [M+H]⁺.

Step 3: (2S,3S,4S,5R,6R)-6-(((S)-12-((S)-2-amino-3-methylbutanamido)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,8,11-trioxo-2,5-dioxa-7,10-diazatridecan-13-yl)oxy)-3,4,5-trihydroxytetrahydro-2H-pyran-2-carboxylic acid (1-21d). To the solution of 1-21c (80 mg, 0.072 mmol) in MeOH (2 mL) and water (2 mL) was added K₂CO₃ (50.2 mg, 0.360 mmol), stirred at r.t. for 30 min. The pH was adjusted to around 4 by progressively adding 20% aq. HOAc. The solution was purified by prep-HPLC (Method: column XBridge Prep C18 OBD 5 μm 19*150 mm; Mobile phase: A-water (0.1% FA): B-acetonitrile; Flow rate: 20 mL/min), the fraction was lyophilized to give 1-21d (25 mg, 35.8%) as a white solid. MS (EST) m/z: 973.5 [M+H]⁺.

Step 4: (2S,3S,4S,5R,6R)-6-(((S)-12-((S)-2-(6-(2,5-dioxo-2,5-dihydro-1H-pyrrol-1-yl)hexanamido)-3-methylbutanamido)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,8,11-trioxo-2,5-dioxa-7,10-diazatridecan-13-yl)oxy)-3,4,5-trihydroxytetrahydro-2H-pyran-2-carboxylic acid (1-21). To the solution of 1-1q (5.8 mg, 0.027 mmol) in dry DMF (1.0 mL) were added HATU (12 mg, 0.031 mmol) and DIPEA (0.009 mL, 0,051 mmol), stirred at r.t. for 10 min. The to the above mixture was added 1-21d (25 mg, 0.026 mmol), stirred at r.t. for 10 min. The resulting solution was purified by prep-HPLC (Method: column XBridge Prep C18 OBD 5 μm 19*150 mm; Mobile phase: A-water (0.1% FA): B-acetonitrile; Flow rate: 20 mL/min), the fraction was lyophilized to give 1-21 (7.4 mg, 24.7% yield) as a white solid. MS (ESI) m/z: 1187.8 [M+Na].

### Example 1-22

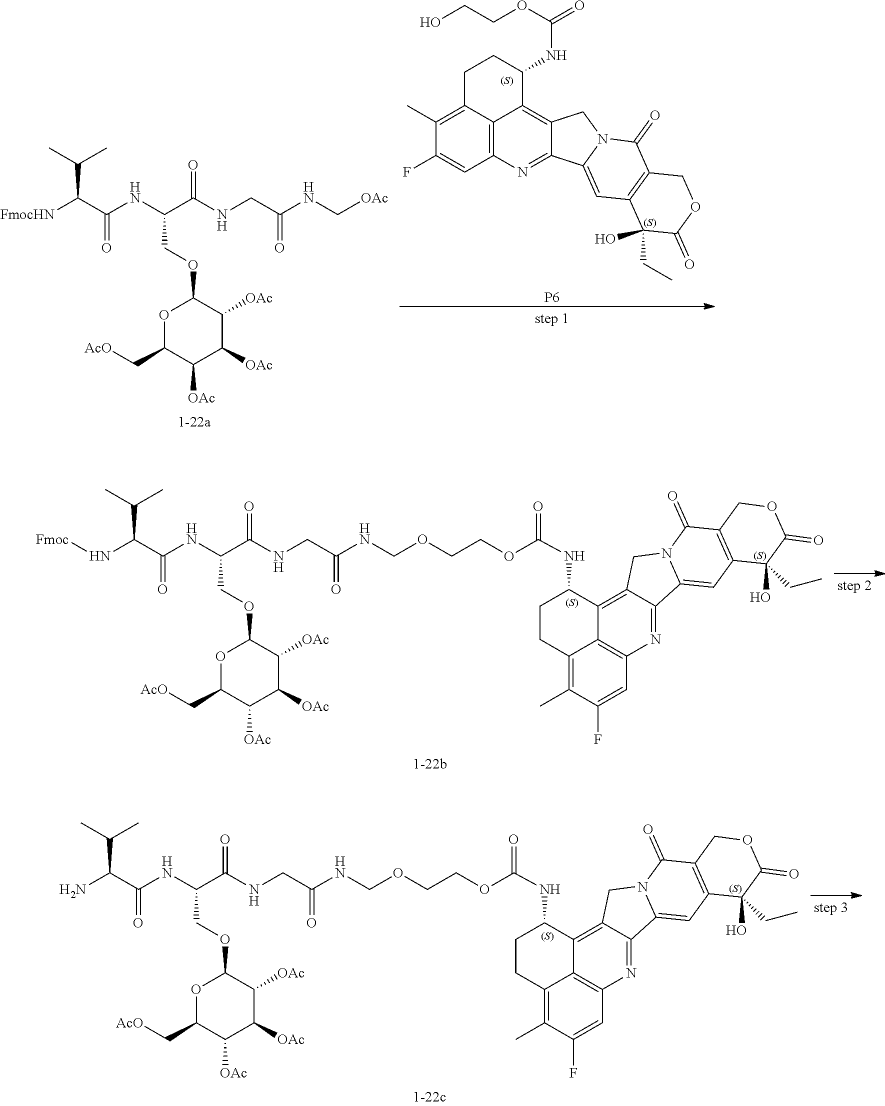

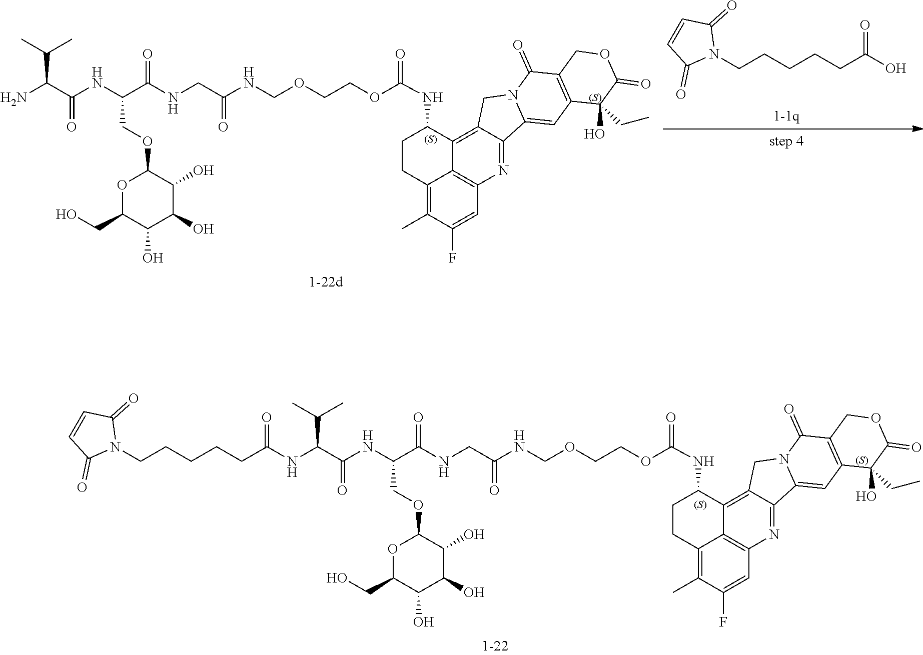

Step 1: (2R,3R,4S,5R,6R)-2-(((S)-12-((S)-2-((((9H-fluoren-9-yl)methoxy)carbonyl)amino)-3-methylbutanamido)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,8,11-trioxo-2,5-dioxa-7,10-diazatridecan-13-yl)oxy)-6-(acetoxymethyl)tetrabydro-2H-pyran-3,4,5-triyl triacetate (1-22b). To a solution of 1-22a (60 mg, 0.068 mmol), P6 (36 mg, 0.068 mmol) and 4 A MS (150 mg) in dry THF (1 mL) was added Sc(OTf)₃ (40 mg, 0.081 mmol) under N₂ atmosphere, stirred at r.t. for 16 h. The solution was filtrated and washed with THF (50 mL). The pH of the filtrate was adjusted to 8-9 with aq. NaHCO₃ (10 mL) and extracted with CH₂Cl₂ (20 mL*3). The organic phase was concentrated and purified by silica gel column chromatography (CH₂Cl₂/MeOH=90/10) to give 1-22b (45 mg, 49.2% yield) as a yellow solid. MS (ESI) m/z: 1348.9 [M+H]⁺.

Step 2: (2R,3R,4S,5R,6R)-2-(acetoxymethyl)-6-(((S)-12-((S)-2-amino-3-methylbutanamido)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,8,11-trioxo-2,5-dioxa-7,10-diazatridecan-13-yl)oxy)tetrahydro-2H-pyran-3,4,5-triyl triacetate (1-22c). To a solution of 1-22b (45 mg, 0.033 mmol) in dry DMF (1 mL) was added Et₂NH (49 mg, 0.67 mmol) at r.t., stirred at r.t. for 20 min. 1-22c (35 mg, crude) was co-evaporated with toluene five times to remove H₂O. MS (ESI) m/z: 1127.1 [M+H]⁺.

Step 3: (2S,3S,4S,5R,6R)-6-(((S)-12-((S)-2-amino-3-methylbutanamido)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,8,11-trioxo-2,5-dioxa-7,10-diazatridecan-13-yl)oxy)-3,4,5-trihydroxytetrahydro-2H-pyran-2-carboxylic acid (1-21d). To the solution of 1-22c (35 mg, 0.031 mmol) in MeOH (1 mL) and water (1 mL) was added K₂CO₃ (22 mg, 0.16 mmol) at 0 °C, stirred at r.t. for 30 min. The pH was adjusted to around 4 by progressively adding 20% aq. HOAc. The solution was purified by prep-HPLC (Method: column XBridge Prep C18 OBD 5 pam 19*150 mm; Mobile phase: A-water (0.1% FA): B-acetonitrile; Flow rate: 20 mL/min), the fraction was lyophilized to give 1-22d (9 mg, 30.2%) as a white solid. MS (ESI) m/z: 958.7 [M+H]⁺.

Step 4: (10S,13S)-20-(2,5-dioxo-2,5-dihydro-1H-pyrrol-1-yl)-13-isopropyl-6,9,12,15-tetraoxo-10-((((2R,3R,4S,5S,6R)-3,4,5-trihydroxy-6-(hydroxymethyl)tetrahydro-2H-pyran-2-yl)oxy)methyl)-3-oxa-5,8,11,14-tetraazaicosyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (1-22). To a solution of 1-1q (2.1 mg, 0.009 mmol) in dry DMF (1.0 mL) were added HATU (0.011 mg, 0.011 mmol) and DIPEA (0.003 mL, 0.011 mmol), stirred at r.t. for 10 min. To the above mixture was added 1-22d (9.0 mg, 0.009 mmol), stirred at r.t. for 10 min. The resulting solution was purified by prep-HPLC (Method: column XBridge Prep C18 OBD 5 μm 19*150 mm; Mobile phase: A-water (0.1% FA): B-acetonitrile; Flow rate: 20 mL/min), the fraction was lyophilized to give 1-22 (2.8 mg, 25.9% yield) as a white solid. MS (ESI) m/z: 1152.2 [M+H]⁺.

### Example 1-23

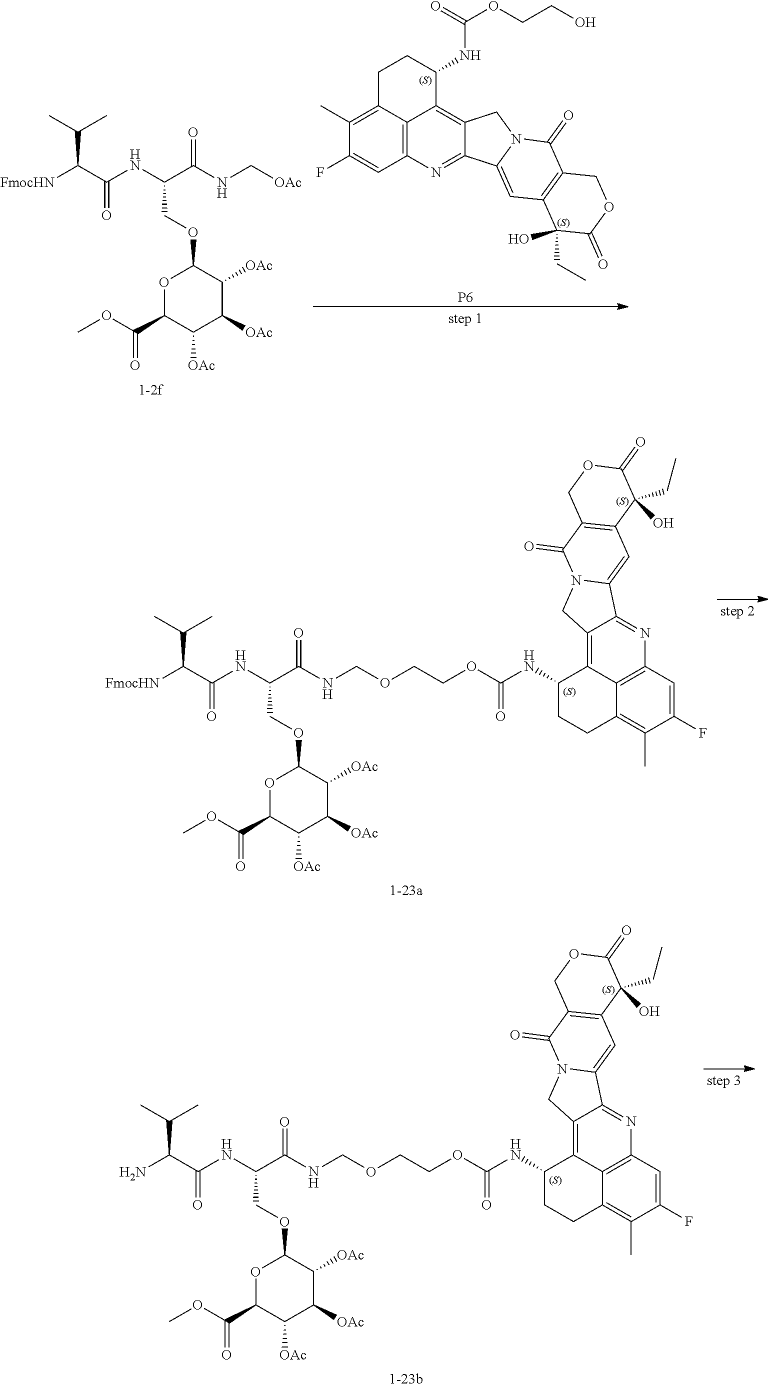

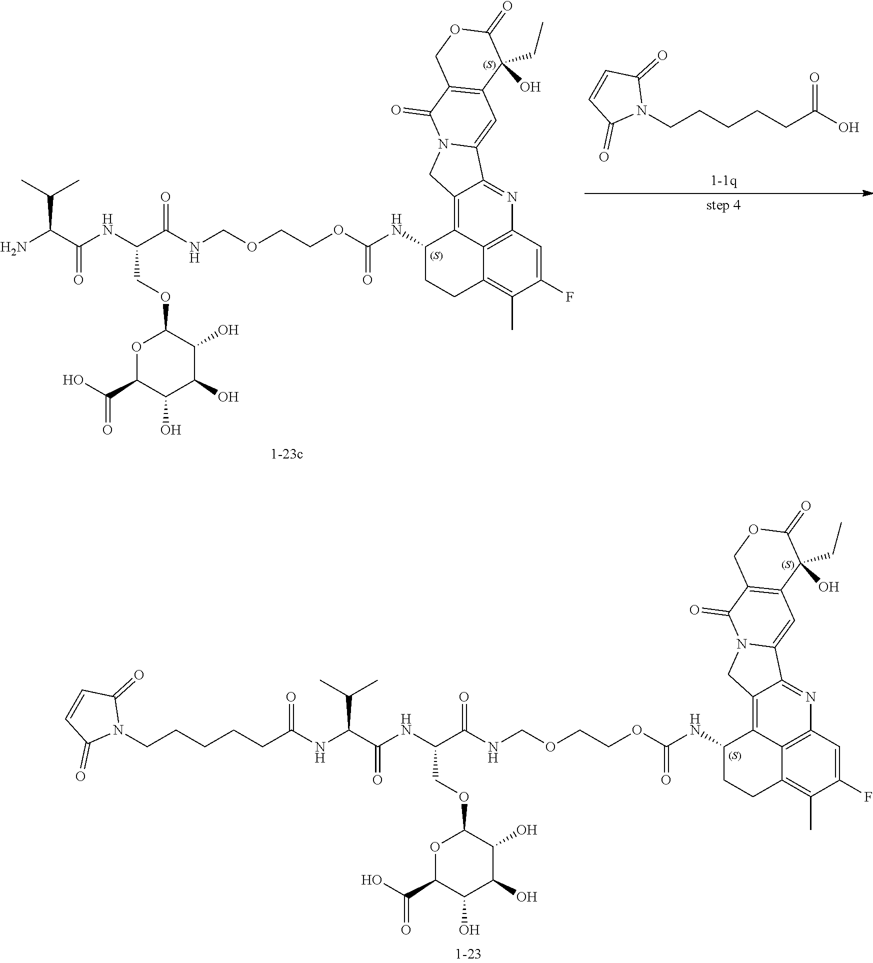

Step 1: (2R,3R,4S,5S,6S)-2-((S)-2-((S)-2-((((9H-fluoren-9-yl)methoxy)carbonyl)amino)-3-methylbutanamido)-3-(((2-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)ethoxy)methyl)amino)-3-oxopropoxy)-6-(methoxycarbonyl)tetrahydro-2H-pyran-3,4,5-triyl triacetate (1-23a). To a solution of 1-2f (153 mg, 0.18 mmol) and P6 (150 mg, 0.18 mmol) in dry THF (2 mL) was added 4A MS (840 mg), stirred for 20 min. Sc(OTf)₃ (120 mg, 0.24 mmol) was added, stirred at 35 °C for 14 h and at 50 °C for 24 h. The mixture was filtered and the filtrate was washed with sat. NaHCO₃ (5 mL). The water phase was extracted with DCM/MeOH (10:1, 5.5 mL*3). The organic phase was concentrated and purified by flash column chromatography (petroleum ether/EtOAc=70/30 to 0/100) to give the product 1-23a (165 mg, 70% yield, 95% purity) as a yellow solid and r.S.M. P6 (60 mg). LCMS (EST): 1299.8 [M+Na]⁺.

Step 2: (2R,3R,4S,5S,6S)-2-((S)-2-((S)-2-amino-3-methylbutanamido)-3-(((2-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)ethoxy)methyl)amino)-3-oxopropoxy)-6-(methoxycarbonyl)tetrahydro-2H-pyran-3,4,5-triyl triacetate (1-23b). To the solution of 1-23a (160 mg, 0.15 mmol) in dry DMF (2 mL) was added Et₂NH (0.305 mL, 2.93 mmol), stirred at r.t. for 15 min. The solution was diluted with toluene, concentrated, and used directly without further purification. LCMS (ESI): 893.6 [M-Na]⁺.

Step 3: (2S,3S,4S,5R,6R)-6-((S)-2-((S)-2-amino-3-methylbutanamido)-3-(((2-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)ethoxy)methyl)amino)-3-oxopropoxy)-3,4,5-trihydroxytetrahydro-2H-pyran-2-carboxylic acid (1-23c). To a solution of 1-23b (136 mg, 0.13 mmol) in MeOH/1H₂O (1/0.5 mL) was added Na₂CO₃ (42 mg, 0.39 mmol), stirred at r.t. for 3 h. K₂CO₃ (18 mg) was added and stirred for 1 h. Water (1 mL) was added, the solution was adjusted to pH 4 with HOAc. The solution was purified by prep-HPLC (Mobile phase A: 0.1% FA in water, B: MeCN; Gradient: 20-32-35-90% B; Flow rate: 20n/min; Column: Xbridge Prep C18 OBD 5 μm, 19*150 mm), lyophilized to give the title compound 1-23c as a yellow solid (37 mg, 31.4% yield, 93% purity). LCMS (ESI): 915.7 [M+H]⁺.

Step 4: (2S,3S,4S,5R,6R)-6-((S)-2-((S)-2-(6-(2,5-dioxo-2,5-dihydro-1H-pyrrol-1-yl)hexanamido)-3-methylbutanamido)-3-(((2-((((1S,9S)-9-ethyl-5-fluoro-9-hydroxy-4-methyl-10,13-dioxo-2,3,9,10,13,15-hexahydro-11H,12H-benzo[de]pyrano[3′,4′:6,7]indolizino[1,2-b]quinolin-1-yl)carbamoyl)oxy)ethoxy)methyl)amino)-3-oxopropoxy)-3,4,5-trihydroxytetrahydro-2H-pyran-2-carboxylic acid (1-23). To the solution of 1-1q (10 mg, 0.046 mmol) and HATU (16.4 mg, 0.042 mmol) in dry DMF (0.5 mL) was added DIEA (14 μL, 0.077 mmol), stirred at r.t. for 5 min. The reaction solution was added to the solution of 1-23c (37 mg, 0.039 mmol) in dry DMF (0.5 mL), stirred at r.t. for 10 min. The solution was purified by prep-HPLC (Mobile phase A: 0.1% FA in water, B: MeCN; Gradient: 32-43-95% B; Flow rate: 20 mL/min; Column: Xbridge Prep C18 OBD 5 μm, 19*150 mm), lyophilized to give the title compound 1-23 as a pink solid (17 mg, 60% yield, 98% purity). LCMS (ESI): 1108.9 [M+H]⁺.

### Example 1-24

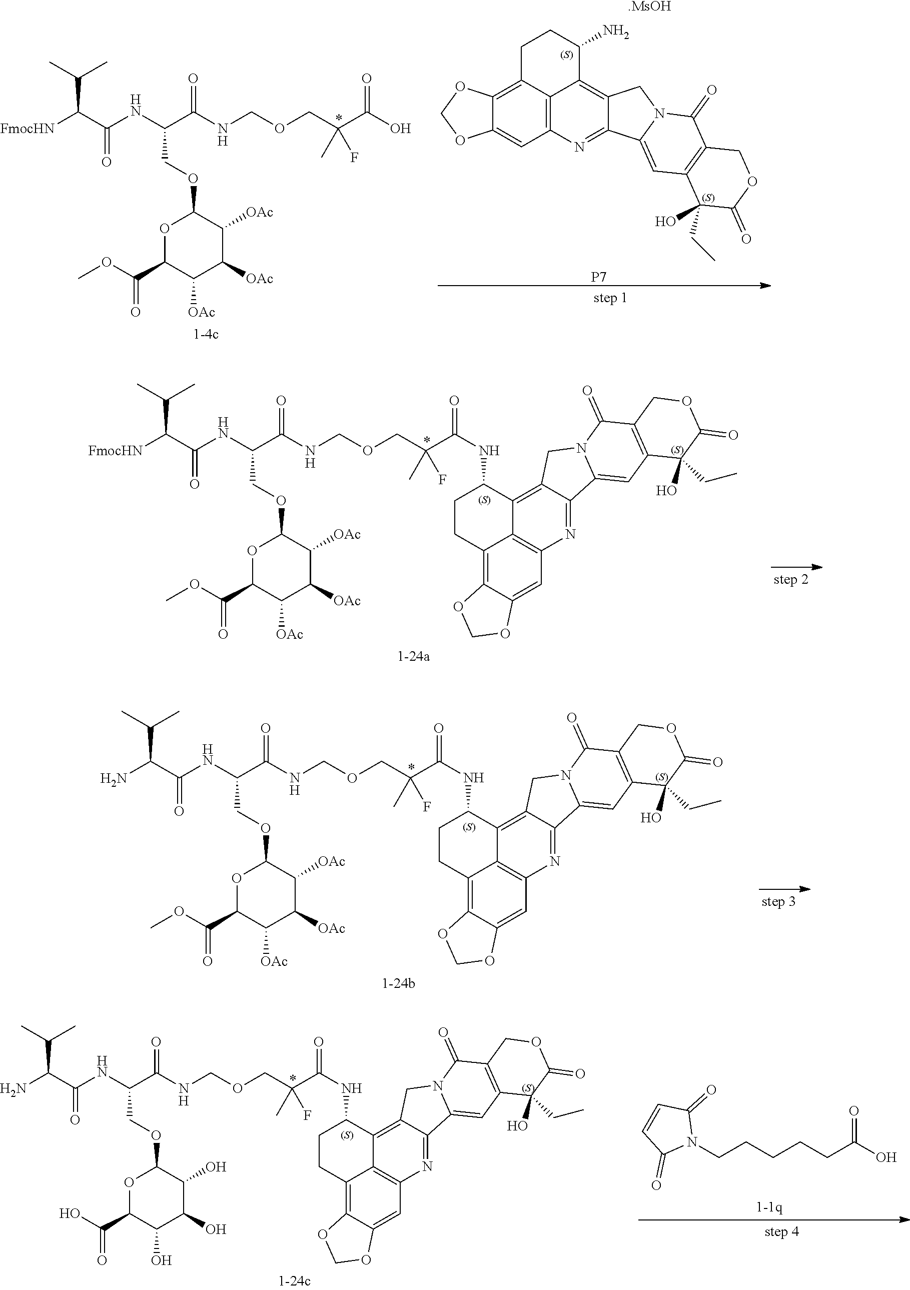

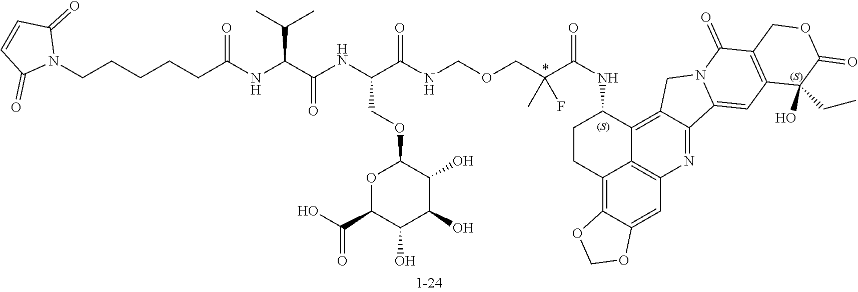

Step 1: (2R,3R,4S,5S,6S)-2-((2S)-2-b((S)-2-((((91H-fluoren-9-yl)methoxy)carbonyl)amino)-3-methylbutamanido)-3-(((3-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-2-fluoro-2-methyl-3-oxopropoxy)methyl)amino)-3-oxopropoxy)-6-(methoxycarbonyl)tetrahydro-21H-pyran-3,4,5-triyl triacetate (1-24a). To a solution of 1-4c (180 ng, 0.21 mmol) and P7 (132 mg, 0.25 mmol) in dry DMF (3 mL) were added HATU (95 mg, 0.25 mmol) and DIPEA (0.11 mL, 0.62 mmol), stirred at r.t. for 30 min. The solution was concentrated and purified by silica gel column chromatography (MeOH/CH₂Cl₂=5:95) to give 1-24a (210 mg, 78.3% yield) as a yellow solid. MS (ESI) m/z: 1305.8 [M+H]⁺.

Step 2: (2R,3R,4S,5S,6S)-2-((2S)-2-((S)-2-amino-3-methyl-butanamido)-3-(((3-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-2-fluoro-2-methyl-3-oxopropoxy)methyl)amino)-3-oxopropoxy)-6-(methoxy carbonyl)tetrahydro-2H-pyran-3,4,5-triyl triacetate (1-24b). To a solution of 1-24a (210 mg, 0.16 mmol) in dry DMF (3 mL) was added Et₂NH (0.34 mL, 3.22 mmol), stirred at r.t. for 30 min. The resulting solution was concentrated to give 1-24b (170 mg, 97.6% yield), which was directly used in the next step. MS (ESI) rm/z: 1084.5 [M+H]⁺.

Step 3: (2S,3S,4S,5R,6R)-6-((2S)-2-((S)-2-amino-3-methylbutanamido)-3-(((3-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-2-fluoro-2-methyl-3-oxopropoxy)methyl)amino)-3-oxopropoxy)-3,4,5-trihydroxytetrahydro-2H-pyran-2-carboxylic acid (1-24c) trifluoroacetate. To a solution of 1-24b (170 mg, 0.16 mmol) in MeOH (2 mL) and water (1 mL) was added IN K₂CO₃ (0.55 mL, 0.55 nmol), stirred at r.t. for 30 min. The resulting solution was quenched with 1HOAc, concentrated to remove MeOH and purified by prep-HPLC (Method: column XBridge Prep C18 OBD 5 μm 19*150 mm; Mobile phase: A-water (0.1% TFA): B-acetonitrile; Flow rate: 20 mL/min), the fraction was lyophilized to give 1-24c (trifluoroacetate) (90 mg, 54.2% yield) as a white solid. MS (ESI) m/z: 943.5 [M+H]⁺.

Step 4: (2S,3S,4S,5R,6R)-6-((2S)-2-((S)-2-(6-(2,5-dioxo-2,5-dihydro-1H-pyrrol-1-yl)hexanamido)-3-methylbutanamido)-3-(((3-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-2-fluoro-2-methyl-3-oxopropoxy)methyl)amino)-3-oxopropoxy)-3,4,5-trihydroxytetrahydro-2H-pyran-2-carboxylic acid (1-24). To the solution of 1-1q (2.2 mg, 0.01 mmol) in dry DMF (1.0 mL) were added HATU (4.3 mg, 0.011 mmol) and DIPEA (0.005 mL, 0.028 mmol), stirred at r.t. for 10 min. To the above mixture was added 1-24c (10 mg, 0.01 mmol), stirred at r.t. for 10 min. The resulting solution was purified by prep-HPLC (Method: column XBridge Prep C18 OBD 5 μm 19*150 mm; Mobile phase: A-water (0.1% FA): B-acetonitrile; Flow rate: 20 mL/min), the fraction was lyophilized to give 1-24 (5 mg, 46.5% yield) as a white solid. MS (ESI) m/z: 1137.6 [M+H]⁺.

### Example 1-25

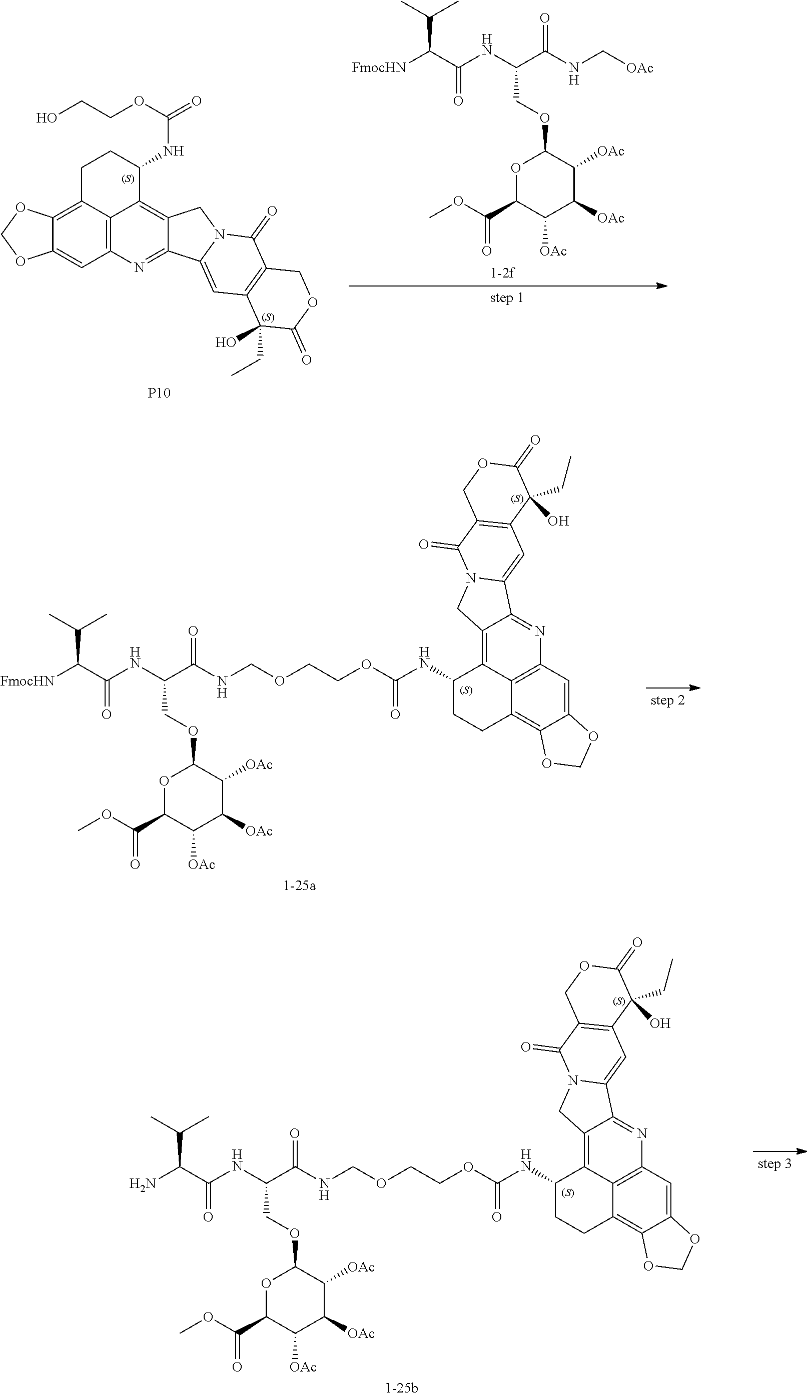

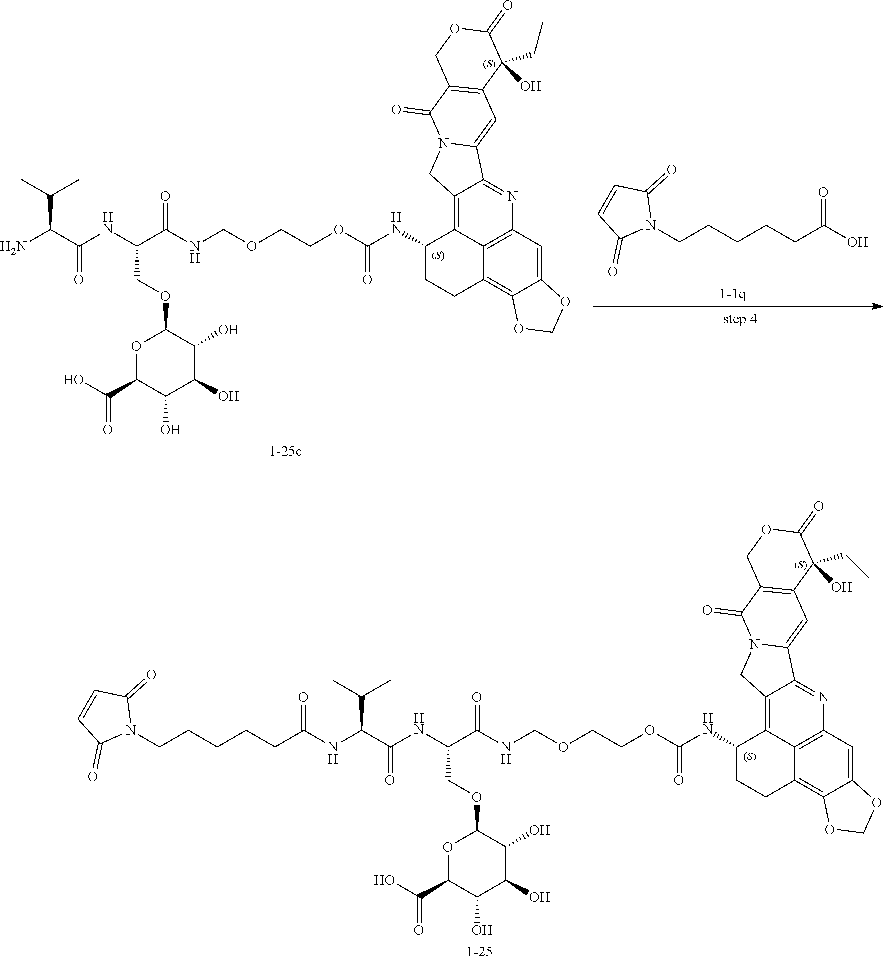

Step 1: (2R,3R,4S,5S,6S)-2-((S)-2-((S)-2-((((9H-fluoren-9-yl)methoxy)carbonyl)amino)-3-methylbutanamido)-3-(((2-((((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)carbamoyl)oxy)ethoxy)methyl)amino)-3-oxopropoxy)-6-(methoxycarbonyl)tetrahydro-2H-pyran-3,4,5-triyl triacetate (1-25a). To a solution of P10 (60 mg, 0.11 mmol) and 1-2f (111 mg, 0.13 mmol) in dry THF (3 mL) was added 4A MS (200 mg) stirred at r.t. for 20 min under N₂. To the above mixture was added Sc(OTf)₃ (73 mg, 0.15 mmol) under N₂ atmosphere, stirred at 60 °C for 16 h. The solution was filtrated and washed with THF (50 mL). The pH of filtrate was adjusted to 8-9 with sat. NaHCO₃ (20 mL) and extracted with CH₂Cl₂ (20 mL*3). The organic phase was concentrated and purified by silica gel column chromatography (Ethyl acetate/CH₂Cl₂=50/50) to give 1-25a (30 mg, 20.8% yield) as a yellow solid. MS (EST) m/z: 1289.7 [M+Na]⁺.

Step 2: (2R,3R,4S,5S,6S)-2-((S)-2-((S)-2-amino-3-methylbutanamido)-3-(((2-((((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)carbamoyl)oxy)ethoxy)methyl)amino)-3-oxopropoxy)-6-(methoxy carbonyl)tetrahydro-2H-pyran-3,4,5-triyl triacetate (1-25b). To a solution of 1-25a (30 mg, 0.023 mmol) in dry DMF (1 mL) was added Et₂NH (0.049 mL, 0.47 mmol), stirred at r.t. for 30 min. The resulting solution was concentrated to give 1-25b (24 mg, 96.7% yield), which was directly used in the next step. MS (ESI) m/z: 1067.5 [M+H]⁺.

Step 3: (2S,3S,4S,5R,6R)-6-((S)-2-((S)-2-amino-3-methylbutanamido)-3-(((2-((((1S,10S)-10-ethyl-10-hydroxy-11,14-dioxo-2,3,10,11,14,16-hexahydro-1H,131H-benzo[de][1,3]dioxolo[4,5-g]pyrano[3′,4′:6,7]indolizino[1,2-b]quinolin-1-yl)carbamoyl)oxy)ethoxy)methyl)amino)-3-oxopropoxy)-3,4,5-trihydroxytetrahydro-2H-pyran-2-carboxylic acid (1-25c) trifluoroacetate. To a solution of 1-25b (24 mg, 0.022 mmol) in MeOH (0.5 ml) and water (0.2 ml) was added IN K₂CO₃ (0.079 mL, 0.079 mmol), stirred at r.t. for 30 min. The resulting solution was quenched with HOAc, concentrated to remove MeOH and purified by prep-HPLC (Method: column XBridge Prep C18 OBD 5 μm 19*150 mm; Mobile phase: A-water (0.1% TFA): B-acetonitrile; Flow rate: 20 mL/min), the fraction was lyophilized to give 1-25c (trifluoroacetate) (12 mg, 51.3% yield) as a white solid. MS (ESI) m/z: 927.5 [M+H]⁺.

Step 4: (2S,3S,4S,5R,6R)-6-((S)-2-((S)-2-(6-(2,5-dioxo-2,5-dihydro-1H-pyrrol-1-yl)hexanamido)-3-methylbutanamido)-3-(((2-((((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)carbamoyl)oxy)ethoxy)methyl)amino)-3-oxopropoxy)-3,4,5-trihydroxytetrahydro-2H-pyran-2-carboxylic acid (1-25). To the solution of 1-1q (2.3 mg, 0.01 mmol) in dry DMF (1 ml) were added HATU (4.4 mg, 0.012 mmol) and DIPEA (0.005 ml, 0.029 mmol), stirred at r.t. for 10 min. To the above mixture was added 1-25c (10 mg, 0.01 mmol), stirred at r.t. for 10 min. The resulting solution was purified by prep-HPLC (Method: column XBridge Prep C18 OBD 5 μm 19*150 mm; Mobile phase: A-water (0.1% FA): B-acetonitrile; Flow rate: 20 mL/min), the fraction was lyophilized to give 1-25 (3.7 mg, 34.4% yield) as a white solid. MS (EST) m/z: 1121.5 [M+H1].

### Example 1-26

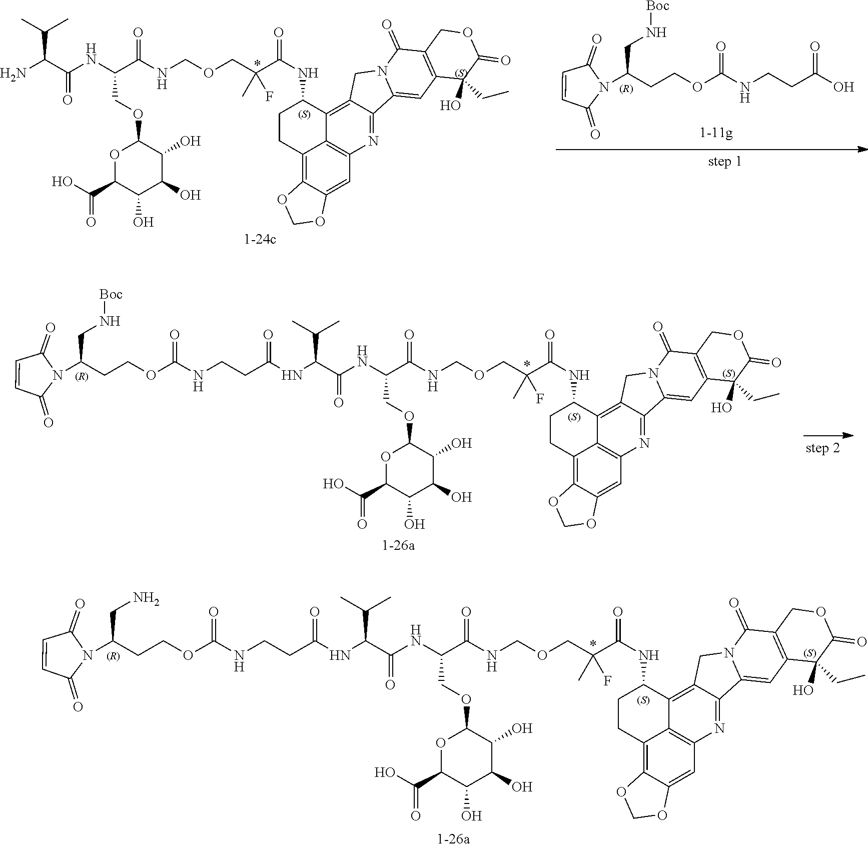

Step 1: (2S,3S,4S,5R,6R)-6-(((7R,17S,20S)-7-(2,5-dioxo-2,5-dihydro-1H-pyrrol-1-yl)-20-(((3-(((1S,10S)-10-ethyl-10-hydroxy-11,14-dioxo-2,3,10,11,14,16-hexahydro-111,1311-benzo[de][1,3]dioxolo[4,5-g]pyrano[3′,4′:6,7]indolizino[1,2-b]quinolin-1-yl)amino)-2-fluoro-2-methyl-3-oxopropoxy)methyl)carbamoyl)-17-isopropyl-2,2-dimethyl-4,11,15,18-tetraoxo-3,10-dioxa-5,12,16,19-tetraazahenicosan-21-yl)oxy)-3,4,5-trihydroxytetrahydro-2H-pyran-2-carboxylic acid (1-26a). To a solution of 1-11g (9.2 mg, 0.023 mmol) in dry DMF (1 mL) were added HATU (8.7 mg, 0.023 mmol) and DIPEA (0.007 ml, 0.038 mmol), stirred at r. for 10 min. Then to the above mixture was added 1-24c (20 mg, 0.019 mmol), stirred at r.t. for 10 min. The resulting solution was purified by prep-HPLC (Method: column XBridge Prep C18 OBD 5 μm 19*150 mm; Mobile phase: A-water (0.1% FA): B-acetonitrile; Flow rate: 20 mL/min), the fraction was lyophilized to give 1-26a (18 mg, 71.8% yield) as a white solid. MS (ESI) m/z: 1324.8 [M+H]⁺.

Step 2: (2S,3S,4S,5R,6R)-6-(((2S,5S,15R)-16-amino-15-(2,5-dioxo-2,5-dihydro-1H-pyrrol-1-yl)-2-(((3-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-2-fluoro-2-methyl-3-oxopropoxy)methyl)carbamoyl)-5-isopropyl-4,7,11-trioxo-12-oxa-3,6,10-triazahexadecyl)oxy)-3,4,5-trihydroxytetrahydro-2H-pyran-2-carboxylic acid (1-26). To the solution of 1-26a (18 mg, 0.014 mmol) in DCM (0.4 mL) was added ZnBr₂ (124 mg, 0.54 mmol), stirred at reflux for 16 h. The resulting solution was quenched with 0.1% aq. FA at 0 °C. The resulting solution was purified by prep-HPLC (Method: column XBridge Prep C18 OBD 5 μm 19*150 mm; Mobile phase: A-water (0.1% FA): B-acetonitrile; Flow rate: 20 mL/min), the fraction was lyophilized to give 1-26 (12 mg, 72.1% yield) as a white solid. MS (ESI) m/z: 1224.6 [M+H]⁺.

### Example 1-27

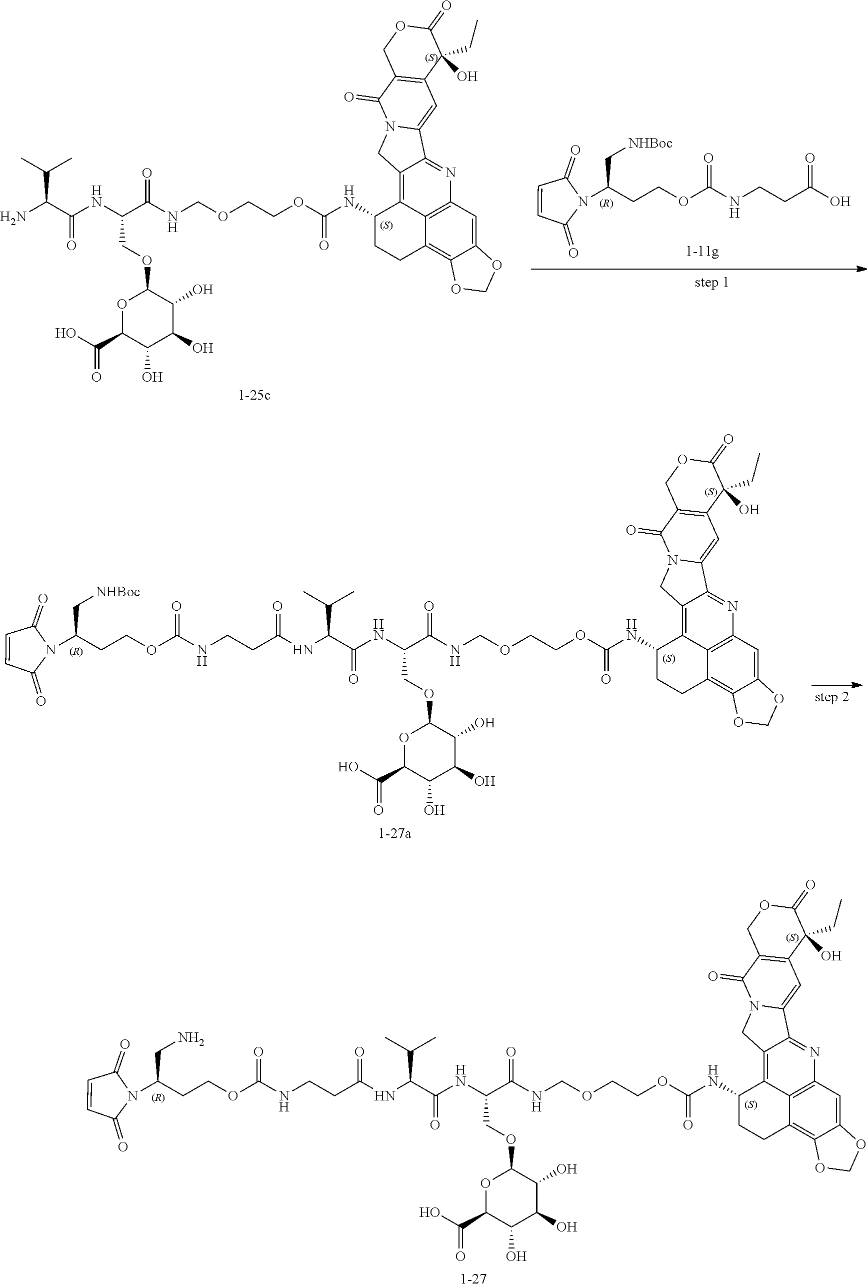

Step 1: (2S,3S,4S,5R,6R)-6-(((7R,17S,20S)-7-(2,5-dioxo-2,5-dihydro-1H-pyrrol-1-yl)-20-(((2-((((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)carbamoyl)oxy)ethoxy)methyl)carbamoyl)-17-isopropyl-2,2-dimethyl-4,11,15,18-tetraoxo-3,10-dioxa-5,12,16,19-tetraazahenicosan-21-yl)oxy)-3,4,5-trihydroxytetrahydro-2H-pyran-2-carboxylic acid (1-27a). To a solution of 1-11g (4.7 mg, 0.012 mmol) in dry DMF (0.5 mL) were added HATU (4.4 mg, 0.012 mmol) and DIPEA (0.003 mL, 0.019 mmol), stirred at r.t. for 10 min. Then to the above mixture was added 1-25c (10 mg, 0.01 mmol), stirred at r. for 10 min. The resulting solution was purified by prep-HPLC (Method: column XBridge Prep C18 OBD 5 μm 19*150 mm; Mobile phase: A-water (0.1% FA): B-acetonitrile; Flow rate: 20 mL/min), the fraction was lyophilized to give 1-27a (7.3 mg, 58.1% yield) as a white solid. MS (ESI) m/z: 1308.6 [M+H]⁺.

Step 2: (2S,3S,4S,5R,6R)-6-(((2S,5S,15R)-16-amino-15-(2,5-dioxo-2,5-dihydro-1H-pyrrol-1-yl)-2-(((2-((((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)carbamoyl)oxy)ethoxy)methyl)carbamoyl)-5-isopropyl-4,7,11-trioxo-12-oxa-3,6,10-triazahexadecyl)oxy)-3,4,5-trihydroxytetrahydro-2H-pyran-2-carboxylic acid (1-27). To a solution of 1-27a (7.3 mg, 0.006 mmol) in DCM (0.2 mL) was added ZnBr₂ (51 mg, 0.22 mmol), stirred at reflux for 16 h. The resulting solution was quenched with 0.1% aq. FA at 0 °C. The resulting solution was purified by prep-HPLC (Method: column XBridge Prep C18 OBD 5 μm 19*150 mm; Mobile phase: A-water (0.1% FA): B-acetonitrile; Flow rate: 20 mL/min), the fraction was lyophilized to give 1-27 (4.2 mg, 62.3% yield) as a white solid. MS (ESI) m/z: 1208.7 [M+H]⁺.

### Example 9-28

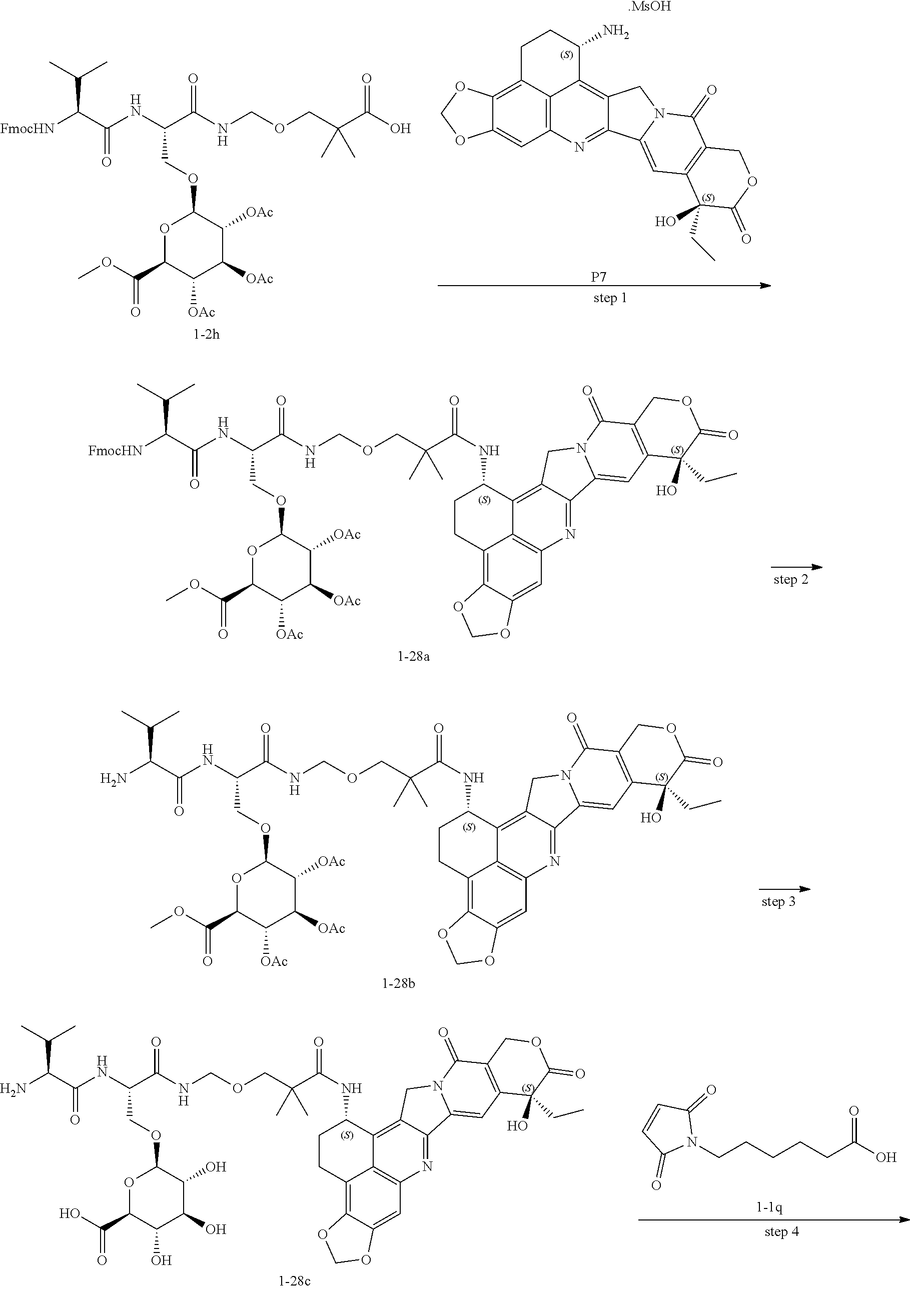

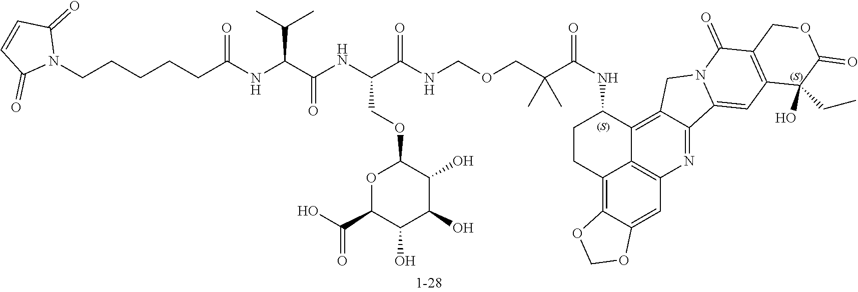

Step 9: (2R,3R,4S,5S,6S)-2-((S)-2-((S)-2-((((9H-fluoren-9-yl)methoxy)carbonyl)amino)-3-methylbutanamido)-3-(((3-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-2,2-dimethyl-3-oxopropoxy)methyl)amino)-3-oxopropoxy)-6-(methoxycarbonyl)tetrahydro-2H-pyran-3,4,5-triyl triacetate (1-28a). To a solution of 1-2h (450 mg, 0.52 mmol) and P7 (340 mg, 0.62 nmol) in dry DMF (10 mL) were added HATU (238 mg, 0.62 mmol) and DIPEA (0.28 mL, 1.55 mmol), stirred at r.t. for 30 min. The solution was concentrated and purified by silica gel column chromatography (MeOH/CH₂Cl₂=5/95) to give 1-28a (440 mg, 65.5% yield) as a yellow solid. MS (ESI) m/z: 1301.6[M+H]⁺.

Step 2: (2R,3R,4S,5S,6S)-2-((S)-2-((S)-2-amino-3-methylbutanamido)-3-(((3-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-2,2-dimethyl-3-oxopropoxy)methyl)amino)-3-oxopropoxy)-6-(methoxy carbonyl)tetrahydro-2H-pyran-3,4,5-triyl triacetate (1-28b). To a solution of 1-28a (440 mg, 0.34 mmol) in dry DMF (5 mL. was added Et₂NH (0.71 mL, 6.76 mmol), stirred at r.t. for 30 min. The resulting solution was concentrated to give 1-28b (350 mg, 95.9% yield), which was directly used in the next step. MS (EST) m/z: 1079.5 [M+H]⁺.

Step 3: (2S,3S,4S,5R,6R)-6-((S)-2-((S)-2-amino-3-methylbutanamido)-3-(((3-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-2,2-dimethyl-3-oxopropoxy)methyl)amino)-3-oxopropoxy)-3,4,5-trihydroxytetrahydro-2H-pyran-2-carboxylic acid (1-28c) trifluoroacetate. To a solution of 1-28b (350 mg, 0.32 mmol) in MeOH (5 mL) and water (2 mL) was added 1N K₂CO₃ (1.14 mL, 1.14 mmol) at 0 °C, stirred at r.t. for 30 min. The resulting solution was quenched with HOAc, concentrated to remove MeOH, and purified by prep-HPLC (Method: column XBridge Prep C18 OBD 5 μm 19*150 mm; Mobile phase: A-water (0.1% TFA): B-acetonitrile; Flow rate: 20 mt/min), the fraction was lyophilized to give 1-28c (trifluoroacetate) (130 mg, 38.1% yield) as a white solid. MS (ESI) m/z: 939.5 [M+H]⁺.

Step 4: (2S,3S,4S,5R,6R)-6-((S)-2-((S)-2-(6-(2,5-dioxo-2,5-dihydro-1H-pyrrol-1-yl)hexanamido)-3-methylbutanamido)-3-(((3-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-2,2-dimethyl-3-oxopropoxy)methyl)amino)-3-oxopropoxy)-3,4,5-trihydroxytetrahydro-2H-pyran-2-carboxylic acid (1-28). To a solution of 1-1q (7.2 mg, 0.034 mmol) in dry DMF (1.0 mL) were added HATU (14.7 mg, 0.038 mmol) and DIPEA (0.011 mL, 0.064 mmol), stirred at r.t. for 10 min. To the above mixture was added 1-28c (30.0 mg, 0.032 mmol), stirred at r.t. for 10 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min), the fraction was lyophilized to give 1-28 (15.0 mg, 41.5% yield) as a white solid. MS (ESI) m/z: 1132.8 [M+H]⁺.

### Example 1-29

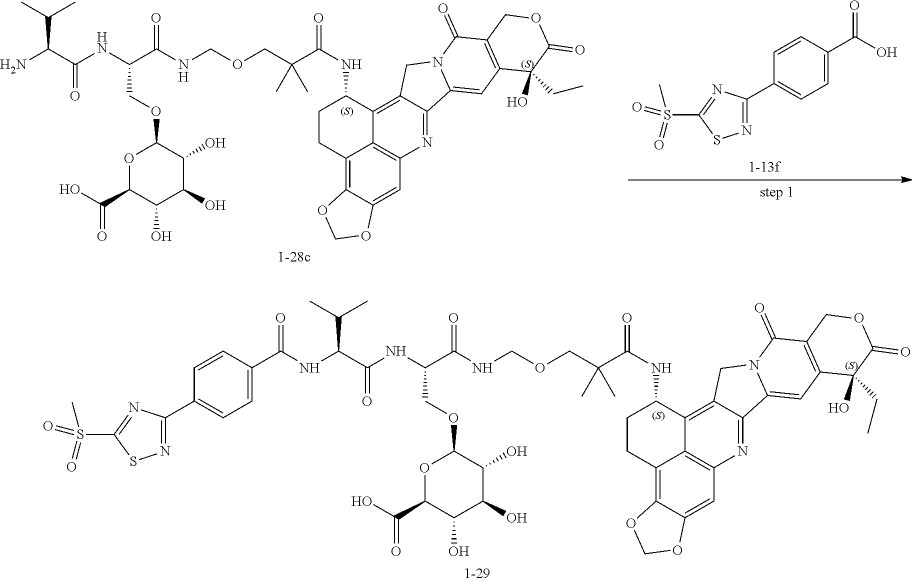

Step 1: (2S,3S,4S,5R,6R)-6-((S)-3-(((3-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-2,2-dimethyl-3-oxopropoxy)methyl)amino)-2-((S)-3-methyl-2-(4-(5-(methylsulfonyl)-1,2,4-thiadiazol-3-yl)benzamido)butanamido)-3-oxopropoxy)-3,4,5-trihydroxytetrahydro-2H-pyran-2-carboxylic acid (1-29). To a solution of 1-13f (5.9 mg, 0.021 mmol) in dry DMF (0.5 mL) were added HATU (7.9 mg, 0.021 mmol) and DIPEA (0.008 mL, 0.043 mmol), stirred at r.t. for 15 min. Then to the above mixture was added 1-28c (trifluoroacetate) (18.0 mg, 0.017 mmol), stirred at r.t. for 10 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min), the fraction was lyophilized to give 1-29 (5.5 mg, 26.7% yield) as a yellow solid. MS (ESI) m/z: 1206.0 [M+H]⁺.

### Example 1-30

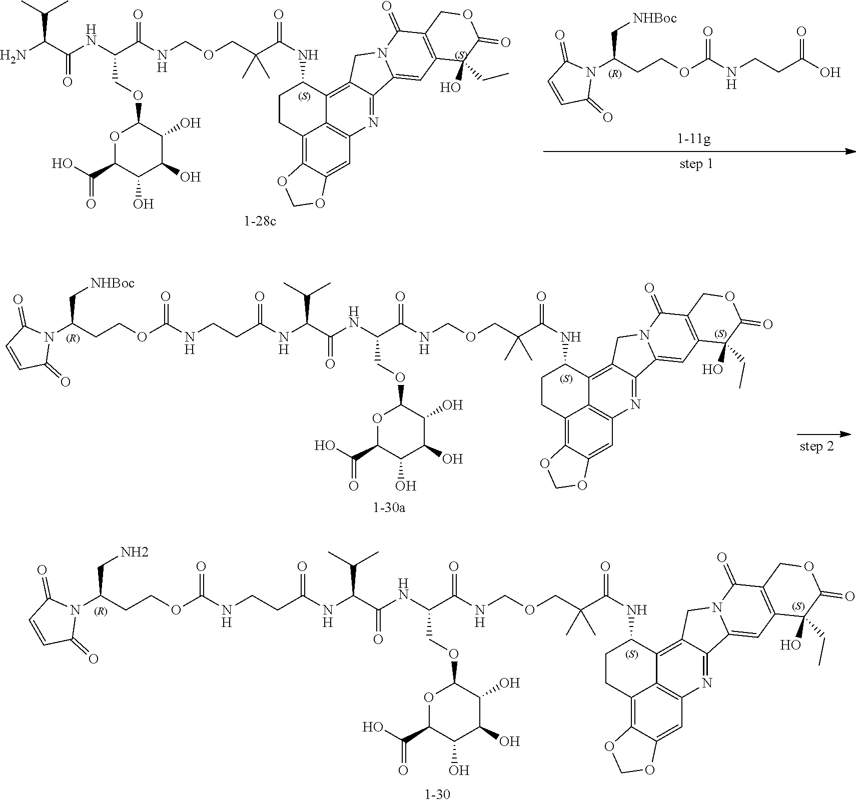

Step 1: (2S,3S,4S,5,6R)-6-(((7R,17S,20S)-7-(2,5-dioxo-2,5-dihydro-1H-pyrrol-1-3yl)-20-(((3-(((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)amino)-2,2-dimethyl-3-oxopropoxy)methyl)carbamoyl)-17-isopropyl-2,2-dimethyl-4,11,15,18-tetraoxo-3,10-dioxa-5,12,16,19-tetraazahenicosan-21-yl)oxy)-3,4,5-trihydroxytetrahydro-2H-pyran-2-carboxylic acid (1-30a). To a solution of 1-11g (5.9 mg, 0.021 mmol) in dry DMF (0.5 mL) were added HATU (7.9 mg, 0.021 mmol) and DIPEA (0.008 mL, 0.043 mmol), stirred at r.t. for 15 min. Then to the above mixture was added 1-28c (trifluoroacetate) (18.0 mg, 0.017 mmol), stirred at r.t. for 10 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min), the fraction was lyophilized to give 1-30a (5.5 mg, 26.7% yield) as a yellow solid. MS (ESI) m/z: 1320.8 [M+H]⁺.

Step 2: (2S,3S,4S,5R,6R)-6-(((2S,5S,15R)-16-amino-15-(2,5-dioxo-2,5-dihydro-1H-pyrrol-1-yl)-2-(((3-(((1S,10S)-10-ethyl-10-hydroxy-11,14-dioxo-2,3,10,11,14,16-hexahydro-1H,13H-benzo[de][1,3]dioxolo[4,5-g]pyrano[3,4:6,7]indolizino[1,2-b]quinolin-1-yl)amino)-2,2-dimethyl-3-oxopropoxy)methyl)carbamoyl)-5-isopropyl-4,7,11-trioxo-12-oxa-3,6,10-triazabexadecyl)oxy)-3,4,5-trihydroxytetrahydro-2H-pyran-2-carboxylic acid (1-30). To the solution of 1-30a (16 mg, 0.012 mmol) in DCM (0.4 mL) was added ZnBr₂ (110 mg, 0.49 mmol), stirred at reflux for 16 h. The resulting solution was quenched with 0.1% aq. FA at 0 °C. The resulting solution was purified by prep-HPLC (Method: column XBridge Prep C18 OBD 5 μm 19*150 mm; Mobile phase: A-water (0.1% FA): B-acetonitrile; Flow rate: 20 mL/min), the fraction was lyophilized to give 1-30 (12 mg, 72.1% yield) as a white solid. MS (ESI) m/z: 1220.6 [M+H]⁺.

### Example 1-31

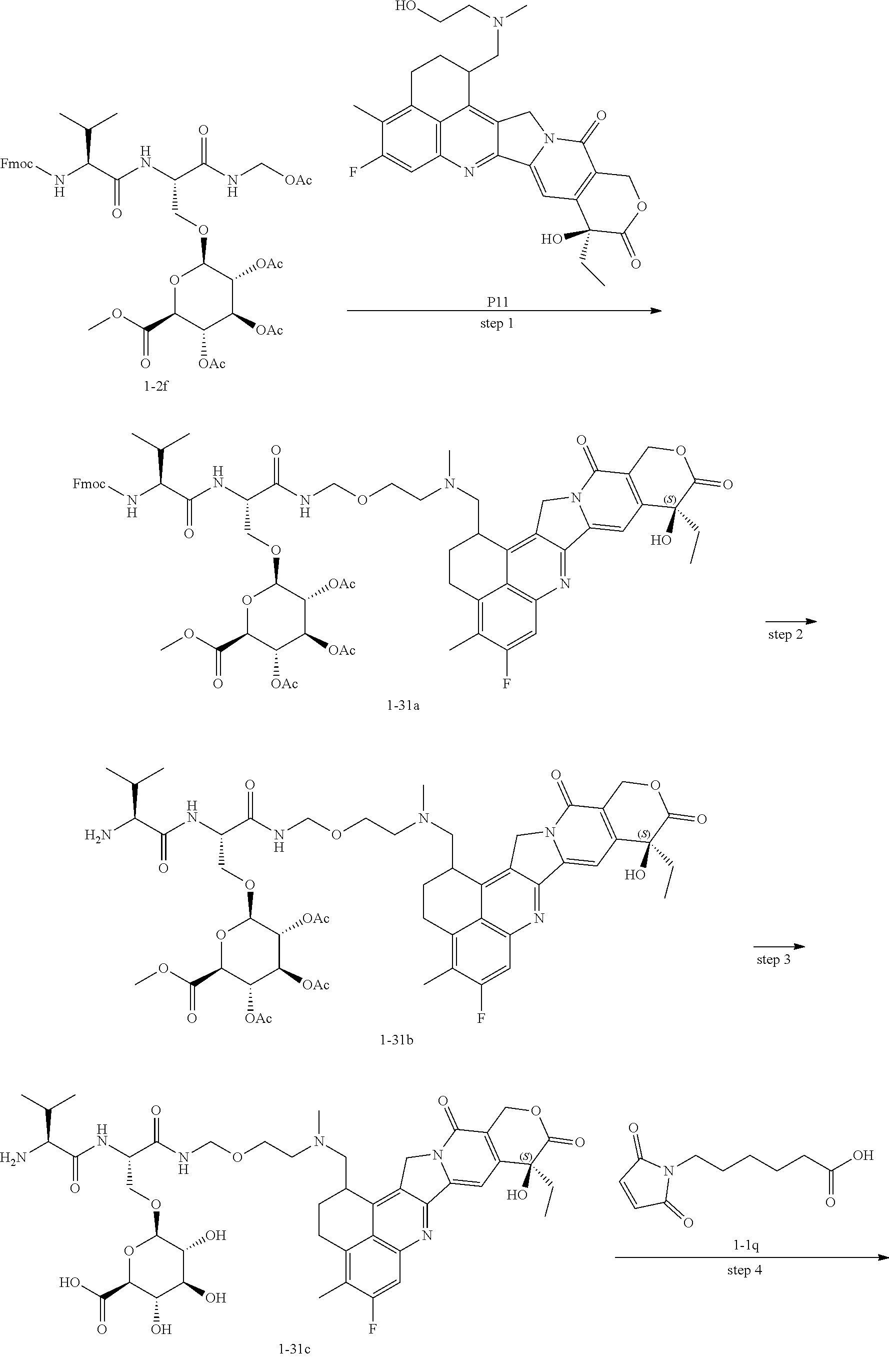

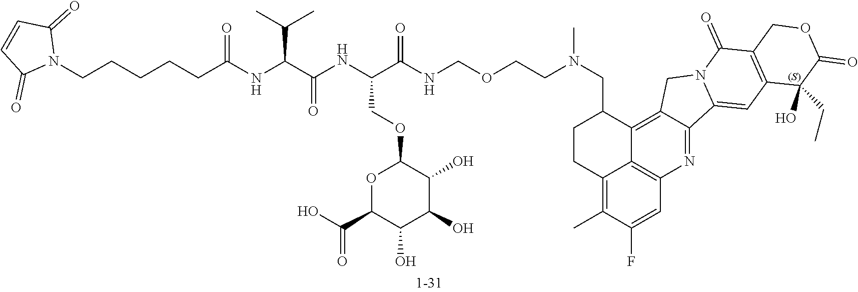

Step 1: (2R,3R,4S,5S,6S)-2-((2S)-2-((S)-2-((((9H-fluoren-9-yl)methoxy)carbonyl)amino)-3-methylbutanamido)-3-(((2-((((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)methyl)(methyl)amino)ethoxy)methyl)amino)-3-oxopropoxy)-6-(methoxycarbonyl)tetrahydro-2H-pyran-3,4,5-triyl triacetate (1-31a). To a solution of 1-2f (55 mg, 0.066 mmol) and P11 (45 mg, 0.055 mmol) in dry THF (2 mL) was added 4A MS (200 mg), and stirred for 20 min. Sc(OTf)₃ (41.7 mg, 0.083 mmol) was added, and the solution was stirred at 40 °C for 15 h. 1-2f (23 mg) was added, and the solution was stirred at 40 °C for 8 h. 4A MS (100 mg) and Sc(OTf)₃ (15 mg) were added, and then stirred at 40 °C for another 14 h. The mixture was filtered and the filtrate was washed with sat. NaHCO₃ (5 mL). The water phase was extracted with DCM/MeOH (10:1, 5.5 mL*3). The organic phase was concentrated and purified by flash column chromatography (DCM/MeOH=100/0 to 90/10) to give the crude product 1-31a (84 mg, theoretical 55 mg, 78.9% yield) as a yellow solid. LCMS (ESI): 1283.9 [M+H]⁺.

Step 2: (2R,3R,4S,5S,6S)-2-((2S)-2-((S)-2-amino-3-methylbutanamido)-3-(((2-((((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)methyl)(methyl)amino)ethoxy)methyl)amino)-3-oxopropoxy)-6-(methoxycarbonyl)tetrahydro-2H-pyran-3,4,5-triyl triacetate (1-31b). To a solution of 1-31a (84 mg crude, theoretical 55 mg, 0.044 mmol) in dry DMF (1 mL) was added Et₂NH (91 μL, 0.87 mmol), and the solution was stirred at r.t. for 10 min. The solution was diluted with toluene, concentrated and used directly without further purification. LCMS (ESI): 1061.8 [M+Na].

Step 3: (2S,3S,4S,5R,6R)-6-((2S)-2-((S)-2-amino-3-methylbutanamido)-3-(((2-((((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)methyl)(methyl)amino)ethoxy)methyl)amino)-3-oxopropoxy)-3,4,5-trihydroxytetrahydro-2H-pyran-2-carboxylic acid (1-31c). To a solution of 1-31b (45 mg, crude, 0.043 mmol) in MeOH/H₂O (1/0.5 mL) was added K₂CO₃ (18 mg, 0.13 mmol), and then stirred at r.t. for 2 h.

Water (1 mL) was added and the solution was adjusted to pH 6 with IN HCl. The solution was purified by prep-HPLC (Mobile phase A: 0.1% FA in water, B: MeCN; Gradient: 20-28-32% B; Flow rate: 20 mL/min; Column: Xbridge Prep C18 OBD 5 μm, 19*150 mm), lyophilized to give the title compound 1-31c as a white solid (11 mg, 28.3% yield, 89% purity). LCMS(ESI): 899.8 [M+H]⁺.

Step 4: (2S,3S,4S,5R,6R)-6-((2S)-2-((S)-2-(6-(2,5-dioxo-2,5-dihydro-11H-pyrrol-1-yl)hexanamido)-3-methylbutanamido)-3-(((2-((((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)methyl)(methyl)amino)ethoxy)methyl)amino)-3-oxopropoxy)-3,4,5-trihydroxytetrahydro-2H-pyran-2-carboxylic acid (1-31). To a solution of 1-1q (3.2 mg, 0.015 mmol) and HATU (5.2 mg, 0.013 mmol) in dry DMF (0.5 mL) was added DIEA (4 μL, 0.024 mmol), stirred at r.t. for 5 min. The reaction solution was added to the solution of 1-31c (11 mg, 0.012 nmol) in dry DMF (0.5 mL), and stirred at r.t. for 15 min. The solution was purified by prep-HPLC (Mobile phase A: 0.1% FA in water, B: MeCN; Gradient: 25-40-95% B: Flow rate: 20 mL/min; Column: Xbridge Prep C18 OBD 5 μm, 19*150 mm), lyophilized to give the title compound 1-31 as an off-white solid (4.3 mg, 32% yield, 96% purity). LCMS (ESI): 1092.8 [M+H]⁺.

### Example 1-32

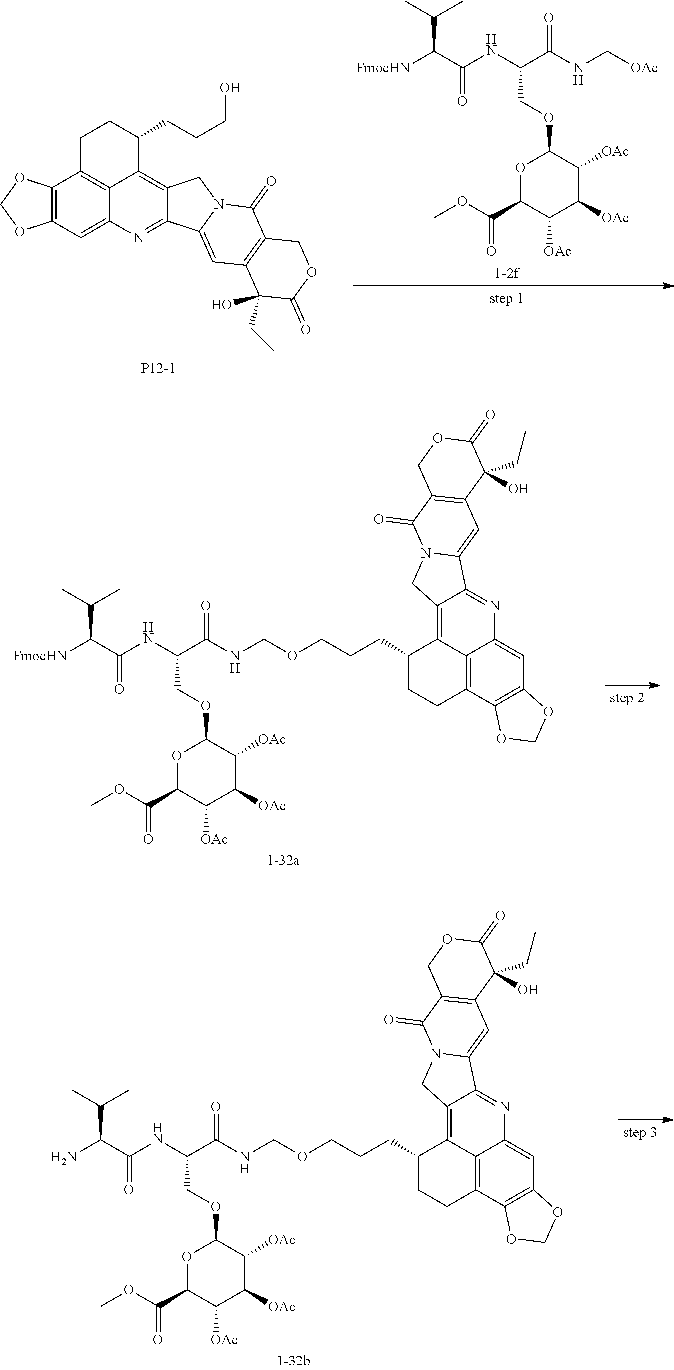

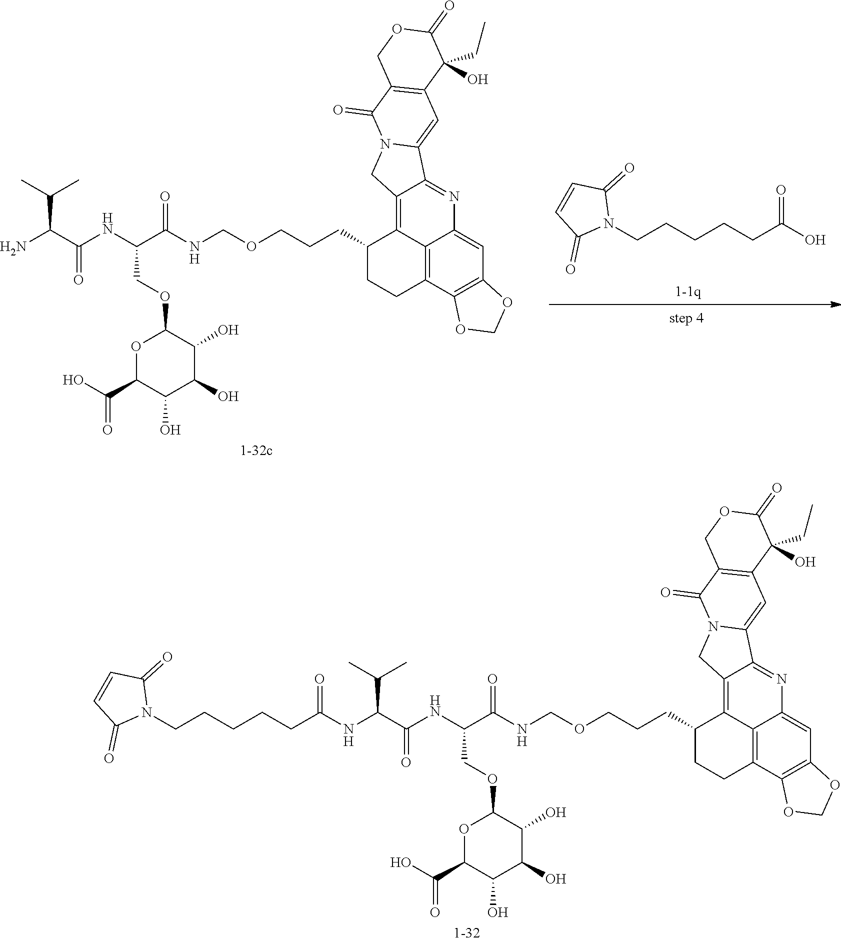 [SMILES: CC[C@@]1(O)C(=O)OCc2c1cc1n(c2=O)Cc2c-1nc1cc3c(c4c1c2[C@@H](CCCOCNC(=O)[C@H](CO[C@@H]1O[C@H](C(=O)O)[C@@H](O)[C@H](O)[C@H]1O)NC(=O)[C@@H](N)C(C)C)CC4)OCO3 ; O=C(O)CCCCCN1C(=O)C=CC1=O ; CC[C@@]1(O)C(=O)OCc2c1cc1n(c2=O)Cc2c-1nc1cc3c(c4c1c2[C@@H](CCCOCNC(=O)[C@H](CO[C@@H]1O[C@H](C(=O)O)[C@@H](O)[C@H](O)[C@H]1O)NC(=O)[C@@H](NC(=O)CCCCCN1C(=O)C=CC1=O)C(C)C)CC4)OCO3]

Step 1: (2R,3R,4S,5S,6S)-2-((S)-2-((S)-2-((((9H-fluoren-9-yl)methoxy)carbonyl)amino)-3-methylbutanamido)-3-(((3-((1R,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)propoxy)methyl)amino)-3-oxopropoxy)-6-(methoxycarbonyl)tetrahydro-2H-pyran-3,4,5-triyl triacetate (1-32a). To a solution of P12-1 (50 mg, 0.10 mmol) and 1-2f (99 mg, 0.12 mmol) in dry THF (2 mL) was added 4A MS (50 mg), stirred for 20 min. Sc(OTf)₃ (74 mg, 0.15 mmol) was added, and stirred at 35 °C for 14 h and at 50 °C for 16 h. The mixture was filtered and the filtrate was washed with sat. NaHCO₃ (5 mL). The water phase was extracted with DCM/MeOH (10:1, 5.5 mL*3). The organic phase was concentrated and purified by flash column chromatography (petroleum ether/EtOAc=70/30 to 0/100) to give the compound 1-32a (50 mg, 39.4% yield) as white solid. MS (ESI) m/z: 1245.0 [M+H]⁺.

Step 2: (2R,3R,4S,5S,6S)-2-((S)-2-((S)-2-amino-3-methylbutanamido)-3-(((3-((1R,10S)-1 O-ethyl-10-hydroxy-11,14-dioxo-2,3,10,11,14,16-hexahydro-1H,13H-benzo[de][1,3]dioxolo[4,5-g]pyrano[3′,4′:6,7]indolizinol 1,2-b]quinolin-1-yl)propoxy)methyl)amino)-3-oxopropoxy)-6-(methoxycarbonyl)tetrahydro-2H-pyran-3,4,5-triyl triacetate (1-32b). To a solution of 1-32a (50 mg, 0.040 mmol) in dry DMF (2 mL) was added Et₂NH (0.083 mL, 0.81 mmol), stirred at r.t. for 15 min. The solution was diluted with toluene, concentrated and used directly without further purification. MS (ESI) m/z: 1022.8 [M+H]⁺.

Step 3: (2S,3S,4S,5R,6R)-6-((S)-2-((S)-2-amino-3-methylbutanamido)-3-(((3-((1R,10S)-1 O-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)propoxy)methyl)amino)-3-oxopropoxy)-3,4,5-trihydroxytetrahydro-2H-pyran-2-carboxylic acid (1-32c). To a solution of 1-32b (50 mg, 0.049 mmol) in MeOH/H₂O (5/2 ml) was added Na₂CO₃ (36 mg, 0.34 mmol), stirred at r.t. for 1 h. The solution was adjusted to pH 4 with HOAc. The solution was purified by prep-HPLC (Mobile phase A: 0.1% FA in water, B: MeCN; Gradient: 20-32-35-90% B; Flow rate: 20 mL/min; Column: Xbridge Prep C18 OBD 5 μm, 19*150 mm), lyophilized to give the title compound 1-32c (20 m, 47.6% yield) as a yellow solid. MS (EST) m/z: 882.7 [M+H]⁺.

Step 4: (2S,3S,4S,5R,6R)-6-((S)-2-((S)-2-(6-(2,5-dioxo-2,5-dihydro-TH-pyrrol-1-yl)hexanamido)-3-methylbutanamido)-3-(((3-((1R,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)propoxy)methyl)amino)-3-oxopropoxy)-3,4,5-trihydroxytetrahydro-2H-pyran-2-carboxylic acid (1-32). To a solution of 1-1q (26 mg, 0.12 mmol) and HATU (35 mg, 0.093 mmol) in dry DMF (3 mL) was added DIEA (33 μL, 0.12 mmol), stirred at r.t. for 5 min. The reaction solution was added to the solution of 1-32c (55 mg, 0.062 mmol) in dry DMF (2 mL), stirred at r.t. for 10 min. The solution was purified by prep-HPLC (Mobile phase A: 0.1% FA in water, B: MeCN; Gradient: 32-43-95% B; Flow rate: 20 mL/min; Column: Xbridge Prep C18 OBD 5 μm, 19*150 mm), lyophilized to give the title compound 1-32 (9.4 mg, 38.5%) as a yellow solid. MS (EST) m/z: 1075.9 [M+H]1

TABLE 1

| Compound ID | Linker-Payload Structure |
|---|---|
| 1-1 | 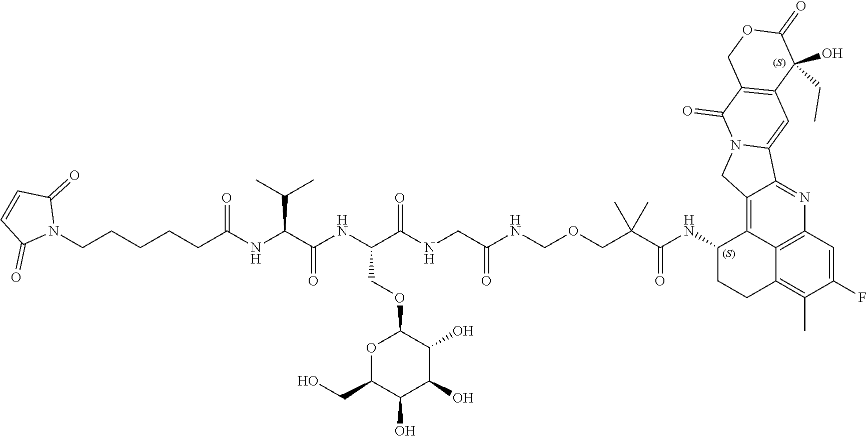 |
| 1-2 | 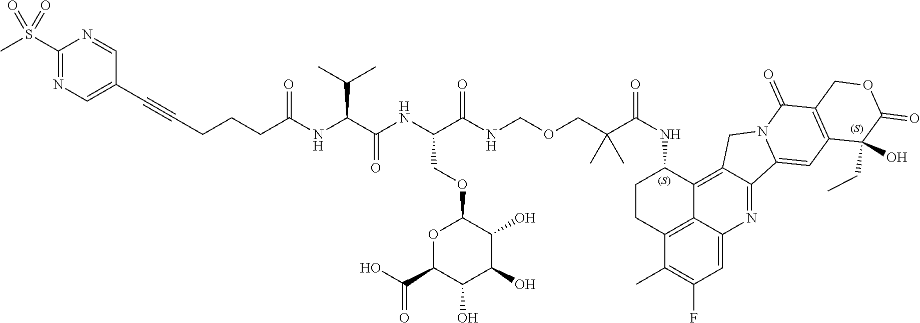 |
| 1-3 | 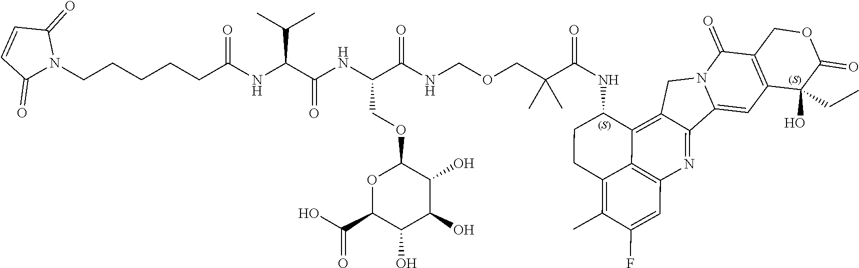 |
| 1-4 | 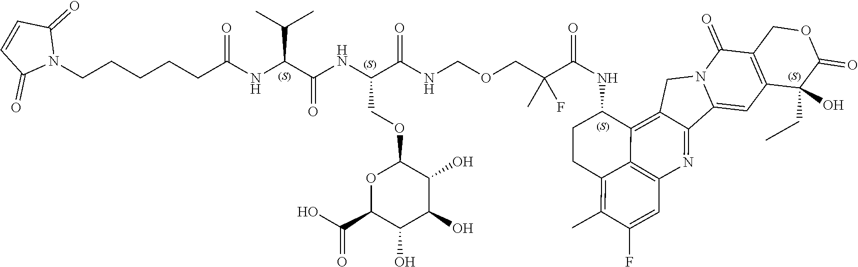 |
| 1-5 | 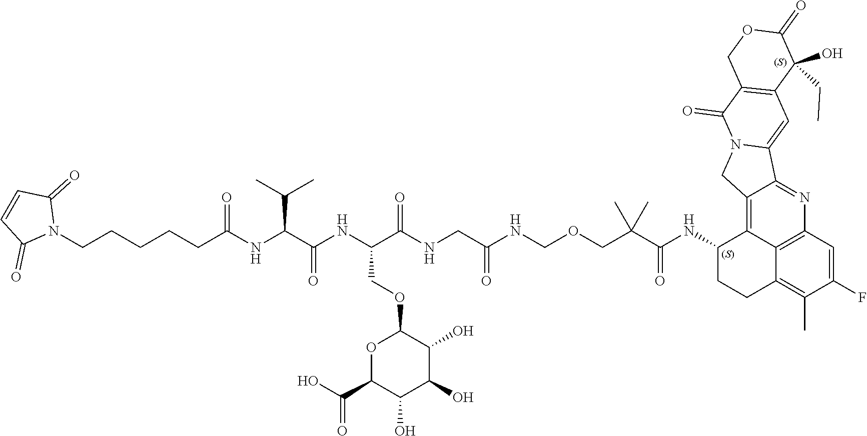 |
| 1-6 | 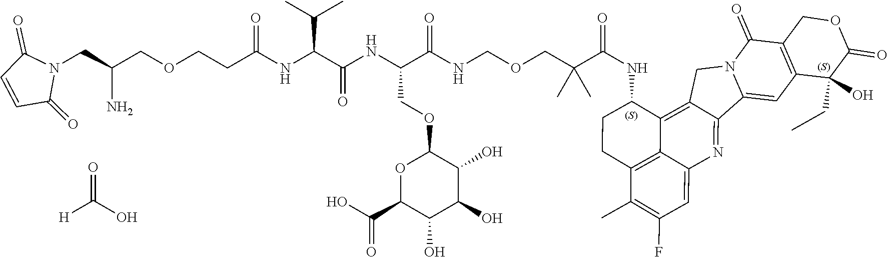 |
| 1-7 | 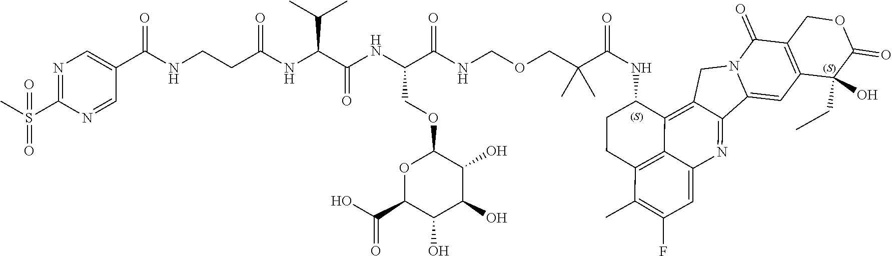 |
| 1-8 | 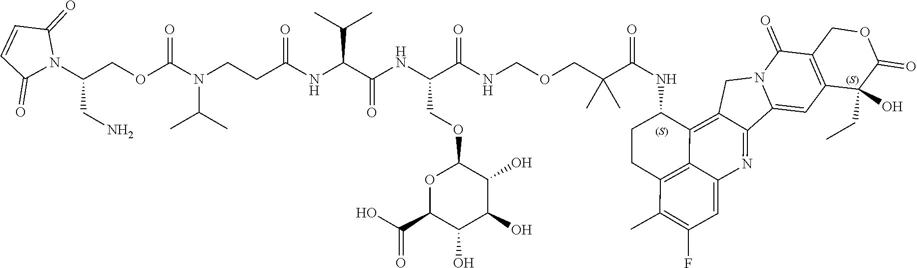 |
| 1-9 | 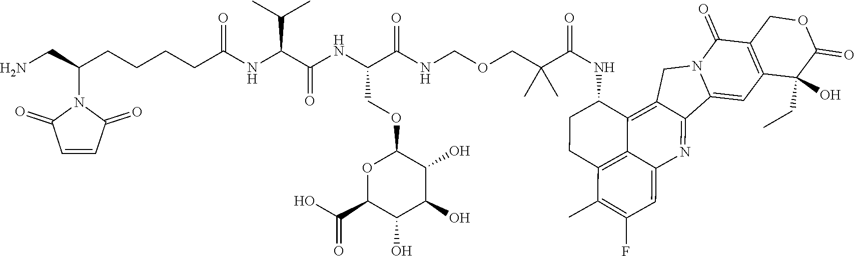 [SMILES: CC[C@@]1(O)C(=O)OCc2c1cc1n(c2=O)Cc2c-1nc1cc(F)c(C)c3c1c2[C@@H](NC(=O)C(C)(C)COCNC(=O)[C@H](CO[C@@H]1O[C@H](C(=O)O)[C@@H](O)[C@H](O)[C@H]1O)NC(=O)[C@@H](NC(=O)CCCC[C@H](CN)N1C(=O)C=CC1=O)C(C)C)CC3] |
| 1-10 | 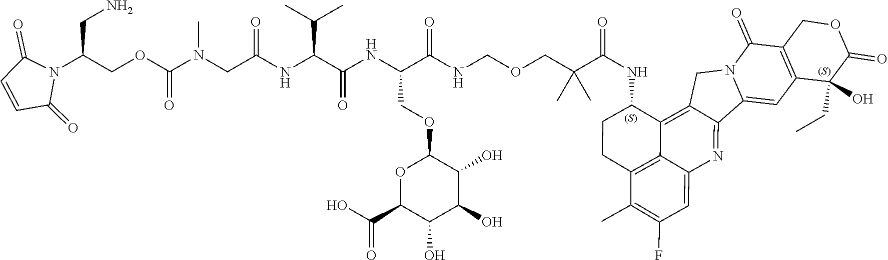 |
| 1-11 | 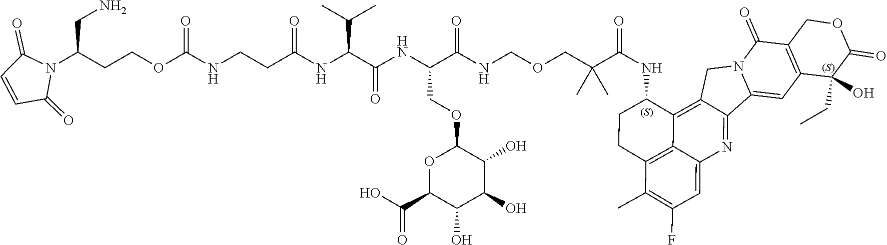 |
| 1-12 | 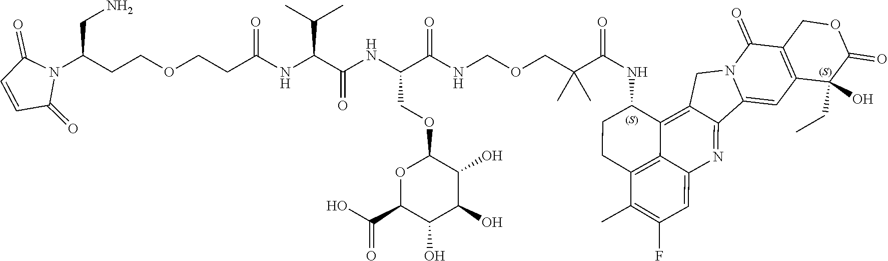 |
| 1-13 | 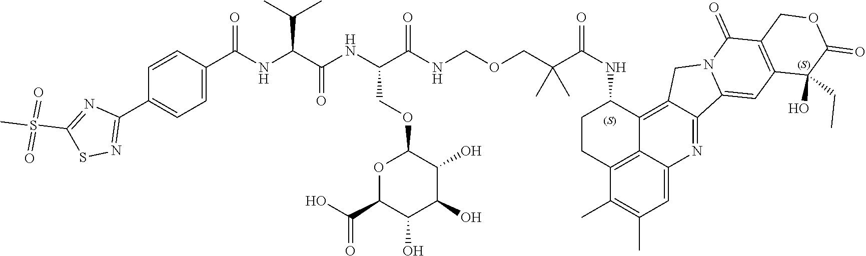 |
| 1-14 | 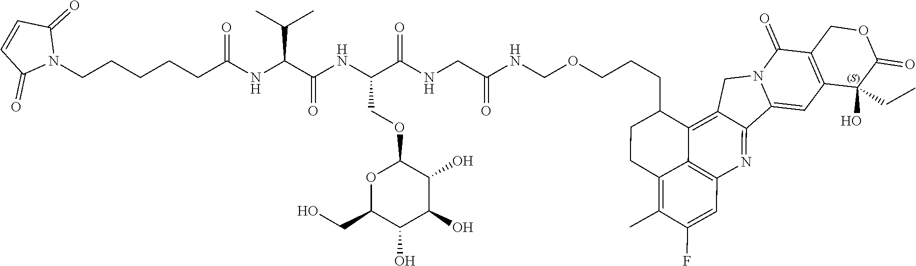 |
| 1-15 | 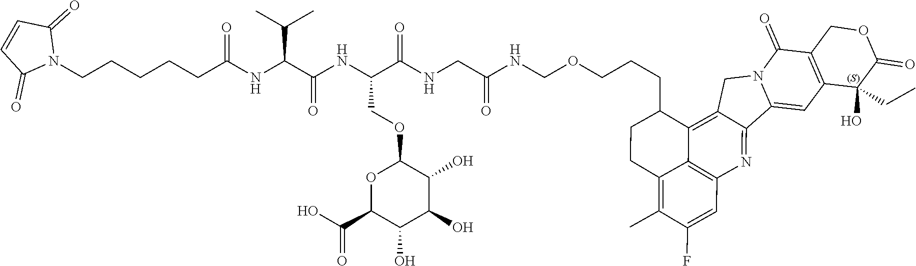 |
| 1-16 | 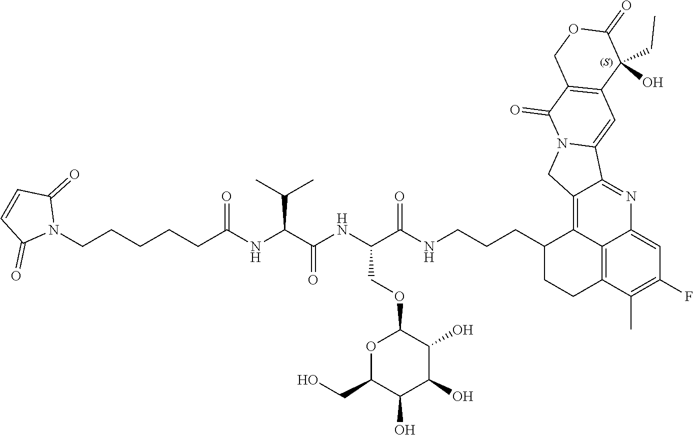 |
| 1-17 | 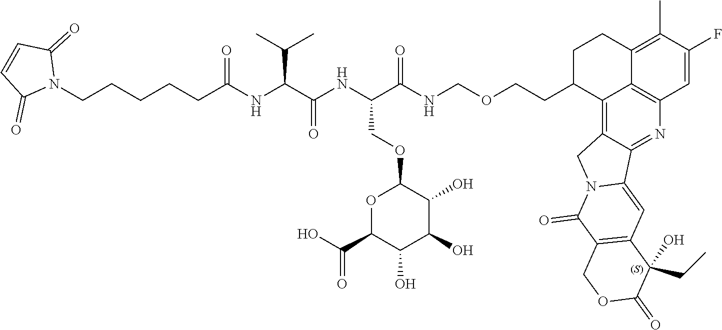 |
| 1-18 | 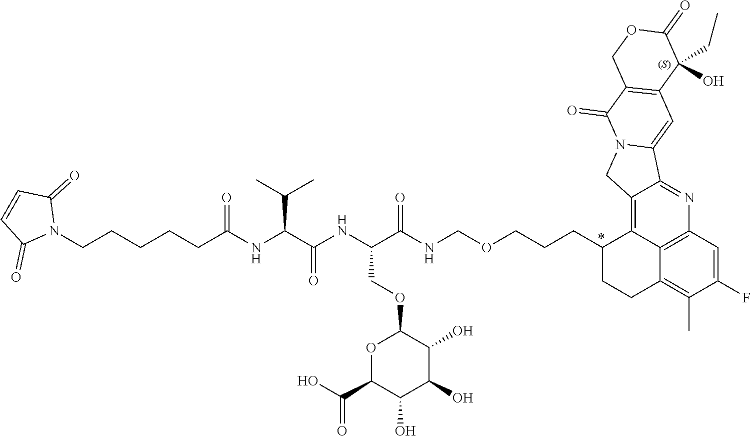 |
| 1-19 | 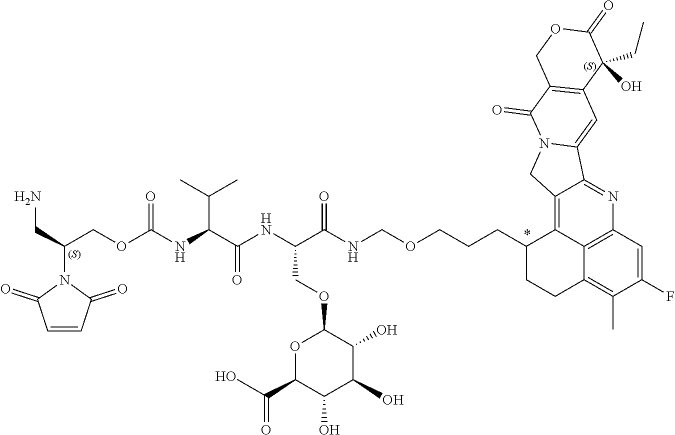 |
| 1-20 | 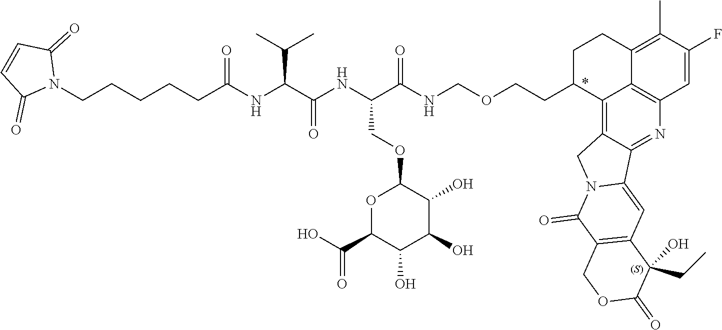 |
| 1-21 | 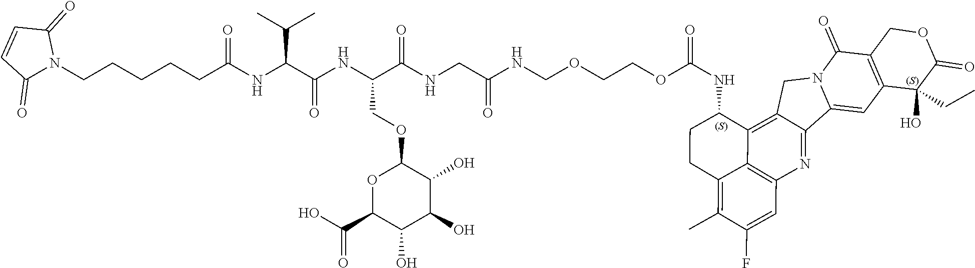 |
| 1-22 | 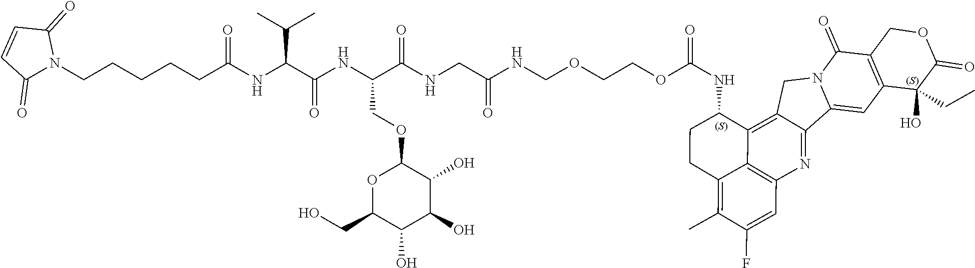 |
| 1-23 | 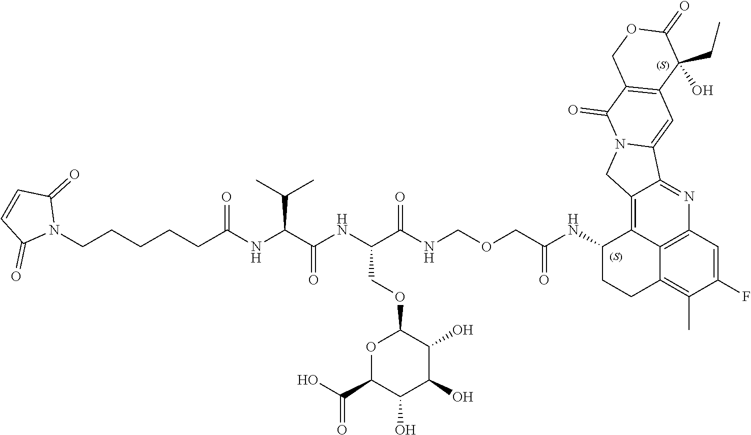 |
| 1-24 | 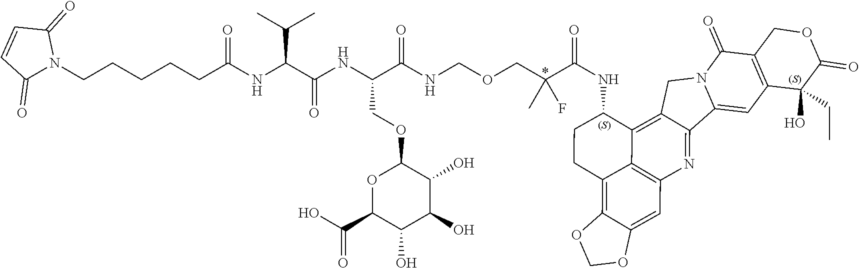 |
| 1-25 | 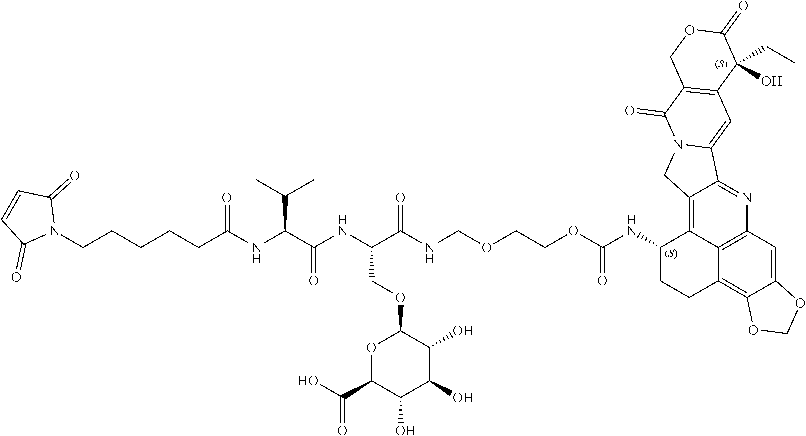 |
| 1-26 | 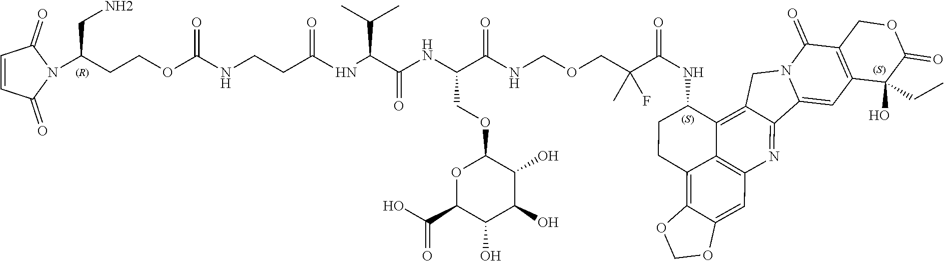 |
| 1-27 | 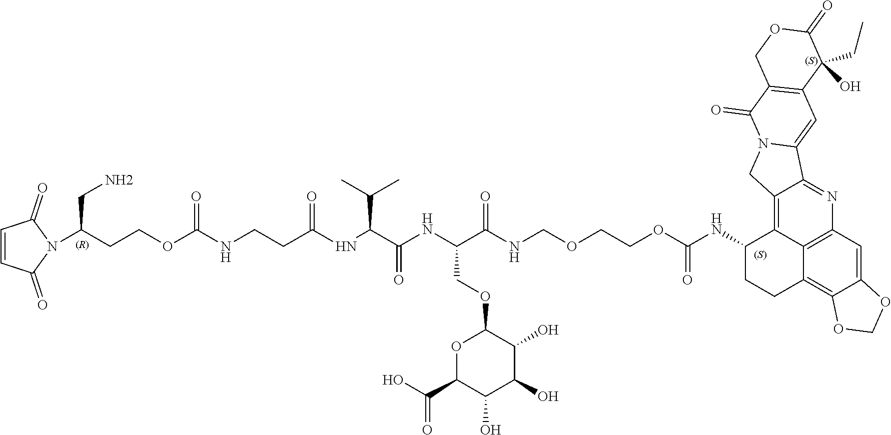 |
| 1-28 | 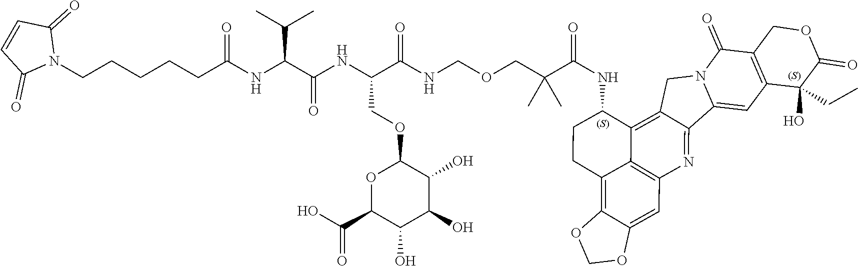 |
| 1-29 | 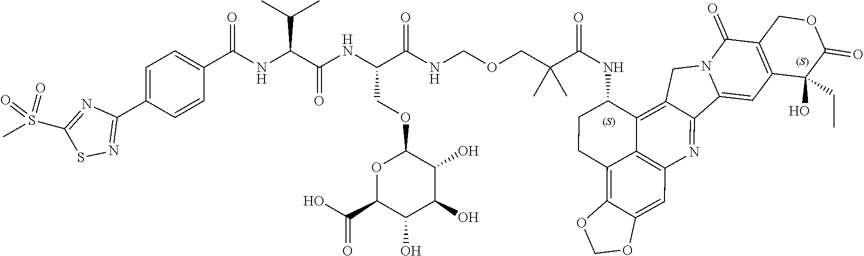 |
| 1-30 | 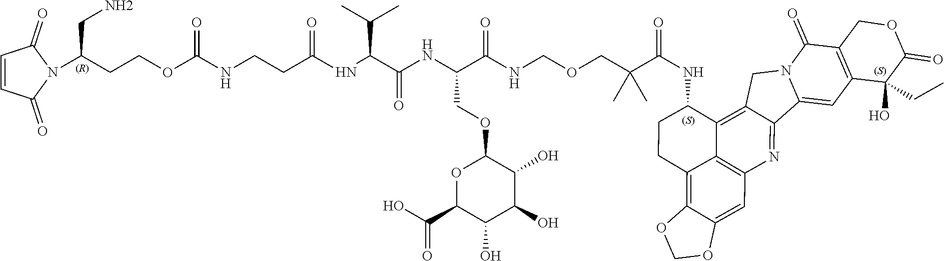 |
| 1-31 | 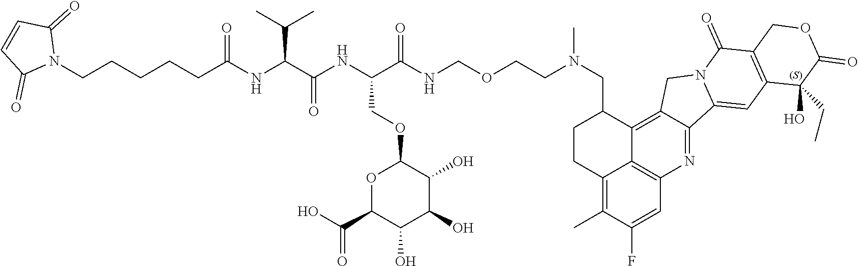 |
| 1-32 | 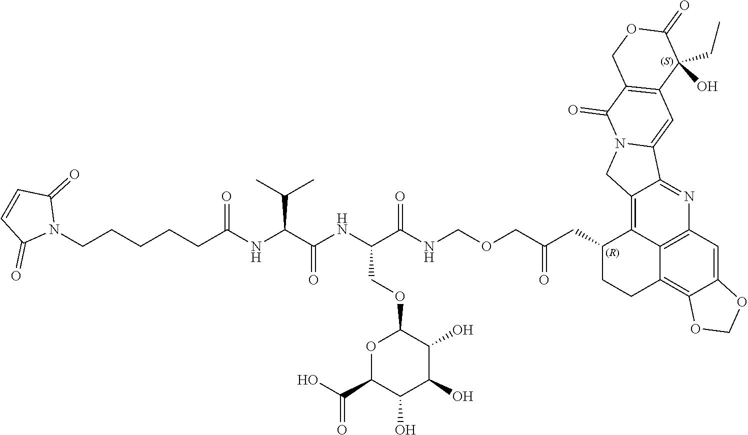 |

### ADC Preparation and Characterization

DARS antibody drug conjugate preparation. Antibody (e.g., ifinatimab) in conjugation buffer (with concentration 0.5-25 mg/mL, PBS buffer pH 6.0-8.5) was incubated under reduction temperature (0-40 °C) for 10 min. 8-15 eq. TECP solution (5 mM stock in PBS buffer) was added into the reaction mixture, and the reduction reaction was left for 1-8 hours at reduction temperature. Organic solvent (e.g., DMSO, DMF, DMA, PG, acetonitrile, 0-25% v/v) and linker-payload stock (10-25 eq, 10 mM stock in organic solvent) were added stepwise after the reduction mixture was cooled to 0-25 °C. Conjugation solution was left for 1-3 h at 0-25 °C, and the reaction was quenched with N-acetyl cysteine (1 mM stock). The solution was submitted to buffer exchange (spin desalting column, ultrafiltration, and dialysis) into storage buffer (for example, pH 5.5-6.5 histidine acetate buffer, with optional additive such as sucrose, trehalose, tween 20, 60, 80).

ADC characterization. ADCs were characterized using the following analytical methods. Drug to antibody ratios (DAR) of the ADCs were determined by LCMS method or HIC method. SEC purity of ADCs were all >95% purity.

LCMS method: LC-MS analysis was carried out under the following measurement conditions:
LC-MS system: Vanquish Flex UHPLC and Orbitrap Exploris 240 Mass Spectrometer
Column: MAbPac™ RP, 2.1*50 mm, 4 μm, 1,500 Å, Thermo Scientific™
Column temperature: 80 °C
Mobile phase A: 0.1% formic acid (FA) aqueous solution
Mobile phase B: Acetonitrile solution containing 0.1% formic acid (FA)
Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)
Injected sample amount: 1 μg
MS parameters: Intact and denaturing MS data were acquired in HMR mode at setting of
R=15k and deconvolved using the ReSpect™ algorithm and Sliding Window integration in Thermo Scientific™ BioPharma Finder™ 4.0 software.

HIC method: HPLC analysis was carried out under the following measurement conditions:

### Method I

HPLC system: Waters ACQUITY ARC HPLC System
Detector: measurement wavelength: 280 nm
Column: Tosoh Bioscience 4.6 μm ID×3.5 cm, 2.5 μm butyl-nonporous resin column
Column temperature: 25 °C
Mobile phase A: 1.5 M ammonium sulfate, 50 mM Phosphate buffer, pH 7.0
Mobile phase B: 50 mM Phosphate buffer, 25% (V/V) Isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20g

### Method 2

HPLC system: Waters ACQUITY ARC HPLC System
Detector: measurement wavelength: 280 nm
Column: MABPac HIC-10, 5 μm, 4.6×10 mm (Thermo)
Column temperature: 25 °C
Mobile phase A: 1.5 M ammonium sulfate, 50 mM sodium phosphate, pH 7.0
Mobile phase B: 50 mM sodium phosphate, pH 7.0
Gradient program: 20% B-20% B (0 min-1 min), 0% B-0% B (1 min-35 min), 20% B-20% B (35 min-40 min)
Flow rate: 0.5 mL/min
Sample preparation: The sample was diluted with initial mobile phase to 0.5 mg/mL.

SEC method: HPLC analysis was carried out under the following measurement conditions:
HPLC system: Waters H4-Class UPLC System
Detector: measurement wavelength: 280 nm
Column: ACQUITY UPLC BEH200 SEC 1.7 μm 4.6×150 mm, Waters
Column temperature: room temperature
Mobile phase A: 200 mM Phosphate buffer, 250 mM potassium chloride, 15% isopropyl alcohol, pH 7.0
Gradient program: under 10 min isocratic elutions with the flow rate of 0.3 mL/min
Injected sample amount: 20 μg

ADC hydrophobicity evaluation. An ADC with higher hydrophobic property would appear with later retention time from HIC (hydrophobicity interaction column) chromatography.

TABLE 2

| | | | DAR8 |
|---|---|---|---|
| | | | peak HIC |
| | | | retention |
| Example | Antibody | Structure | time (min) |
| Isotype ADC BGB-C-268 | Isotype IgG1 | 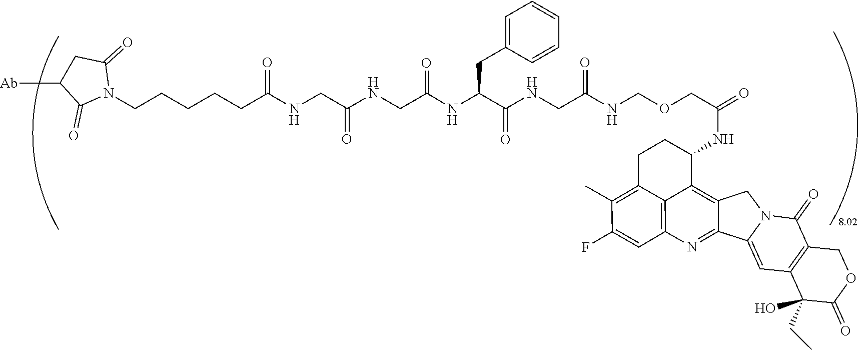 | |
| ADC1-A BGB-C-221 | Ifinatamab | 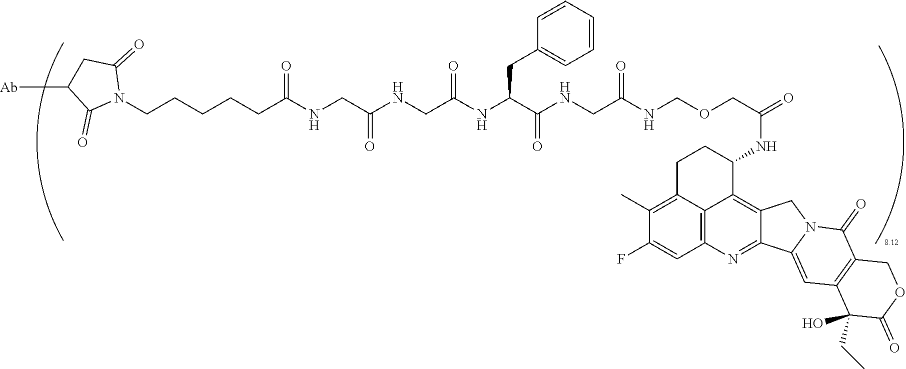 | Method 1 10.86 |
| ADC1-1 BGB-C-344 | Ifinatamab | 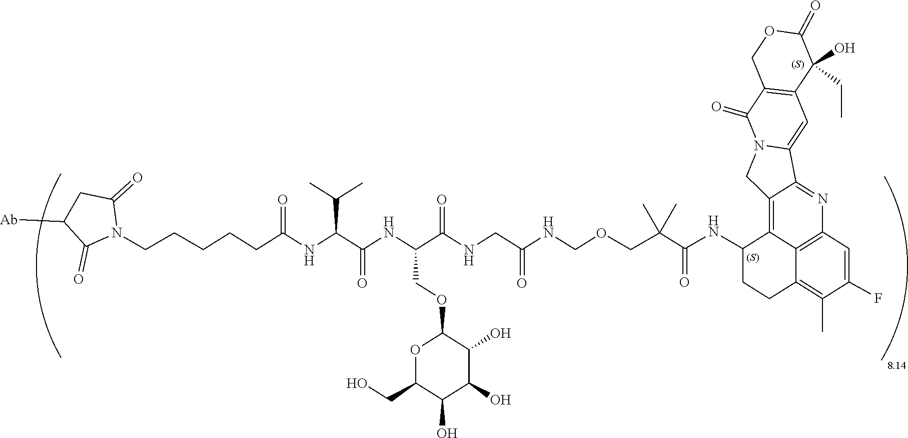 | Method 1 9.934 |
| ADC1-2 BGB-C-447 | Ifinatamab | 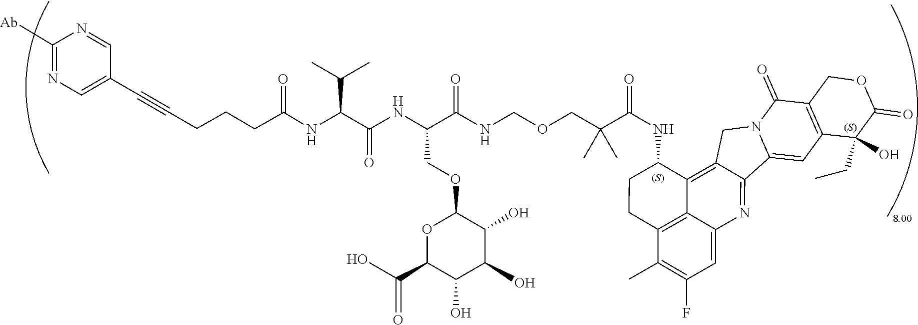 | Method 1 9.765 |
| ADC1-3 BGB-C-347 | Ifinatamab | 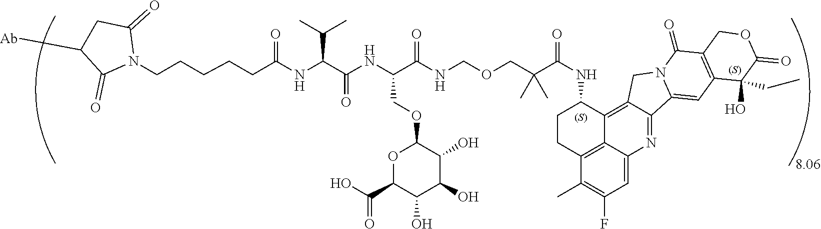 | Method 1 9.809 |
| ADC1-4 BGB-C-383 | Ifinatamab | 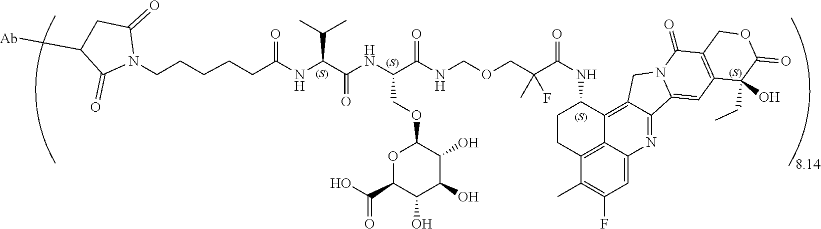 | Method 1 9.502 |
| ADC1-5 BGB-C-345 | Ifinatamab | 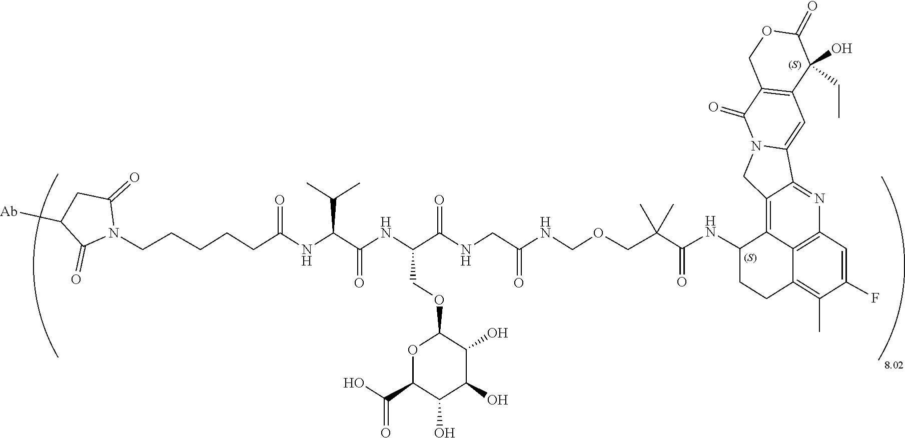 | Method 1 9.829 |
| ADC1-6 BGB-C-446 | Ifinatamab | 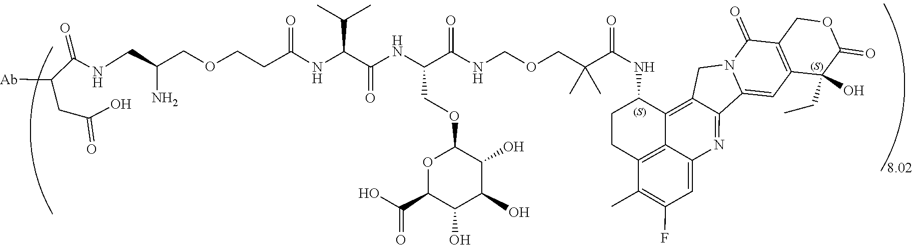 | Method 1 9.898 |
| ADC1-7 BGB-C-453 | Ifinatamab | 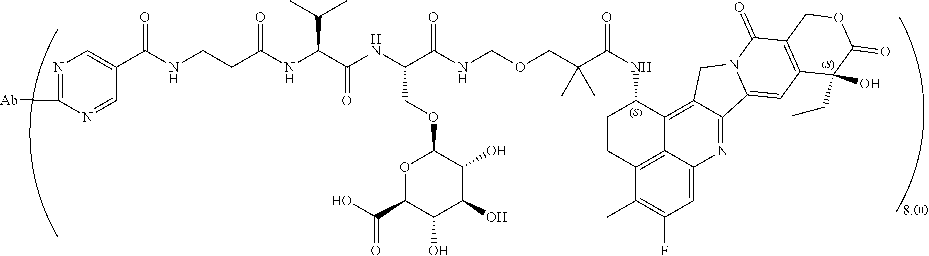 | Method 1 9.704 |
| ADC1-8 BGB-C-479 | Ifinatamab | 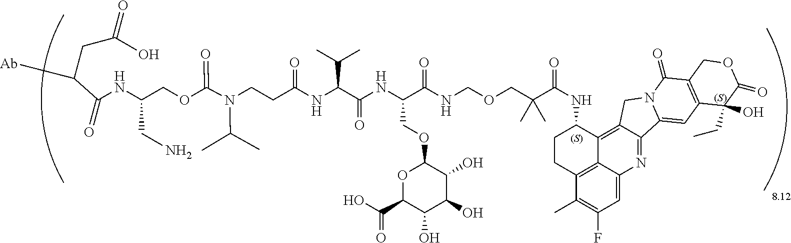 | Method 1 9.948 |
| ADC1-9 BGB-C-480 | Ifinatamab | 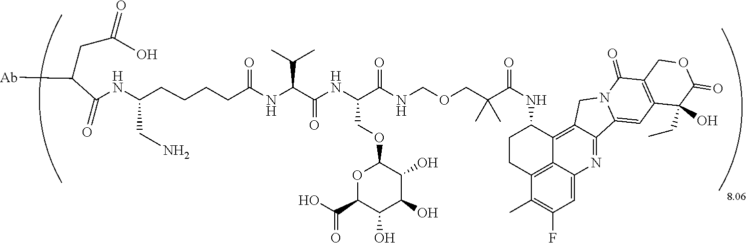 | Method 1 9.801 |
| ADC1-10 BGB-C-484 | Ifinatamab | 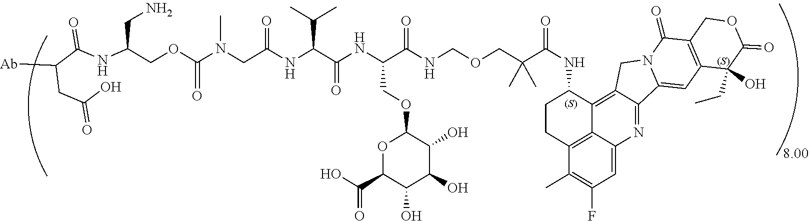 | Method 1 9.877 |
| ADC1-11 BGB-C-491 | Ifinatamab | 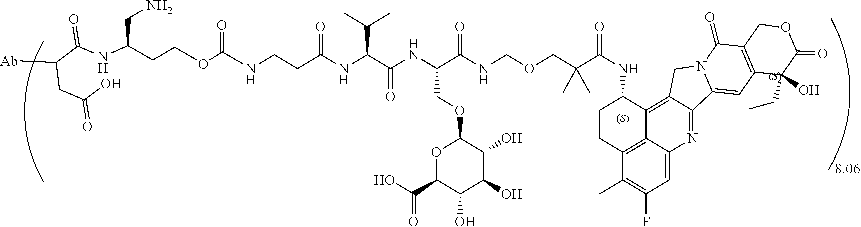 | Method 1 9.977 |
| ADC1-12 BGB-C-496 | Ifinatamab | 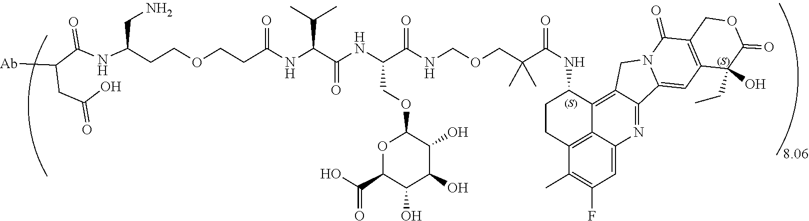 | Method 1 9.795 |
| ADC1-13 BGB-C-501 | Ifinatamab | 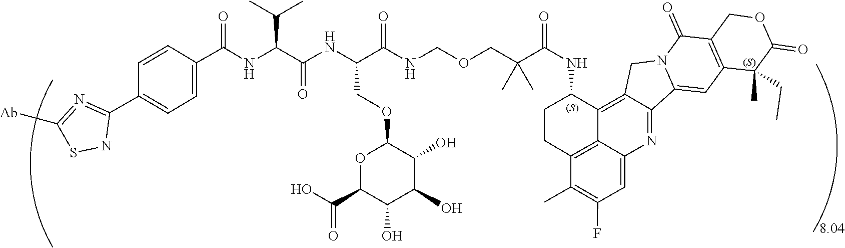 | Method 1 9.756 |
| ADC1-14 BGB-C-364 | Ifinatamab | 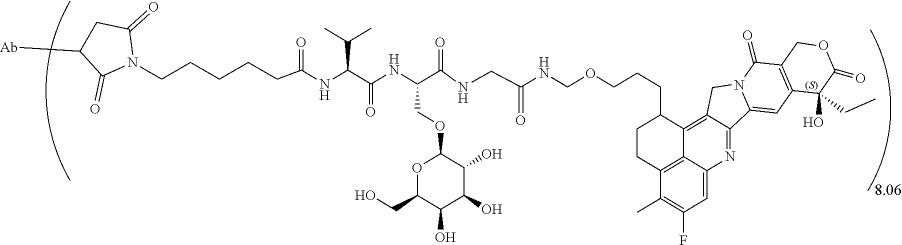 | Method 1 10.109 |
| ADC1-15 BGB-C-366 | Ifinatamab | 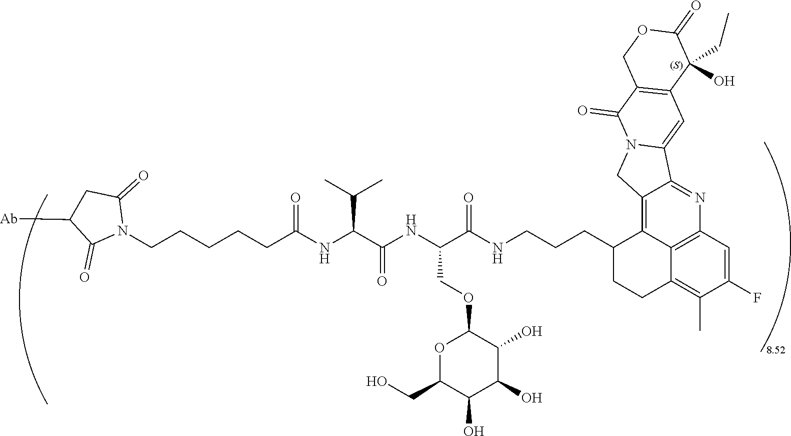 | Method 1 9.822 |
| ADC1-16 BGB-C-380 | Ifinatamab | 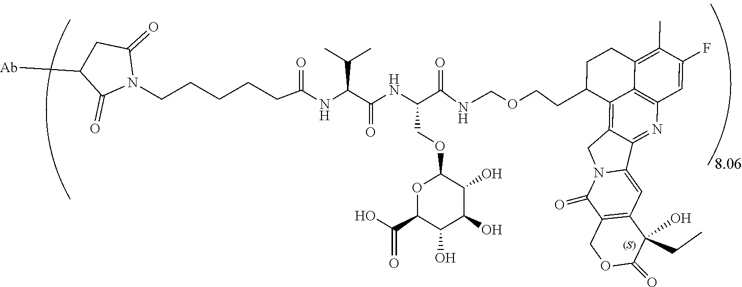 | Method 1 9.616 |
| ADC1-17 BGB-C-381 | Ifinatamab | 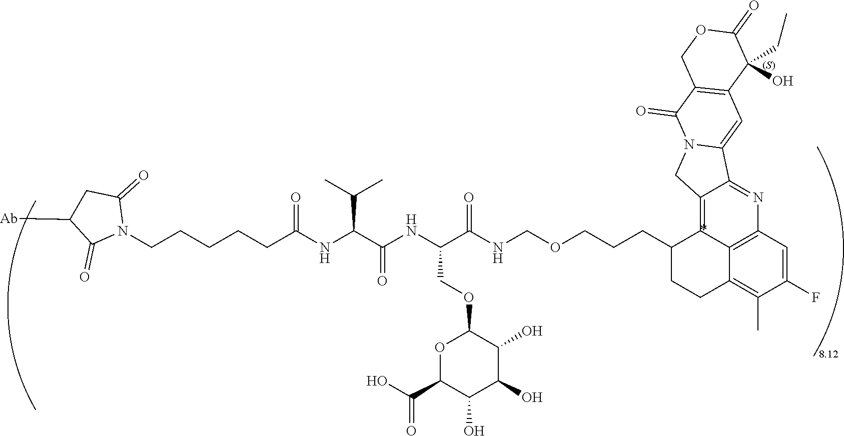 | Method 1 9.613 |
| ADC1-18 BGB-C-400 | Ifinatamab | 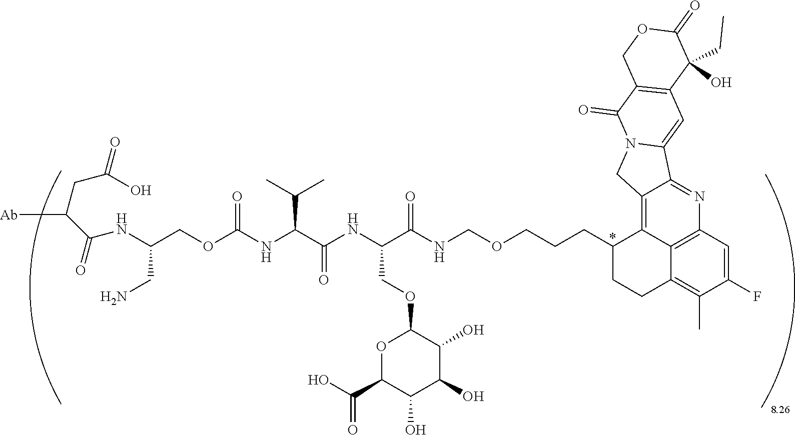 | Method 1 9.637 |
| ADC1-19 BGB-C-439 | Ifinatamab | 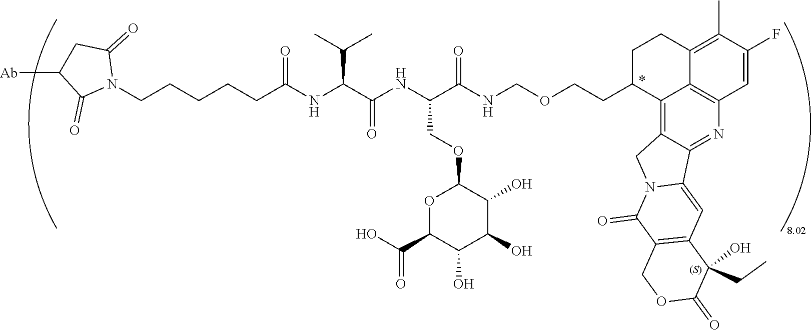 | Method 1 9.785 |
| ADC1-20 BGB-C-367 | Ifinatamab | 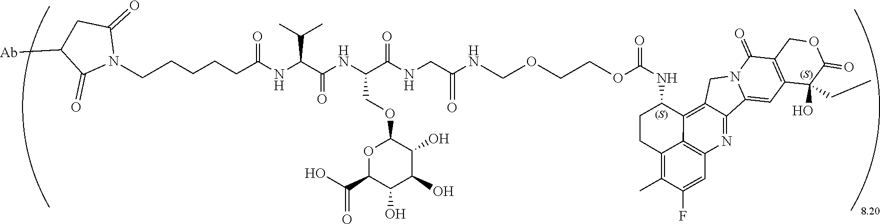 | Method 1 9.571 |
| ADC1-21 BGB-C-384 | Ifinatamab | 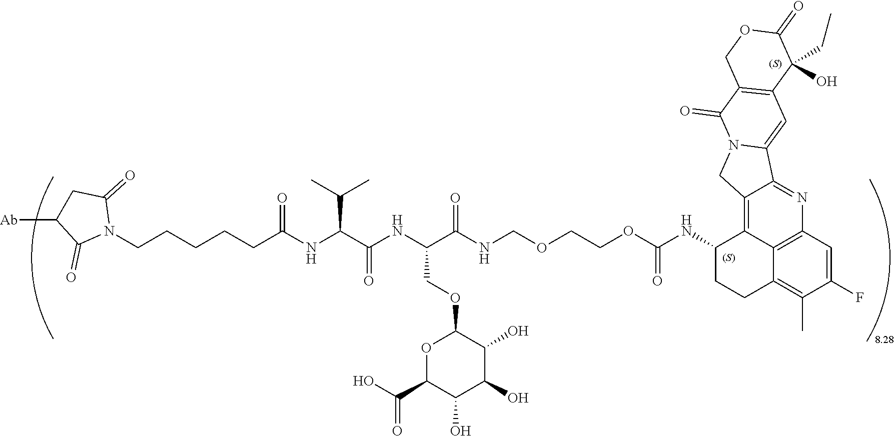 | Method 1 9.436 |
| ADC1-22 BGB-C-523 | Ifinatamab | 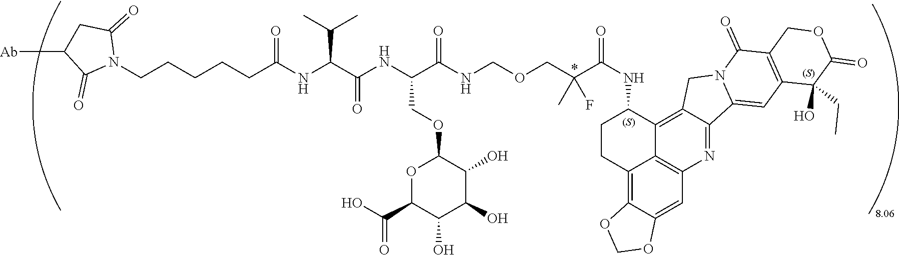 | Method 1 9.607 |
| ADC1-23 BGB-C-524 | Ifinatamab | 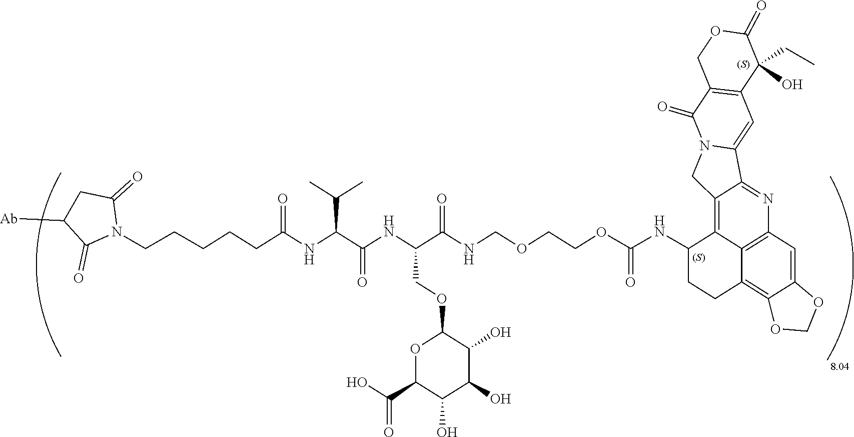 | Method 1 9.610 |
| ADC1-24 BGB-C-521 | Ifinatamab | 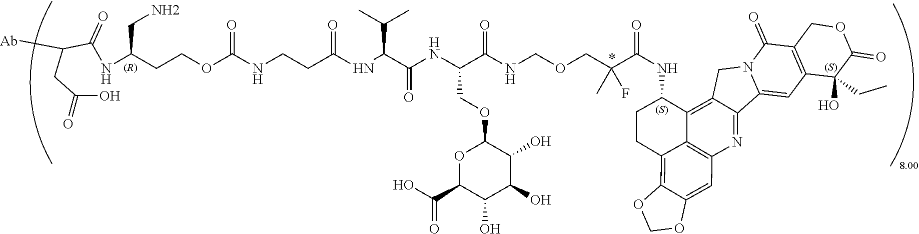 | Method 1 9.540 |
| ADC1-25 BGB-C-522 | Ifinatamab | 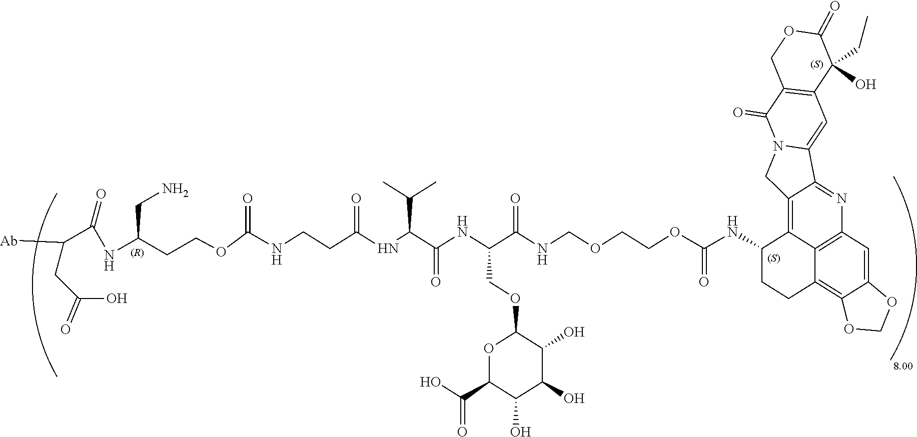 | Method 1 9.598 |
| ADC1-26 BGB-C-394 | Ifinatamab | 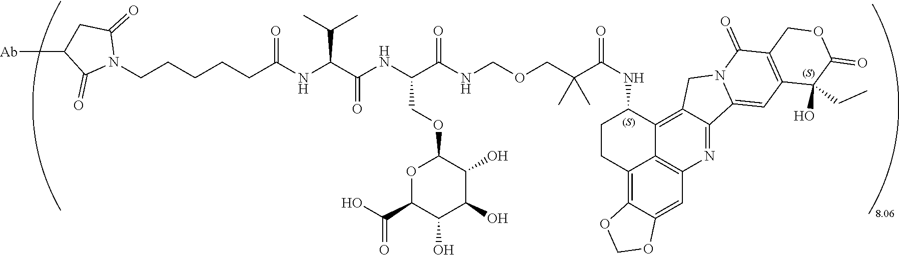 | Method 1 9.634 |
| ADC1-27 BGB-C-536 | Ifinatamab | 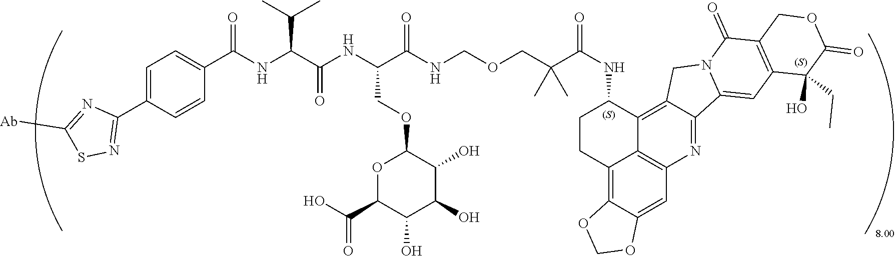 | Method 1 9.487 |
| ADC1-28 BGB-C-535 | Ifinatamab | 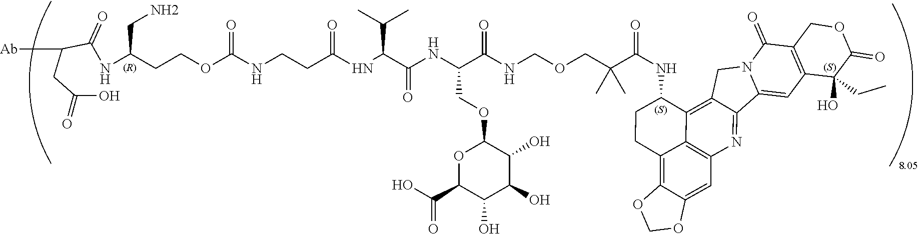 | Method 1 9.594 |
| ADC1-29 BGB-C-438 | Ifinatamab | 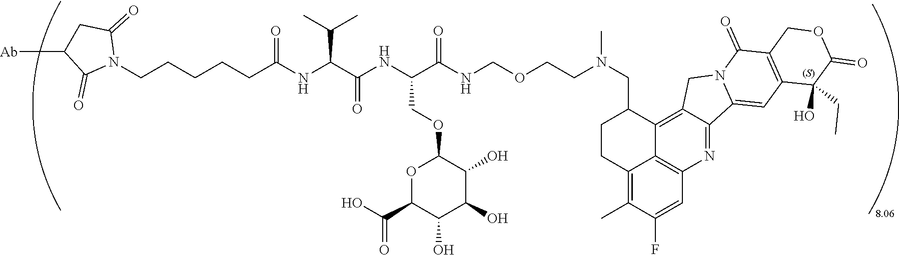 | Method 1 9.729 |
| ADC1-30 BGB-C-437 | Ifinatamab | 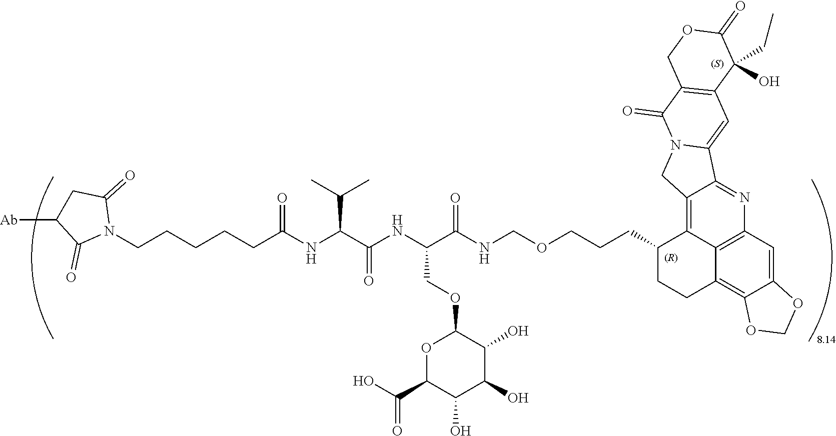 | Method 1 9.808 |

### Antibody Information

### Ifinatamab (Anti-B7H3 Antibody)

(SEQ ID NO:1)
Light Chain sequence
EIVLTQSPATLSLSPGERATLSCRASSRLIYMHWYQQKPGQAPRPLIYATSNLASGIPARFSGSGSGTDFTLTISSLEPE
DFAVYYCQQWNSNPPTFGQGTKVEIKRTVAAPSVFIFPPS
DEQLKSGTASVVCLLNNFYPREAKVQWKVDNALQSGNSQE
SVTEQDSKDSTYSLSSTLTLSKADYEKHKVYACEVTHQGL
SSPVTKSFNRGEC
(SEQ ID NO:2)
Heavy Chain sequence
QVQLVQSGAEVKKPGSSVKVSCKASGYTFTNYVMHWVRQA
PGQGLEWMGYINPYNDDVKYNEKFKGRVTITADESTSTAY
MELSSLRSEDTAVYYCARWGYYGSPLYYFDYWGQGTLVTV
SSASTKGPSVFPLAPSSKSTSGGTAALGCLVKDYFPEPVT
VSWNSGALTSGVHTFPAVLQSSGLYSLSSVVTVPSSSLGT
QTYICNVNHKPSNTKVDKRVEPKSCDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLYSKLTVDKSRWQQGNVFSCSVMHEALHNH
YTQKSLSLSPGK

### Cell Lines

A375 (ATCC, CRL-1619). A-375 is a cell line exhibiting epithelial morphology that was isolated from the skin of a 54-year-old female patient with malignant melanoma, and A375 was purchased from ATCC. The base medium for A375 is DMEM, high glucose, GlutaMAX™ Supplement (Gibco, 10566024). To make the complete growth medium, fetal bovine serum to a final concentration of 10% (Gibco, 10099-141C) was added to the base medium. The cell line was grown in a humidified 5% CO₂ atmosphere at 37 °C, and was regularly tested for the presence of mycoplasma with MycoAlert™ PLUS Mycoplasma Detection Kit (Lonza, LT07-710).

Calu-6 (ATCC, HTB-56). Calu-6 is a cell line exhibiting epithelial morphology that was isolated from anaplastic carcinoma, and Calu-6 was purchased from ATCC. The base medium for Calu-6 is Eagle's Minimum Essential Medium (ATCC, 30-2003). To make the complete growth medium, fetal bovine serum to a final concentration of 10% (Gibco, 10099-141C) was added to the base medium. The cell line was grown in a humidified 5% CO₂ atmosphere at 37 °C, and was regularly tested for the presence of mycoplasma with MycoAlert™ PLUS Mycoplasma Detection Kit (Lonza, LT07-710).

NCI-H358 (ATCC, CRL-5807). NCT-H358 is an epithelial-like cell that was isolated from the bronchiole of a male patient with bronchioalveolar carcinoma, and NCI-H358 was purchased from ATCC. The base medium for NCI-H358 is ATCC-formulated RPMI-1640 Medium, ATCC 30-2001. To make the complete growth medium, fetal bovine serum to a final concentration of 10% (Gibco, 10099-141C) was added to the base medium. The cell line was grown in a humidified 5% CO₂ atmosphere at 37 °C, and was regularly tested for the presence of mycoplasma with MycoAlert™ PLUS Mycoplasma Detection Kit (Lonza, LT07-710).

NCI-H1650 (ATCC, CRL-5883). NCI-H1650 is a cell line exhibiting epithelial morphology that was isolated in 1987 from the lung tissue of a 27-year-old male smoker with stage 3, bronchoalveolar carcinoma, and NCI-H1650 was purchased from ATCC. The base medium for NCT-H1650 is ATCC-formulated RPMI-1640 Medium, ATCC 30-2001. To make the complete growth medium, fetal bovine serum to a final concentration of 10% (Gibco, 10099-141C) was added to the base medium. The cell line was grown in a humidified 5% CO₂ atmosphere at 37 °C, and was regularly tested for the presence of mycoplasma with MycoAlert™ PLUS Mycoplasma Detection Kit (Lonza, LT07-710).

NCI-H1048 (ATCC, CRL-5853). NCI-H1048 is a cell line exhibiting epithelial morphology, and NCI-H1048 was purchased from ATCC. The base medium for NCI-H1048 is ATCC-formulated DMEM:F12 Medium Catalog No. 30-2006. To make the complete growth medium, fetal bovine serum to a final concentration of 10% (Gibco, 10099-141C) was added to the base medium. The cell line was grown in a humidified 5% CO₂ atmosphere at 37 °C, and was regularly tested for the presence of mycoplasma with MycoAlert™ PLUS Mycoplasma Detection Kit (Lonza, LT07-710).

Capan-1 (ATCC, HTB-79). Capan-1 is a cell line with epithelial morphology that was isolated from the pancreas of a 40-year-old white male with pancreatic adenocarcinoma, and Capan-1 was purchased from ATCC. The base medium for Capan-1 is ATCC-formulated Iscoves Modified Dulbecco's Medium, Catalog No. 30-2005. To make the complete growth medium, fetal bovine serum to a final concentration of 20% (Gibco, 10099-141C) was added to the base medium. The cell line was grown in a humidified 5% CO₂ atmosphere at 37 °C, and was regularly tested for the presence of mycoplasma with MycoAlert™ PLUS Mycoplasma Detection Kit (Lonza, LT07-710).

MDA-MB-453 (SIBS). MDA-MB-453 was derived from an effusion of a 48-year-old female patient with metastatic carcinoma of the breast, involving the nodes, brain, and both pleural and pericardial cavities, and MDA-MB-453 was purchased from SIBS. The base medium for MDA-MB-453 is RPMI 1640 Medium, HEPES (Gibco, 22400105). To make the complete growth medium, fetal bovine serum to a final concentration of 10% (Gibco, 10099-141C) was added to the base medium. The cell line was grown in a humidified 5% CO₂ atmosphere at 37 °C, and was regularly tested for the presence of mycoplasma with MycoAlert™ PLUS Mycoplasma Detection Kit (Lonza, LT07-710).

Antibody binding capacity for various cell lines was determined using a Quantum™ Simply Cellular® (“QSC”) microsphere kit according to the manufacturer's protocol. (Bangs Laboratories, Inc.). Data are reported in Table 3.

TABLE 3
B7H3 expression level

| Cell line | Molecular No. (QSC kit) |
|---|---|
| NCI-H358 | 603012 |
| NCI-H1650 | 505998 |
| NCI-H1048 | 100687 |
| CAPAN-1 | 63062 |
| MDA-MB-453 | 0 |

### Payload Cellular Killing in A375 and Calu-6 Cancer Lines

Payload cellular killing was assessed in A375 and Calu-6 cancer lines. Cells were seeded (A375 (1E3/well) or Calu-6 (2E3/well)) into a 96-well plate (Greiner: 655090), at 100 μl/well and incubated at 37 °C, 5% CO₂, overnight. Fresh growth medium was added containing varying concentrations of compounds, 50 μl/well, and incubated at 37 °C, 5% CO₂, for 6 days. The cell viability was detected by Cell Titer-Glo (Promega, G7573). 70 μL of CellTiter-Glo® Reagent was added to each well. The plates were allowed to incubate at room temperature for 10 minutes to stabilize the luminescent signal. The plates were analyzed with a microplate reader.

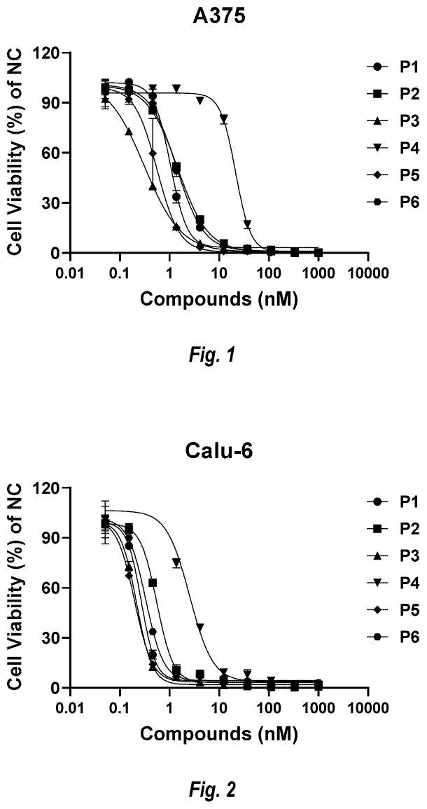

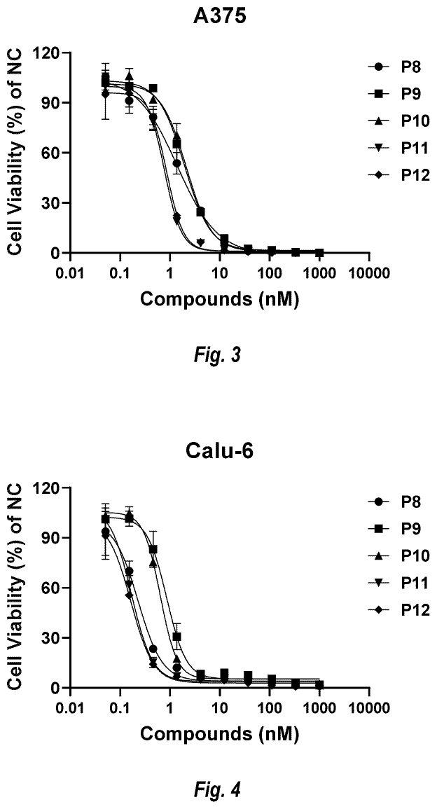

Data are summarized in Tables 4A and B, and FIGS. 1-4.

TABLE 4A

| | A375 | | Calu-6 | |
|---|---|---|---|---|
| Compounds | Emax (%) | EC50 (nM) | Emax (%) | EC50 (nM) |
| P1 | 99.9 | 1.1 | 97.3 | 0.3 |
| P2 | 99.9 | 1.5 | 98.2 | 0.6 |
| P3 | 100.0 | 0.1 | 99.2 | 0.2 |
| P4 | 99.6 | 20.7 | 99.2 | 2.8 |
| P5 | 100.0 | 0.7 | 98.1 | 0.2 |
| P6 | 99.9 | 1.4 | 96.9 | 0.3 |

TABLE 4B

| | A375 | | Calu-6 | |
|---|---|---|---|---|
| Compounds | Emax (%) | EC50 (nM) | Emax (%) | EC50 (nM) |
| P8 | 100.0 | 1.6 | 98.6 | 0.2 |
| P9 | 100.0 | 2.1 | 98.3 | 0.9 |
| P10 | 100.0 | 2.2 | 97.4 | 0.7 |
| P11 | 100.0 | 0.8 | 98.0 | 0.2 |
| P12 | 100.0 | 0.8 | 98.4 | 0.2 |

### ADC Direct Killing in NCI-H358, Capan-1, and MDA-MB-453 Cancer Cell Lines

ADCs direct killing was assessed in NCI-H358, Capan-1, and MDA-MB-453 cancer lines. Cells were seeded (NCI-H358 or MDA-MB-453 (2E3/well) or Capan-1 (4E3/well)) into 3D 96-well plates (Corning: 4520), 80 μl/well, and incubated at 37 °C, 5% CO₂, overnight. Fresh growth medium was added containing varying concentrations of ADCs, 40 μl/well, and incubated at 37 °C, 5% CO₂, for 6 days. The cell viability was detected by 3D reagent (Promega, 09683), 100 μl/well. The 3D plates were allowed to incubate at room temperature for 30 minutes to stabilize the luminescent signal. The plates were analyzed with a Microplate Reader.

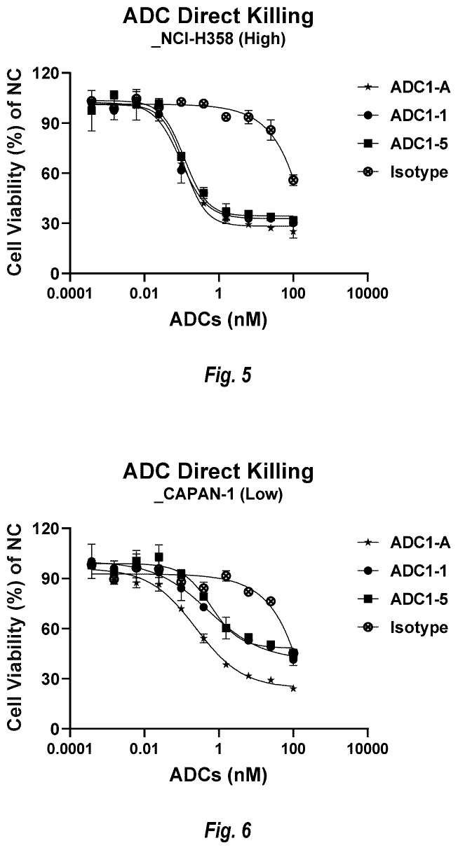

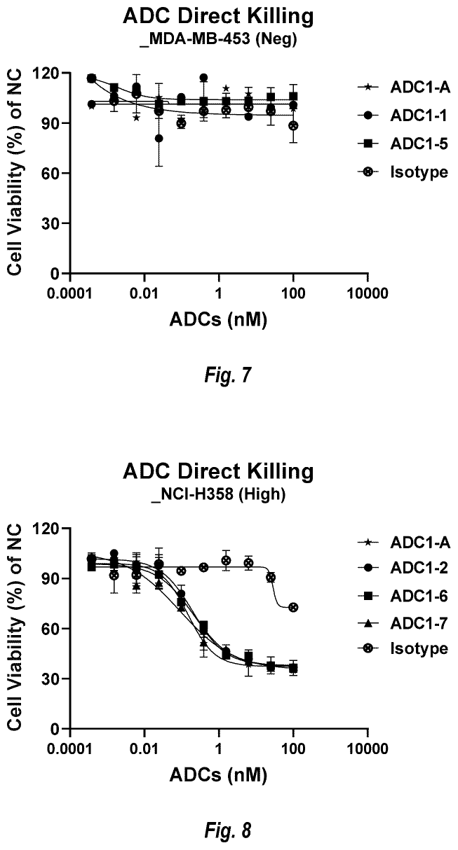

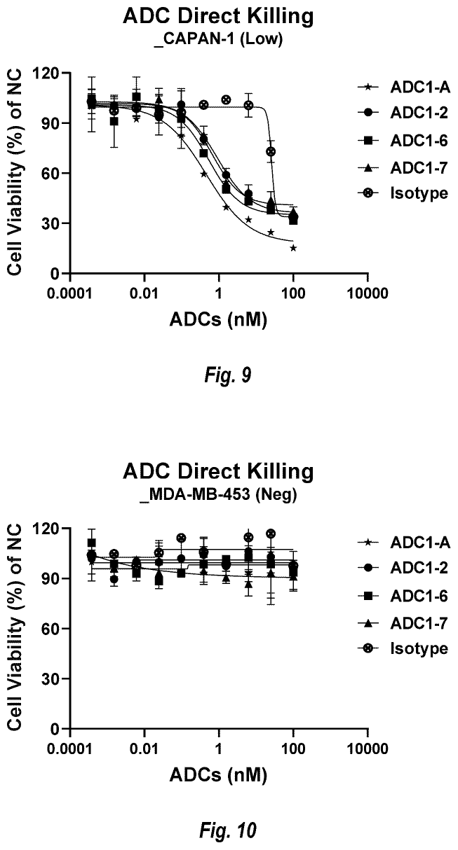

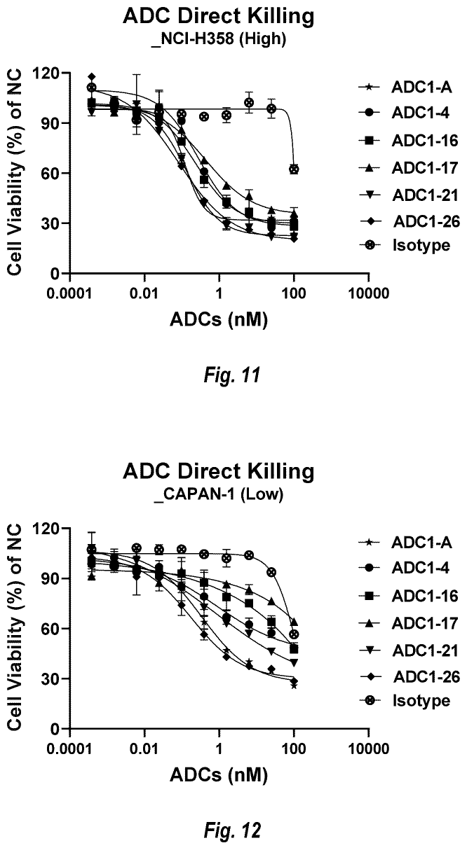

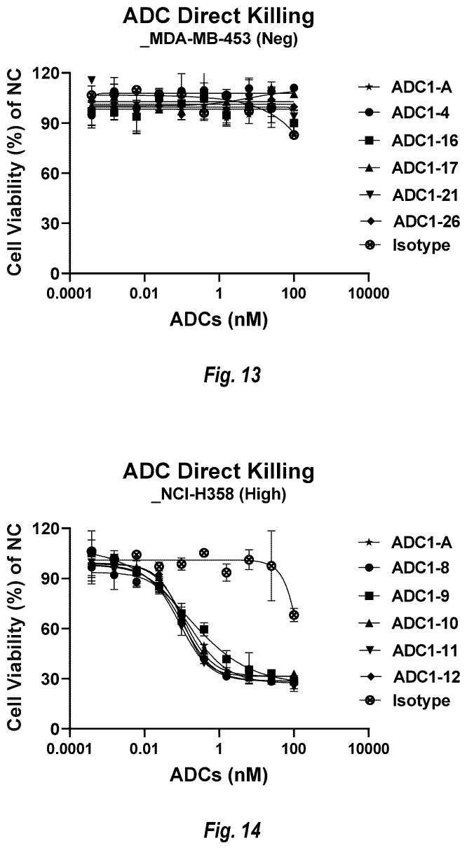

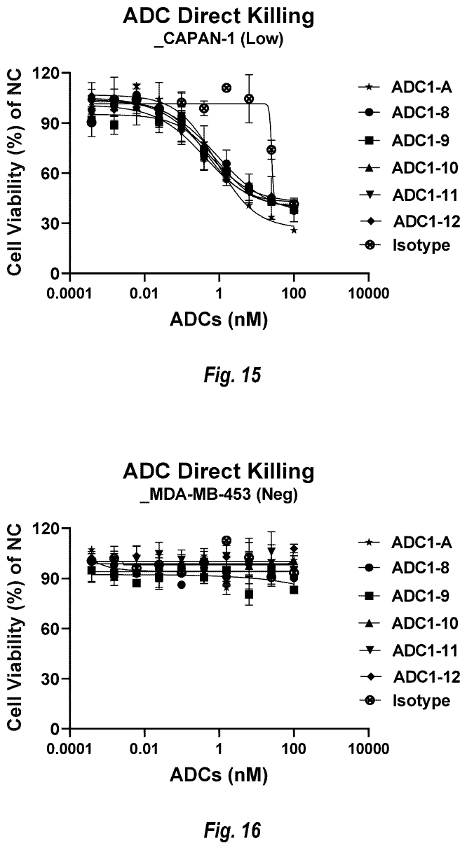

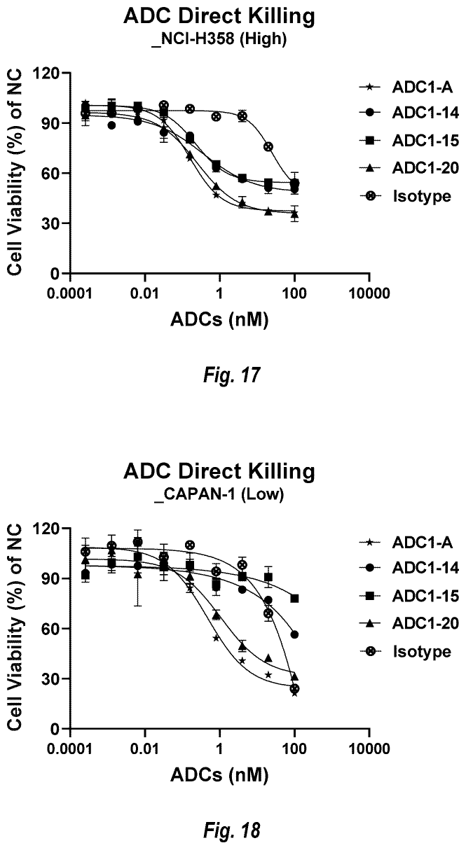

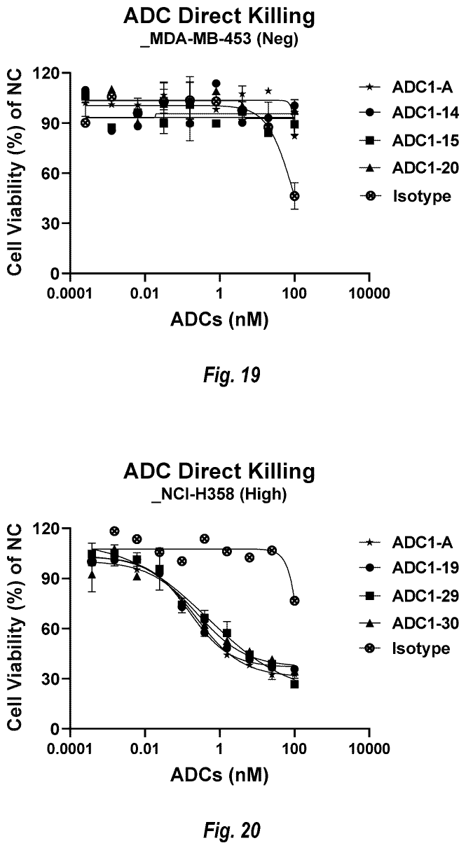

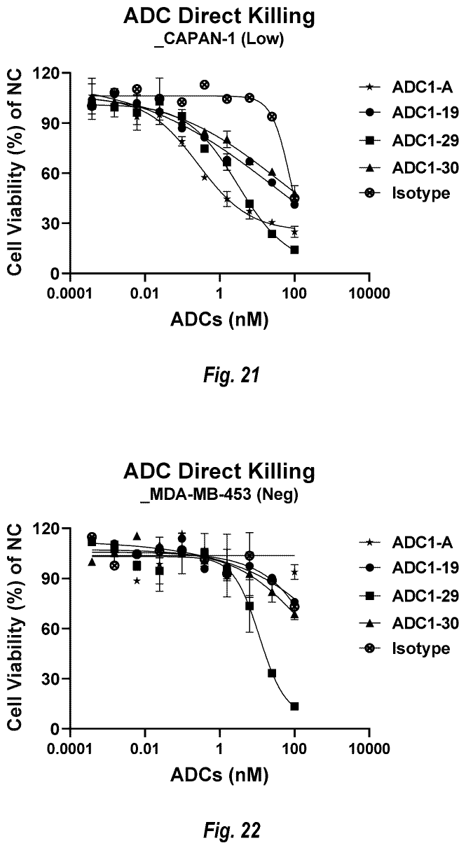

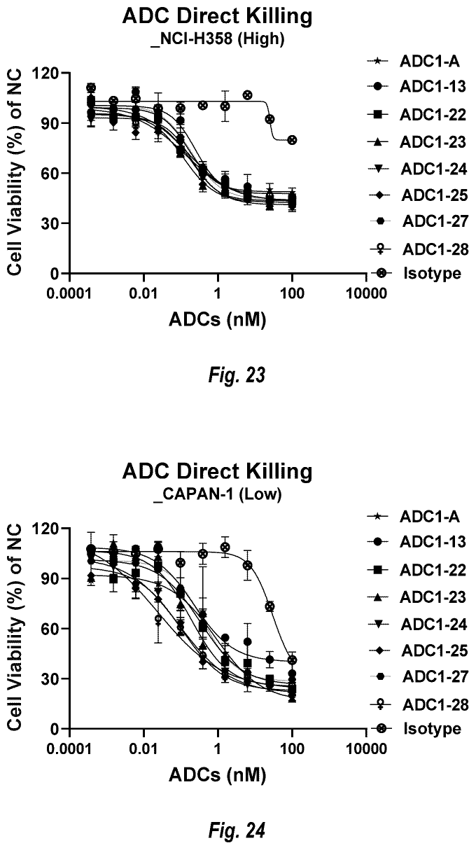

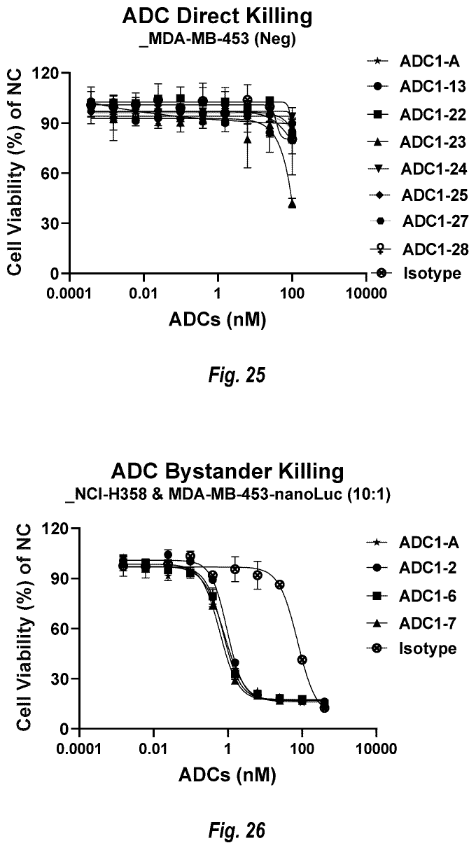

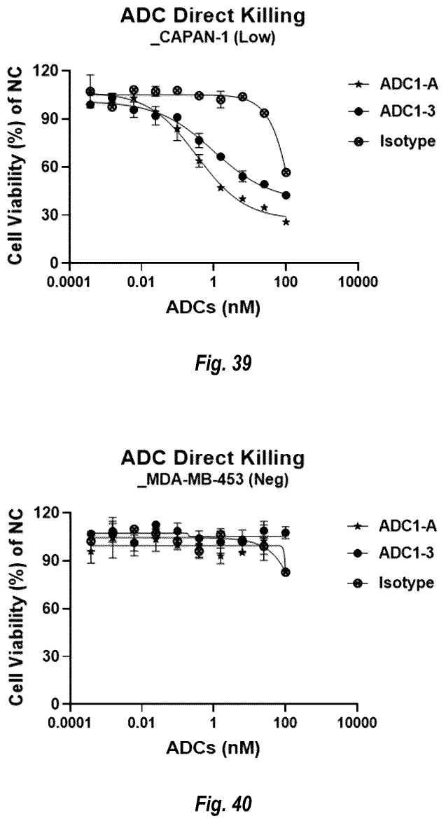

Data are summarized in Tables 5A-5H, and FIGS. 5-25 and 38-40. Isotype ADC is an antivirus Ab not recognizing B71H3, and was used as a negative control.

TABLE 5A

| | NCI-H358 (High), 3D | | CAPAN-1 (Low), 3D | |
|---|---|---|---|---|
| ADCs | Emax (%) | EC50 (nM) | Emax (%) | EC50 (nM) |
| ADC1-A | 75.0 | 0.1 | 75.9 | 0.2 |
| ADC1-1 | 69.9 | 0.1 | 58.6 | 0.5 |
| ADC1-5 | 68.1 | 0.1 | 55.0 | 0.7 |

TABLE 5B

| | NCI-H358 (High), 3D | | CAPAN-1 (Low), 3D | |
|---|---|---|---|---|
| ADCs | Emax (%) | EC50 (nM) | Emax (%) | EC50 (nM) |
| ADC1-A | 65.0 | 0.2 | 84.9 | 0.5 |
| ADC1-2 | 63.4 | 0.2 | 66.4 | 1.1 |
| ADC1-6 | 63.4 | 0.2 | 68.4 | 0.6 |
| ADC1-7 | 62.7 | 0.1 | 62.8 | 0.8 |

TABLE 5C

| | NCI-H358 (High), 3D | | CAPAN-1 (Low), 3D | |
|---|---|---|---|---|
| ADCs | Emax (%) | EC50 (nM) | Emax (%) | EC50 (nM) |
| ADC1-A | 76.4 | 0.1 | 74.3 | 0.5 |
| ADC1-4 | 68.7 | 0.3 | 51.6 | 0.7 |
| ADC1-16 | 71.5 | 0.3 | 52.1 | 6.0 |
| ADC1-17 | 64.1 | 0.4 | 35.9 | 6.5 |
| ADC1-21 | 78.6 | 0.1 | 60.6 | 0.8 |
| ADC1-26 | 79.2 | 0.1 | 71.5 | 0.2 |

TABLE 5D

| | NCI-H358 (High), 3D | | CAPAN-1 (Low), 3D | |
|---|---|---|---|---|
| ADCs | Emax (%) | EC50 (nM) | Emax (%) | EC50 (nM) |
| ADC1-A | 74.4 | 0.1 | 74.3 | 1.1 |
| ADC1-8 | 72.2 | 0.1 | 62.4 | 1.0 |
| ADC1-9 | 71.6 | 0.2 | 61.9 | 0.7 |
| ADC1-10 | 67.0 | 0.1 | 58.5 | 0.5 |
| ADC1-11 | 74.6 | 0.1 | 61.3 | 0.4 |
| ADC1-12 | 71.7 | 0.1 | 62.2 | 0.6 |

TABLE 5E

| | NCI-H358 (High), 3D | | CAPAN-1 (Low), 3D | |
|---|---|---|---|---|
| ADCs | Emax (%) | EC50 (nM) | Emax (%) | EC50 (nM) |
| ADC1-A | 63.8 | 0.2 | 78.8 | 0.5 |
| ADC1-14 | 49.6 | 0.4 | 43.5 | >100 |
| ADC1-15 | 45.9 | 0.2 | 22.0 | >100 |
| ADC1-20 | 64.2 | 0.3 | 68.5 | 1.1 |

TABLE 5F

| | NCI-H358 (High), 3D | | CAPAN-1 (Low), 3D | |
|---|---|---|---|---|
| ADCs | Emax (%) | EC50 (nM) | Emax (%) | EC50 (nM) |
| ADC1-A | 67.1 | 0.2 | 75.1 | 0.3 |
| ADC1-19 | 64.2 | 0.2 | 58.8 | 2.2 |
| ADC1-29 | 73.3 | 0.7 | 85.8 | 2.3 |
| ADC1-30 | 65.6 | 0.3 | 52.1 | 3.2 |

TABLE 5G

| | NCI-H358 (High), 3D | | CAPAN-1 (Low), 3D | |
|---|---|---|---|---|
| ADCs | Emax (%) | EC50 (nM) | Emax (%) | EC50 (nM) |
| ADC1-A | 51.6 | 0.2 | 81.7 | 0.4 |
| ADC1-13 | 54.0 | 0.3 | 66.9 | 0.4 |
| ADC1-22 | 56.7 | 0.2 | 76.3 | 0.3 |
| ADC1-23 | 58.0 | 0.1 | 81.7 | 0.3 |
| ADC1-24 | 59.5 | 0.2 | 79.1 | 0.1 |
| ADC1-25 | 60.0 | 0.2 | 79.5 | 0.1 |
| ADC1-27 | 55.1 | 0.2 | 70.4 | 0.4 |
| ADC1-28 | 58.3 | 0.1 | 75.3 | 0.1 |

TABLE 5H

| | NCI-H358 (High), 3D | | CAPAN-1 (Low), 3D | |
|---|---|---|---|---|
| ADCs | Emax (%) | EC50 (nM) | Emax (%) | EC50 (nM) |
| ADC1-A | 76.4 | 0.1 | 74.3 | 0.5 |
| ADC1-3 | 76.8 | 0.2 | 57.6 | 0.9 |

ADC Bystander Killing: NCI-H358 Co-Culture with MDA-MB-453-nanoLuc

MDA-MB-453-nanoLuc cell line construction. PT67-nanoLuc cells were cultured, then the cell-culture medium (containing the virus/nano-Luc gene) was collected and filtered. MDA-MB-453 cells were seeded in 6-well plates at 1E5 cells/well. The plates were incubated at 37 °C, 5% CO₂ overnight. The PT67-nanoLuc cell medium was added with 8 pig/ml polybrene. The infection was repeated once a day for three days. The MDA-MB-453-nanoLuc cells were cultured with the addition of 1 mg/nil Geneticin for 5 days. The MDA-MB-453-nanoLuc cells were collected, and Nano-Glo reagent (Promega: NI 120) was added to test the nano-Luc transfection efficiency.

ADC bystander killing. NCI-H358 and MDA-MB-453-nanoLuc (10:1). or MDA-MB-453-nanoLuc only, were co-cultured or cultured, respectively, in 3D 96-well plates (Corning: 4520) at 80 μl/well. Cells were incubated at 37 °C, 5% CO₂, overnight. Fresh growth medium was added containing varying concentrations of ADCs at 40 μl/well. Cells were incubated at 37 °C, 5% CO₂, for 6 days. The 3D plates were centrifuged at 1500 rpm, 25 °C, for 5 min, then the supernatant was discarded. The Calu-6-nanoLuc cell viability was detected by Nano-Glo reagent (Promega: N1120), 150 μl/well. The 3D plates were allowed to incubate at room temperature for 10 minutes to stabilize the luminescent signal. The plates were analyzed with a microplate reader.

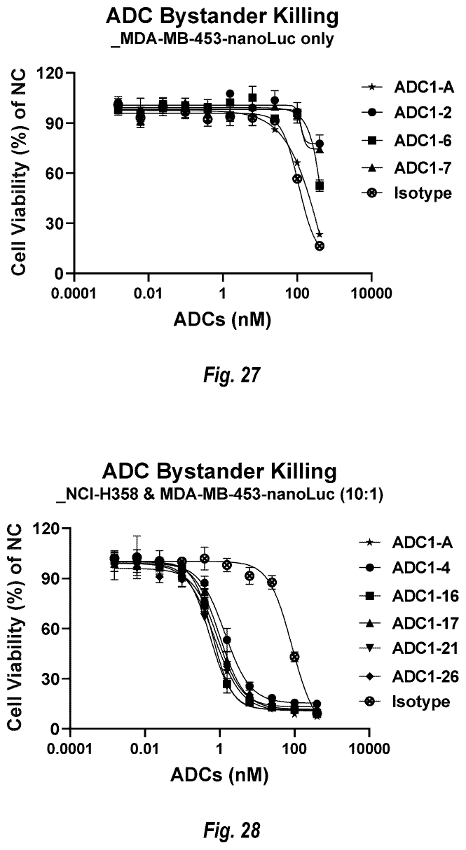

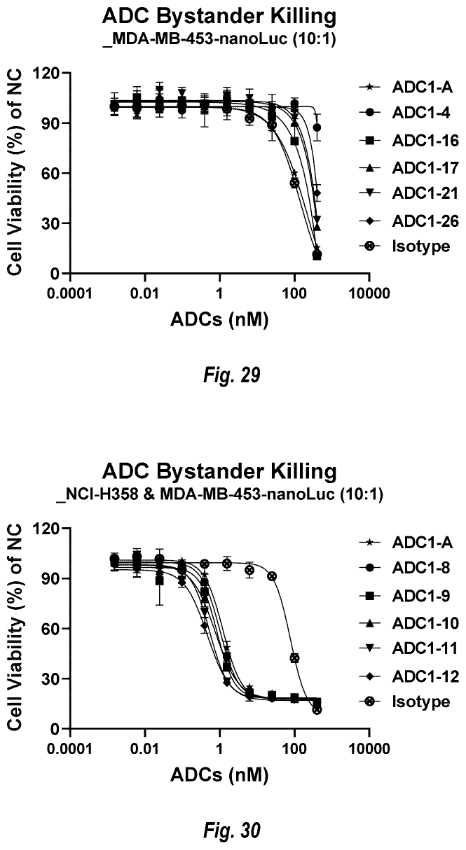

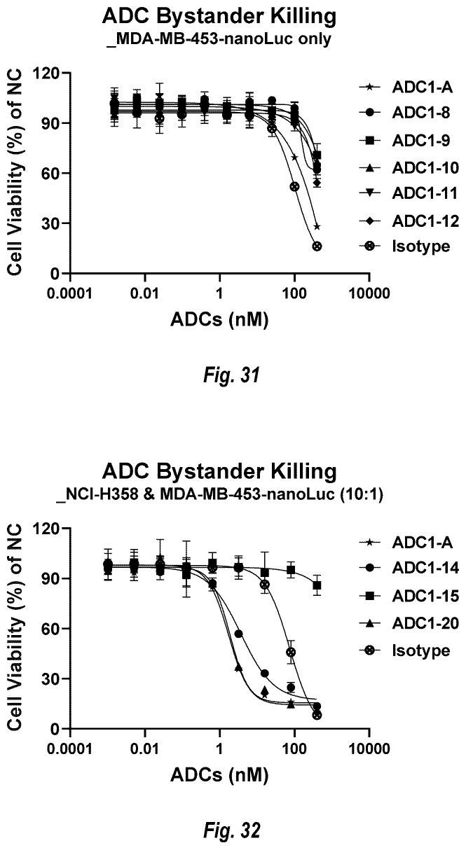

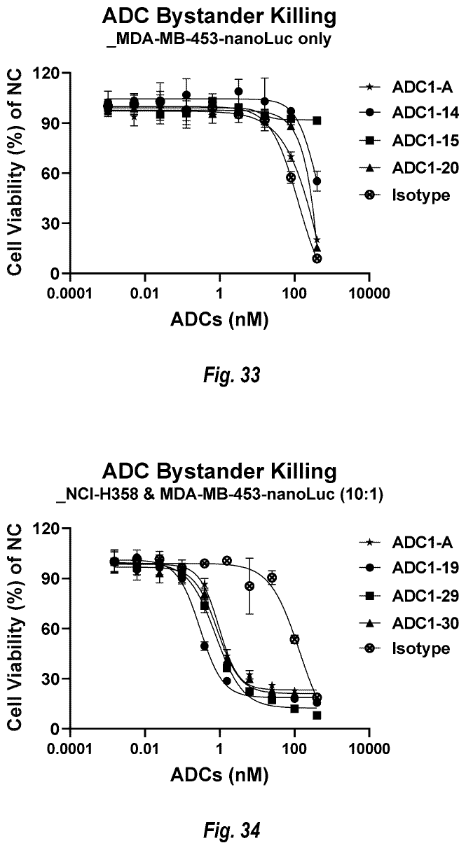

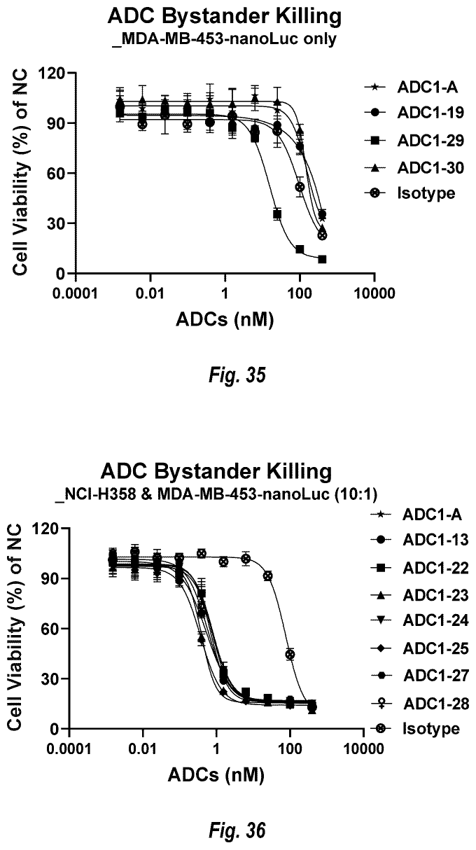

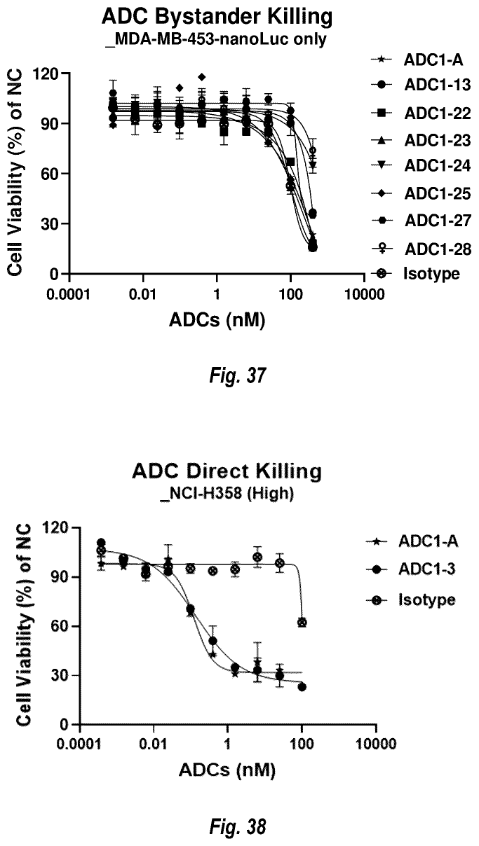

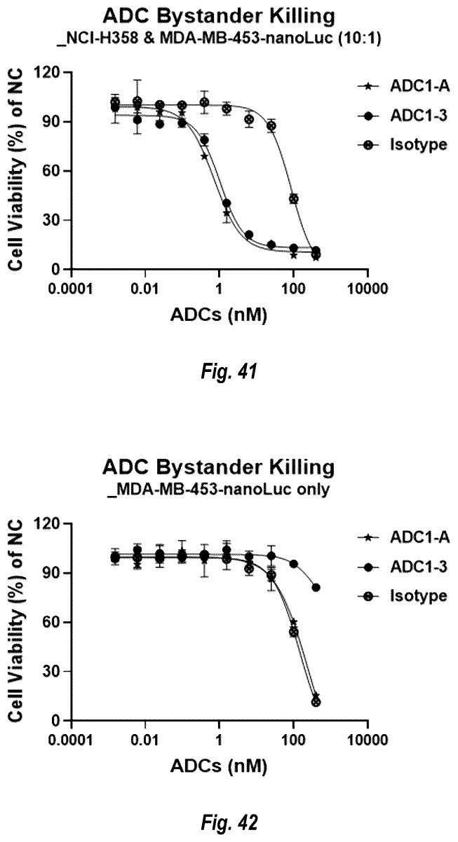

Data are summarized in Tables 6A-6G, and FIGS. 26-37, 41, and 42.

TABLE 6A

NCI-H358:MDA-MB-453-nanoLuc = 10:1, 3D

| ADC | Emax (%) | EC50 (nM) |
|---|---|---|
| ADC1-A | 88.1 | 0.8 |
| ADC1-2 | 83.7 | 1.0 |
| ADC1-6 | 84.9 | 0.7 |
| ADC1-7 | 83.8 | 0.6 |

TABLE 6B

NCI-H358:MDA-MB-453-nanoLuc = 10:1, 3D

| ADC | Emax (%) | EC50 (nM) |
|---|---|---|
| ADC1-A | 92.7 | 0.8 |
| ADC1-4 | 85.0 | 1.4 |
| ADC1-16 | 91.1 | 0.7 |
| ADC1-17 | 90.2 | 1.0 |
| ADC1-21 | 90.6 | 0.6 |
| ADC1-26 | 88.5 | 0.8 |

TABLE 6C

NCI-H358:MDA-MB-453-nanoLuc = 10:1, 3D

| ADC | Emax (%) | EC50 (nM) |
|---|---|---|
| ADC1-A | 87.9 | 1.3 |
| ADC1-8 | 84.3 | 1.0 |
| ADC1-9 | 84.0 | 0.8 |
| ADC1-10 | 84.2 | 0.8 |
| ADC1-11 | 83.8 | 0.5 |
| ADC1-12 | 84.4 | 0.4 |

TABLE 6D

NCI-H358:MDA-MB-453-nanoLuc = 10:1, 3D

| ADC | Emax (%) | EC50 (nM) |
|---|---|---|
| ADC1-A | 89.9 | 1.7 |
| ADC1-14 | 86.5 | 3.7 |
| ADC1-15 | 14.0 | >400 |
| ADC1-20 | 88.6 | 1.9 |

TABLE 6E

NCI-H358:MDA-MB-453-nanoLuc = 10:1, 3D

| ADC | Emax (%) | EC50 (nM) |
|---|---|---|
| ADC1-A | 83.2 | 1.0 |
| ADC1-19 | 84.3 | 0.3 |
| ADC1-29 | 92.0 | 0.8 |
| ADC1-30 | 83.1 | 0.9 |

TABLE 6F

NCI-H358:MDA-MB-453-nanoLuc = 10:1, 3D

| ADC | Emax (%) | EC50 (nM) |
|---|---|---|
| ADC1-A | 89.2 | 0.7 |
| ADC1-13 | 86.9 | 0.6 |
| ADC1-22 | 86.2 | 0.8 |
| ADC1-23 | 88.7 | 0.4 |
| ADC1-24 | 85.0 | 0.8 |
| ADC1-25 | 88.4 | 0.4 |
| ADC1-27 | 85.6 | 0.8 |
| ADC1-28 | 86.3 | 0.6 |

TABLE 6G

NCI-H358:MDA-MB-453-nanoLuc = 10:1, 3D

| ADC | Emax (%) | EC50 (nM) |
|---|---|---|
| ADC1-A | 92.7 | 0.8 |
| ADC1-3 | 88.3 | 0.9 |

### ADOs In Vivo Efficacy Study

Female BALB/c Nude mice are subcutaneously implanted with 3×10⁶H1650/capan-1/H441/H11048/HCC827/H1975 cells per 200 μL PBS/matrigel in the right flank. After inoculation, tumor volumes are determined twice weekly in two dimensions using a caliper, and are expressed in mm³ using the formula: V=0.5(a×b²) where a and b are the long and short diameters of the tumor, respectively. When tumors reach a mean volume of approximately 200 mm³ in size, mice are randomly allocated into three groups with eight animals in each group, and are intravenously treated with vehicle or ADC at a dose of 1, 3, or 10 mpk on day 1. Partial regression (PR) is defined as tumor volume smaller than 50% of the starting tumor volume on the first day of dosing in three consecutive measurements and complete regression (CR) is defined as tumor volume less than 14 mm³ in three consecutive measurements. Data are presented as mean tumor volume±standard error of the mean (SEM). Tumor growth inhibition (TGI) is calculated using the following formula:

$$\% growth inhibition = 100 \times ( 1 - ( \frac{( treated t ) - treated to )}{( placebo t ) - placebo to )} ) )$$

treated t=treated tumor volume at time t
treated t₀=treated tumor volume at time 0
placebo t=placebo tumor volume at time t
placebo t₀=placebo tumor volume at time 0

### ADC Plasma Stability

Incubation of ADC with plasma:
1. ADC is diluted into mouse or human plasma to yield a final solution of 100 μg/mL ADC in plasma
2. The samples are incubated at 37 °C
3. Aliquots (100 μl) are taken at four time points (0, 4, 24, 72, 96, or 168 h)
4. Samples are frozen at −80 °C until analysis.

Plasma payload concentrations are carried out under the following measurement conditions:
Instrument: LC-MS/MS (Triple Quad 5500)
Monitor: MRM
Column: Advanced Materials Technology, HALO AQ-C18 2.7 μm 90A, 50*2.1 mm
Column temperature: 40 °C
Mobile phase A: H₂O-0.1% FA
Mobile phase B: ACN-0.1% FA
Gradient program for MMAE: 20% B-20% B (0 min-0.2 min), 20% B-80% B (0.2 min-1.5 min), 80% B-80% B (1.5 min m 2.2 min), 80% B-20% B (2.20 min-2.21 min), 20% B-20% B (2.21 min-3.0 min); Gradient program for Dxd: 2% B-2% B (0 min-0.2 min), 2% B-98% B (0.2 min-1.2 min), 98% B-98% B (1.2 min-2.0 min), 98% B-2% B (2.0 min-2.01 min), 2% B-2% B (2.01 min-4.0 min)t
Injected sample amount: 10 L(DXd or DXd analogues)

Plasma ADC and total Ab (Tab) concentrations are carried out under the following measurement conditions:
Assay: Ligand binding assay (ELISA)
Capture reagent: B7H3 ECD
Detection reagent: anti-payload Ab for ADC and anti-human IgG polyclonal Ab for Total Ab.

In mice PK study, blood samples are collected at 0, 2, 4, 8, 24, 72, and 168 h after one dose intravenous administration of ADCs from H1650/capan-1/H441/H1048/HCC827/H1975 tumor bearing mice or non-tumor bearing mice, followed by centrifugation (4 °C, 3000×g, 7 min) to separate plasma. The concentrations of ADCs and total Abs are measured by in-house developed Meso Scale Discovery (MSD) ligand binding methods. Briefly, His tagged B7H3 extracellular domain fusion protein is used as a capture reagent, biotin-labelled anti-payload Ab, or goat anti-human kappa Ab are used as the detection reagents for ADCs or total Ab measurement, respectively.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

1. A compound of Formula (I): 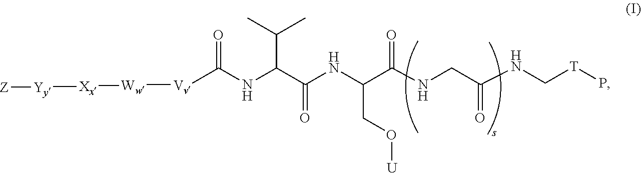
or a pharmaceutically acceptable salt, tautomer, solvate, or stereoisomer thereof wherein:
Z is 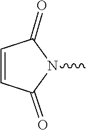
Y is an arylene or —C═C—(CH₂)ₐ—;
subscript a is 1, 2, 3, or 4;
subscript y′ is 0;
X is an unsubstituted C₁₋₅ alkyl;
subscript x′ is 1;
W is oxygen;
subscript w′ is 0;
V is a C₁₋₄ alkyl or —C(═O)—NH—(CH₂)_{b} wherein the N is optionally substituted with a C₁₋₅ alkyl and subscript b is 1 or 2;
subscript v′ is 0;
U is 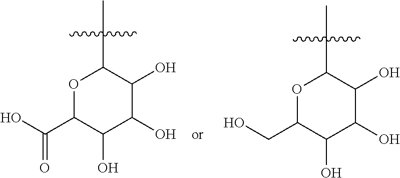 [SMILES: *C1OC(C(=O)O)C(O)C(O)C1O ; *C1OC(CO)C(O)C(O)C1O]
T is oxygen or a bond;
P is 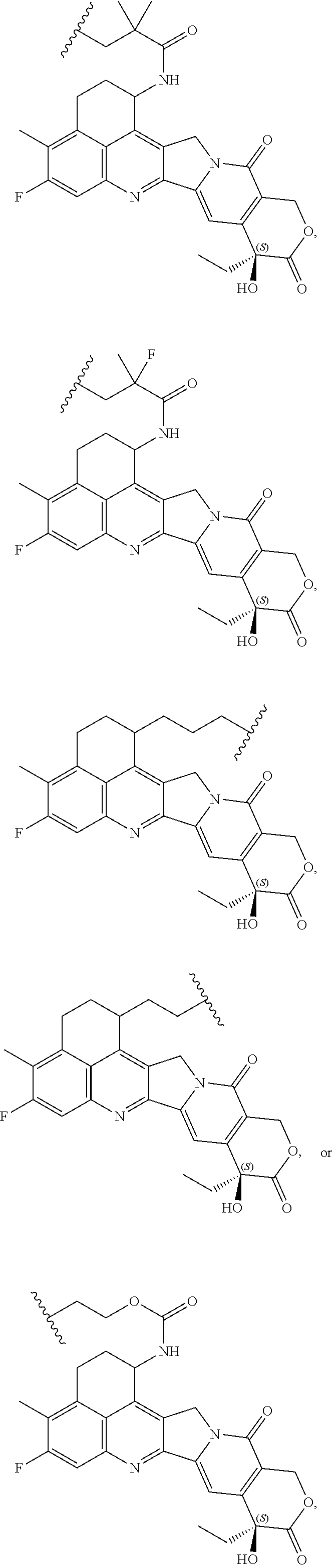
and
subscript s is 0 or 1.

2. (canceled)

3. (canceled)

4. The compound of claim 1, wherein X is —(CH₂)₅—.

5. (canceled)

6. (canceled)

7. (canceled)

8. (canceled)

9. (canceled)

10. The compound of claim 1, wherein U is

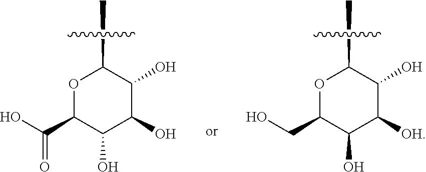

11. The compound of claim 1, wherein T is oxygen.

12. (canceled)

13. The compound of claim 1, selected from any one of the following, or a pharmaceutically acceptable salt, tautomer, solvate, stereoisomer, enantiomer, or isotopologue thereof:

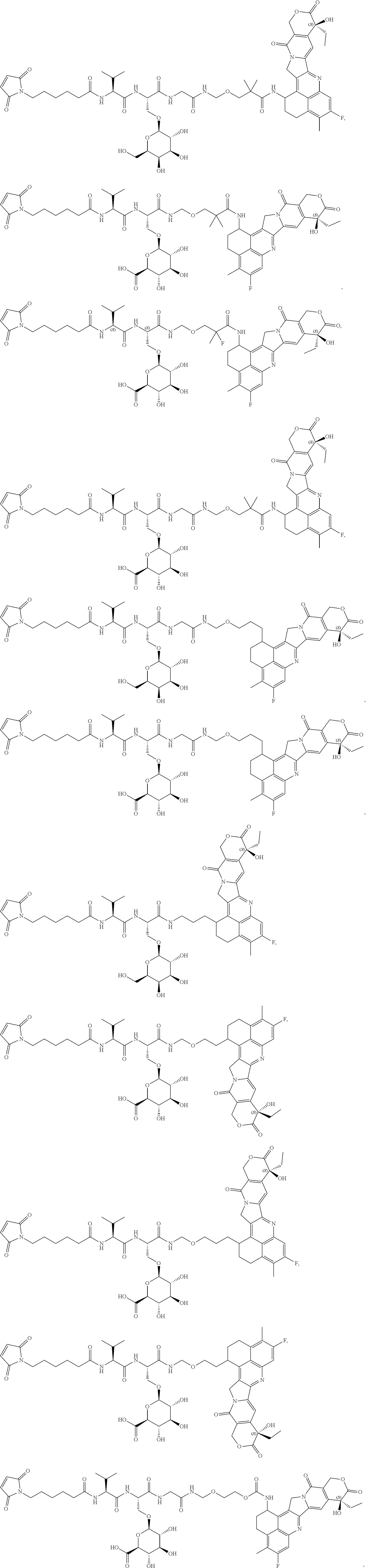

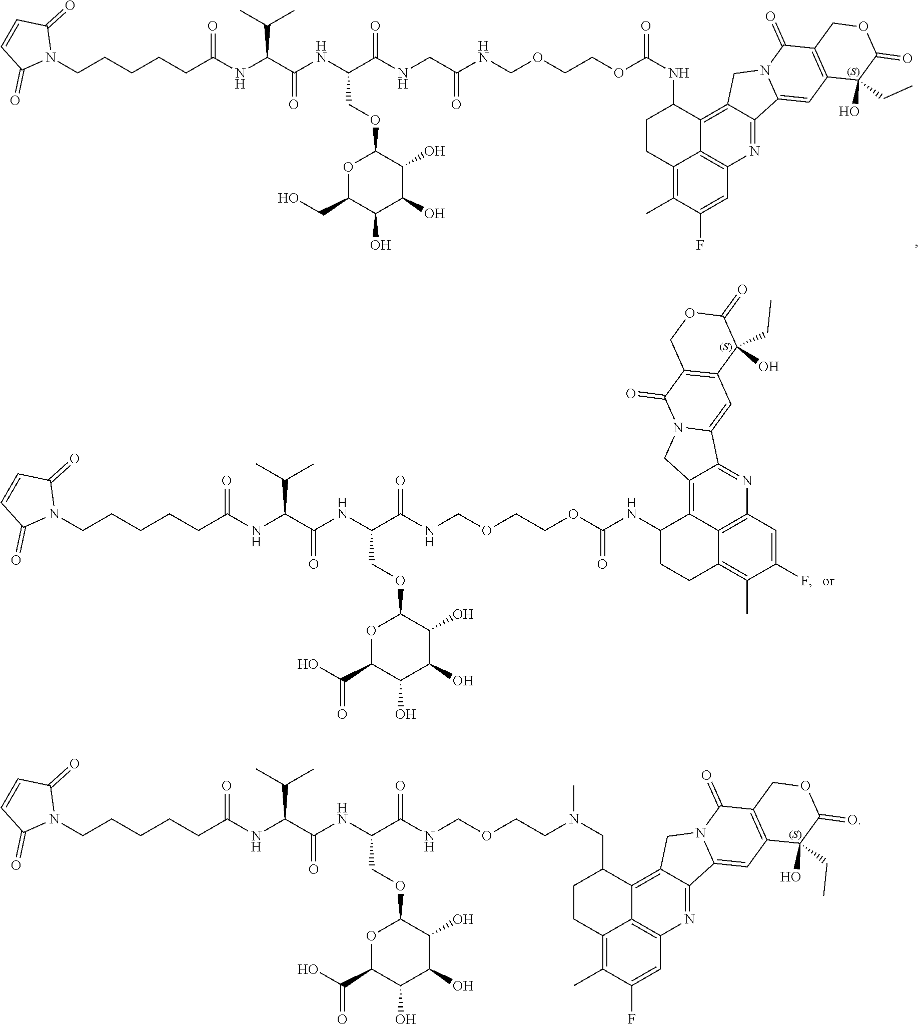

14. An antibody drug conjugate of Formula (II): 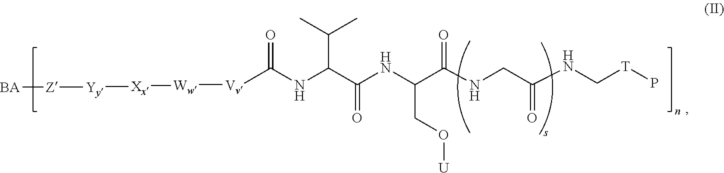
or a pharmaceutically acceptable salt, tautomer, solvate, or stereoisomer thereof, wherein
BA is a binding agent selected from a humanized chimeric, or a human antibody or an antigen binding fragment thereof;
Z′ is 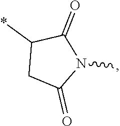
and * indicates the bond where Z′ attaches to BA;
Y is an arylene or —C≡C—(CH₂)ₐ—;
subscript a is 1, 2, 3, or 4;
subscript y′ is 0;
X is an unsubstituted C₁₋₅ alkyl;
subscript x′ is 1;
W is oxygen;
subscript w′ is 0;
V is a C₁₋₄ alkyl or —C(═O)—NH—(CH₂)_{b} wherein the N is optionally substituted with a C₁₋₅ alkyl and subscript b is 1 or 2;
subscript v′ is 0;
U is 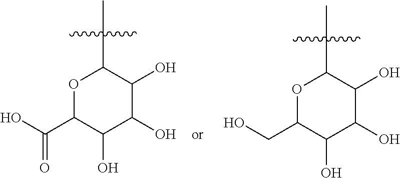 [SMILES: *C1OC(C(=O)O)C(O)C(O)C1O ; *C1OC(CO)C(O)C(O)C1O]
T is oxygen or a bond;
P is 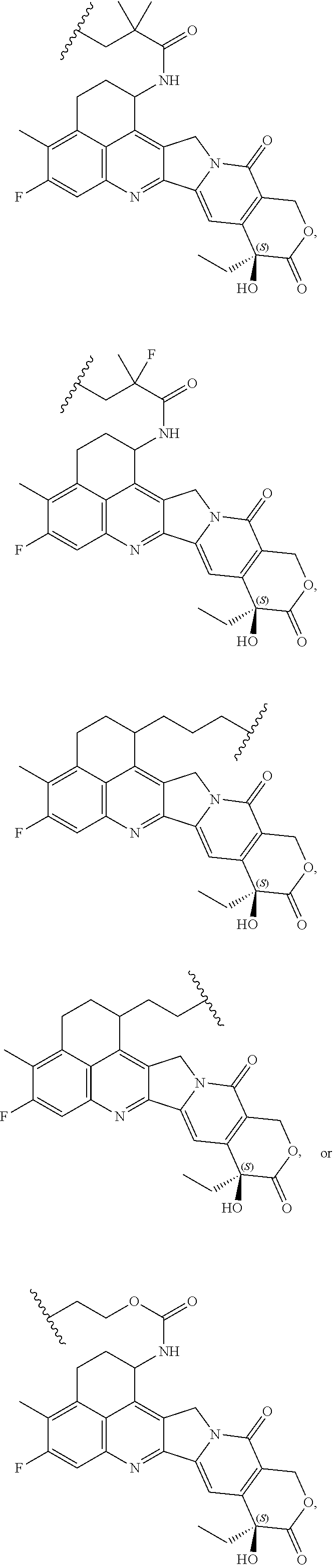
subscript s is 0 or 1; and
subscript n is from 1 to 15.

15. (canceled)

16. (canceled)

17. The antibody drug conjugate of claim 14, wherein X is —(CH₂)₅.

18. (canceled)

19. (canceled)

20. (canceled)

21. (canceled)

22. (canceled)

23. The antibody drug conjugate of claim 14, wherein U is

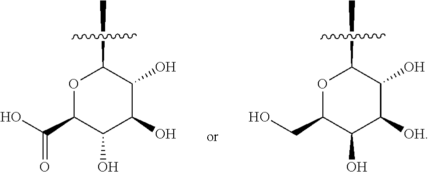

24. The antibody drug conjugate of claim 14, wherein T is oxygen.

25. (canceled)

26. (canceled)

27. The antibody drug conjugate of claim 14, selected from an one of the following, or a pharmaceutically acceptable salt, solvate, or hydrate thereof: 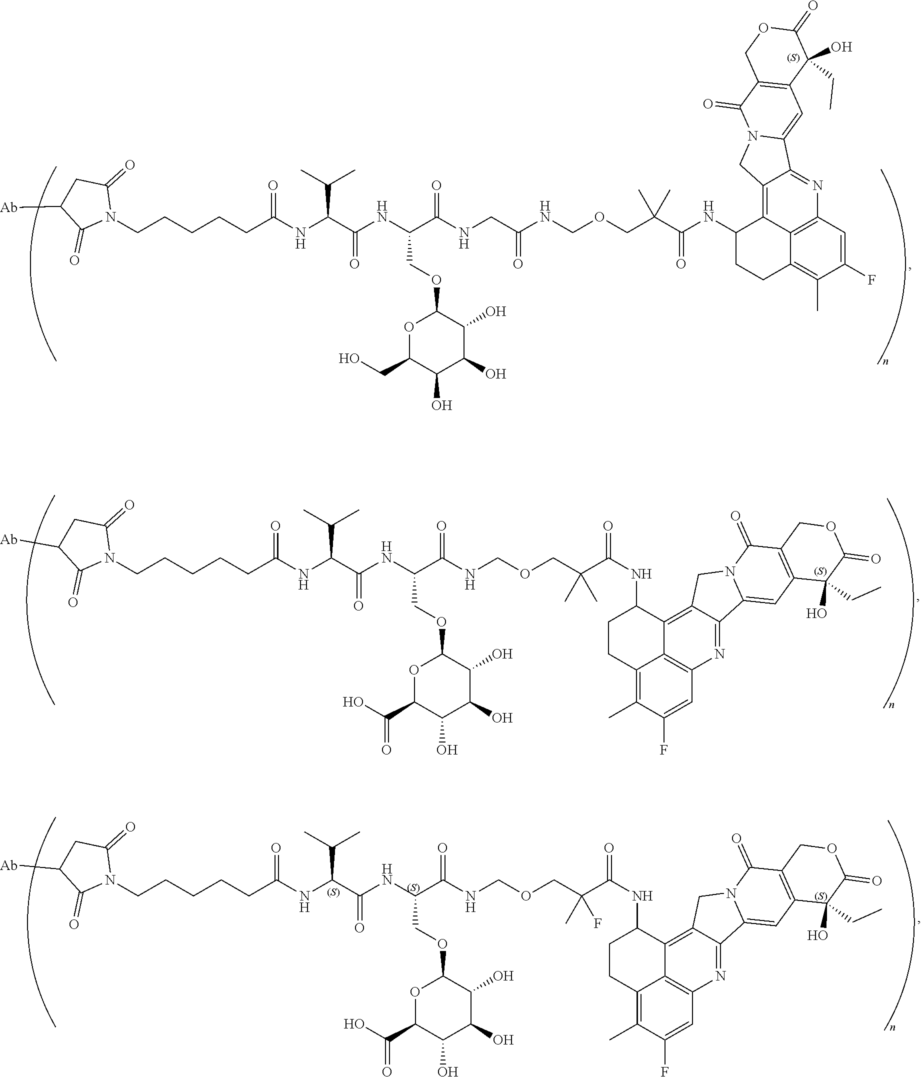 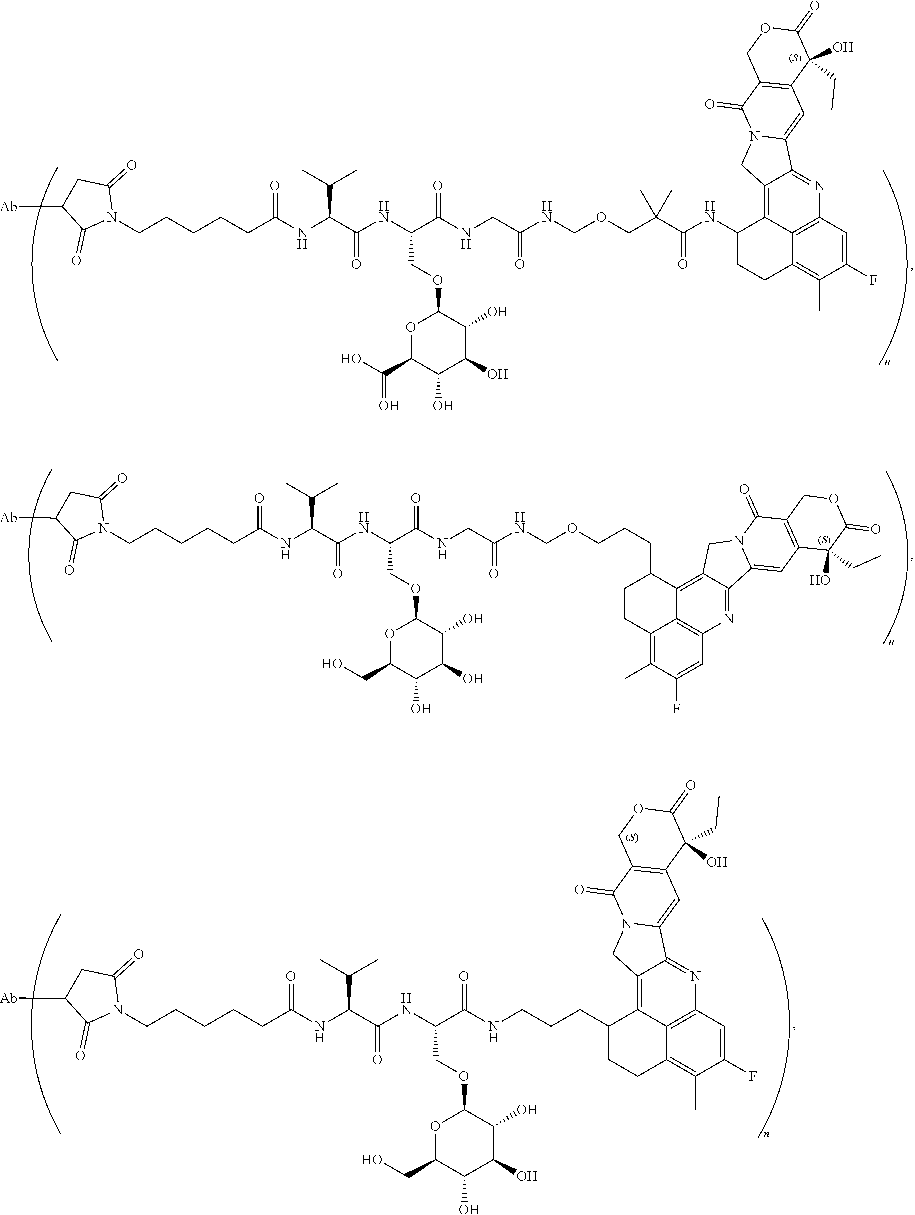 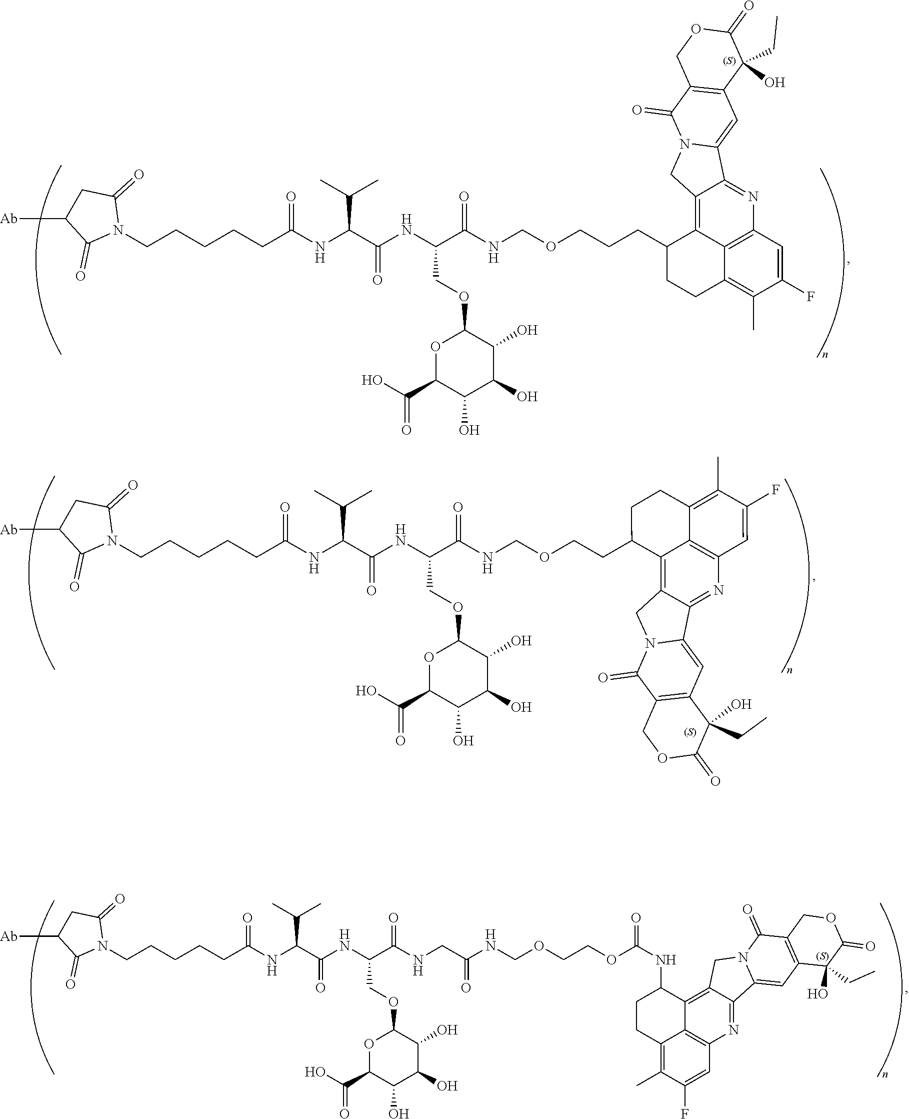 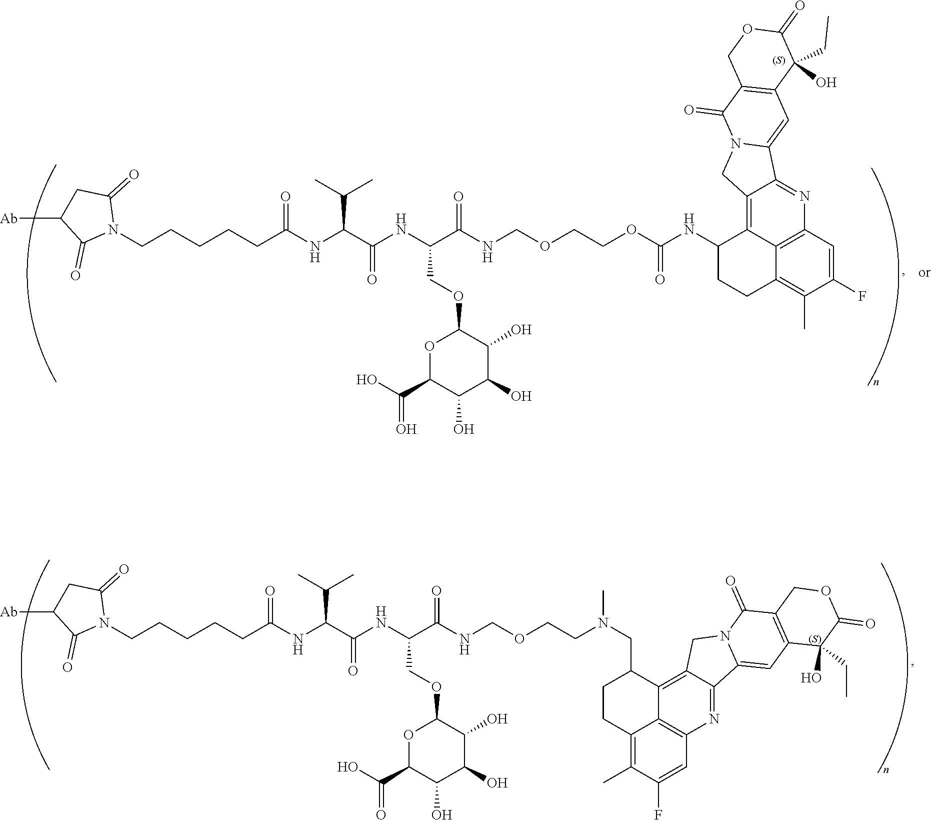
wherein Ab is BA.

28. (canceled)

29. A pharmaceutical composition comprising the antibody drug conjugate of claim 14, or a pharmaceutically acceptable salt, tautomer, solvate, or stereoisomer thereof, and a pharmaceutically acceptable excipient.

30. A compound of Formula (I): 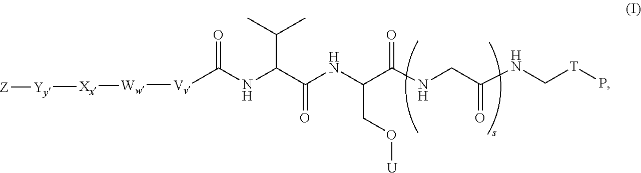
or a pharmaceutically acceptable salt, tautomer, solvate, or stereoisomer thereof, wherein:
Z is 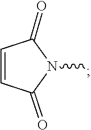
Y is an arylene or —C═C—(CH₂)ₐ—;
subscript a is 1, 2, 3, or 4;
subscript y′ is 0;
X is a substituted or unsubstituted C₁₋₅ alkyl;
subscript x′ is 0 or 1;
W is oxygen;
subscript w′ is 0 or 1;
V is —C(═O)—NH—(CH₂)_{b} and subscript b is 2;
subscript V is 0 or 1;
U is 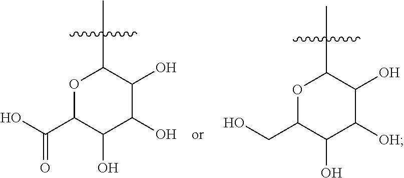 [SMILES: *C1OC(C(=O)O)C(O)C(O)C1O ; *C1OC(CO)C(O)C(O)C1O]
T is oxygen or a bond;
P is 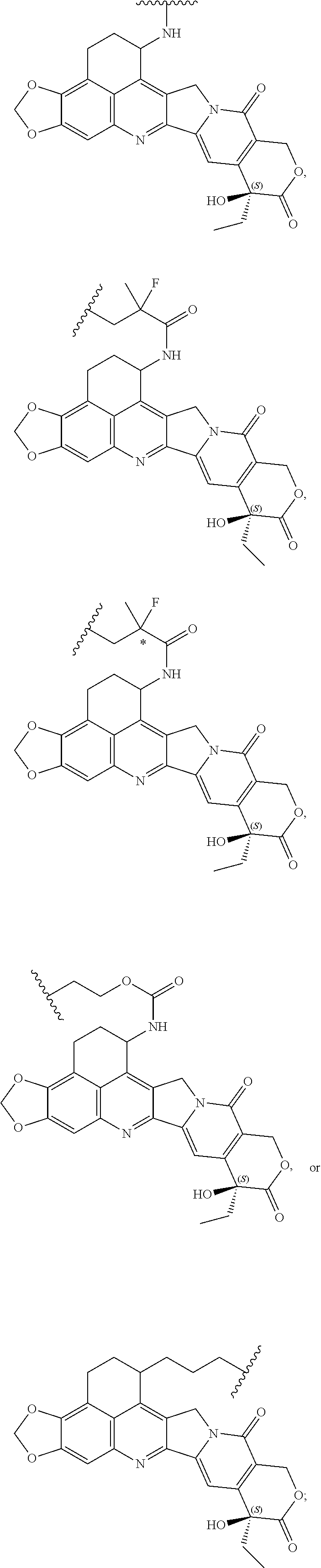
and
subscript s is 0 or 1.

31. The compound of claim 30, wherein X is —(CH₂)₅— or —CH(CH₂NH₂)(CH₂)₂—.

32. The compound of claim 30, wherein U is

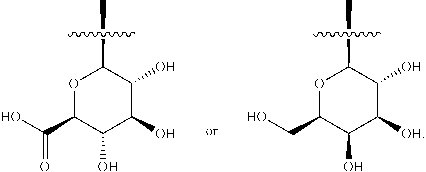

33. The compound of claim 30, wherein T is oxygen.

34. The compound of claim 30, selected from any one of the following, or a pharmaceutically acceptable salt, tautomer, solvate, stereoisomer, enantiomer, or isotopologue thereof:

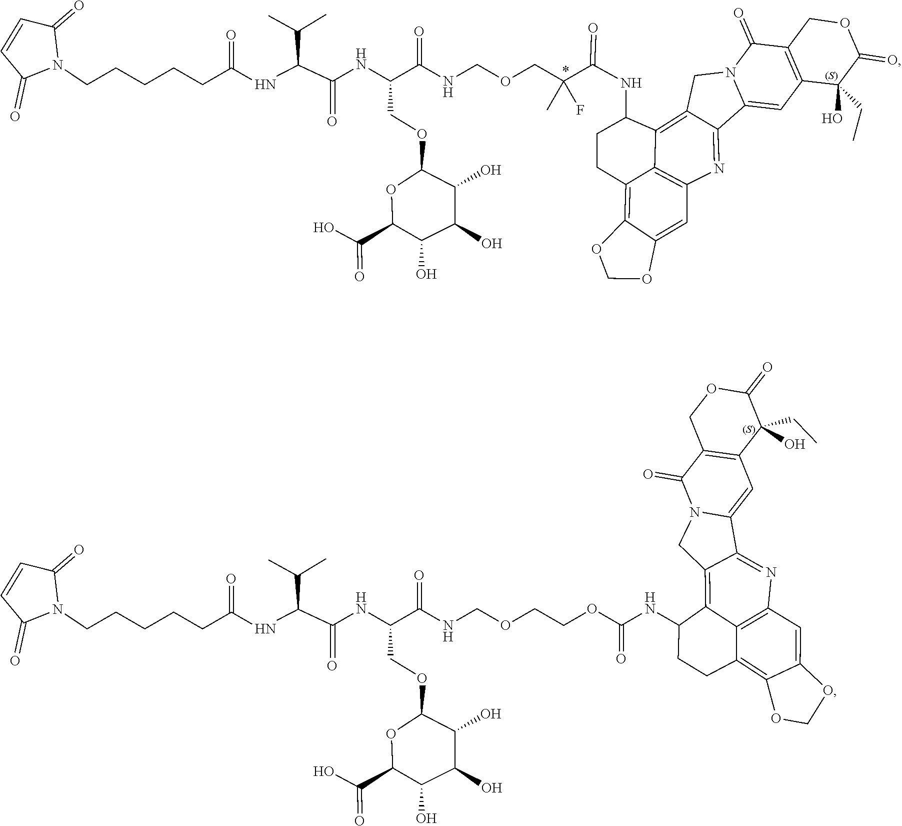

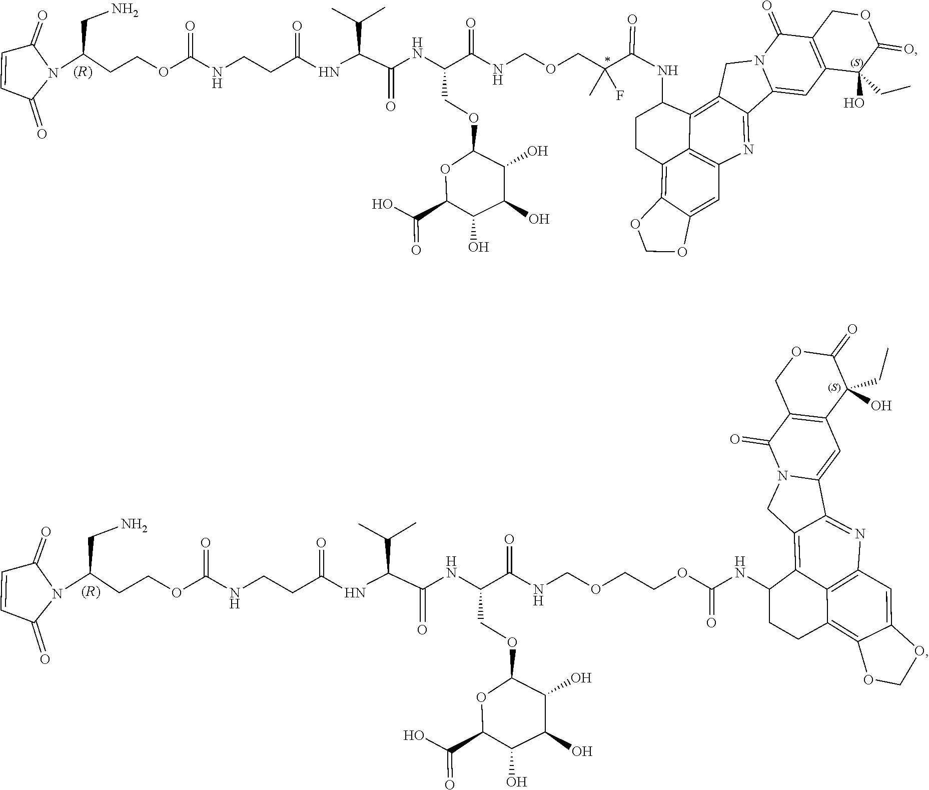

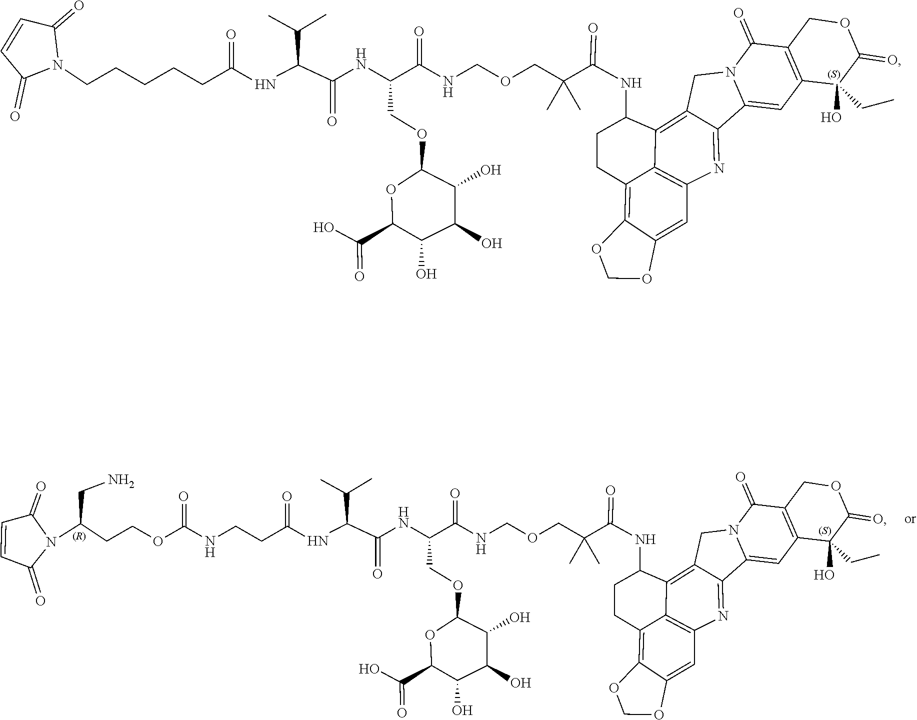

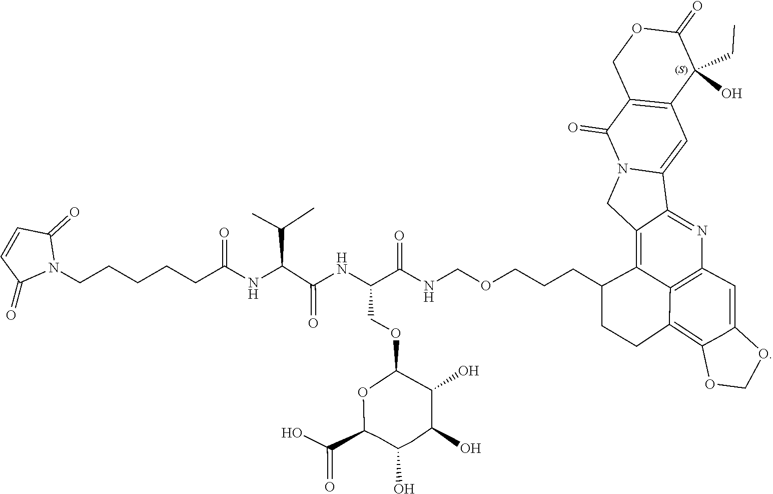

35. An antibody drug conjugate of Formula (II): 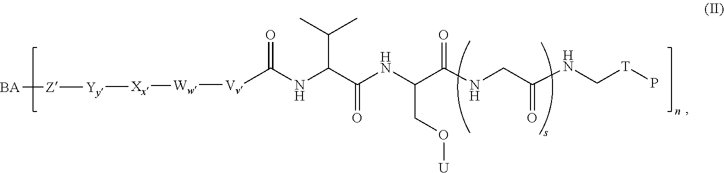
or a pharmaceutically acceptable salt, tautomer, solvate, or stereoisomer thereof, wherein:
BA is a binding agent selected from a humanized, chimeric, or human antibody or an antigen binding fragment thereof;
Z′ is 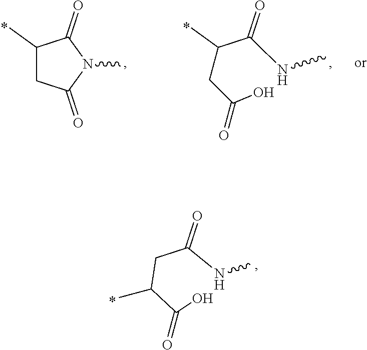
and * indicates the bond where Z′ attaches to BA;
Y is an arylene or —C═C—(CH₂)ₐ—
subscript a is 1, 2, 3, or 4;
subscript y′ is 0;
X is a substituted or unsubstituted C₁₋₅ alkyl;
subscript x′ is 0 or 1;
W is oxygen;
subscript w′ is 0 or 1;
V is —C(═O)—NH—(CH₂)_{b} and subscript b is 2;
subscript V is 0 or 1;
U is 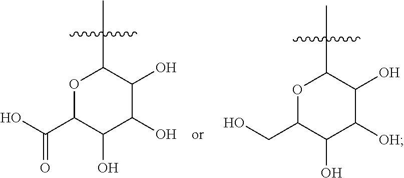 [SMILES: *C1OC(C(=O)O)C(O)C(O)C1O ; *C1OC(CO)C(O)C(O)C1O]
T is oxygen or a bond;
P is 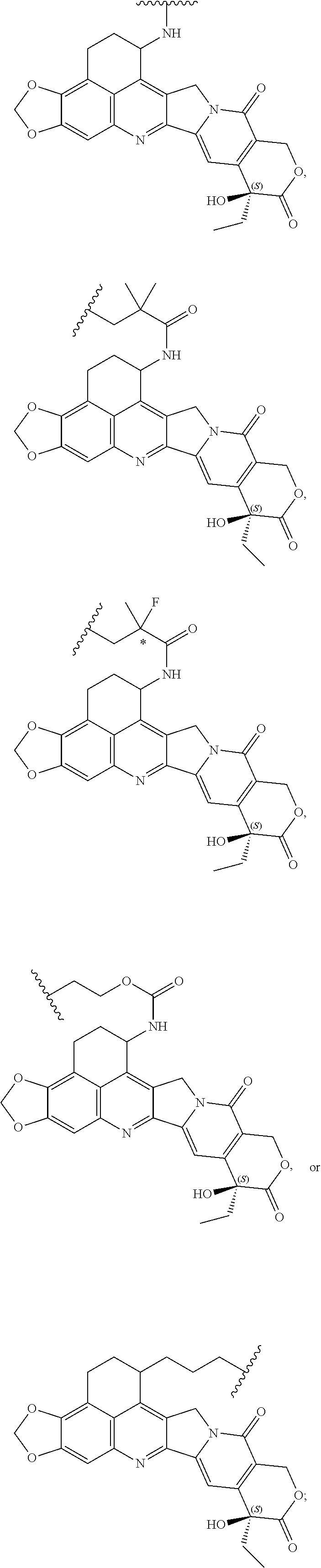
subscript s is 0 or 1; and
subscript n is from 1 to 15.

36. The antibody drug conjugate of claim 35, wherein X is —(CH₂)₅— or —CH(CH₂NH₂)(CH₂)₂—.

37. The antibody drug conjugate of claim 35, wherein U is

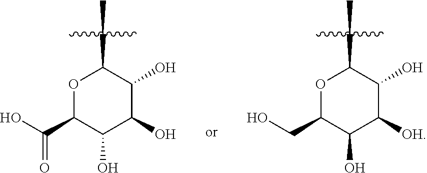 [SMILES: *[C@@H]1O[C@H](C(=O)O)[C@@H](O)[C@H](O)[C@H]1O ; "or" ; *[C@@H]1O[C@H](CO)[C@H](O)[C@H](O)[C@H]1O]

38. The antibody drug conjugate of claim 35, wherein T is oxygen.

39. The antibody drug conjugate of claim 35, selected from any one of the following, or a pharmaceutically acceptable salt, solvate, or hydrate thereof:

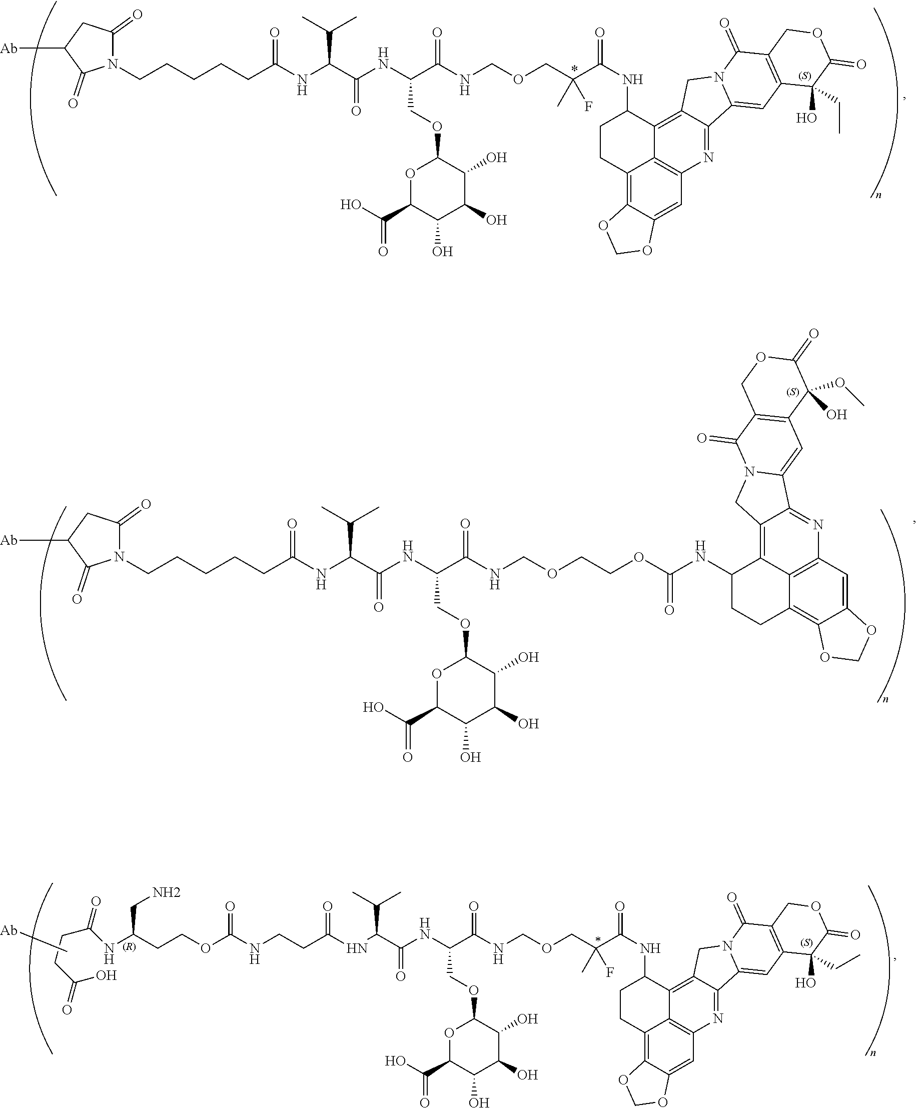

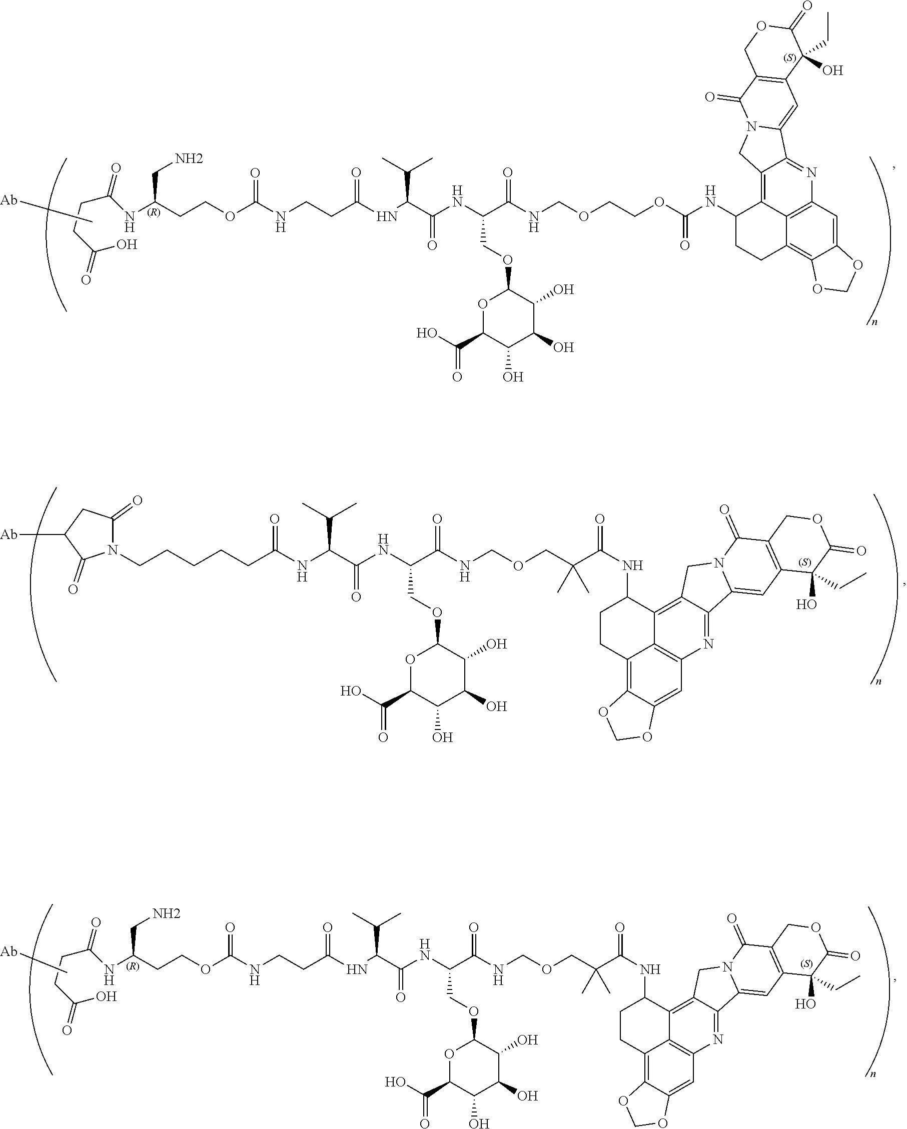

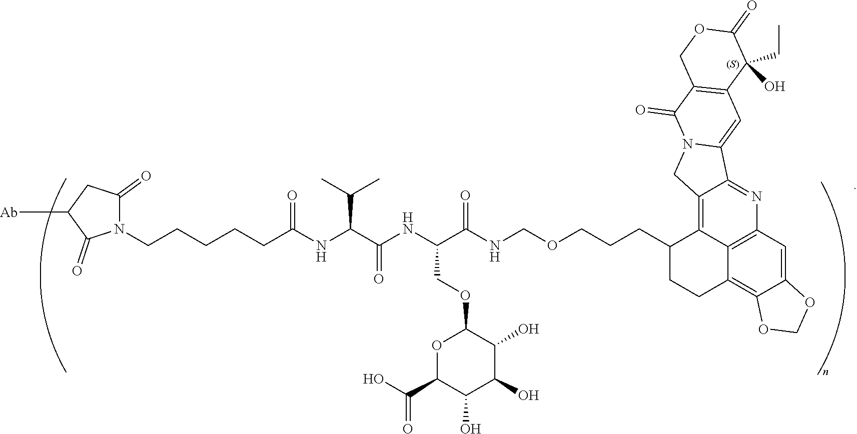

40. A pharmaceutical composition comprising the antibody drug conjugate of claim 35, or a pharmaceutically acceptable salt, tautomer, solvate, or stereoisomer thereof, and a pharmaceutically acceptable excipient.
